(19) United States
(12) Patent Application Publication
(10) Pub. No.: US 2022/0001014 A1
(43) Pub. Date: Jan. 6, 2022

(54) COMPOSITIONS AND METHODS FOR INDUCING BIOLOGICAL MIMICRY IN A MAMMAL FOR THE PREVENTION AND/OR TREATMENT OF COVID-19 AND OTHER DISEASES

(71) Applicant: Lida Ghaderi, Santa Monica, CA (US)

(72) Inventor: Lida Ghaderi, Santa Monica, CA (US)

(21) Appl. No.: 17/366,007

(22) Filed: Jul. 1, 2021

Related U.S. Application Data
(60) Provisional application No. 63/046,702, filed on Jul. 1, 2020.

(51) Int. Cl. A61K 45/06; C12Q 1/70

(52) U.S. Cl. CPC A61K 45/06; C12Q 1/70

(57) ABSTRACT

The present invention is directed to compositions and methods for inducing artificial biological mimicry in a mammal to mimic the favorable immune advantages present in a bat in another mammal, particularly a human subject, and thereby prevent and/or treat COVID-19 and other diseases by administering to the subject a combination of active agents comprising two or more active agents comprising an inhibitor of inflammasome NLRP3, an inhibitor of one or more priming or activation signals of inflammasome NLRP3, and/or an activator of the inhibitor of one or more priming or activation signals of inflammasome NLRP3 alone or in combination with one or more modulators of Kallikrein-Kinin System (KKS) dysregulation, one or more modulators of Renin Aldosterone Angiotensin System (RAAS) dysregulation, one or more modulators of protease dysregulation, and/or one or more modulators of cytokine dysregulation. Pharmaceutical compositions, methods of treatment, methods of genetic testing, and packaged kits are provided.

### CROSS-REFERENCE TO RELATED APPLICATIONS

The present invention is related to U.S. Provisional Patent App. No. 63/046,702, entitled “Compositions, Methods, Uses and Gene Polymorphism Testing to Induce Biological Mimicry in a Mammal by Modulating PRRS, Cytokines, RAAS-KKS, Proteases for the Prevention and/or Personalized Treatment of COVID19 and Other Diseases,” filed on Jul. 1, 2020; the entire disclosure of which is incorporated herein by reference.

### TECHNICAL FIELD

The subject matter of the present invention relates generally to compositions and methods in the field of medicine for inducing artificial biological mimicry in a mammal to mimic the favorable physiological advantages (e,g., immune, genetic, cellular) advantages present in a bat in another mammal, particularly a human subject, and thereby prevent and/or treat COVID-19 and other diseases.

### BACKGROUND

At the end of 2019, a cluster of pneumonia patients with an unidentified cause emerged in Wuhan, Hubei Province, China. The infectious organism was found to be a novel Severe Acute Respiratory Syndrome Coronavirus 2 (SARS-CoV-2), which causes Coronavirus Disease 2019 (COVID-19; Astuti and Ysrafil (2020) Diabetes Metab. Syndr., 14:407-412). On Jan. 30, 2020, SARS-CoV-2 infections caused the World Health Organization (WHO) to declare a Public Health Emergency of International Concern, and on Mar. 11, 2020, WHO declared COVID-19 a global pandemic (Cucinotta and Vanelli (2020) Acta Bio-Medica Atenei Parmensis, 91:157-60). As of May 6, 2020, there were 3,755,341 confirmed cases of patients who had tested positive for SARS-CoV-2 globally, with 263,831 world-wide deaths. Due to limited testing and lack of mass testing globally, the prevalence of SARS-CoV-2 and mortality rates of the infections are unreliable and underestimated.

The clinical presentation of SARS-CoV-2 infections and COVID-19 disease is heterogenous, with clinical manifestations and phenotypes ranging from asymptomatic to mild symptoms of cough, fever, fatigue, myalgias, diarrhea, anosmia to acute respiratory distress syndrome, acute lung injury, renal, hepatic, gastrointestinal disease, cardiovascular, neurologic, gastrointestinal, thromboembolic disease including arterial, venous and small vessel thromboembolism, and other clinical disease presentations.

Infected individuals can also be asymptomatic yet can transmit the disease to others. A study published in JAMA described 131 SARS-COV-2 tests performed on patients who presented to the Los Angeles County University of Southern California Emergency and Urgent care departments over 4 days (Spellberg et al. (2020) JAMA, 323:1966-1967). The patients presented with mild influenza-like illness and had no travel history, ill contact exposure, or symptoms of severe illness (Spellberg et al. (2020) JAMA, 323:1966-1967). However, even without risk factors, 5.3% of these patients tested positive for SARS-CoV-2 (Spellberg et al. (2020) JAMA, 323:1966-1967). This was particularly concerning since these patients were active in the community and could increase community transmission.

New coronavirus populations are generally susceptible, but elderly people with underlying chronic diseases are more susceptible. The underlying diseases are diabetes, hypertension, cardiovascular disease, cerebro-vascular disease, obesity, autoimmune disease, cancers, and others (See i.e., Li et al. (2020) Microbes Infect., 22:80-85). The mortality of elderly patients with COVID-19 is higher than that of young and middle-aged patients, and the proportion of patients with PSI (Community-Acquired Pneumonia Severity Index), PSI grade IV and V is significantly higher than that of young and middle-aged patients. Elderly patients with COVID-19 are more likely to progress to severe disease. The elderly are more susceptible to severe illness and are admitted to intensive care units (ICUs), and the mortality of elderly patients is higher (Liu et al. (2020) J. Infect., 80:e14-e18).

Several viruses of the coronavirus family circulate in the human population. Four of these coronaviruses cause mild illness (NL-63, 229E, HKU1, and OC43), while three others lead to serious disease (SARS-CoV, MERS-CoV, and SARS-CoV-2; Zhu et al. (2020) New Engl. J. Med., 382:727-733). An outbreak of SARS-CoV occurred in 2002 and an outbreak of MERS-CoV occurred in 2012, making SARS-CoV-2 the third coronavirus to emerge in the human population. SARS-CoV-2 has threatened the entire world as a global pandemic and has caused severe health and socioeconomic devastation.

The coronavirus family is a class of enveloped, positive-sense, single-stranded RNA viruses. SARS-CoV-2 is genetically similar to other coronaviruses in the Sarbecovirus subgenus, Orthocoronaviridae subfamily, which is a clade of betacoronaviruses formed by the coronavirus that causes SARS (SARS-CoV) and other SARS-CoV-like coronaviruses found in bats. Recombination between coronaviruses are common, and SARS-CoV is believed to be a recombinant between bat sarbecorviruses. Interestingly, the whole genome of SARS-CoV-2 is highly similar to that of a bat coronavirus detected in 2013 (>96% sequence identity), which suggests that the immediate ancestor of SARS-CoV-2 has been circulating in bats for at least several years. This may indicate that the COVID-19 pandemic was caused by a single zoonotic introduction and that the virus is relatively stable, genetically, in humans. SARS-CoV and SARS CoV-2's spike protein uses the human receptor ACE-2 to bind to human cells. The spike of SARS-CoV-2 can also theoretically interact with ACE-2 from other animal species. SARS-CoV-2, is experimentally capable of using human, swine, bat, and civet ACE-2 for entry, but not mouse ACE-2 (Poon and Peiris (2020) Nat. Med., 26:317-319).

Few antiviral drugs or therapies have been proven effective for the prevention or treatment of individuals infected with SARS-Cov-2 or those individuals who have COVID-19 disease. Additionally, there are no approved antiviral drugs or therapies for patients infected with SARS-CoV or MERS-CoV. Patients who develop severe hypoxemia and acute lung injury, acute respiratory distress are managed with high flow oxygen or ventilators. During the SARS-CoV-2 global pandemic, the public health strategy is premised on the assumption to limit community transmission by limiting contact and quarantining strategies while the prevalence and death toll of SARS-COV-2 infections are rising daily.

On May 1, 2020, the U.S. Food and Drug Administration (FDA) issued an emergency use authorization for the investigational antiviral drug, Remdesivir, for the treatment of suspected or laboratory-confirmed COVID-19 disease in adults and children hospitalized with severe disease. Remdesivir (GS-5734) is a nucleoside analogue prodrug, has inhibitory effects on pathogenic animal and human coronaviruses including SARS-CoV-2 in vitro, and inhibits MERS-CoV, SARS-CoV-1, and SARS-CoV-2 replication in animal models. However, in a human randomized control study of 237 patients, Remdesivir was not associated with statistically significant clinical benefits (Wang et al. (2020) Lancet, 395:P1569-1578).

Due to the COVID-19 global pandemic, its severe world-wide health and socioeconomic impacts, and the high viral transmission rate of SARS-Cov-2, it is imperative to expeditiously develop preventive and therapeutic treatments for SARS-Cov-2 viral infections and for these therapies to be safe, inexpensive, and available worldwide. Furthermore, it is imperative to mitigate chronic disease risks and chronic diseases which increase susceptibility to SARS-CoV-2. Also, it is imperative to favorably enhance the biological and immune health of mammals that are infected or exposed to SARS-CoV-2 in order to enable improved outcomes.

Accordingly, there is a need for novel compositions and methods for the treatment or prevention of acute or chronic disease, particularly SARS-Cov-2 viral infections. There is a further need for such compositions and methods to be relatively inexpensive and available worldwide by repurposing existing drugs with known safety profiles.

### SUMMARY

To address the foregoing problems, in whole or in part, and/or other problems that may have been observed by persons skilled in the art, the present disclosure provides compositions and methods as described by way of example as set forth below.

In one embodiment, a pharmaceutical composition is provided comprising:
a) two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3; and/or
b) one or more additional active agents comprising:
   i) one or more modulators of Renin Aldosterone Angiotensin System (RAAS) dysregulation, comprising an inhibitor of the ACE/Angiotensin II arm of RAAS and/or an activator of the ACE2/Ang(1-7)/Mas1 arm of RAAS comprising an inhibitor of ACE, an inhibitor of Angiotensin II activation, an activator of ACE2, an activator of Ang(1-7), an inhibitor of AT1R, an inhibitor of ACE gene expression and/or activity, an inhibitor of Renin, an inhibitor Chymase, a mast cell stabilizer, an inhibitor of histamin, an inhibitor of neprolysin, a combined inhibitor of At1R and Neprilysin, an activator of ADAM17 activity and/or gene expression, an activator of MAS1, an activator of MAS1 gene expression and/or activity, an activator of AT2R, and/or an activator of AT2R;
   ii) one or more modulators of Kallikrein-Kinin System (KKS) dysregulation;
   iii) one or more modulators of protease dysregulation; and/or
   iv) one or more modulators of cytokine dysregulation;
      wherein the pharmaceutical composition further comprises a pharmaceutically acceptable carrier; and wherein the active agents are each present in therapeutically effective amounts to prevent and/or treat a health condition related to inflammasome NLRP3 dysregulation, RAAS dysregulation, KKS dysregulation, protease dysregulation, and/or cytokine dysregulation in a subject in need thereof.

In other aspects, the pharmaceutical composition is further characterized wherein:
a) the two or more active agents of 1(a) comprise a full or partial inhibitor of NLRP3, NLRC4, NLRP1, NLRP6, IFI16, Pyrin, and/or AIM2;
b) the one or more modulators of KKS dysregulation comprise an inhibitor of KKS activation comprising an inhibitor of Neprilysin, a combined inhibitor of At1R and Neprilysin, an inhibitor of ACE and Neprilysin, an inhibitor of Bradykinin B2, an inhibitor of Bradykinin B1, inhibitor of Kallikrein B1(KLKB1), an inhibitor of Kininogen 1 (KNG1), an inhibitor of High-molecular-weight kininogen (HMWK), an inhibitor of Low-molecular-weight kininogen (LMWK), an activator of Kallistatin, an inhibitor of histamine, an activator of SERPINA1, an activator of SERPINA4, and/or an activator of Cystatin A;
c) the one or more modulators of protease dysregulation comprise an inhibitor of a protease comprising Furin, Cathepsin B, Cathepsin C, Cathepsin D, Cathepsin F, Cathepsin G, Cathepsin K, Cathepsin O, Cathepsin S, Cathepsin L, Cathepsin V, Cathepsin X, Cathepsin Z, TMPRSS2, CLEC4M, CAPN2, CAPN1, CD209, and/or TMPRSS4, and/or the one or more modulators of protease dysregulation comprise an activator of CAST (calpastatin) gene activity and/or gene expression, an activator of Calpastatin, and/or an activator of Cystatin A; and
d) the one or more modulator of cytokine dysregulation comprises an activator of IFN-λ, an activator of Type I interferon, an inhibitor of CXCs, an inhibitor of CCL2, and/or an activator of IL-10.

In other aspects, the two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3 comprise an activator of cellular autophagy, an activator of mitophagy, an activator of purine salvage pathways, an activator of mitochondrial cellular bioenergetics, an activator of NRF2 gene expression and/or activity, an activator of SIRT1, an activator of ACE2/MAS/ANG(1-7), an ANG(1-7) mimetic, an activator of NLRP3 ubiquitination and degradation, an activator of dopamine receptor-1 (DRD1), and/or an activator of NAD+, further wherein the activator of NAD+ comprises an activator of nicotinamide salvage pathways, a precursor of NAD+, an agonist of NAD+, an NAD+ mimetic, an inhibitor of ROS and oxidative stress, an inhibitor of PARP1, an activator of NAD+ dependent SIRT2, an activator of mitochondrial uncoupling, an activator of mitochondrial biogenesis, and/or an activator of transcription and/or activity of genes comprising SIRT2, SIRT1, NAMPT, and/or NMNAT1.

In other aspects, the two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3 also comprise one or more full or partial inhibitors of one or more priming or activation signals of one or more activated Pattern Recognition Receptors (PRRs) and/or a modulator of PRR-activation-induced dysregulation in inflammasome signal transduction pathways, wherein the PRR is an inflammasome.

In other aspects, the one or more full or partial inhibitors of one or more priming or activation signals of one or more activated PRRs comprises a full or partial inhibitor of NLRP1, NLRX1, NLRC5; and/or cGAS-STING.

In other aspects, the one or more full or partial inhibitors of one or more priming or activation signals of one or more activated PRRs comprises a full or partial inhibitor of NLRP3, NLRC4, NLRP1, NLRP6, IFI16, Pyrin, and/or AIM2 and/or their related adaptors or effectors, comprising a phenothiazine dye, 4-phenylbutyrate (4-PBA), 4-phenylbutyric acid, sodium phenyl butyrate, methylene blue, glycine, a xanthine oxidoreductase inhibitor, a bile acid, taurodeoxychcolic acid (TUDCA), naltrexone, a microtubule disruptor/inhibitor, demcolcine, colchicine, an HMG-CoA reductase inhibitors/Statin, simvastatin, an antihistamine, a fibric acid derivative, metformin, propylthiouracil, thiazolidinedione, ketamine, nicotine, ivermectin, albendazole, an H/K inhibitor, amitriptyline, a corticosteroid, niclosamide, riluzole, sunitinib, valproic acid, glibenclamide bortezomib, an antibiotic, a tetracycline, minocycline, gentamycine, levofloxacine, cyclosporin, diminazen or diminazene aceturate, a calcium channel blocker, felodipine, a leukotriene D4 inhibitor, an ACE inhibitor, enalapril, an Angiotensin Receptor 1 inhibitor, resveratrol, anthocyanin, taurine, thioctic acid, rosmarinic acid, sulforaphane, celastrol, berberine, Epigallocatechin gallate, catechin, glucosamine, melatonin, pregnenolone, testosterone, estradiol, estriol, mangiferin, linarin, fisetin, rutin, rutecarpin, selenium, selenomethionin, progesterone, n-acetyl cysteine, acetyl cysteine, Mitotempo, tempol, catalase, SOD, apigenin, quercetin, tempol, silybin/silymarin, apocynin, a zinc compound, silybin, curcumin, alpha lipoic acid, genistein, gedunin, astaxanthin, copper, vitamin C, vitamin E, Vitamin D, calcitriol, niacin, tretinoin, acetaminophen, pregnenolone, a non-steroidal anti-inflammatory drug, β-Ursolic acid, Schinol, glycyrrhizic acid, trichostatinA, docasohexaenoic acid, pathenolide and/or any compound listed in Tables 1, 2, 3, or 4 in FIGS. 1, 2, 3, and 4.

In other aspects, the health condition is SARS-CoV-2 infection and/or COVID-19.

In other aspects, the pharmaceutical composition comprises one or more modulators of SARS-CoV-2 associated RAAS dysregulation and one or more modulators of KKS dysregulation, wherein the one or more modulators of SARS-CoV-2 associated RAAS dysregulation and the one or more modulators of KKS dysregulation are each present in therapeutically effective amounts to prevent or treat SARS-CoV-2 infection and/or COVID-19 in a subject in need thereof.

In other aspects, the pharmaceutical composition comprises one or more inhibitors of SARS-CoV-2 associated inflammasome activators, wherein the inflammasome is NLRP3, AIM2, and/or NLRP1, and further wherein the one or more inhibitors of SARS-CoV-2 associated inflammasome activators is present in therapeutically effective amounts to prevent or treat SARS-CoV-2 infection and/or COVID-19 in a subject in need thereof.

In other aspects, the pharmaceutical composition comprises one or more inhibitors of SARS-CoV-2 associated cytokine dysregulation, comprising a modulator of SARS-CoV-2 associated Interferon I and/or III antiviral response.

In other aspects, the modulator of SARS-CoV-2 associated Interferon antiviral response is an activator of Interferon III (Lambda) comprising:
a) an activator/stimulator of IFN-λ;
b) a pegylated-interferon-lambda;
c) an activator of IRF3 gene expression and/or activity;
d) an activator of IRF7 gene expression and/or activity;
e) an activator of Interferon Lambda receptor INFLR1 gene expression and/or activity for early infection or an inhibitor of Interferon Lambda receptor INFLR1, IFR3, IRF7, and/or IL10RB for after early infection; and/or
f) an activator of Interferon Lambda receptor IL10RB gene expression and/or activity.

In other aspects, the modulator of SARS-CoV-2 associated Interferon antiviral response is an activator of Il-15 comprising:
a) an activator of IL-15 gene mRNA expression and/or activity;
b) an activator of Interferon lambda for early infection or an inhibitor of Interferon Lambda receptor INFLR1, IFR3, IRF7, and/or IL10RB for after early infection;
c) an activator of IL-12 gene expression and/or activity; and/or
d) an activator of IL-21 gene expression and/or activity.

In other aspects, the modulator of SARS-CoV-2 associated Interferon antiviral response is an activator of Interferon I comprising:
i) an activator of Interferon1 beta;
ii) a pegylated-interferon-1beta;
iii) an Interferon Lambda mimetic, agonist, and/or activator;
iv) an Interferon I mimetic, agonist, and/or activator;
v) an activator of IRF3 gene expression and/or activity; and/or
vi) an activator of expression of interferon 1 receptor gene IFNAR1 or IFNAR2.
   In other aspects, the modulator of SARS-CoV-2 associated Interferon antiviral response is an inhibitor of IFNAR1 or IFNAR2, comprising:
i) an inhibitor of IFNAR1 gene expression and/or activity, and
ii) an inhibitor of IFNAR2 gene expression and/or activity.

In other aspects, the modulator of SARS-CoV-2 associated Interferon antiviral response is an activator of IL-10 comprising:
i) an inhibitor of AT1R; and/or
ii) an activator of Heat Shock Factor 1.

In other aspects, the pharmaceutical composition comprises:
a) the two or more full or partial inhibitors of one or more priming or activation signals of inflammasome NLRP3;
b) an activator of Interferon beta and/or IFN-λ for early infection;
c) an inhibitor of Interferon beta and/or IFN-λ for late infection; and
d) an activator of anti-inflammatory interleukin-10 (IL-10)-related cytokines comprising IL-10, IL-19, IL-20, IL-22, IL-24, and IL-26.

In other aspects, the pharmaceutical composition comprises:
a) the two or more full or partial inhibitors of one or more priming or activation signals of inflammasome NLRP3; and
b) the one or more modulators of cytokine dysregulation comprises an activator of IFN-β, an activator of IFN-λ and an activator of IL-10.

In other aspects, the pharmaceutical composition modulates inflammasome priming and/or activation, wherein the pharmaceutical composition inhibits activators of inflammasomes, inhibits activators of inflammasome priming, inhibits inflammasome sensors, inhibits inflammasome adaptors, inhibits inflammasome effectors, inhibits pyroptosis, inhibits, necroptosis, inhibits apoptosis, and/or inhibits panoptosis.

In other aspects, the pharmaceutical composition inhibits ASC activation, inhibits Caspase 1 activation, inhibits Gasdermin D activation, inhibits Interleukin 1β (IL-1β) activation, and/or inhibits Interleukin 18 (IL-18) activation.

In other aspects, the pharmaceutical composition modulates gene transcription, gene translation, gene activity, gene expression, post-transcriptional, and/or post-translational modification of a protein encoded by a gene, wherein the gene comprises ACE, ACE2, ADAM17, ADAM9, ADAMTS13, ADORA2A, AGT, AGTR, AGTR1, AGTR2, AIM2, ANAPC1, ANO6, BDKRB1, BDKRB2, BID, BIRC2, BIRC3, BIRC5, BIRC6, BRCC3, BTK, BTK, CAMK2A, CAMP, CAPN1, CAPN2, CASP1, CASP10, CASP11, CASP12, CASP14, CASP2, CASP3, CASP4, CASP5, CASP6, CASP7, CASP8, CASP9, CAST, CAT, CBL, CCL2, CD209, CFL2, CFLAR, CFLAR-AS1, CHMP4A, CHMP4B, CHMP4C, CHUK, CLEC4M, CSTA, CTSB, CTSC, CTSD, CTSF, CTSG, CTSH, CTSK, CTSL, CTSO, CTSS, CTSV, CTSX, CTSZ, CXCL10, CXCL2, CXCL3, CYLD, DDX15, DDX19, DDX5, DNMT1, DPP4, FADD, FURIN, GSDMA, GSDMB, GSDMC, GSDMD, GSDME, GSPT1, GSPT2, H1-1, HIF1A, HIF1AN, HIF3A, HMGB1, HSP90, HSP90AA1, HSP90AB4P, HSP90B1, HSP90B2P, IFIT1, IFNA1, IFNAR1, IFNAR2, IFNLR1, IKBKB, IKBKE, IKBKG, IKK, IL10, IL10RA, IL10RB, IL15, IL17, IL17A, IL18, IL18BP, IL1B, IL1R1, IL1RL1, IL21, IL9, IRF3, IRF7, KEAP, KL, KLKB1, KNG1, LRRK2, LTB4R, MAP3K7, MAPK1, MAPK3, MAPKAPK2, MAS1, MAVS, MIR20A, MIR20B, MIR21, MIR223, MIR30C, MIR30C1, MIR30C5, MIR495, MIR9, MLKL, MME, NAMPT, NEK6, NEK7, NEK9, NFATC3, NFE2L2, NFKB, NLRC3, NLRC4, NLRC5, NLRP1, NLRP10, NLRP11, NLRP12, NLRP13, NLRP14, NLRP2, NLRP3, NLRP4, NLRP5, NLRP6, NLRP7, NLRP8, NLRP9, NLRX1, NMNAT1, NOX, NOX2, NOX4, NR1H4, P2RX1, P2RX2, P2RX2, P2RX3, P2RX3, P2RX4, P2RX5, P2RX5-TAX1BP3, P2RX6, P2RX6P, P2RX7, P2RY1, P2RY10, P2RY11, P2RY12, P2RY13, P2RY14, P2RY2, P2RY4, P2RY6, P2RY8, PADI4, PARP, PARP1, PELI1, PELI2, PELI3, PEP, PRCP, PYCARD, RIPK1, RIPK2, RIPK3, RNF31, SENP6, SENP7, SERPINA1, SERPINA4, SESN2, SIRT1, SIRT2, SLC44A2, SOCS1, SOD, SOD1, SUGT1, TAB1, TAB3, TBK1, TBKBP1, TF, TFP1, TFPI, THOP1, TICAM1, TMPRSS4, TMRPSS2, TNF, TNFAIP1, TNFAIP2, TNFAIP3, TRADD, TRAF2, TRIB3, TRIM31, TRPA4, TRPM2, TRPV1, TRPV4, USP18, VDAC1, VDR, XDH, and/or ZBP1.

In other aspects, the pharmaceutical composition inhibits inflammasome sensor activation, ASC activation, inhibits Caspase 1 activation, inhibits Caspase 8 activation, Caspase 9 activation, inhibits RIPK1 activation, inhibits RIPK3 activation, inhibits FADD activation, inhibits MLKL activation, inhibits Gasdermin D activation, inhibits IL-1β activation, and/or inhibits IL-18 activation; and further wherein the inflammasome comprises NLRP3, NLRC4, NLRP1, and/or AIM2.

In other aspects, the pharmaceutical composition inhibits inflammasome priming and/or activation signals comprising inhibiting DAMPS and/or mitochondrial DAMPS, inhibiting PAMPS and/or PAMPS associated TLR activation, modulating RAAS activation, modulates Kallikrein Kinin dysregulation, inhibiting NFKB pathways, inhibiting ER stress and/or ER associated UPR, inhibiting microtubule assembly and or function, inhibiting intracellular NAD+ release, inhibiting mitochondrial stress, mitochondrial fission, and/or mitochondrial apoptosis, inhibiting diminished mitophagy, inhibiting mtDNA damage, inhibiting diminished mitochondrial membrane potential (Δψm), inhibiting oxidoreductase enzyme, inhibiting TXNIP, inhibiting lysosomal rupture, inhibiting cathepsins, inhibiting cathepsin B, inhibiting angiotensin II, inhibiting cyclic GMP-AMP synthase (cGAS) and stimulator of interferon genes (STING) activation, inhibiting HMGB1/RAGE, inhibiting reactive oxygen species (ROS) and superoxide, inhibiting HSP90, inhibiting SUGT1 (SGT1), inhibiting NEK7, inhibiting intracellular calcium increase and/or intracellular rise of particulate/crystals, inhibiting necroptosis via inhibiting TNFR1, MLKL, ZBP1, RIPK3, and/or RIPK1, inhibiting CASP1, inhibiting CASP8, inhibiting CASP4, inhibiting CASP5, inhibiting CASP9, inhibiting ASC oligomerization, inhibiting Gasdermin D (GSDMD), inhibiting DUBs (deubiquitinase enzyme), inhibiting non-canonical inflammasome activation, inhibiting the purinoreceptors P2RX1-7, inhibiting P2RX7, and/or P2RY, inhibiting CXCR2-PKCm-ILK axis, inhibiting IL-17, inhibiting IL-21, inhibiting the PI3K/AKT/mTOR pathway, and/or inhibiting Micro RNA 21.

In other aspects, the pharmaceutical composition further comprises a therapeutically effective amount of a modulator of SARS-CoV-2-associated spike protein receptor binding, cleavage, fusion, and/or viral entry into host cells comprising a protease inhibitor comprising TMPRSS2, TMPRSS4, Cathepsin B, Cathepsin L, Cathepsin S, Cathepsin Z, Cathepsin C, DPP4, CD209, CLEC4M and/or Furin.

In other aspects, the pharmaceutical composition further comprises a therapeutically effective amount of a modulator of SARS-CoV-2-associated spike protein receptor binding, cleavage, fusion, and/or viral entry into host cells comprising an inhibitor of ACE2, an activator of ADAM17, an activator of Ang(1-7), an activator of MAS1, an agonist and/or mimetic of Kallistatin, an activator of SERPIN4a, and/or an activator of SERPIN1a.

In other aspects, the pharmaceutical composition comprises a pharmaceutically acceptable salt, hydrate, cocrystal, solvate, ion charged compound, and/or prodrug of any of the active agents described herein.

In other aspects, the pharmaceutical composition is formulated for a route of administration comprising oral, intramuscular, subcutaneous, intradermal, parenteral, intraperitoneal, intranasal, inhalational, nebulized, intramucosal, sublingual, rectal, topical, transdermal, intravitreal, conjunctival, intra ocular, mucosal, intratracheal, intra-urethral, vaginal, labial, intra-aural, and/or intravenous routes.

In other aspects, the pharmaceutical composition comprises a formulation comprising solids, liquids, gaseous, vaporized, syrup, tablets, gel capsules, vegetarian gel capsules, pastilles, dry powder, sachets, emulsion, pellets, inhalational solution, drops, ampules, gummy bears, candy bars, power drinks, food powders, effervescent tablets, nebulized, nasal sprays, disks, mucosal preparations, anal preparations, rectal preparations, creams, lotions, gels, serums, colloidal preparations, liposomal preparations, topical preparations, transdermal preparations, immediate release preparations, delayed preparations, extended release preparations, controlled release preparations, and/or sustained release preparations.

In another embodiment, a method is provided for preventing and/or treating a health condition related to inflammasome NLRP3 dysregulation, RAAS dysregulation, KKS dysregulation, protease dysregulation, and/or cytokine dysregulation in a subject in need thereof, comprising administering a pharmaceutical composition to the subject, wherein the pharmaceutical composition comprises:
a) two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3; and/or
b) one or more additional active agents comprising:
   i) one or more modulators of Renin Aldosterone Angiotensin System (RAAS) dysregulation, comprising an inhibitor of the ACE/Angiotensin II arm of RAAS and/or an activator of the ACE2/Ang(1-7)/Mas1 arm of RAAS comprising an inhibitor of ACE, an inhibitor of Angiotensin II activation, an activator of ACE2, an activator of Ang(1-7), an inhibitor of AT1R, an inhibitor of ACE gene expression and/or activity, an inhibitor of Renin, an inhibitor Chymase, a mast cell stabilizer, an inhibitor of histamine, an inhibitor of neprolysin, a combined inhibitor of At1R and Neprilysin, an activator of ADAM17 activity and/or gene expression, an activator of MAS1, an activator of MAS1 gene expression and/or activity, an activator of AT2R, and/or an activator of AT2R;
   ii) one or more modulators of Kallikrein-Kinin System (KKS) dysregulation;
   iii) one or more modulators of protease dysregulation; and/or
   iv) one or more modulators of cytokine dysregulation;
      wherein the pharmaceutical composition further comprises a pharmaceutically acceptable carrier; and wherein the active agents are each present in therapeutically effective amounts to prevent and/or treat the health condition related to inflammasome NLRP3 dysregulation, RAAS dysregulation, KKS dysregulation, protease dysregulation, and/or cytokine dysregulation in the subject in need thereof.

In other aspects, the pharmaceutical composition within the method is further characterized wherein:
a) the two or more active agents of 1(a) comprise a full or partial inhibitor of NLRP3, NLRC4, NLRP1, NLRP6, IFI16, Pyrin, and/or AIM2;
b) the one or more modulators of KKS dysregulation comprise an inhibitor of KKS activation comprising an inhibitor of Neprilysin, a combined inhibitor of At1R and Neprilysin, an inhibitor of ACE and Neprilysin, an inhibitor of Bradykinin B2, an inhibitor of Bradykinin B1, inhibitor of Kallikrein B1(KLKB1), an inhibitor of Kininogen 1 (KNG1), an inhibitor of High-molecular-weight kininogen (HMWK), an inhibitor of Low-molecular-weight kininogen (LMWK), an activator of Kallistatin, an inhibitor of histamine, an activator of SERPINA1, an activator of SERPINA4, and/or an activator of Cystatin A;
c) the one or more modulators of protease dysregulation comprise an inhibitor of a protease comprising Furin, Cathepsin B, Cathepsin C, Cathepsin D, Cathepsin F, Cathepsin G, Cathepsin K, Cathepsin O, Cathepsin S, Cathepsin L, Cathepsin V, Cathepsin X, Cathepsin Z, TMPRSS2, CLEC4M, CAPN2, CAPN1, CD209, and/or TMPRSS4, and/or the one or more modulators of protease dysregulation comprise an activator of CAST (calpastatin) gene activity and/or gene expression, an activator of Calpastatin, and/or an activator of Cystatin A; and
d) the one or more modulator of cytokine dysregulation comprises an activator of IFN-λ, an activator of Type I interferon, an inhibitor of CXCs, an inhibitor of CCL2, and/or an activator of IL-10.

In other aspects, the activator of IFN-λ and/or activator of Type I interferon are administered to the subject less than 48 hours post-infection.

In other aspects, the two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3 comprise an activator of cellular autophagy, an activator of mitophagy, an activator of purine salvage pathways, an activator of mitochondrial cellular bioenergetics, an activator of NRF2 gene expression and/or activity, an activator of SIRT1, an activator of ACE2/MAS/ANG(1-7), an ANG(1-7) mimetic, an activator of NLRP3 ubiquitination and degradation, an activator of dopamine receptor-1 (DRD1), and/or an activator of NAD+, further wherein the activator of NAD+ comprises an activator of nicotinamide salvage pathways, a precursor of NAD+, an agonist of NAD+, an NAD+ mimetic, an inhibitor of ROS and oxidative stress, an inhibitor of PARP1, an activator of NAD+ dependent SIRT2, an activator of mitochondrial uncoupling, an activator of mitochondrial biogenesis, and/or an activator of transcription and/or activity of genes comprising SIRT2, SIRT1, NAMPT, and/or NMNAT1.

In other aspects, the two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3 also comprise one or more full or partial inhibitors of one or more priming or activation signals of one or more activated Pattern Recognition Receptors (PRRs) and/or a modulator of PRR-activation-induced dysregulation in inflammasome signal transduction pathways, wherein the PRR is an inflammasome.

In other aspects, the one or more full or partial inhibitors of one or more priming or activation signals of one or more activated PRRs comprises a full or partial inhibitor of NLRP1, NLRX1, NLRC5; and/or cGAS-STING.

In other aspects, the one or more full or partial inhibitors of one or more priming or activation signals of one or more activated PRRs comprises a full or partial inhibitor of NLRP3, NLRC4, NLRP1, NLRP6, IFI16, Pyrin, and/or AIM2 and/or their related adaptors or effectors, comprising a phenothiazine dye, 4-phenylbutyrate (4-PBA), 4-phenylbutyric acid, sodium phenyl butyrate, methylene blue, glycine, a xanthine oxidoreductase inhibitor, a bile acid, taurodeoxychcolic acid (TUDCA), naltrexone, a microtubule disruptor/inhibitor, demcolcine, colchicine, an HMG-CoA reductase inhibitors/Statin, simvastatin, an antihistamine, a fibric acid derivative, metformin, propylthiouracil, thiazolidinedione, ketamine, nicotine, ivermectin, albendazole, an H/K inhibitor, amitriptyline, a corticosteroid, niclosamide, riluzole, sunitinib, valproic acid, glibenclamide bortezomib, an antibiotic, a tetracycline, minocycline, gentamycine, levofloxacine, cyclosporin, diminazen or diminazene aceturate, a calcium channel blocker, felodipine, a leukotriene D4 inhibitor, an ACE inhibitor, enalapril, an Angiotensin Receptor 1 inhibitor, resveratrol, anthocyanin, taurine, thioctic acid, rosmarinic acid, sulforaphane, celastrol, berberine, Epigallocatechin gallate, catechin, glucosamine, melatonin, pregnenolone, testosterone, estradiol, estriol, mangiferin, linarin, fisetin, rutin, rutecarpin, selenium, selenomethionin, progesterone, n-acetyl cysteine, acetyl cysteine, Mitotempo, tempol, catalase, SOD, apigenin, quercetin, tempol, silybin/silymarin, apocynin, a zinc compound, silybin, curcumin, alpha lipoic acid, genistein, gedunin, astaxanthin, copper, vitamin C, vitamin E, Vitamin D, calcitriol, niacin, tretinoin, acetaminophen, pregnenolone, a non-steroidal anti-inflammatory drug, β-Ursolic acid, Schinol, glycyrrhizic acid, trichostatinA, docasohexaenoic acid, pathenolide and/or any compound listed in Tables 1, 2, 3, or 4 in FIGS. 1, 2, 3, and 4.

In other aspects, the health condition is SARS-CoV-2 infection and/or COVID-19.36.

In other aspects, the method comprises preventing and/or treating a symptom of SARS-CoV-2 infection and/or COVID-19 comprising stroke, myocardial infarction, arterial thromboembolism, venous thromboembolism, microvascular thromboembolism, lung injury, acute respiratory distress syndrome, pulmonary fibrosis, hypoxemia, retinopathy, vasculitis, antiphospholipid syndrome, thrombocytopenia, cardiomyopathy, myocarditis, encephalopathy, renal disease, gastrointestinal disease, skin rash, skin ulcer, infertility, fatigue, and/or ventilator induced lung injury.

In other aspects, the pharmaceutical composition within the method comprises one or more modulators of SARS-CoV-2 associated RAAS dysregulation and one or more modulators of KKS dysregulation, wherein the one or more modulators of SARS-CoV-2 associated RAAS dysregulation and the one or more modulators of KKS dysregulation are each present in therapeutically effective amounts to prevent or treat SARS-CoV-2 infection and/or COVID-19 in a subject in need thereof.

In other aspects, the pharmaceutical composition within the method comprises one or more inhibitors of SARS-CoV-2 associated inflammasome activators, wherein the inflammasome is NLRP3, AIM2, and/or NLRP1, and further wherein the one or more inhibitors of SARS-CoV-2 associated inflammasome activators is present in therapeutically effective amounts to prevent or treat SARS-CoV-2 infection and/or COVID-19 in a subject in need thereof.

In other aspects, the pharmaceutical composition within the method comprises one or more inhibitors of SARS-CoV-2 associated cytokine dysregulation, comprising a modulator of SARS-CoV-2 associated Interferon I and/or III antiviral response.

In other aspects, the modulator of SARS-CoV-2 associated Interferon antiviral response is an activator of IFN-λ comprising:
a) an activator/stimulator of IFN-λ;
b) a pegylated-IFN-λ;
c) an activator of IRF3 gene expression and/or activity;
d) an activator of IRF7 gene expression and/or activity;
e) an activator of IFN-λ receptor INFLR1 gene expression and/or activity, or an inhibitor of IFN-λ receptor INFLR1, IFR3, IRF7, and/or IL10RB; and/or
f) an activator of IFN-λ receptor IL10RB gene expression and/or activity.

In other aspects, the activator of IFN-λ receptor INFLR1 gene expression and/or activity is administered to the subject less than 48 hours post-infection and the inhibitor of IFN-λ receptor INFLR1, IFR3, IRF7, and/or IL10RB is administered to the subject 48 hours or more post-infection.

In other aspects, the modulator of SARS-CoV-2 associated Interferon antiviral response is an activator of Il-15 comprising:
a) an activator of IL-15 gene mRNA expression and/or activity;
b) an activator of IFN-λ for early infection or an inhibitor of IFN-λ receptor INFLR1, IFR3, IRF7, and/or IL10RB for after early infection;
c) an activator of IL-12 gene expression and/or activity; and/or
d) an activator of IL-21 gene expression and/or activity.

In other aspects, the modulator of SARS-CoV-2 associated Interferon antiviral response is an activator of Interferon I comprising:
i) an activator of Interferon1 beta;
ii) a pegylated-interferon-1beta;
iii) an IFN-λ mimetic, agonist, and/or activator;
iv) an Interferon I mimetic, agonist, and/or activator;
v) an activator of IRF3 gene expression and/or activity; and/or
vi) an activator of expression of interferon 1 receptor gene IFNAR1 or IFNAR2.

In other aspects, the modulator of SARS-CoV-2 associated Interferon antiviral response is an inhibitor of IFNAR1 or IFNAR2, comprising:
i) an inhibitor of IFNAR1 gene expression and/or activity, and
ii) an inhibitor of IFNAR2 gene expression and/or activity.

In other aspects, the modulator of SARS-CoV-2 associated Interferon antiviral response is an activator of IL-10 comprising:
i) an inhibitor of AT1R; and/or
ii) an activator of Heat Shock Factor 1.

In other aspects, the pharmaceutical composition within the method comprises:
a) the two or more full or partial inhibitors of one or more priming or activation signals of inflammasome NLRP3;
b) an activator of Interferon beta and/or IFN-λ for early infection;
c) an inhibitor of Interferon beta and/or IFN-λ for late infection; and
d) an activator of anti-inflammatory interleukin-10 (IL-10)-related cytokines comprising IL-10, IL-19, IL-20, IL-22, IL-24, and IL-26.

In other aspects, the pharmaceutical composition within the method comprises:
a) the two or more full or partial inhibitors of one or more priming or activation signals of inflammasome NLRP3; and
b) the one or more modulators of cytokine dysregulation comprises an activator of IFN-β, an activator of IFN-λ and an activator of IL-10.

In other aspects, the pharmaceutical composition within the method modulates inflammasome priming and/or activation, wherein the pharmaceutical composition inhibits activators of inflammasomes, inhibits activators of inflammasome priming, inhibits inflammasome sensors, inhibits inflammasome adaptors, inhibits inflammasome effectors, inhibits pyroptosis, inhibits, necroptosis, inhibits apoptosis, and/or inhibits panoptosis.

In other aspects, the pharmaceutical composition within the method inhibits ASC activation, inhibits Caspase 1 activation, inhibits Gasdermin D activation, inhibits Interleukin 1β (IL-1β) activation, and/or inhibits Interleukin 18 (IL-18) activation.

In other aspects, the pharmaceutical composition within the method modulates gene transcription, gene translation, gene activity, gene expression, post-transcriptional, and/or post-translational modification of a protein encoded by a gene, wherein the gene comprises ACE, ACE2, ADAM17, ADAM9, ADAMTS13, ADORA2A, AGT, AGTR, AGTR1, AGTR2, AIM2, ANAPC1, ANO6, BDKRB1, BDKRB2, BID, BIRC2, BIRC3, BIRC5, BIRC6, BRCC3, BTK, BTK, CAMK2A, CAMP, CAPN1, CAPN2, CASP1, CASP10, CASP11, CASP12, CASP14, CASP2, CASP3, CASP4, CASP5, CASP6, CASP7, CASP8, CASP9, CAST, CAT, CBL, CCL2, CD209, CFL2, CFLAR, CFLAR-AS1, CHMP4A, CHMP4B, CHMP4C, CHUK, CLEC4M, CSTA, CTSB, CTSC, CTSD, CTSF, CTSG, CTSH, CTSK, CTSL, CTSO, CTSS, CTSV, CTSX, CTSZ, CXCL10, CXCL2, CXCL3, CYLD, DDX15, DDX19, DDX5, DNMT1, DPP4, FADD, FURIN, GSDMA, GSDMB, GSDMC, GSDMD, GSDME, GSPT1, GSPT2, H1-1, HIF1A, HIF1AN, HIF3A, HMGB1, HSP90, HSP90AA1, HSP90AB4P, HSP90B1, HSP90B2P, IFIT1, IFNA1, IFNAR1, IFNAR2, IFNLR1, IKBKB, IKBKE, IKBKG, IKK, IL10, IL10RA, IL10RB, IL15, IL17, IL17A, IL18, IL18BP, IL1B, IL1R1, IL1RL1, IL21, IL9, IRF3, IRF7, KEAP, KL, KLKB1, KNG1, LRRK2, LTB4R, MAP3K7, MAPK1, MAPK3, MAPKAPK2, MAS1, MAVS, MIR20A, MIR20B, MIR21, MIR223, MIR30C, MIR30C1, MIR30C5, MIR495, MIR9, MLKL, MME, NAMPT, NEK6, NEK7, NEK9, NFATC3, NFE2L2, NFKB, NLRC3, NLRC4, NLRC5, NLRP1, NLRP10, NLRP11, NLRP12, NLRP13, NLRP14, NLRP2, NLRP3, NLRP4, NLRP5, NLRP6, NLRP7, NLRP8, NLRP9, NLRX1, NMNAT1, NOX, NOX2, NOX4, NR1H4, P2RX1, P2RX2, P2RX2, P2RX3, P2RX3, P2RX4, P2RX5, P2RX5-TAX1BP3, P2RX6, P2RX6P, P2RX7, P2RY1, P2RY10, P2RY11, P2RY12, P2RY13, P2RY14, P2RY2, P2RY4, P2RY6, P2RY8, PADI4, PARP, PARP1, PELI1, PELI2, PELI3, PEP, PRCP, PYCARD, RIPK1, RIPK2, RIPK3, RNF31, SENP6, SENP7, SERPINA1, SERPINA4, SESN2, SIRT1, SIRT2, SLC44A2, SOCS1, SOD, SOD1, SUGT1, TAB1, TAB3, TBK1, TBKBP1, TF, TFP1, TFPI, THOP1, TICAM1, TMPRSS4, TMRPSS2, TNF, TNFAIP1, TNFAIP2, TNFAIP3, TRADD, TRAF2, TRIB3, TRIM31, TRPA4, TRPM2, TRPV1, TRPV4, USP18, VDAC1, VDR, XDH, and/or ZBP1.

In other aspects, the pharmaceutical composition within the method inhibits inflammasome priming and/or activation signals comprising inhibiting DAMPS and/or mitochondrial DAMPS; inhibiting PAMPS and/or PAMPS associated TLR activation; modulating RAAS activation; modulates Kallikrein Kinin dysregulation; inhibiting NFKB pathways; inhibiting ER stress and/or ER associated UPR; inhibiting microtubule assembly and or function; inhibiting intracellular NAD+ release; inhibiting mitochondrial stress, mitochondrial fission, and/or mitochondrial apoptosis; inhibiting diminished mitophagy; inhibiting mtDNA damage; inhibiting diminished mitochondrial membrane potential (Δψm); inhibiting oxidoreductase enzyme; inhibiting TXNIP; inhibiting lysosomal rupture; inhibiting cathepsins; inhibiting cathepsin B, inhibiting angiotensin II; inhibiting cyclic GMP-AMP synthase (cGAS) and stimulator of interferon genes (STING) activation; inhibiting HMGB1/RAGE; inhibiting reactive oxygen species (ROS) and superoxide; inhibiting HSP90; inhibiting SUGT1 (SGT1); inhibiting NEK7; inhibiting intracellular calcium increase and/or intracellular rise of particulate/crystals; inhibiting necroptosis via inhibiting TNFR1, MLKL, ZBP1, RIPK3, and/or RIPK1; inhibiting CASP8; inhibiting CASP4; inhibiting CASP5, inhibiting CASP9; inhibiting ASC oligomerization; inhibiting Gasdermin D (GSDMD); inhibiting DUBs (deubiquitinase enzyme); inhibiting non-canonical inflammasome activation, inhibiting the purinoreceptors P2RX1-7, inhibiting P2RX7, and/or P2RY; inhibiting CXCR2-PKCm-ILK axis; inhibiting IL-17; inhibiting IL-21; inhibiting the PI3K/AKT/mTOR pathway; and/or inhibiting Micro RNA 21.

In other aspects, the pharmaceutical composition within the method further comprises a therapeutically effective amount of a modulator of SARS-CoV-2-associated spike protein receptor binding, cleavage, fusion, and/or viral entry into host cells comprising a protease inhibitor comprising TMPRSS2, TMPRSS4, Cathepsin B, Cathepsin L, Cathepsin S, Cathepsin Z, Cathepsin C, DPP4, CD209, CLEC4M and/or Furin.

In other aspects, the pharmaceutical composition within the method further comprises a therapeutically effective amount of a modulator of SARS-CoV-2-associated spike protein receptor binding, cleavage, fusion, and/or viral entry into host cells comprising an inhibitor of ACE2, an activator of ADAM17, an activator of Ang(1-7), an activator of MAS1, an agonist and/or mimetic of Kallistatin, an activator of SERPIN4a, and/or an activator of SERPIN1a.

In other aspects, the pharmaceutical composition within the method further comprises a pharmaceutically acceptable salt, hydrate, cocrystal, solvate, ion charged compound, and/or prodrug of any of the active agents described herein.

In other aspects, the pharmaceutical composition within the method is formulated for a route of administration comprising oral, intramuscular, subcutaneous, intradermal, parenteral, intraperitoneal, intranasal, inhalational, nebulized, intramucosal, sublingual, rectal, topical, transdermal, intravitreal, conjunctival, intra ocular, mucosal, intratracheal, intra-urethral, vaginal, labial, intra-aural, and/or intravenous routes.

In other aspects, the pharmaceutical composition within the method comprises a formulation comprising solids, liquids, gaseous, vaporized, syrup, tablets, gel capsules, vegetarian gel capsules, pastilles, dry powder, sachets, emulsion, pellets, inhalational solution, drops, ampules, gummy bears, candy bars, power drinks, food powders, effervescent tablets, nebulized, nasal sprays, disks, mucosal preparations, anal preparations, rectal preparations, creams, lotions, gels, serums, colloidal preparations, liposomal preparations, topical preparations, transdermal preparations, immediate release preparations, delayed preparations, extended release preparations, controlled release preparations, and/or sustained release preparations.

In other aspects, any of the active agents are administered alone or together and can be combined and administered as a sequential therapy and/or a concurrent therapy with other medications and therapies as determined by health care providers and/or treating physicians.

In other aspects, the health condition is Cancer, Cardiovascular disease, Thromboembolic disease, Pulmonary disease, Infectious disease, sepsis, Systemic inflammatory response syndrome (SIRS), cytokine storm, Autoimmune and Inflammatory diseases, Inflammatory arthritis, Psoriatic skin disease, Psoriatic arthritis, Systemic lupus erythematosus (SLE), Vasculitis, Osteoarthritis, Multiple Sclerosis (MS), Inflammatory and or genetic and or rare diseases, Chronic pain, Neuropathic pain, Migraine, Glossodynia, Retinopathy, Retinal detachment, Age related Macular degeneration (AMD), Neurologic disorders comprising Dementia and or neurodegenerative disease, Alzheimer's disease, Parkinson's disease, Prion disease, Huntington's disease, Meningitis, Epilepsy, Skin Disease comprising skin ulcers, skin infections, Tinea Infections, onychomycosis, Tinea Versicolor, cutaneous abscess, Cellulitis, alopecia, Renal disease, Interstitial Cystitis (IC), benign prostatic hypertrophy, Gastrointestinal (GI) disease, Hepatitis Liver Cirrhosis, Inflammatory bowel disease, GI Dysbiosis, Aging and age related diseases, cellular senescence, Mitochondrial stress, mitochondrial disease, mitochondrial cytopathies, Osteoporosis, Osteopenia, periodontal bone loss, dyslipidemia, hypercholesterolemia, Mood Disorders, Metabolic disease, Chronic Fatigue Syndrome, and/or Fibromyalgia.

In other aspects, the health condition is a cardiovascular disease comprising atherosclerosis, dyslipidemia, hypercholesterolemia, hypertension, cerebrovascular accident, transient ischemic attack, stroke, vascular dementia, peripheral arterial disease (PAD), venous disease, myocardial infarction, coronary artery disease, carotid artery disease, dilated cardiomyopathy, hypertrophic cardiomyopathy, ischemic cardiomyopathy, cardiac amyloidosis, congestive heart failure, arrythmia, atrial fibrillation, pericardial disease, pericarditis, pericardial effusion, myocarditis, aortic ectasia, aortic aneurysm, abdominal aortic aneurysm, splenic aneurysm, mesenteric ischemia, pulmonary hypertension, renovascular disease, portocaval hypertension, vascular dementia, peripheral vascular disease, peripheral venous disease, peripheral arterial disease, and/or vasculitis, good pastures, Kawasaki, giant cell arteritis, poly arteritis nodosa, antiphospholipid syndrome, Henoch shconline purpura, and/or Churg-Strauss.

In other aspects, the health condition is a thromboembolic disease comprising arterial or venous thromboembolism, small or large vessel thromboembolism, microvascular thrombosis, sepsis associated thromboembolism, stroke, myocardial infarction, mesenteric embolism, antiphospholipid syndrome, vasculitis, Kawasaki disease, deep venous thrombosis, and/or pulmonary embolism.

In other aspects, the health condition is a pulmonary disease comprising acute lung injury, acute respiratory distress syndrome (ARDS), neonatal ARDS, chronic obstructive pulmonary disease, emphysema, anti-alpha trypsin deficiency, asthma, bronchitis, pneumonia, interstitial lung disease, infectious lung disease, granulomatous lung disease, autoimmune lung disease, mesothelioma, pneumoconiosis, asbestosis, bronchiolitis obliterans organizing pneumonia (BOOP), ventilator associated lung damage, hyperoxia lung damage, pleural effusion, pleuritis, fibrotic lung disease, high altitude pulmonary edema, Idiopathic interstitial pneumonias, lymphoid interstitial pneumonia (LIP), idiopathic interstitial pneumonia: unspecified, Hyper-eosinophilic lung diseases, Allergic bronchopulmonary aspergillosis (ABPA), Pulmonary vasculitis, Alveolar hemorrhage syndromes, Pulmonary lymphangioleiomyomatosis (LAM), Alveolar proteinosis, Pulmonary amyloidosis, Pulmonary Langerhans cell histiocytosis (histiocytosis x), Primary pulmonary lymphoma, Primary ciliary dyskinesia (without or with situs inversus), Rare cause of hypersensitivity pneumonitis (all causes other than farmer's lung disease and pigeon breeder's lung disease), Pulmonary arteriovenous malformations in hereditary hemorrhagic telangiectasia (HHT), Pulmonary manifestations of gastro-intestinal disorders, Interstitial lung disease in connective tissue diseases, Bronchiolitis obliterans in non-transplanted patients, Sarcoidosis, Chronic beryllium disease, Waterproofing spray pneumonitis, Vaping related pneumonitis, Combined pulmonary fibrosis and emphysema, and/or Alpha-1-antitrypsin deficiency emphysema.

In other aspects, the health condition is an infectious disease comprising a viral, bacterial, parasitic, and/or fungal infectious disease, wherein the viral infectious disease comprises a disease caused by SARS-CoV, SARS-CoV-2, MERS-CoV, Hepatitis A, Hepatitis B, Hepatitis C, Hepatitis D, Hepatitis E, Hepatitis F, HIV, Ebola, Herpes, Influenza, Roseola, Variola, rubella, measles, chikungunya, Cytomegalovirus, Respiratory syncytial virus, Epstein bar virus, Adeno virus, Coxsackie A, or Coxsackie B, Marburg, Rabies, Hanta, Dengue, and/or Rota, and wherein the bacterial infectious disease comprises disease caused by Escherichia coli, Klebsiella pneumonia, Enterococcus faecalis, Enterococcus faecium, Vibrio cholera, Staphylococcus aureus, Staphylococcus epidermidis, Bacteroides, Neisseria gonorrhea, Neisseria meningitis, Proteus Mirablis, Hemophilus influenza, Haemophilus ducreyi, Campylobacter jejuni, Salmonella, Citrobacter ferundii, Streptococcus pyogenes, Group A streptococcus, Group B streptococcus, Chlamydia psittaci, Francisella tularensis, Coxiella burnetii, Chlamydia pneumoniae, Mycoplasma pneumoniae, Legionella, Chlamydia trachomatis, Salmonella, Shigella, Clostridium perfringens, Borrelia burgdorferi, Rickettsia, and/or mycobacteria, and wherein the parasitic infectious disease comprises a disease caused by giardia and/or Entamoeba histolytica, and wherein the fungal infectious disease comprises a disease caused by cryptococcosis, Aspergillosis, Coccidioidomycosis, Histoplasmosis, Blastomycosis, Pneumocystis jirovecii, candida, dermatophytes, Malassezia globosa, Malassezia furfur, Tinea Infections, onychomycosis, Tinea Versicolor, and/or cutaneous abscess. In other aspects, the infectious disease comprises bacteremia, sepsis, urosepsis, severe sepsis, septic shock, and systemic inflammatory response syndrome (SIRS), and where in the infectious disease comprises cutaneous abscess, furuncles, carbuncles, Bartholin abscess, sterile abscess, perirectal abscess, perianal abscess, vulvar abscess, perineal abscess, perioral abscess, Mycobacterium abscess, epidermoid abscess and wherein the infectious disease comprises a cellulitis disease caused by Staphylococcus epidermidis, Staphylococcus aureus, MRSA, Pasteurella multocida and Capnocytophaga canimorsus, Aeromonas hydrophila, Vibrio vulnificus, Pseudomonas aeruginosa, Group B Streptococcus, Clostridium species (i.e., Clostridial myonecrosis), Erysipelothrix rhusiopathiae, S. pneumoniae, Cryptococcus neoformans, Streptococcus iniae, and/or Helicobacter cinaedi.

In other aspects, the health condition is a renal disease comprising chronic kidney disease (CKD), end stage kidney disease ESRD, nephrocalcinosis, diabetic nephropathy, hypertensive nephropathy, acute renal failure, acute tubular necrosis, nephrotic syndrome, glomerular nephritis, aldosterone-induced renal tubular cell injury, nephrotoxic renal injury, microalbuminuria, proteinuria, renal amyloidosis, amyloid nephropathy, immunoglobulin A nephropathy, polycystic kidney disease urinary, hepatorenal syndrome, interstitial cystitis, urinary tract infection, pyelonephritis, interstitial cystitis (IC), Ulcerative IC and/or non-ulcerative IC.

In other aspects, the health condition is a gastrointestinal (GI) disease comprising peptic ulcer disease (PUD), gastroesophageal reflux disease (GERD), esophagitis, Barrette's esophagitis, gastric dysmotility disease, small intestinal bacterial over growth (SIB), malabsorption, pancreatitis, pancreatic pseudocyst, cholecystitis, cholelithiasis, cirrhosis, ascites, NALD, hepato-steatosis, steatohepatitis, inflammatory bowel disease, hepatitis, cholestatic liver injury, ischemic-perfusion liver disease, liver cirrhosis, inflammatory bowel disease, ulcerative colitis, microscopic colitis, C-diff colitis (pseudomembranous colitis), and/or GI dysbiosis.

In other aspects, the health condition is a metabolic disease comprising prediabetes, diabetes, obesity, steatohepatitis, hepato-steatosis, non-alcoholic steatohepatitis (NASH), pancreatitis, pancreatic pseudocyst, Chronic Fatigue Syndrome, Fibromyalgia, dyslipidemia, and/or hypercholesterolemia.

In other aspects, the health condition is an autoimmune or inflammatory disease comprising systemic lupus erythematosus (SLE), Wegener's granulomatosis, Sjogren's syndrome, vasculitis, anti-phospholipid syndrome, rheumatoid arthritis, osteoarthritis, myositis, dermatomyositis, psoriasis, spondylo-negative arthritis, autoimmune hepatitis, good pastures, vasculitis, mixed connective tissue disease, Inflammatory arthritis, Ankylosing spondylitis, Gout, pseudogout, Lyme's disease, sarcoid arthritis, rheumatoid arthritis, Psoriatic skin disease, Psoriatic arthritis, Osteoarthritis, Multiple Sclerosis (MS), and/or autoimmune encephalitis.

In other aspects, the health condition is a genetic or rare disease comprising Mediterranean fever, Bechet disease, Muckle-Wells syndrome (MWS), and/or Amyloidosis.

In other aspects, the health condition is an acute or chronic pain comprising Neuropathic pain, trigeminal neuralgia, post herpetic neuralgia, cancer pain, chronic neuropathic pain, bone fracture related pain, bone metastasis related pain, pleuritis related pain, pericarditis related pain, Migraine, Glossodynia, oral dysesthesia, glossopyrosis, glossalgia, stomatodynia, stomatopyrosis, chronic pain syndrome, and/or fibromyalgia.

In other aspects, the health condition is an eye disease comprising Retinopathy, non-proliferative or proliferative diabetic retinopathy, Retinal detachment, Retinitis Pigmentosa, Age related Macular degeneration (AMD), wet AMD, uveitis, iritis, cataracts, dry eyes, blepharitis, dry eye blepharitis syndrome (DEBS), glaucoma, conjunctivitis, chalazion, and/or a sty.

In other aspects, the health condition is a neurologic disorder comprising neurodegenerative disease, dementia, senile dementia, Alzheimer's dementia, Parkinson's dementia, dementia of Lewy body, amyloidosis, mild cognitive impairment, frontotemporal dementia, Huntington's chorea dementia, vascular dementia, Creutzfeldt-Jakob disease, other prion disease dementia, mixed dementia, Dementia in HIV/AIDS, dementia associated with Traumatic Brain Injury, Wernicke-Korsakoff Syndrome, alcohol induced dementia, normal pressure hydrocephalus, amyotrophic lateral sclerosis, Alzheimer's disease (AD), Parkinson's disease, Prion disease, Motor neuron, diseases (MND), Huntington's disease (HD), Spinocerebellar ataxia (SCA), Spinal muscular atrophy (SMA), wolfram syndrome (WFS1, WFS2), memory loss, traumatic brain injury, post-traumatic stress disease (PTSD)), Meningitis, Epilepsy, tonic-clinic or convulsive seizures, absence seizures, atonic seizures, tonic seizures, clonic seizures, myoclonic seizures, jacksonian seizure, partial focal seizures and/or Migraine.

In other aspects, the health condition is a skin disease comprising skin ulcers, non-healing ulcers, decubitus ulcers, pressure ulcers, aphthous ulcers, venous stasis ulcers, Stasis ulcers, neuropathic skin ulcers, ischemic arterial ulcers, buruli ulcers, infectious ulcers, pyoderma gangrenosum, vasculitis ulcers, cryoglobulinemic ulcers, diabetic ulcers, warfarin induced skin ulcers, stab wound ulcers, fungal ulcers, bacterial ulcers, viral skin ulcers, herpes ulcers, protozoal skin infections, Rickettsial skin ulcers, epidermal parasitic skin ulcers, renal induced skin ulcers, vesicular skin lesions, candida oral lesions, angular cheilitis, glossitis, hairy tongue, lichen planus, recurrent aphthous stomatitis, herpes labialis, herpes genitalis, erythema migrans, hairy tongue, herpetiform ulcers, recurrent aphthous stomatitis, major and minor aphthous ulcers, gingivitis, pharyngitis, rosacea, acne, Acne vulgaris, Hidradenitis suppurativa/acne inversa (HS), alopecia, androgenic alopecia, alopecia areata, telogen effluvium, anagen effluvium, traction alopecia, trichotillomania, and/or achromotrichia

In other aspects, the health condition is aging and/or age-related diseases comprising diabetes, diabetes type II, obesity, insulin resistance, inflammatory disease, cardiovascular disease, atherosclerosis, thromboembolism, neurodegenerative disease, Alzheimer's disease, Parkinson's disease, Pick's disease, dementia, Lewy body dementias, renal disease, cancer, osteoarthritis, osteopenia, osteoporosis, periodontal bone loss, sarcopenia, periodontitis, rheumatoid arthritis, maculopathy, chronic obstructive pulmonary disease, hair loss, Hutchinson-Gilford progeria, cellular senescence, Mitochondrial stress, mitochondrial disease, and/or dyslipidemia.

In other aspects, the health condition is a mood disorder comprising Bipolar I Disorder, Bipolar II Disorder, Cyclothymic Disorder, Major Depressive Disorder, Persistent Depressive Disorder, Premenstrual Dysphoric Disorder, Anxiety, post-traumatic stress disorder, and/or an addiction disorder.

In other aspects, the health condition is a cancer comprising lung cancer, gastrointestinal cancer, salivary cancer, oropharyngeal cancer, tongue cancer, salivary cancer, esophageal cancer, hepatoma, liver cancer, gastric cancer, pancreatic cancer, gallbladder cancer, colorectal cancer, renal cell carcinoma, bladder cancer, prostate cancer, multiple endocrine neoplasia, cervical cancer, ovarian cancer, endometrial cancer, uterine cancer, vulvar cancer, breast cancer, Paget's disease, skin cancers, basal cell carcinoma, squamous cell carcinoma, melanoma, head and neck cancers, lymphoma, Hodgkin's lymphoma, non-Hodgkin's lymphoma, multiple myeloma, smoldering myeloma, Waldenstrom's macroglobulinemia, lymphoma, leukemia, glioma, glioblastoma multiforme, retinal cancer, meningeal cancer, sarcoma, osteosarcoma, bone cancer, parathyroid cancer, adrenal cancer, squamous cell cancer, pheochromocytoma, adenocarcinoma, mucinous cancer, Fibrolamellar carcinoma, Gastric adenocarcinoma and proximal polyposis of the stomach, hereditary diffuse gastric cancer, SDH-deficient gastrointestinal stromal tumor, rare endocrine tumors, adrenocortical carcinoma, anaplastic thyroid cancer, medullary thyroid cancer, and/or paraganglioma.

In another embodiment, a method is provided for detecting the presence of at least one SNP in a subject to assess a risk of having or developing a health condition related to inflammasome NLRP3 dysregulation, RAAS dysregulation, KKS dysregulation, protease dysregulation, and/or cytokine dysregulation, comprising the steps of:
i) collecting a sample from the subject;
ii) detecting the presence of at least one SNP within a gene for a modulator of inflammasome NLRP3 dysregulation, RAAS dysregulation, KKS dysregulation, protease dysregulation, and/or cytokine dysregulation, wherein the gene is comprising
   ACE, ACE2, ADAM17, ADAM9, ADAMTS13, ADORA2A, AGT, AGTR, AGTR1, AGTR2, AIM2, ANAPC1, ANO6, BDKRB1, BDKRB2, BID, BIRC2, BIRC3, BIRC5, BIRC6, BRCC3, BTK, BTK, CAMK2A, CAMP, CAPN1, CAPN2, CASP1, CASP10, CASP11, CASP12, CASP14, CASP2, CASP3, CASP4, CASP5, CASP6, CASP7, CASP8, CASP9, CAST, CAT, CBL, CCL2, CD209, CFL2, CFLAR, CFLAR-AS1, CHMP4A, CHMP4B, CHMP4C, CHUK, CLEC4M, CSTA, CTSB, CTSC, CTSD, CTSF, CTSG, CTSH, CTSK, CTSL, CTSO, CTSS, CTSV, CTSX, CTSZ, CXCL10, CXCL2, CXCL3, CYLD, DDX15, DDX19, DDX5, DNMT1, DPP4, FADD, FURIN, GSDMA, GSDMB, GSDMC, GSDMD, GSDME, GSPT1, GSPT2, H1-1, HIF1A, HIF1AN, HIF3A, HMGB1, HSP90, HSP90AA1, HSP90AB4P, HSP90B1, HSP90B2P, IFIT1, IFNA1, IFNAR1, IFNAR2, IFNLR1, IKBKB, IKBKE, IKBKG, IKK, IL10, IL10RA, IL10RB, IL15, IL17, IL17A, IL18, IL18BP, IL1B, IL1R1, IL1RL1, IL21, IL9, IRF3, IRF7, KEAP, KL, KLKB1, KNG1, LRRK2, LTB4R, MAP3K7, MAPK1, MAPK3, MAPKAPK2, MAS1, MAVS, MIR20A, MIR20B, MIR21, MIR223, MIR30C, MIR30C1, MIR30C5, MIR495, MIR9, MLKL, MME, NAMPT, NEK6, NEK7, NEK9, NFATC3, NFE2L2, NFKB, NLRC3, NLRC4, NLRC5, NLRP1, NLRP10, NLRP11, NLRP12, NLRP13, NLRP14, NLRP2, NLRP3, NLRP4, NLRP5, NLRP6, NLRP7, NLRP8, NLRP9, NLRX1, NMNAT1, NOX, NOX2, NOX4, NR1H4, P2RX1, P2RX2, P2RX2, P2RX3, P2RX3, P2RX4, P2RX5, P2RX5-TAX1BP3, P2RX6, P2RX6P, P2RX7, P2RY1, P2RY10, P2RY11, P2RY12, P2RY13, P2RY14, P2RY2, P2RY4, P2RY6, P2RY8, PADI4, PARP, PARP1, PELI1, PELI2, PELI3, PEP, PRCP, PYCARD, RIPK1, RIPK2, RIPK3, RNF31, SENP6, SENP7, SERPINA1, SERPINA4, SESN2, SIRT1, SIRT2, SLC44A2, SOCS1, SOD, SOD1, SUGT1, TAB1, TAB3, TBK1, TBKBP1, TF, TFP1, TFPI, THOP1, TICAM1, TMPRSS4, TMRPSS2, TNF, TNFAIP1, TNFAIP2, TNFAIP3, TRADD, TRAF2, TRIB3, TRIM31, TRPA4, TRPM2, TRPV1, TRPV4, USP18, VDAC1, VDR, XDH, and/or ZBP1, and combinations thereof in the sample; wherein the presence of the SNP indicates a risk of having or developing a health condition related to inflammasome NLRP3 dysregulation, RAAS dysregulation, KKS dysregulation, protease dysregulation, and/or cytokine dysregulation in the subject.

In some aspects, the health condition is SARS-CoV-2 infection and/or COVID-19.

In some aspects, the SNP is selected from the group consisting of: 84R polymorphism of TRIB3, Q705k, rs4612666, rs35829419, rs10754558 polymorphism in NLRP3, rs2043211 polymorphism in PYCARD, 489C>T as a gain-of-function polymorphism of the P2X7R, and combinations thereof.

In some embodiments, a method is provided for selectively treating a subject with a higher risk for having or developing a health condition related to inflammasome NLRP3 dysregulation, RAAS dysregulation, KKS dysregulation, protease dysregulation, and/or cytokine dysregulation comprising the steps of:
a) detecting at least one SNP associated with a higher risk for having or developing a health condition related to inflammasome NLRP3 dysregulation, RAAS dysregulation, KKS dysregulation, protease dysregulation, and/or cytokine dysregulation in the subject by a method comprising the steps of:
   i) obtaining a sample from a subject;
   ii) detecting the presence of at least one SNP in a gene comprising:
      ACE, ACE2, ADAM17, ADAM9, ADAMTS13, ADORA2A, AGT, AGTR, AGTR1, AGTR2, AIM2, ANAPC1, ANO6, BDKRB1, BDKRB2, BID, BIRC2, BIRC3, BIRC5, BIRC6, BRCC3, BTK, BTK, CAMK2A, CAMP, CAPN1, CAPN2, CASP1, CASP10, CASP11, CASP12, CASP14, CASP2, CASP3, CASP4, CASP5, CASP6, CASP7, CASP8, CASP9, CAST, CAT, CBL, CCL2, CD209, CFL2, CFLAR, CFLAR-AS1, CHMP4A, CHMP4B, CHMP4C, CHUK, CLEC4M, CSTA, CTSB, CTSC, CTSD, CTSF, CTSG, CTSH, CTSK, CTSL, CTSO, CTSS, CTSV, CTSX, CTSZ, CXCL10, CXCL2, CXCL3, CYLD, DDX15, DDX19, DDX5, DNMT1, DPP4, FADD, FURIN, GSDMA, GSDMB, GSDMC, GSDMD, GSDME, GSPT1, GSPT2, H1-1, HIF1A, HIF1AN, HIF3A, HMGB1, HSP90, HSP90AA1, HSP90AB4P, HSP90B1, HSP90B2P, IFIT1, IFNA1, IFNAR1, IFNAR2, IFNLR1, IKBKB, IKBKE, IKBKG, IKK, IL10, IL10RA, IL10RB, IL15, IL17, IL17A, IL18, IL18BP, IL1B, IL1R1, IL1RL1, IL21, IL9, IRF3, IRF7, KEAP, KL, KLKB1, KNG1, LRRK2, LTB4R, MAP3K7, MAPK1, MAPK3, MAPKAPK2, MAS1, MAVS, MIR20A, MIR20B, MIR21, MIR223, MIR30C, MIR30C1, MIR30C5, MIR495, MIR9, MLKL, MME, NAMPT, NEK6, NEK7, NEK9, NFATC3, NFE2L2, NFKB, NLRC3, NLRC4, NLRC5, NLRP1, NLRP10, NLRP11, NLRP12, NLRP13, NLRP14, NLRP2, NLRP3, NLRP4, NLRP5, NLRP6, NLRP7, NLRP8, NLRP9, NLRX1, NMNAT1, NOX, NOX2, NOX4, NR1H4, P2RX1, P2RX2, P2RX2, P2RX3, P2RX3, P2RX4, P2RX5, P2RX5-TAX1BP3, P2RX6, P2RX6P, P2RX7, P2RY1, P2RY10, P2RY11, P2RY12, P2RY13, P2RY14, P2RY2, P2RY4, P2RY6, P2RY8, PADI4, PARP, PARP1, PELI1, PELI2, PELI3, PEP, PRCP, PYCARD, RIPK1, RIPK2, RIPK3, RNF31, SENP6, SENP7, SERPINA1, SERPINA4, SESN2, SIRT1, SIRT2, SLC44A2, SOCS1, SOD, SOD1, SUGT1, TAB1, TAB3, TBK1, TBKBP1, TF, TFP1, TFPI, THOP1, TICAM1, TMPRSS4, TMRPSS2, TNF, TNFAIP1, TNFAIP2, TNFAIP3, TRADD, TRAF2, TRIB3, TRIM31, TRPA4, TRPM2, TRPV1, TRPV4, USP18, VDAC1, VDR, XDH, and/or ZBP1, and combinations thereof in the sample, wherein the presence of the at least one SNP confirms a higher risk for having or developing a health condition related to inflammasome NLRP3 dysregulation, RAAS dysregulation, KKS dysregulation, protease dysregulation, and/or cytokine dysregulation; and
b) treating the subject with a therapy for the treatment of a health condition related to inflammasome NLRP3 dysregulation, RAAS dysregulation, KKS dysregulation, protease dysregulation, and/or cytokine dysregulation, wherein the treatment comprises personalized pharmacogenomics and/or nutrigenomics therapies comprising administration of any compound listed in Tables 1, 2, 3, or 4 in FIGS. 1, 2, 3, and 4.

In some aspects, the health condition is SARS-CoV-2 infection and/or COVID-19.

In another embodiment, a packaged kit is provided for use in the treatment of a health condition related to inflammasome NLRP3 dysregulation, RAAS dysregulation, KKS dysregulation, protease dysregulation, and/or cytokine dysregulation in a subject in need thereof comprising:
a) any of the pharmaceutical compositions disclosed herein;
b) a container housing the pharmaceutical composition during storage and prior to administration; and
c) instructions for carrying out administration of the pharmaceutical composition in a manner effective to treat the health condition.

Additional features of the invention will be or will become apparent to one with skill in the art upon examination of the following figures and detailed description. It is intended that all such additional features and advantages be included within this description, be within the scope of the invention, and be protected by the accompanying claims.

### BRIEF DESCRIPTION OF THE DRAWINGS

Having thus described the subject matter of the present invention in general terms, reference will now be made to the accompanying drawings, which are not necessarily drawn to scale, and wherein:

FIG. 1 shows Table 1, which provides inhibitors of one or many activators of inflammasome priming and/or activation and/or hyperactivation;

FIG. 2 shows Table 2, which provides a non-limiting list of modulators of cytokine dysregulation;

FIG. 3 shows Table 3, which provides a non-limiting list of modulators of Renin Aldosterone Angiotensin System (RAAS) dysregulation and modulators of Kallikrein-Kinin System (KKS) dysregulation;

FIG. 4 shows Table 4, which provides a non-limiting list of modulators of protease dysregulation; and

FIG. 5 shows full names for abbreviated genes and proteins described in the present application.

### DETAILED DESCRIPTION

The subject matter of the present invention now will be described more fully hereinafter with reference to the accompanying drawings, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions and the associated drawings. Therefore, it is to be understood that the subject matter of the present invention is not to be limited to the specific embodiments disclosed and that modifications and other embodiments are intended to be included within the scope of the appended claims.

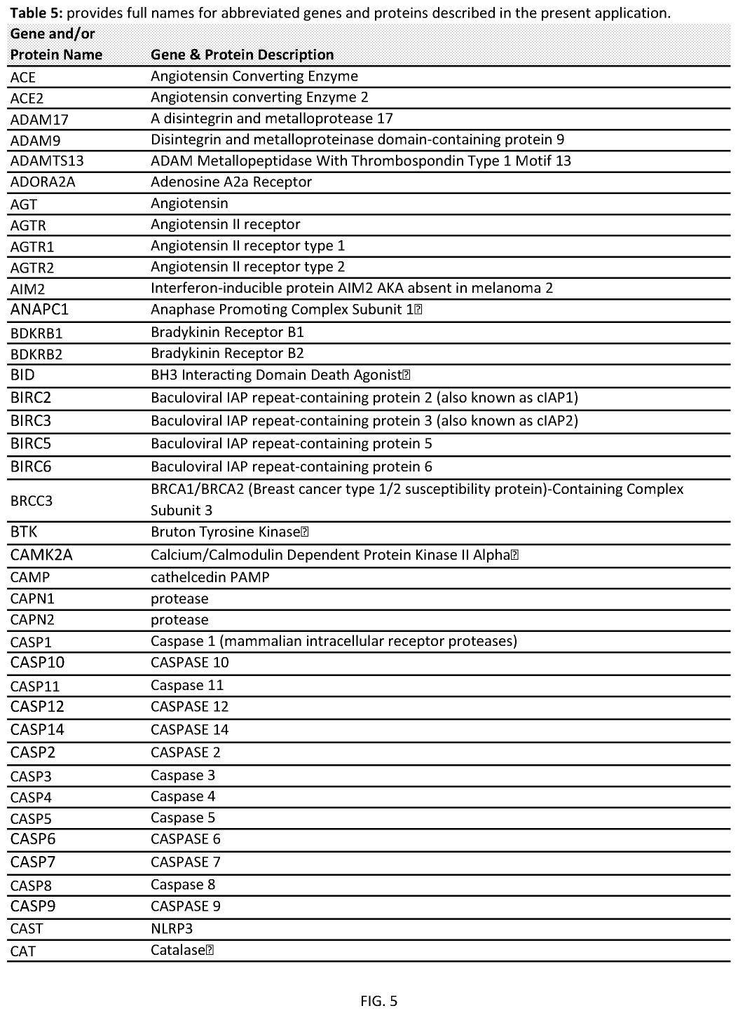

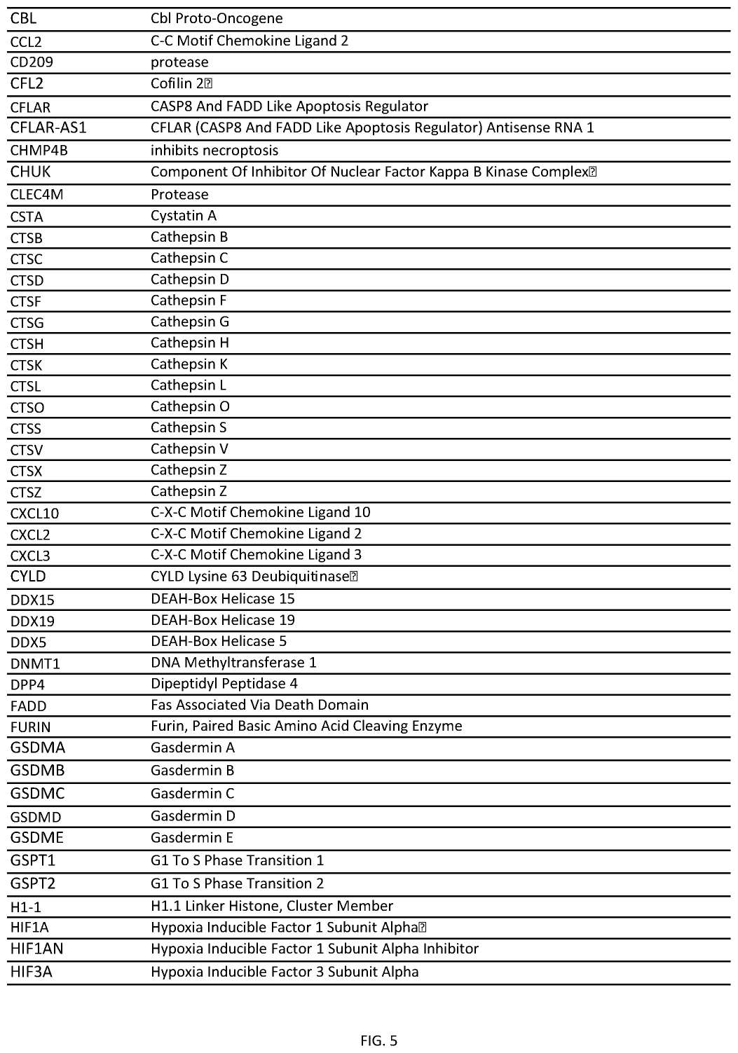

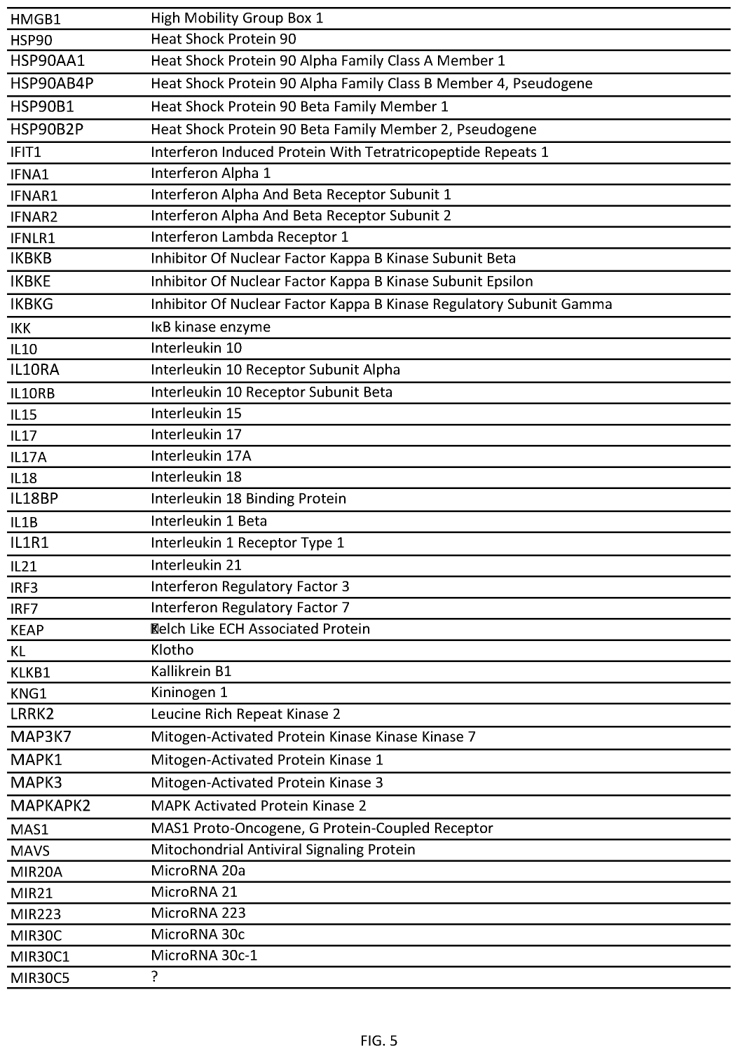

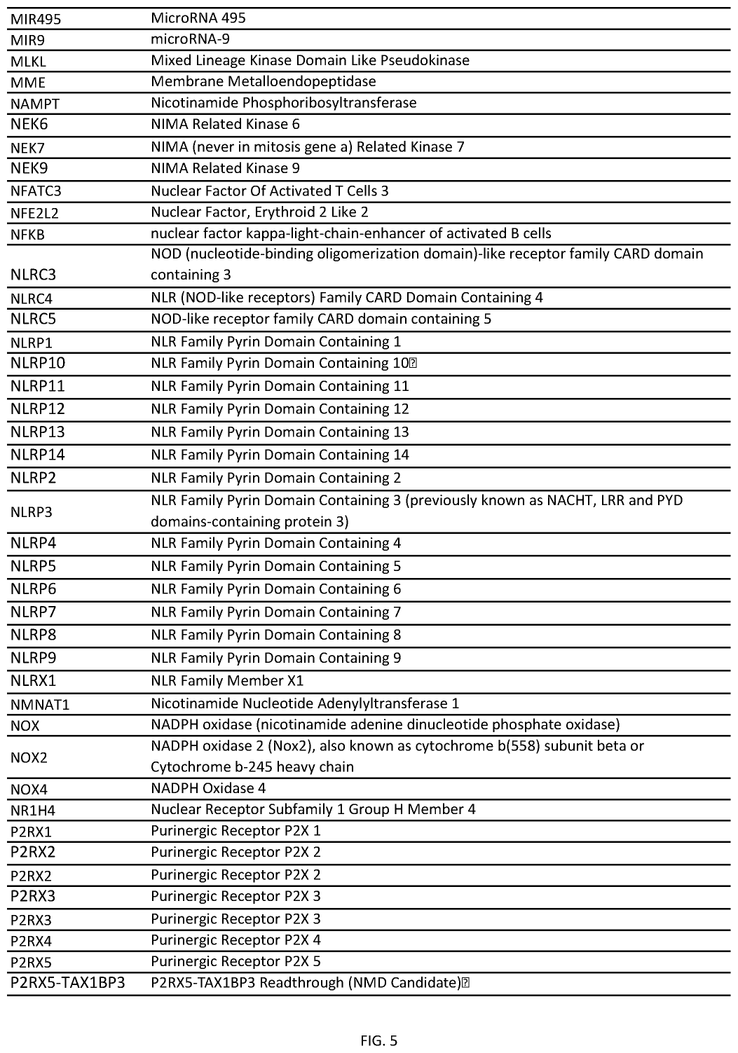

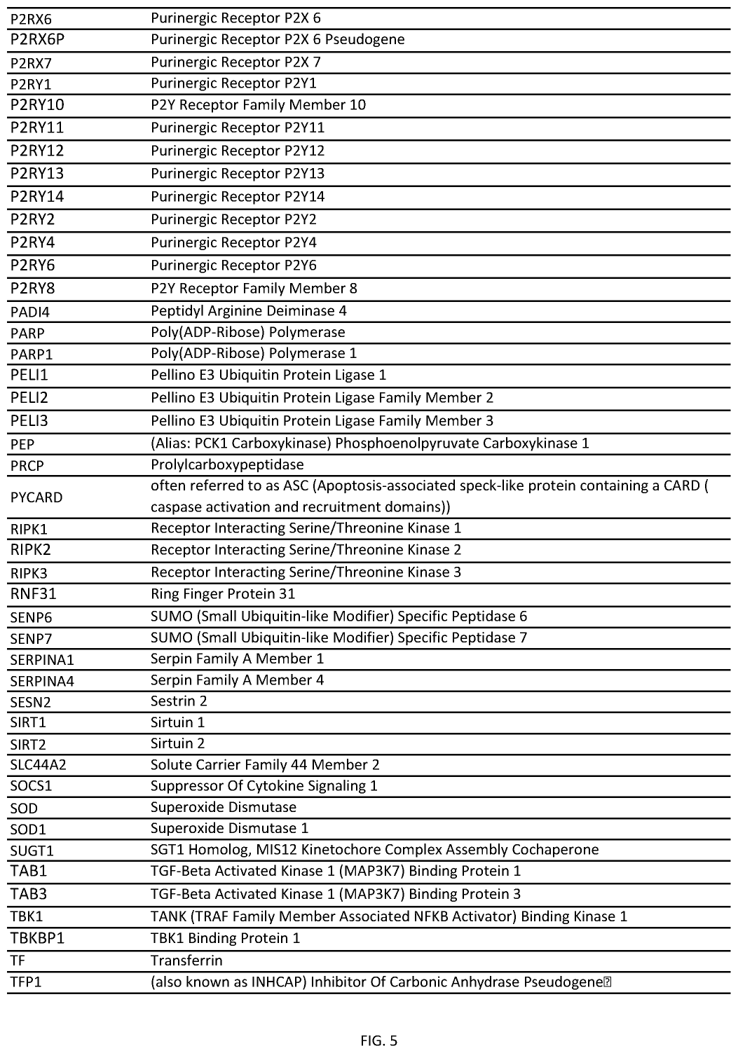

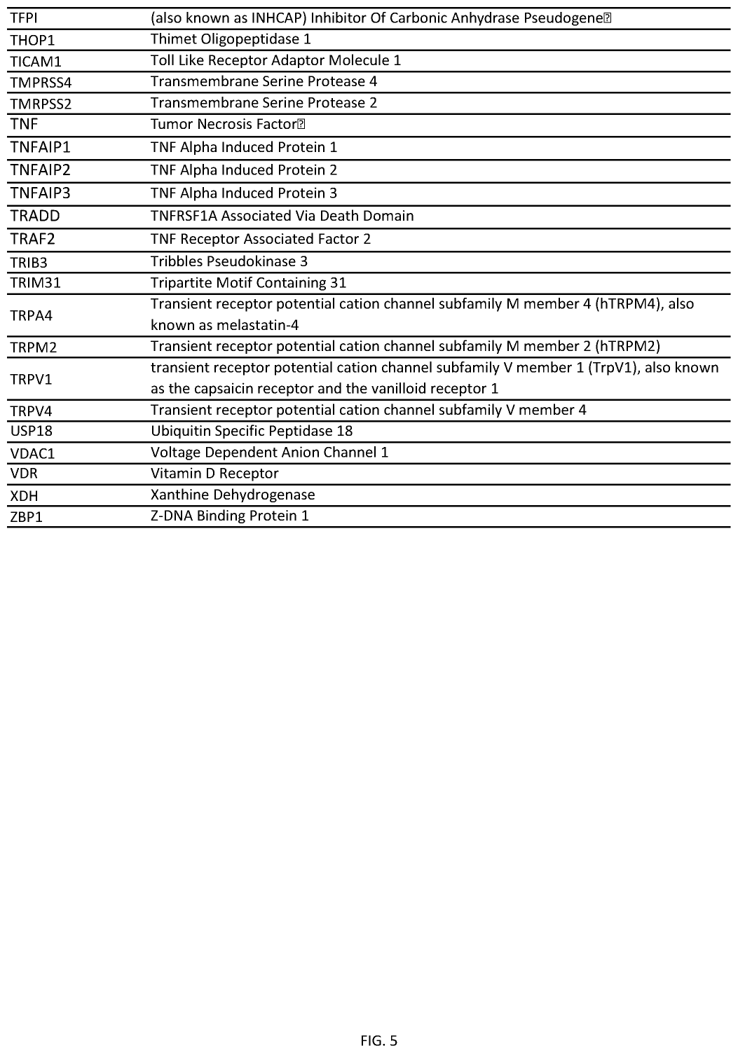

While dysregulation of NLRP3 (FIG. 5 shows full names for abbreviated genes and proteins described in the present application) inflammasome priming and activation appears to be involved in many disease processes, no approved medications for NLRP3 inhibition are currently available. Medications are available and approved to inhibit the downstream cytokine, IL-1 beta of NLRP3 (such as Canakinumab). Interleukin 1 receptor antagonists (i.e., Anakinra) are also available. These IL-1beta and Interleukin 1 receptor inhibitor drugs do not address the dysregulations of NLRP3 activation as there are many varied activators of NLRP3 inflammasome priming and/or activation which this invention addresses. Furthermore:

1) Current treatments with IL-1B inhibitors do not address the pyroptosis which is highly immunogenic and can lead to severe immune dysregulation which this invention solves.

2) Existing drugs do not prevent NLRP3 mediated Casp activation, GSDMD mediated pyroptosis, Casp-mediated IL-18 secretion, and the severe immune dysregulation, cytokine storm, and inflammation that can occur with dysregulated NLRP3 activation.

3) At present there are no approved CASp-1 inhibitors. While in preclinical studies, these compounds were withdrawn from clinical trials because of liver toxicity after long-term administration in animal studies. As such, no caspase inhibitors have yet reached Phase III.

4) Currently there are no interferon lambda drugs available. Eiger biopharmaceuticals has a pegylated-interferon-lambda in phase II clinical studies with an orphan drug status for Hepatitis D. This formulation has already been used to treat more than 3,000 people in clinical trials for hepatitis B, C and D.

5) There are several pharmaceutical drugs based on recombinant interferon I; however, they are exceedingly expensive and not available worldwide and can have severe side effects.

6) There are no currently approved treatments for ACE2 dysregulation. There are currently inhibitors of enzymes that metabolize Bradykinins such as Thiorphan, Phosphoramidon, Candoxatrilat, Entresto (sacubitril/valsartan), and sacubitril.

7) There are ACE and Neprilysin Inhibitors comprising Ecadotril (99% Neprilysin and 23% ACE inhibition), Lisinopril (83% ACE inhibition), Ecadotril and Lisinopril, Omapatrilat (>90% ACE and 53% Neprilysin inhibition). These combination drugs have higher rates of angioedema than ACE inhibitors alone. There are Angiotensin receptor blockers (ARBs) and Neprilysin inhibitors: Valsartan and candoxatril.

8) ARB's and ACE Inhibitors increase Bradykinin levels by 2-fold in arterial blood. Icatibant is a selective Bradykinin B2 antagonist. Icatibant at lower dose of (18 μg/kg/h iv for 6 h) did not attenuate the hypotensive effects of either acute or chronic administration of valsartan in sodium-deplete normotensive and hypertensive subjects. Currently there are no Bradykinin B1 antagonists approved. Several antagonists were being developed and at various stages of the drug approval process, but they were abandoned.

9) While there are treatments for RAAS dysregulation including ACE inhibitors (ACEI) and Angiotensin receptor blockers, ACEI can cause KKS dysregulation.

10) There are 2 approved drugs that inhibit TMPRSS2 comprising Camostat mesylate and Nafamostat. Camostat Mesylate is licensed in Japan and South Korea to treat pancreatitis. Nafamostat is US FDA approved and this drug is used in Asia as an anticoagulant therapy for patients undergoing continuous renal replacement therapy due to acute kidney injury. Nafamostat is effective at lower concentration than Camostat, but it is administered intravenous. In mice, camostat mesylate dosed at concentrations similar to the clinically achievable concentration in humans reduced mortality following SARS-CoV (not SARS-COV2) infection from 100% to 30-35%. In recent in vitro studies, Camostate Mesylate did not fully inhibit the SARS-COV2 viral entry as other proteases are involved in cleaving the SARScov2 S protein and allowing membrane fusion and cell entry. Camostat Mesylate is in clinical trials in Denmark now for SARS-COV2.

11) There are FDA approved DPP4 inhibitors used for the treatment of diabetes mellitus type II comprising sitagliptin, saxagliptin, linagliptin, and alogliptin. These medications can cause severe joint pains.

12) There are commercial Furin inhibitors available. The issues with Furin inhibitors instability as it can dissociate from Furin in Golgi apparatus, subcellular localization, and cytotoxicity. Because of its pleiotropic effects, variations in Furin expression levels and/or its enzymatic activity may have detrimental effects and promote the pathogenesis of a variety of disorders, including rheumatoid arthritis, amyloid dementia and cancer if systemic treatments of Furin are given.

13) Although several classes of cathepsin inhibitors are presently available, there are still some problems which will limit their use in clinics. First, cathepsin inhibitors exert toxicity in cells. When a cathepsin inhibitor is combined with target proteins by irreversible covalent bonding, its reactive groups tend to bind covalently to some biological macromolecules (proteins or DNA) and/or glutathione. Second, broad-spectrum inhibition of cathepsin B or L to protease may lead to some unpredictable side effects in clinical trials because of complex function of cathepsins in normal physiological processes. Third, some dipeptide inhibitors may exist with low bioavailability problem due to peptide nature. Hence, an inhibitor of cathepsin B or L with a sufficient selectivity over off-target proteins and other ubiquitously expressed cathepsins would negate the undesirable effects.

Because NLRP3 is activated by multiple signals as described herein, methods for preventing and/or treating health conditions related to dysregulation of NLRP3 inflammasome priming and/or activation should address multiple activators pertaining to a subject's unique state of health and disease risks.

Accordingly, as described more fully below, the present invention is directed to compositions and methods involving multiple inhibitors of NLRP3 priming and/or activation, which are administered to a subject (particularly a mammal, more particularly a human) in need thereof to prevent and to treat SARS-COV2 infections. SARS-COV2 infections have a more deleterious presentation in patients with existing chronic disease which would lead to many activators of NLRP3 activation which this invention solves.

Also as described more fully below, the present invention is further directed to compositions and methods involving multiple inhibitors of NLRP3 priming and/or activation are used and administered to a subject (particularly a mammal, more particularly a human) in need thereof to prevent and to treat chronic and/or acute health conditions related to inflammasome NLRP3 dysregulation, RAAS dysregulation, KKS dysregulation, protease dysregulation, and/or cytokine dysregulation including but not limited to: infectious disease, age related chronic disease, immune disease, cardiovascular disease, neurodegenerative disease, renal disease, autoimmune disease, gastrointestinal disease, thromboembolic disease, ophthalmic disease, pulmonary disease, genitourinary disease, dermatologic disease, metabolic disease, and/or mitochondrial disease.

The present invention also relates generally to compositions and methods for inducing artificial biological mimicry in a subject (particularly a mammal, more particularly a human) to mimic the favorable immune advantages present in a bat and thereby prevent and/or treat COVID-19 and other diseases by administering to the subject a combination of active agents as described more fully below. The compositions and methods of the invention are directed to artificially inducing molecular mimicry in the subject by administering any of the compositions disclosed herein to the subject to prevent and/or treat a health condition related to inflammasome NLRP3 dysregulation, RAAS dysregulation, KKS dysregulation, protease dysregulation, and/or cytokine dysregulation.

The compositions and methods of the invention address the strong need that exists for effective and safe treatments to inhibit (partially or completely) multiple activators of NLRP3 inflammasome priming and activation as they are involved in a variety of diseases including COVID19 and the SARS-COV2 infections which has caused a global pandemic. Furthermore, these therapies will abrogate the many cellular dysregulation that leads to NLRP3 inflammasome activation.

### Pharmaceutical Compositions

In one embodiment, a pharmaceutical composition is provided comprising:
a) two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3; and/or
b) one or more additional active agents comprising:
   i) one or more modulators of Renin Aldosterone Angiotensin System (RAAS) dysregulation, comprising an inhibitor of the ACE/Angiotensin II arm of RAAS and/or an activator of the ACE2/Ang(1-7)/Mas1 arm of RAAS comprising an inhibitor of ACE, an inhibitor of Angiotensin II activation, an activator of ACE2, an activator of Ang(1-7), an inhibitor of AT1R, an inhibitor of ACE gene expression and/or activity, an inhibitor of Renin, an inhibitor Chymase, a mast cell stabilizer, an inhibitor of histamin, an inhibitor of neprolysin, a combined inhibitor of At1R and Neprilysin, an activator of ADAM17 activity and/or gene expression, an activator of MAS1, an activator of MAS1 gene expression and/or activity, an activator of AT2R, and/or an activator of AT2R;
   ii) one or more modulators of Kallikrein-Kinin System (KKS) dysregulation;
   iii) one or more modulators of protease dysregulation; and/or
   iv) one or more modulators of cytokine dysregulation;
      wherein the pharmaceutical composition further comprises a pharmaceutically acceptable carrier; and wherein the active agents are each present in therapeutically effective amounts to prevent and/or treat a health condition related to inflammasome NLRP3 dysregulation, RAAS dysregulation, KKS dysregulation, protease dysregulation, and/or cytokine dysregulation in a subject in need thereof.

In other aspects, the pharmaceutical composition is further characterized wherein:
a) the two or more active agents of 1(a) comprise a full or partial inhibitor of NLRP3, NLRC4, NLRP1, NLRP6, IFI16, Pyrin, and/or AIM2;
b) the one or more modulators of KKS dysregulation comprise an inhibitor of KKS activation comprising an inhibitor of Neprilysin, a combined inhibitor of At1R and Neprilysin, an inhibitor of ACE and Neprilysin, an inhibitor of Bradykinin B2, an inhibitor of Bradykinin B1, inhibitor of Kallikrein B1(KLKB1), an inhibitor of Kininogen 1 (KNG1), an inhibitor of High-molecular-weight kininogen (HMWK), an inhibitor of Low-molecular-weight kininogen (LMWK), an activator of Kallistatin, an inhibitor of histamine, an activator of SERPINA1, an activator of SERPINA4, and/or an activator of Cystatin A;
c) the one or more modulators of protease dysregulation comprise an inhibitor of a protease comprising Furin, Cathepsin B, Cathepsin C, Cathepsin D, Cathepsin F, Cathepsin G, Cathepsin K, Cathepsin O, Cathepsin S, Cathepsin L, Cathepsin V, Cathepsin X, Cathepsin Z, TMPRSS2, CLEC4M, CAPN2, CAPN1, CD209, and/or TPRSS4, and/or the one or more modulators of protease dysregulation comprise an activator of CAST (calpastatin) gene activity and/or gene expression, an activator of Calpastatin, and/or an activator of Cystatin A; and
d) the one or more modulator of cytokine dysregulation comprises an activator of IFN-λ, an activator of Type I interferon, an inhibitor of CXCs, an inhibitor of CCL2, and/or an activator of IL-10.

In other aspects, the two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3 comprise an activator of cellular autophagy, an activator of mitophagy, an activator of purine salvage pathways, an activator of mitochondrial cellular bioenergetics, an activator of NRF2 gene expression and/or activity, an activator of SIRT1, an activator of ACE2/MAS/ANG(1-7), an ANG(1-7) mimetic, an activator of NLRP3 ubiquitination and degradation, an activator of dopamine receptor-1 (DRD1), and/or an activator of NAD+, further wherein the activator of NAD+ comprises an activator of nicotinamide salvage pathways, a precursor of NAD+, an agonist of NAD+, an NAD+ mimetic, an inhibitor of ROS and oxidative stress, an inhibitor of PARP1, an activator of NAD+ dependent SIRT2, an activator of mitochondrial uncoupling, an activator of mitochondrial biogenesis, and/or an activator of transcription and/or activity of genes comprising SIRT2, SIRT1, NAMPT, and/or NMNAT1.

In other aspects, the two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3 also comprise one or more full or partial inhibitors of one or more priming or activation signals of one or more activated Pattern Recognition Receptors (PRRs) and/or a modulator of PRR-activation-induced dysregulation in inflammasome signal transduction pathways, wherein the PRR is an inflammasome.

In other aspects, the one or more full or partial inhibitors of one or more priming or activation signals of one or more activated PRRs comprises a full or partial inhibitor of NLRP1, NLRX1, NLRC5; and/or cGAS-STING.

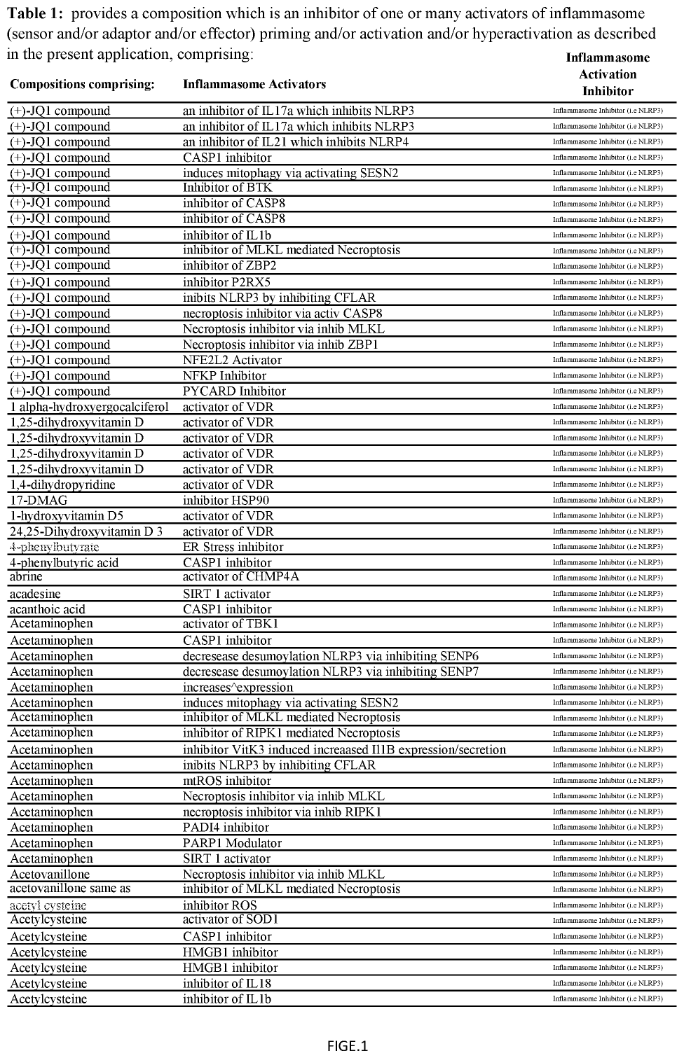

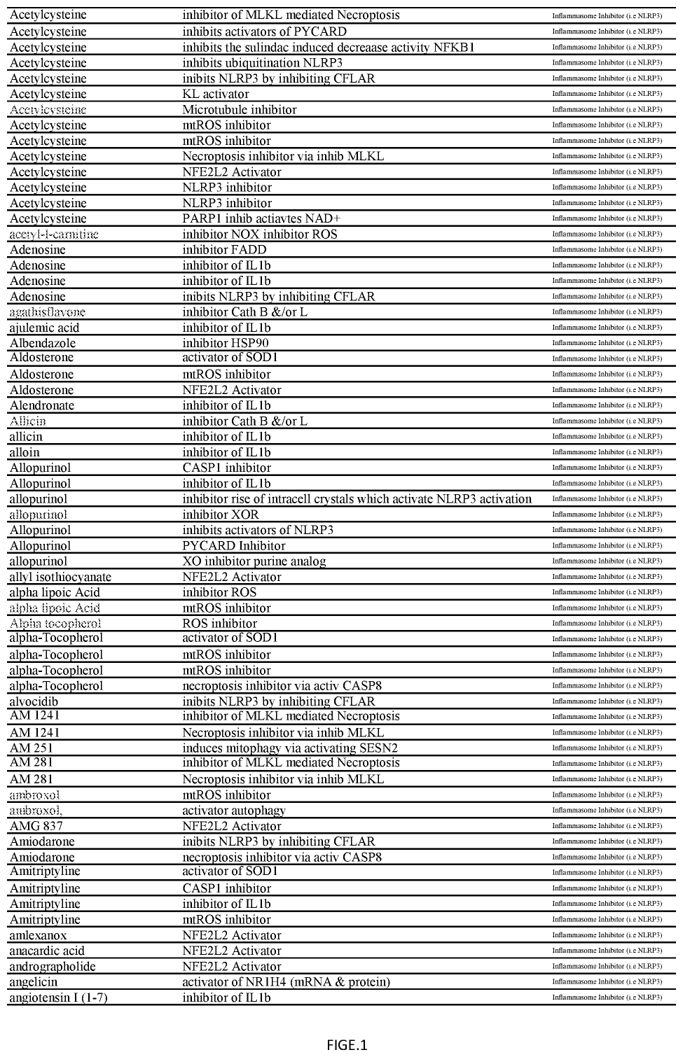

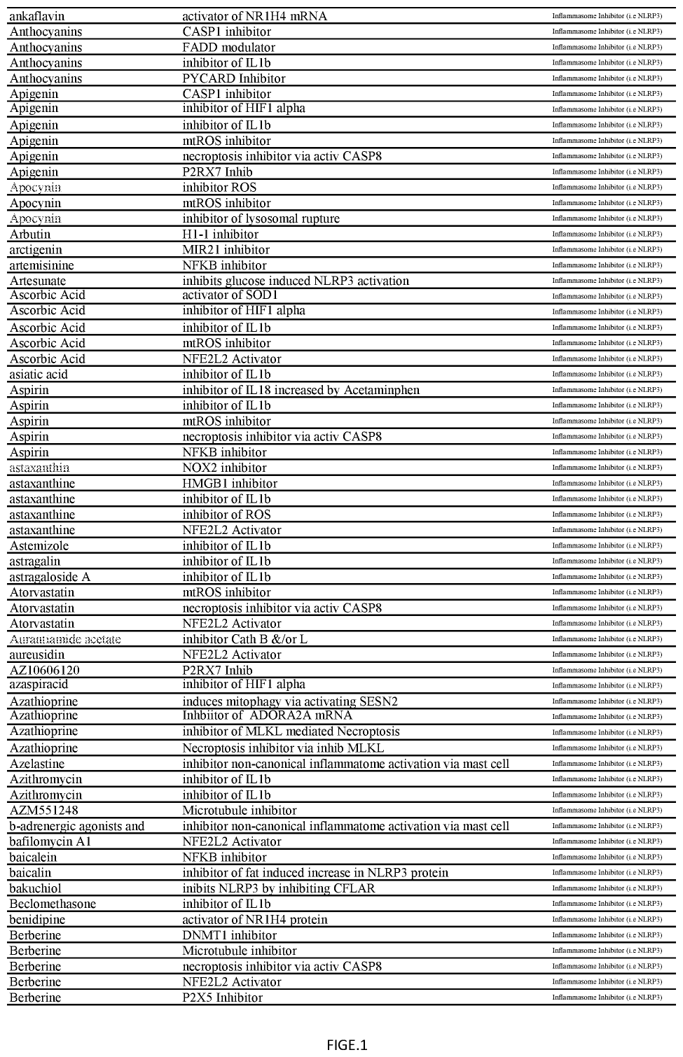

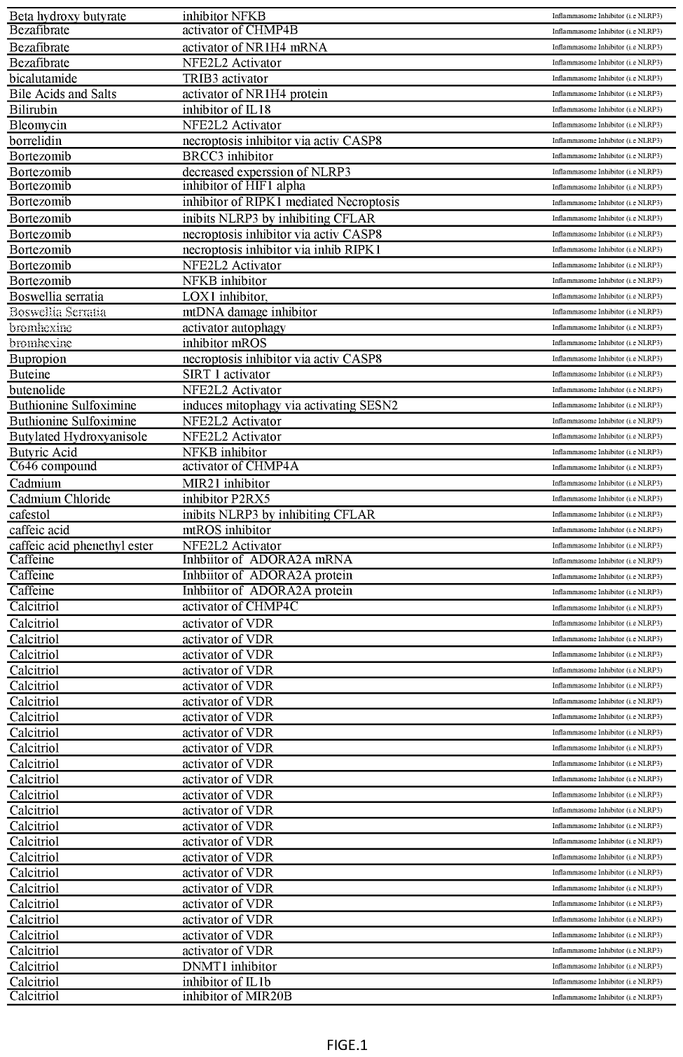

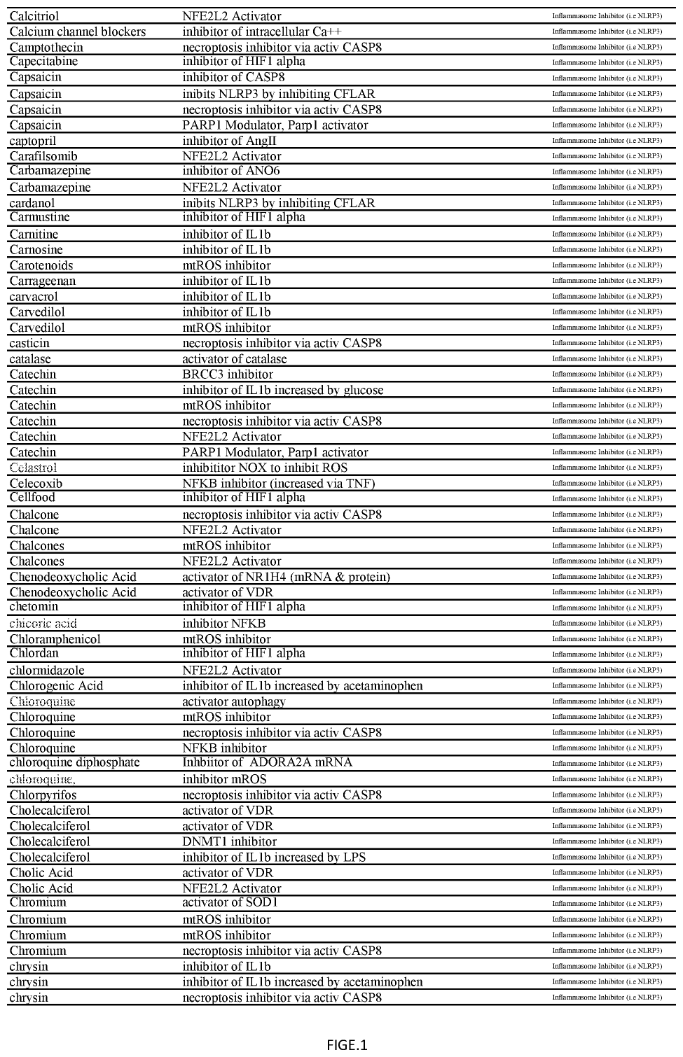

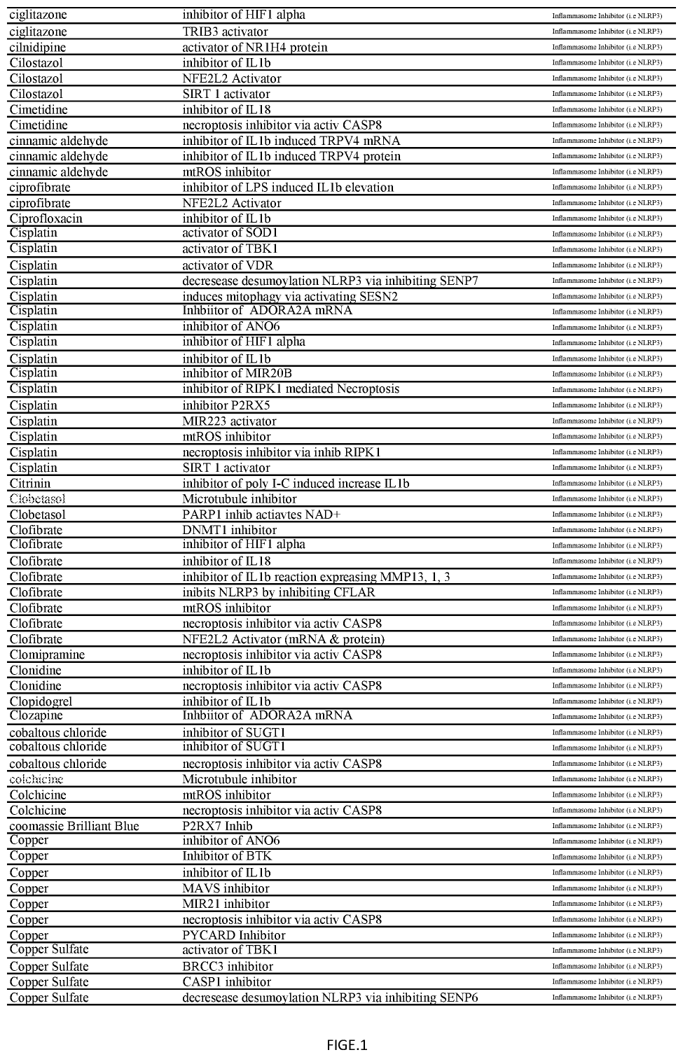

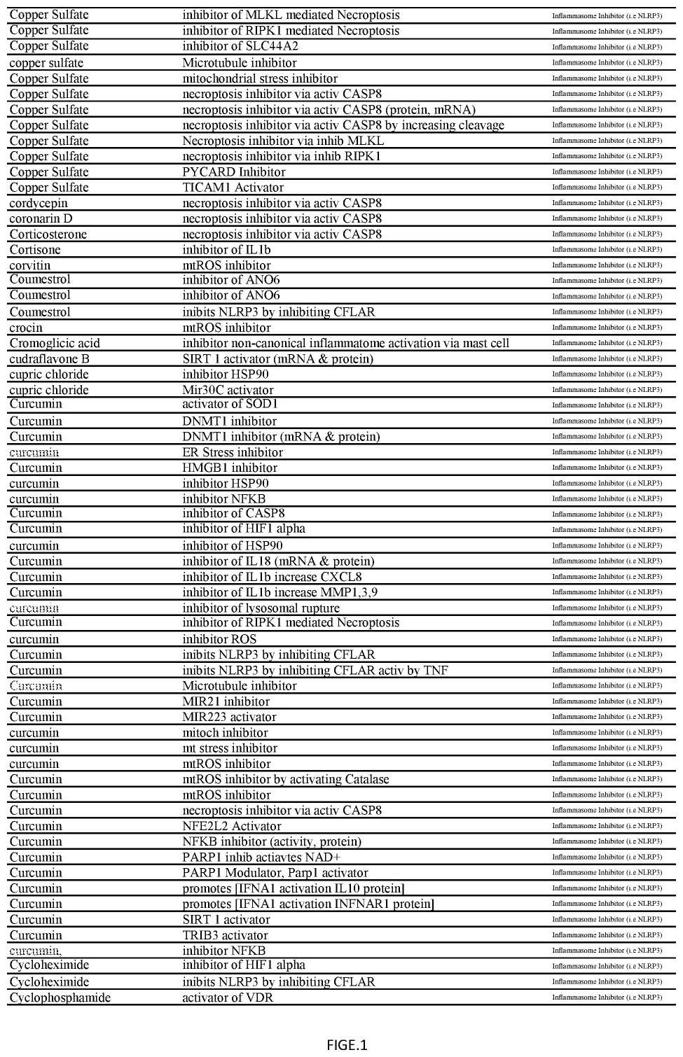

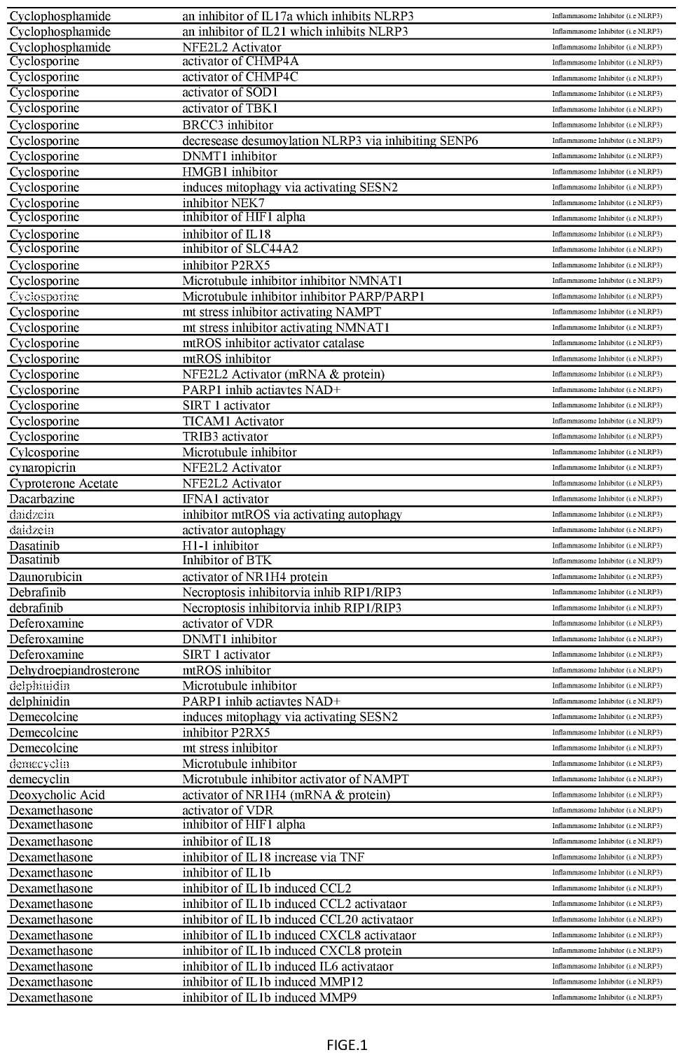

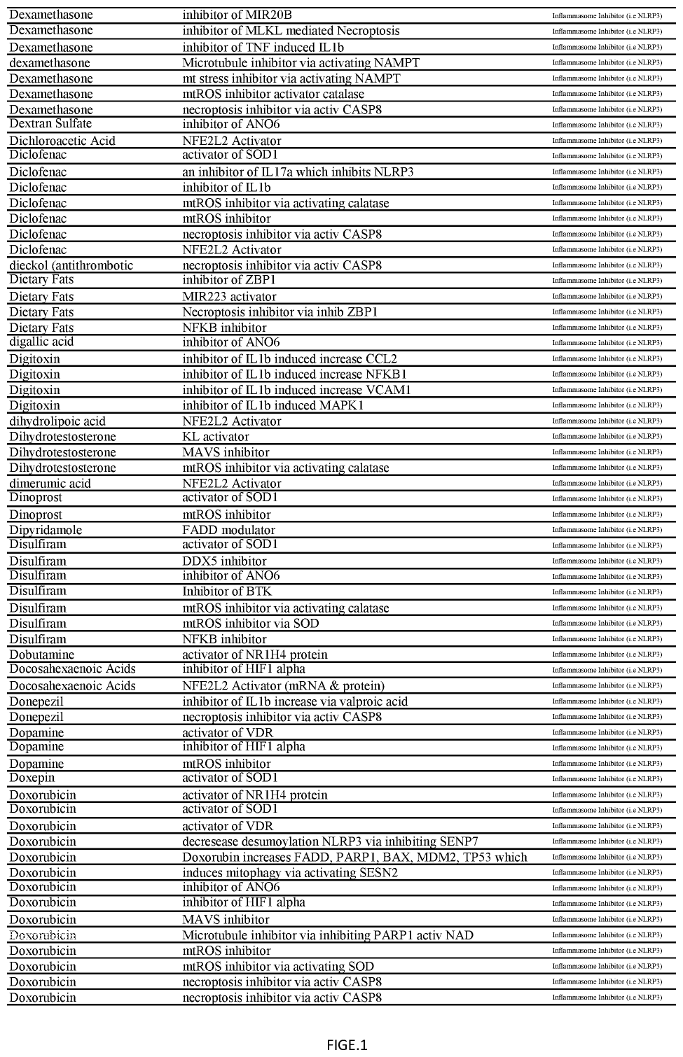

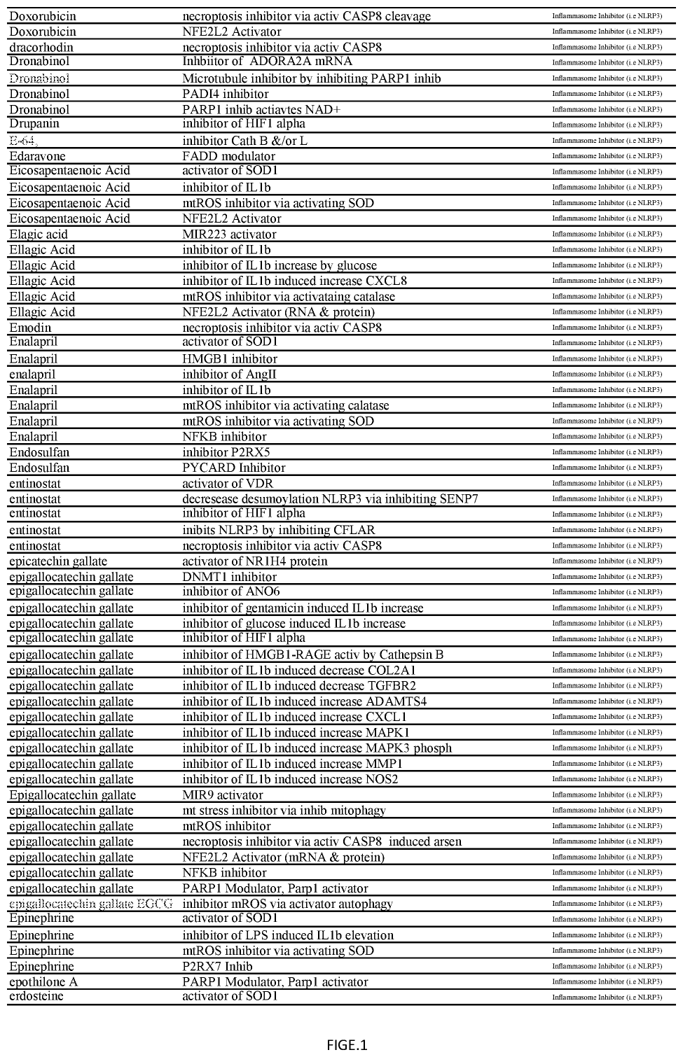

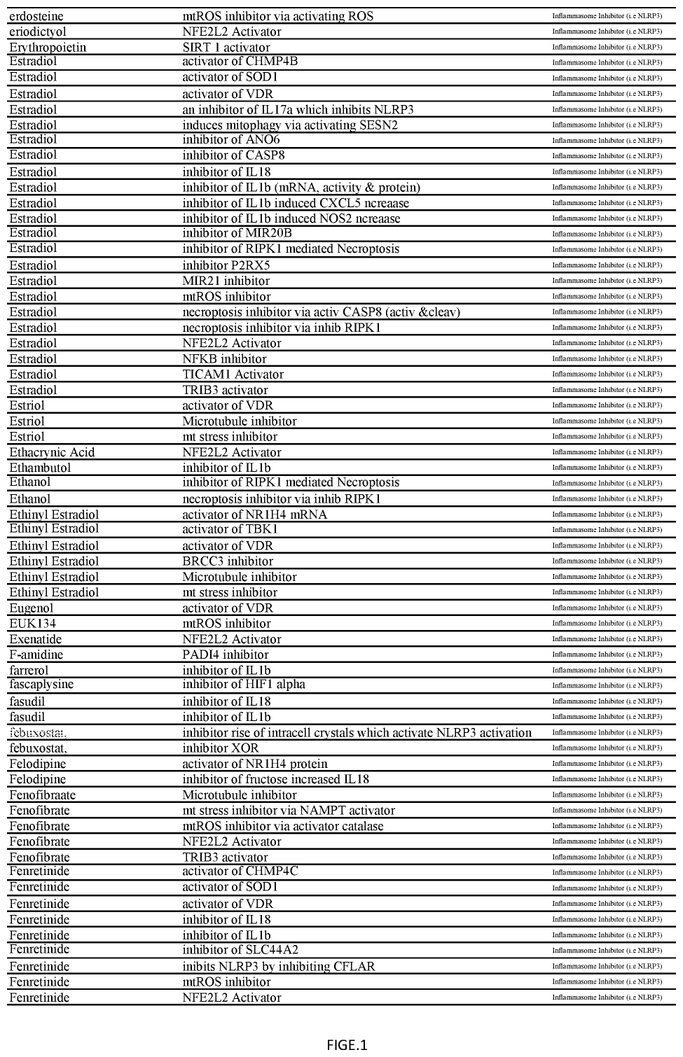

In other aspects, the one or more full or partial inhibitors of one or more priming or activation signals of one or more activated PRRs comprises a full or partial inhibitor of NLRP3, NLRC4, NLRP1, NLRP6, IFI16, Pyrin, and/or AIM2 and/or their related adaptors or effectors, comprising a phenothiazine dye, 4-phenylbutyrate (4-PBA), 4-phenylbutyric acid, sodium phenyl butyrate, methylene blue, glycine, a xanthine oxidoreductase inhibitor, a bile acid, taurodeoxychcolic acid (TUDCA), naltrexone, a microtubule disruptor/inhibitor, demcolcine, colchicine, an HMG-CoA reductase inhibitors/Statin, simvastatin, an antihistamine, a fibric acid derivative, metformin, propylthiouracil, thiazolidinedione, ketamine, nicotine, ivermectin, albendazole, an H/K inhibitor, amitriptyline, a corticosteroid, niclosamide, riluzole, sunitinib, valproic acid, glibenclamide bortezomib, an antibiotic, a tetracycline, minocycline, gentamycine, levofloxacine, cyclosporin, diminazen or diminazene aceturate, a calcium channel blocker, felodipine, a leukotriene D4 inhibitor, an ACE inhibitor, enalapril, an Angiotensin Receptor 1 inhibitor, resveratrol, anthocyanin, taurine, thioctic acid, rosmarinic acid, sulforaphane, celastrol, berberine, Epigallocatechin gallate, catechin, glucosamine, melatonin, pregnenolone, testosterone, estradiol, estriol, mangiferin, linarin, fisetin, rutin, rutecarpin, selenium, selenomethionin, progesterone, n-acetyl cysteine, acetyl cysteine, Mitotempo, tempol, catalase, SOD, apigenin, quercetin, tempol, silybin/silymarin, apocynin, a zinc compound, silybin, curcumin, alpha lipoic acid, genistein, gedunin, astaxanthin, copper, vitamin C, vitamin E, Vitamin D, calcitriol, niacin, tretinoin, acetaminophen, pregnenolone, a non-steroidal anti-inflammatory drug, β-Ursolic acid, Schinol, glycyrrhizic acid, trichostatinA, docasohexaenoic acid, pathenolide and/or any compound listed in Tables 1, 2, 3, or 4 in FIGS. 1, 2, 3, and 4.

In other aspects, the health condition is SARS-CoV-2 infection and/or COVID-19.

In other aspects, the pharmaceutical composition comprises one or more modulators of SARS-CoV-2 associated RAAS dysregulation and one or more modulators of KKS dysregulation, wherein the one or more modulators of SARS-CoV-2 associated RAAS dysregulation and the one or more modulators of KKS dysregulation are each present in therapeutically effective amounts to prevent or treat SARS-CoV-2 infection and/or COVID-19 in a subject in need thereof.

In other aspects, the pharmaceutical composition comprises one or more inhibitors of SARS-CoV-2 associated inflammasome activators, wherein the inflammasome is NLRP3, AIM2, and/or NLRP1, and further wherein the one or more inhibitors of SARS-CoV-2 associated inflammasome activators is present in therapeutically effective amounts to prevent or treat SARS-CoV-2 infection and/or COVID-19 in a subject in need thereof.

In other aspects, the pharmaceutical composition comprises one or more inhibitors of SARS-CoV-2 associated cytokine dysregulation, comprising a modulator of SARS-CoV-2 associated Interferon I and/or III antiviral response.

In other aspects, the modulator of SARS-CoV-2 associated Interferon antiviral response is an activator of Interferon III (Lambda) comprising:
a) an activator/stimulator of IFN-λ;
b) a pegylated-interferon-lambda;
c) an activator of IRF3 gene expression and/or activity;
d) an activator of IRF7 gene expression and/or activity;
e) an activator of Interferon Lambda receptor INFLR1 gene expression and/or activity for early infection or an inhibitor of Interferon Lambda receptor INFLR1, IFR3, IRF7, and/or IL10RB for after early infection; and/or
f) an activator of Interferon Lambda receptor IL10RB gene expression and/or activity.

In other aspects, the modulator of SARS-CoV-2 associated Interferon antiviral response is an activator of Il-15 comprising:
a) an activator of IL-15 gene mRNA expression and/or activity;
b) an activator of Interferon lambda for early infection or an inhibitor of Interferon Lambda receptor INFLR1, IFR3, IRF7, and/or IL10RB for after early infection;
c) an activator of IL-12 gene expression and/or activity; and/or
d) an activator of IL-21 gene expression and/or activity.

In other aspects, the modulator of SARS-CoV-2 associated Interferon antiviral response is an activator of Interferon I comprising:
i) an activator of Interferon1 beta;
ii) a pegylated-interferon-1beta;
iii) an Interferon Lambda mimetic, agonist, and/or activator;
iv) an Interferon I mimetic, agonist, and/or activator;
v) an activator of IRF3 gene expression and/or activity; and/or
vi) an activator of expression of interferon 1 receptor gene IFNAR1 or IFNAR2.

In other aspects, the modulator of SARS-CoV-2 associated Interferon antiviral response is an inhibitor of IFNAR1 or IFNAR2, comprising:
i) an inhibitor of IFNAR1 gene expression and/or activity, and
ii) an inhibitor of IFNAR2 gene expression and/or activity.

In other aspects, the modulator of SARS-CoV-2 associated Interferon antiviral response is an activator of IL-10 comprising:
i) an inhibitor of AT1R; and/or
ii) an activator of Heat Shock Factor 1.

In other aspects, the pharmaceutical composition comprises:
a) the two or more full or partial inhibitors of one or more priming or activation signals of inflammasome NLRP3;
b) an activator of Interferon beta and/or IFN-λ for early infection;
c) an inhibitor of Interferon beta and/or IFN-λ for late infection; and
d) an activator of anti-inflammatory interleukin-10 (IL-10)-related cytokines comprising IL-10, IL-19, IL-20, IL-22, IL-24, and IL-26.

In other aspects, the pharmaceutical composition comprises:
a) the two or more full or partial inhibitors of one or more priming or activation signals of inflammasome NLRP3; and
b) the one or more modulators of cytokine dysregulation comprises an activator of IFN-β, an activator of IFN-λ, and an activator of IL-10.

In other aspects, the pharmaceutical composition modulates inflammasome priming and/or activation, wherein the pharmaceutical composition inhibits activators of inflammasomes, inhibits activators of inflammasome priming, inhibits inflammasome sensors, inhibits inflammasome adaptors, inhibits inflammasome effectors, inhibits pyroptosis, inhibits, necroptosis, inhibits apoptosis, and/or inhibits panoptosis.

In other aspects, the pharmaceutical composition inhibits ASC activation, inhibits Caspase 1 activation, inhibits Gasdermin D activation, inhibits Interleukin 1β (IL-1β) activation, and/or inhibits Interleukin 18 (IL-18) activation.

In other aspects, the pharmaceutical composition modulates gene transcription, gene translation, gene activity, gene expression, post-transcriptional, and/or post-translational modification of a protein encoded by a gene, wherein the gene comprises ACE, ACE2, ADAM17, ADAM9, ADAMTS13, ADORA2A, AGT, AGTR, AGTR1, AGTR2, AIM2, ANAPC1, ANO6, BDKRB1, BDKRB2, BID, BIRC2, BIRC3, BIRC5, BIRC6, BRCC3, BTK, BTK, CAMK2A, CAMP, CAPN1, CAPN2, CASP1, CASP10, CASP11, CASP12, CASP14, CASP2, CASP3, CASP4, CASP5, CASP6, CASP7, CASP8, CASP9, CAST, CAT, CBL, CCL2, CD209, CFL2, CFLAR, CFLAR-AS1, CHMP4A, CHMP4B, CHMP4C, CHUK, CLEC4M, CSTA, CTSB, CTSC, CTSD, CTSF, CTSG, CTSH, CTSK, CTSL, CTSO, CTSS, CTSV, CTSX, CTSZ, CXCL10, CXCL2, CXCL3, CYLD, DDX15, DDX19, DDX5, DNMT1, DPP4, FADD, FURIN, GSDMA, GSDMB, GSDMC, GSDMD, GSDME, GSPT1, GSPT2, H1-1, HIF1A, HIF1AN, HIF3A, HMGB1, HSP90, HSP90AA1, HSP90AB4P, HSP90B1, HSP90B2P, IFIT1, IFNA1, IFNAR1, IFNAR2, IFNLR1, IKBKB, IKBKE, IKBKG, IKK, IL10, IL10RA, IL10RB, IL15, IL17, IL17A, IL18, IL18BP, IL1B, IL1R1, IL1RL1, IL21, IL9, IRF3, IRF7, KEAP, KL, KLKB1, KNG1, LRRK2, LTB4R, MAP3K7, MAPK1, MAPK3, MAPKAPK2, MAS1, MAVS, MIR20A, MIR20B, MIR21, MIR223, MIR30C, MIR30C1, MIR30C5, MIR495, MIR9, MLKL, MME, NAMPT, NEK6, NEK7, NEK9, NFATC3, NFE2L2, NFKB, NLRC3, NLRC4, NLRC5, NLRP1, NLRP10, NLRP11, NLRP12, NLRP13, NLRP14, NLRP2, NLRP3, NLRP4, NLRP5, NLRP6, NLRP7, NLRP8, NLRP9, NLRX1, NMNAT1, NOX, NOX2, NOX4, NR1H4, P2RX1, P2RX2, P2RX2, P2RX3, P2RX3, P2RX4, P2RX5, P2RX5-TAX1BP3, P2RX6, P2RX6P, P2RX7, P2RY1, P2RY10, P2RY11, P2RY12, P2RY13, P2RY14, P2RY2, P2RY4, P2RY6, P2RY8, PADI4, PARP, PARP1, PELI1, PELI2, PELI3, PEP, PRCP, PYCARD, RIPK1, RIPK2, RIPK3, RNF31, SENP6, SENP7, SERPINA1, SERPINA4, SESN2, SIRT1, SIRT2, SLC44A2, SOCS1, SOD, SOD1, SUGT1, TAB1, TAB3, TBK1, TBKBP1, TF, TFP1, TFPI, THOP1, TICAM1, TMPRSS4, TMRPSS2, TNF, TNFAIP1, TNFAIP2, TNFAIP3, TRADD, TRAF2, TRIB3, TRIM31, TRPA4, TRPM2, TRPV1, TRPV4, USP18, VDAC1, VDR, XDH, and/or ZBP1.

In other aspects, the pharmaceutical composition inhibits inflammasome sensor activation, ASC activation, inhibits Caspase 1 activation, inhibits Caspase 8 activation, Caspase 9 activation, inhibits RIPK1 activation, inhibits RIPK3 activation, inhibits FADD activation, inhibits MLKL activation, inhibits Gasdermin D activation, inhibits IL-1β activation, and/or inhibits IL-18 activation; and further wherein the inflammasome comprises NLRP3, NLRC4, NLRP1, and/or AIM2.

In other aspects, the pharmaceutical composition inhibits inflammasome priming and/or activation signals comprising inhibiting DAMPS and/or mitochondrial DAMPS, inhibiting PAMPS and/or PAMPS associated TLR activation, modulating RAAS activation, modulates Kallikrein Kinin dysregulation, inhibiting NFKB pathways, inhibiting ER stress and/or ER associated UPR, inhibiting microtubule assembly and or function, inhibiting intracellular NAD+ release, inhibiting mitochondrial stress, mitochondrial fission, and/or mitochondrial apoptosis, inhibiting diminished mitophagy, inhibiting mtDNA damage, inhibiting diminished mitochondrial membrane potential (Δψm), inhibiting oxidoreductase enzyme, inhibiting TXNIP, inhibiting lysosomal rupture, inhibiting cathepsins, inhibiting cathepsin B, inhibiting angiotensin II, inhibiting cyclic GMP-AMP synthase (cGAS) and stimulator of interferon genes (STING) activation, inhibiting HMGB1/RAGE, inhibiting reactive oxygen species (ROS) and superoxide, inhibiting HSP90, inhibiting SUGT1 (SGT1), inhibiting NEK7, inhibiting intracellular calcium increase and/or intracellular rise of particulate/crystals, inhibiting necroptosis via inhibiting TNFR1, MLKL, ZBP1, RIPK3, and/or RIPK1, inhibiting CASP1, inhibiting CASP8, inhibiting CASP4, inhibiting CASP5, inhibiting CASP9, inhibiting ASC oligomerization, inhibiting Gasdermin D (GSDMD), inhibiting DUBs (deubiquitinase enzyme), inhibiting non-canonical inflammasome activation, inhibiting the purinoreceptors P2RX1-7, inhibiting P2RX7, and/or P2RY, inhibiting CXCR2-PKCm-ILK axis, inhibiting IL-17, inhibiting IL-21, inhibiting the PI3K/AKT/mTOR pathway, and/or inhibiting Micro RNA 21.

In other aspects, the pharmaceutical composition further comprises a therapeutically effective amount of a modulator of SARS-CoV-2-associated spike protein receptor binding, cleavage, fusion, and/or viral entry into host cells comprising a protease inhibitor comprising TMPRSS2, TMPRSS4, Cathepsin B, Cathepsin L, Cathepsin S, Cathepsin Z, Cathepsin C, DPP4, CD209, CLEC4M and/or Furin.

In other aspects, the pharmaceutical composition further comprises a therapeutically effective amount of a modulator of SARS-CoV-2-associated spike protein receptor binding, cleavage, fusion, and/or viral entry into host cells comprising an inhibitor of ACE2, an activator of ADAM17, an activator of Ang(1-7), an activator of MAS1, an agonist and/or mimetic of Kallistatin, an activator of SERPIN4a, and/or an activator of SERPIN1a.

In other aspects, the pharmaceutical composition comprises a pharmaceutically acceptable salt, hydrate, cocrystal, solvate, ion charged compound, and/or prodrug of any of the active agents described herein.

In other aspects, the pharmaceutical composition is formulated for a route of administration comprising oral, intramuscular, subcutaneous, intradermal, parenteral, intraperitoneal, intranasal, inhalational, nebulized, intramucosal, sublingual, rectal, topical, transdermal, intravitreal, conjunctival, intra ocular, mucosal, intratracheal, intra-urethral, vaginal, labial, intra-aural, and/or intravenous routes.

In other aspects, the pharmaceutical composition comprises a formulation comprising solids, liquids, gaseous, vaporized, syrup, tablets, gel capsules, vegetarian gel capsules, pastilles, dry powder, sachets, emulsion, pellets, inhalational solution, drops, ampules, gummy bears, candy bars, power drinks, food powders, effervescent tablets, nebulized, nasal sprays, disks, mucosal preparations, anal preparations, rectal preparations, creams, lotions, gels, serums, colloidal preparations, liposomal preparations, topical preparations, transdermal preparations, immediate release preparations, delayed preparations, extended release preparations, controlled release preparations, and/or sustained release preparations.

### Methods of Treatment and Genetic Testing

In another embodiment, a method is provided for preventing and/or treating a health condition related to inflammasome NLRP3 dysregulation, RAAS dysregulation, KKS dysregulation, protease dysregulation, and/or cytokine dysregulation in a subject in need thereof, comprising administering a pharmaceutical composition to the subject, wherein the pharmaceutical composition comprises:
a) two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3; and/or
b) one or more additional active agents comprising:
   i) one or more modulators of Renin Aldosterone Angiotensin System (RAAS) dysregulation, comprising an inhibitor of the ACE/Angiotensin II arm of RAAS and/or an activator of the ACE2/Ang(1-7)/Mas1 arm of RAAS comprising an inhibitor of ACE, an inhibitor of Angiotensin II activation, an activator of ACE2, an activator of Ang(1-7), an inhibitor of AT1R, an inhibitor of ACE gene expression and/or activity, an inhibitor of Renin, an inhibitor Chymase, a mast cell stabilizer, an inhibitor of histamine, an inhibitor of neprolysin, a combined inhibitor of At1R and Neprilysin, an activator of ADAM17 activity and/or gene expression, an activator of MAS1, an activator of MAS1 gene expression and/or activity, an activator of AT2R, and/or an activator of AT2R;
   ii) one or more modulators of Kallikrein-Kinin System (KKS) dysregulation;
   iii) one or more modulators of protease dysregulation; and/or
   iv) one or more modulators of cytokine dysregulation;
      wherein the pharmaceutical composition further comprises a pharmaceutically acceptable carrier; and wherein the active agents are each present in therapeutically effective amounts to prevent and/or treat the health condition related to inflammasome NLRP3 dysregulation, RAAS dysregulation, KKS dysregulation, protease dysregulation, and/or cytokine dysregulation in the subject in need thereof.

In other aspects, the pharmaceutical composition within the method is further characterized wherein:
a) the two or more active agents of 1(a) comprise a full or partial inhibitor of NLRP3, NLRC4, NLRP1, NLRP6, IFI16, Pyrin, and/or AIM2;
b) the one or more modulators of KKS dysregulation comprise an inhibitor of KKS activation comprising an inhibitor of Neprilysin, a combined inhibitor of At1R and Neprilysin, an inhibitor of ACE and Neprilysin, an inhibitor of Bradykinin B2, an inhibitor of Bradykinin B1, inhibitor of Kallikrein B1(KLKB1), an inhibitor of Kininogen 1 (KNG1), an inhibitor of High-molecular-weight kininogen (HMWK), an inhibitor of Low-molecular-weight kininogen (LMWK), an activator of Kallistatin, an inhibitor of histamine, an activator of SERPINA1, an activator of SERPINA4, and/or an activator of Cystatin A;
c) the one or more modulators of protease dysregulation comprise an inhibitor of a protease comprising Furin, Cathepsin B, Cathepsin C, Cathepsin D, Cathepsin F, Cathepsin G, Cathepsin K, Cathepsin O, Cathepsin S, Cathepsin L, Cathepsin V, Cathepsin X, Cathepsin Z, TMPRSS2, CLEC4M, CAPN2, CAPN1, CD209, and/or TMPRSS4, and/or the one or more modulators of protease dysregulation comprise an activator of CAST (calpastatin) gene activity and/or gene expression, an activator of Calpastatin, and/or an activator of Cystatin A; and
d) the one or more modulator of cytokine dysregulation comprises an activator of IFN-λ, an activator of Type I interferon, an inhibitor of CXCs, an inhibitor of CCL2, and/or an activator of IL-10.

In other aspects, the activator of IFN-λ and/or activator of Type I interferon are administered to the subject less than 48 hours post-infection.

In other aspects, the two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3 comprise an activator of cellular autophagy, an activator of mitophagy, an activator of purine salvage pathways, an activator of mitochondrial cellular bioenergetics, an activator of NRF2 gene expression and/or activity, an activator of SIRT1, an activator of ACE2/MAS/ANG(1-7), an ANG(1-7) mimetic, an activator of NLRP3 ubiquitination and degradation, an activator of dopamine receptor-1 (DRD1), and/or an activator of NAD+, further wherein the activator of NAD+ comprises an activator of nicotinamide salvage pathways, a precursor of NAD+, an agonist of NAD+, an NAD+ mimetic, an inhibitor of ROS and oxidative stress, an inhibitor of PARP1, an activator of NAD+ dependent SIRT2, an activator of mitochondrial uncoupling, an activator of mitochondrial biogenesis, and/or an activator of transcription and/or activity of genes comprising SIRT2, SIRT1, NAMPT, and/or NMNAT1.

In other aspects, the two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3 also comprise one or more full or partial inhibitors of one or more priming or activation signals of one or more activated Pattern Recognition Receptors (PRRs) and/or a modulator of PRR-activation-induced dysregulation in inflammasome signal transduction pathways, wherein the PRR is an inflammasome.

In other aspects, the one or more full or partial inhibitors of one or more priming or activation signals of one or more activated PRRs comprises a full or partial inhibitor of NLRP1, NLRX1, NLRC5; and/or cGAS-STING.

In other aspects, the one or more full or partial inhibitors of one or more priming or activation signals of one or more activated PRRs comprises a full or partial inhibitor of NLRP3, NLRC4, NLRP1, NLRP6, IFI16, Pyrin, and/or AIM2 and/or their related adaptors or effectors, comprising a phenothiazine dye, 4-phenylbutyrate (4-PBA), 4-phenylbutyric acid, sodium phenyl butyrate, methylene blue, glycine, a xanthine oxidoreductase inhibitor, a bile acid, taurodeoxychcolic acid (TUDCA), naltrexone, a microtubule disruptor/inhibitor, demcolcine, colchicine, an HMG-CoA reductase inhibitors/Statin, simvastatin, an antihistamine, a fibric acid derivative, metformin, propylthiouracil, thiazolidinedione, ketamine, nicotine, ivermectin, albendazole, an H/K inhibitor, amitriptyline, a corticosteroid, niclosamide, riluzole, sunitinib, valproic acid, glibenclamide bortezomib, an antibiotic, a tetracycline, minocycline, gentamycine, levofloxacine, cyclosporin, diminazen or diminazene aceturate, a calcium channel blocker, felodipine, a leukotriene D4 inhibitor, an ACE inhibitor, enalapril, an Angiotensin Receptor 1 inhibitor, resveratrol, anthocyanin, taurine, thioctic acid, rosmarinic acid, sulforaphane, celastrol, berberine, Epigallocatechin gallate, catechin, glucosamine, melatonin, pregnenolone, testosterone, estradiol, estriol, mangiferin, linarin, fisetin, rutin, rutecarpin, selenium, selenomethionin, progesterone, n-acetyl cysteine, acetyl cysteine, Mitotempo, tempol, catalase, SOD, apigenin, quercetin, tempol, silybin/silymarin, apocynin, a zinc compound, silybin, curcumin, alpha lipoic acid, genistein, gedunin, astaxanthin, copper, vitamin C, vitamin E, Vitamin D, calcitriol, niacin, tretinoin, acetaminophen, pregnenolone, a non-steroidal anti-inflammatory drug, β-Ursolic acid, Schinol, glycyrrhizic acid, trichostatinA, docasohexaenoic acid, pathenolide and/or any compound listed in Tables 1, 2, 3, or 4 in FIGS. 1, 2, 3, and 4.

In other aspects, the health condition is SARS-CoV-2 infection and/or COVID-19.36.

In other aspects, the method comprises preventing and/or treating a symptom of SARS-CoV-2 infection and/or COVID-19 comprising stroke, myocardial infarction, arterial thromboembolism, venous thromboembolism, microvascular thromboembolism, lung injury, acute respiratory distress syndrome, pulmonary fibrosis, hypoxemia, retinopathy, vasculitis, antiphospholipid syndrome, thrombocytopenia, cardiomyopathy, myocarditis, encephalopathy, renal disease, gastrointestinal disease, skin rash, skin ulcer, infertility, fatigue, and/or ventilator induced lung injury.

In other aspects, the pharmaceutical composition within the method comprises one or more modulators of SARS-CoV-2 associated RAAS dysregulation and one or more modulators of KKS dysregulation, wherein the one or more modulators of SARS-CoV-2 associated RAAS dysregulation and the one or more modulators of KKS dysregulation are each present in therapeutically effective amounts to prevent or treat SARS-CoV-2 infection and/or COVID-19 in a subject in need thereof.

In other aspects, the pharmaceutical composition within the method comprises one or more inhibitors of SARS-CoV-2 associated inflammasome activators, wherein the inflammasome is NLRP3, AIM2, and/or NLRP1, and further wherein the one or more inhibitors of SARS-CoV-2 associated inflammasome activators is present in therapeutically effective amounts to prevent or treat SARS-CoV-2 infection and/or COVID-19 in a subject in need thereof.

In other aspects, the pharmaceutical composition within the method comprises one or more inhibitors of SARS-CoV-2 associated cytokine dysregulation, comprising a modulator of SARS-CoV-2 associated Interferon I and/or III antiviral response.

In other aspects, the modulator of SARS-CoV-2 associated Interferon antiviral response is an activator of IFN-λ comprising:
a) an activator/stimulator of IFN-λ;
b) a pegylated-IFN-λ;
c) an activator of IRF3 gene expression and/or activity;
d) an activator of IRF7 gene expression and/or activity;
e) an activator of IFN-λ receptor INFLR1 gene expression and/or activity, or an inhibitor of IFN-λ receptor INFLR1, IFR3, IRF7, and/or IL10RB; and/or
f) an activator of IFN-λ receptor IL10RB gene expression and/or activity.

In other aspects, the activator of IFN-λ receptor INFLR1 gene expression and/or activity is administered to the subject less than 48 hours post-infection and the inhibitor of IFN-λ receptor INFLR1, IFR3, IRF7, and/or IL10RB is administered to the subject 48 hours or more post-infection.

In other aspects, the modulator of SARS-CoV-2 associated Interferon antiviral response is an activator of Il-15 comprising:
a) an activator of IL-15 gene mRNA expression and/or activity;
b) an activator of IFN-λ for early infection or an inhibitor of IFN-λ receptor INFLR1, IFR3, IRF7, and/or IL10RB for after early infection;
c) an activator of IL-12 gene expression and/or activity; and/or
d) an activator of IL-21 gene expression and/or activity.

In other aspects, the modulator of SARS-CoV-2 associated Interferon antiviral response is an activator of Interferon I comprising:
i) an activator of Interferon1 beta;
ii) a pegylated-interferon-1beta;
iii) an IFN-λ mimetic, agonist, and/or activator;
iv) an Interferon I mimetic, agonist, and/or activator;
v) an activator of IRF3 gene expression and/or activity; and/or
vi) an activator of expression of interferon 1 receptor gene IFNAR1 or IFNAR2.

In other aspects, the modulator of SARS-CoV-2 associated Interferon antiviral response is an inhibitor of IFNAR1 or IFNAR2, comprising:
i) an inhibitor of IFNAR1 gene expression and/or activity, and
ii) an inhibitor of IFNAR2 gene expression and/or activity.

In other aspects, the modulator of SARS-CoV-2 associated Interferon antiviral response is an activator of IL-10 comprising:
i) an inhibitor of AT1R; and/or
ii) an activator of Heat Shock Factor 1.

In other aspects, the pharmaceutical composition within the method comprises:
a) the two or more full or partial inhibitors of one or more priming or activation signals of inflammasome NLRP3;
b) an activator of Interferon beta and/or IFN-λ for early infection;
c) an inhibitor of Interferon beta and/or IFN-λ for late infection; and
d) an activator of anti-inflammatory interleukin-10 (IL-10)-related cytokines comprising IL-10, IL-19, IL-20, IL-22, IL-24, and IL-26.

In other aspects, the pharmaceutical composition within the method comprises:
a) the two or more full or partial inhibitors of one or more priming or activation signals of inflammasome NLRP3; and
b) the one or more modulators of cytokine dysregulation comprises an activator of IFN-β, an activator of IFN-λ, and an activator of IL-10.

In other aspects, the pharmaceutical composition within the method modulates inflammasome priming and/or activation, wherein the pharmaceutical composition inhibits activators of inflammasomes, inhibits activators of inflammasome priming, inhibits inflammasome sensors, inhibits inflammasome adaptors, inhibits inflammasome effectors, inhibits pyroptosis, inhibits, necroptosis, inhibits apoptosis, and/or inhibits panoptosis.

In other aspects, the pharmaceutical composition within the method inhibits ASC activation, inhibits Caspase 1 activation, inhibits Gasdermin D activation, inhibits Interleukin 1β (IL-1β) activation, and/or inhibits Interleukin 18 (IL-18) activation.

In other aspects, the pharmaceutical composition within the method modulates gene transcription, gene translation, gene activity, gene expression, post-transcriptional, and/or post-translational modification of a protein encoded by a gene, wherein the gene comprises ACE, ACE2, ADAM17, ADAM9, ADAMTS13, ADORA2A, AGT, AGTR, AGTR1, AGTR2, AIM2, ANAPC1, ANO6, BDKRB1, BDKRB2, BID, BIRC2, BIRC3, BIRC5, BIRC6, BRCC3, BTK, BTK, CAMK2A, CAMP, CAPN1, CAPN2, CASP1, CASP10, CASP11, CASP12, CASP14, CASP2, CASP3, CASP4, CASP5, CASP6, CASP7, CASP8, CASP9, CAST, CAT, CBL, CCL2, CD209, CFL2, CFLAR, CFLAR-AS1, CHMP4A, CHMP4B, CHMP4C, CHUK, CLEC4M, CSTA, CTSB, CTSC, CTSD, CTSF, CTSG, CTSH, CTSK, CTSL, CTSO, CTSS, CTSV, CTSX, CTSZ, CXCL10, CXCL2, CXCL3, CPLD, DDX15, DDX19, DDX5, DNMT1, DPP4, FADD, FURIN, GSDMA, GSDMB, GSDMC, GSDMD, GSDME, GSPT1, GSPT2, H1-1, HIF1A, HIF1AN, HIF3A, HMGB1, HSP90, HSP90AA1, HSP90AB4P, HSP90B1, HSP90B2P, IFIT1, IFNA1, IFNAR1, IFNAR2, IFNLR1, IKBKB, IKBKE, IKBKG, IKK, IL10, IL10RA, IL10RB, IL15, IL17, IL17A, IL18, IL18BP, IL1B, IL1R1, IL1RL1, IL21, IL9, IRF3, IRF7, KEAP, KL, KLKB1, KNG1, LRRK2, LTB4R, MAP3K7, MAPK1, MAPK3, MAPKAPK2, MAS1, MAVS, MIR20A, MIR20B, MIR21, MIR223, MIR30C, MIR30C1, MIR30C5, MIR495, MIR9, MLKL, MME, NAMPT, NEK6, NEK7, NEK9, NFATC3, NFE2L2, NFKB, NLRC3, NLRC4, NLRC5, NLRP1, NLRP10, NLRP11, NLRP12, NLRP13, NLRP14, NLRP2, NLRP3, NLRP4, NLRP5, NLRP6, NLRP7, NLRP8, NLRP9, NLRX1, NMNAT1, NOX, NOX2, NOX4, NR1H4, P2RX1, P2RX2, P2RX2, P2RX3, P2RX3, P2RX4, P2RX5, P2RX5-TAX1BP3, P2RX6, P2RX6P, P2RX7, P2RY1, P2RY10, P2RY11, P2RY12, P2RY13, P2RY14, P2RY2, P2RY4, P2RY6, P2RY8, PADI4, PARP, PARP1, PELI1, PELI2, PELI3, PEP, PRCP, PYCARD, RIPK1, RIPK2, RIPK3, RNF31, SENP6, SENP7, SERPINA1, SERPINA4, SESN2, SIRT1, SIRT2, SLC44A2, SOCS1, SOD, SOD1, SUGT1, TAB1, TAB3, TBK1, TBKBP1, TF, TFP1, TFPI, THOP1, TICAM1, TMPRSS4, TMRPSS2, TNF, TNFAIP1, TNFAIP2, TNFAIP3, TRADD, TRAF2, TRIB3, TRIM31, TRPA4, TRPM2, TRPV1, TRPV4, USP18, VDAC1, VDR, XDH, and/or ZBP1.

In other aspects, the pharmaceutical composition within the method inhibits inflammasome priming and/or activation signals comprising inhibiting DAMPS and/or mitochondrial DAMPS; inhibiting PAMPS and/or PAMPS associated TLR activation; modulating RAAS activation; modulates Kallikrein Kinin dysregulation; inhibiting NFKB pathways; inhibiting ER stress and/or ER associated UPR; inhibiting microtubule assembly and or function; inhibiting intracellular NAD+ release; inhibiting mitochondrial stress, mitochondrial fission, and/or mitochondrial apoptosis; inhibiting diminished mitophagy; inhibiting mtDNA damage; inhibiting diminished mitochondrial membrane potential (Δψm); inhibiting oxidoreductase enzyme; inhibiting TXNIP; inhibiting lysosomal rupture; inhibiting cathepsins; inhibiting cathepsin B, inhibiting angiotensin II; inhibiting cyclic GMP-AMP synthase (cGAS) and stimulator of interferon genes (STING) activation; inhibiting HMGB1/RAGE; inhibiting reactive oxygen species (ROS) and superoxide; inhibiting HSP90; inhibiting SUGT1 (SGT1); inhibiting NEK7; inhibiting intracellular calcium increase and/or intracellular rise of particulate/crystals; inhibiting necroptosis via inhibiting TNFR1, MLKL, ZBP1, RIPK3, and/or RIPK1; inhibiting CASP8; inhibiting CASP4; inhibiting CASP5, inhibiting CASP9; inhibiting ASC oligomerization; inhibiting Gasdermin D (GSDMD); inhibiting DUBS (deubiquitinase enzyme); inhibiting non-canonical inflammasome activation, inhibiting the purinoreceptors P2RX1-7, inhibiting P2RX7, and/or P2RY; inhibiting CXCR2-PKCm-ILK axis; inhibiting IL-17; inhibiting IL-21; inhibiting the PI3K/AKT/mTOR pathway; and/or inhibiting Micro RNA 21.

In other aspects, the pharmaceutical composition within the method further comprises a therapeutically effective amount of a modulator of SARS-CoV-2-associated spike protein receptor binding, cleavage, fusion, and/or viral entry into host cells comprising a protease inhibitor comprising TMPRSS2, TMPRSS4, Cathepsin B, Cathepsin L, Cathepsin S, Cathepsin Z, Cathepsin C, DPP4, CD209, CLEC4M and/or Furin.

In other aspects, the pharmaceutical composition within the method further comprises a therapeutically effective amount of a modulator of SARS-CoV-2-associated spike protein receptor binding, cleavage, fusion, and/or viral entry into host cells comprising an inhibitor of ACE2, an activator of ADAM17, an activator of Ang(1-7), an activator of MAS1, an agonist and/or mimetic of Kallistatin, an activator of SERPIN4a, and/or an activator of SERPIN1a.

In other aspects, the pharmaceutical composition within the method further comprises a pharmaceutically acceptable salt, hydrate, cocrystal, solvate, ion charged compound, and/or prodrug of any of the active agents described herein.

In other aspects, the pharmaceutical composition within the method is formulated for a route of administration comprising oral, intramuscular, subcutaneous, intradermal, parenteral, intraperitoneal, intranasal, inhalational, nebulized, intramucosal, sublingual, rectal, topical, transdermal, intravitreal, conjunctival, intra ocular, mucosal, intratracheal, intra-urethral, vaginal, labial, intra-aural, and/or intravenous routes.

In other aspects, the pharmaceutical composition within the method comprises a formulation comprising solids, liquids, gaseous, vaporized, syrup, tablets, gel capsules, vegetarian gel capsules, pastilles, dry powder, sachets, emulsion, pellets, inhalational solution, drops, ampules, gummy bears, candy bars, power drinks, food powders, effervescent tablets, nebulized, nasal sprays, disks, mucosal preparations, anal preparations, rectal preparations, creams, lotions, gels, serums, colloidal preparations, liposomal preparations, topical preparations, transdermal preparations, immediate release preparations, delayed preparations, extended release preparations, controlled release preparations, and/or sustained release preparations.

In other aspects, any of the active agents are administered alone or together and can be combined and administered as a sequential therapy and/or a concurrent therapy with other medications and therapies as determined by health care providers and/or treating physicians.

In other aspects, the health condition is Cancer, Cardiovascular disease, Thromboembolic disease, Pulmonary disease, Infectious disease, sepsis, Systemic inflammatory response syndrome (SIRS), cytokine storm, Autoimmune and Inflammatory diseases, Inflammatory arthritis, Psoriatic skin disease, Psoriatic arthritis, Systemic lupus erythematosus (SLE), Vasculitis, Osteoarthritis, Multiple Sclerosis (MS), Inflammatory and or genetic and or rare diseases, Chronic pain, Neuropathic pain, Migraine, Glossodynia, Retinopathy, Retinal detachment, Age related Macular degeneration (AMD), Neurologic disorders comprising Dementia and or neurodegenerative disease, Alzheimer's disease, Parkinson's disease, Prion disease, Huntington's disease, Meningitis, Epilepsy, Skin Disease comprising skin ulcers, skin infections, Tinea Infections, onychomycosis, Tinea Versicolor, cutaneous abscess, Cellulitis, alopecia, Renal disease, Interstitial Cystitis (IC), benign prostatic hypertrophy, Gastrointestinal (GI) disease, Hepatitis Liver Cirrhosis, Inflammatory bowel disease, GI Dysbiosis, Aging and age related diseases, cellular senescence, Mitochondrial stress, mitochondrial disease, mitochondrial cytopathies, Osteoporosis, Osteopenia, periodontal bone loss, dyslipidemia, hypercholesterolemia, Mood Disorders, Metabolic disease, Chronic Fatigue Syndrome, and/or Fibromyalgia.

In other aspects, the health condition is a cardiovascular disease comprising atherosclerosis, dyslipidemia, hypercholesterolemia, hypertension, cerebrovascular accident, transient ischemic attack, stroke, vascular dementia, peripheral arterial disease (PAD), venous disease, myocardial infarction, coronary artery disease, carotid artery disease, dilated cardiomyopathy, hypertrophic cardiomyopathy, ischemic cardiomyopathy, cardiac amyloidosis, congestive heart failure, arrythmia, atrial fibrillation, pericardial disease, pericarditis, pericardial effusion, myocarditis, aortic ectasia, aortic aneurysm, abdominal aortic aneurysm, splenic aneurysm, mesenteric ischemia, pulmonary hypertension, renovascular disease, portocaval hypertension, vascular dementia, peripheral vascular disease, peripheral venous disease, peripheral arterial disease, and/or vasculitis, good pastures, Kawasaki, giant cell arteritis, poly arteritis nodosa, antiphospholipid syndrome, Henoch shconline purpura, and/or Churg-Strauss.

In other aspects, the health condition is a thromboembolic disease comprising arterial or venous thromboembolism, small or large vessel thromboembolism, microvascular thrombosis, sepsis associated thromboembolism, stroke, myocardial infarction, mesenteric embolism, antiphospholipid syndrome, vasculitis, Kawasaki disease, deep venous thrombosis, and/or pulmonary embolism.

In other aspects, the health condition is a pulmonary disease comprising acute lung injury, acute respiratory distress syndrome (ARDS), neonatal ARDS, chronic obstructive pulmonary disease, emphysema, anti-alpha trypsin deficiency, asthma, bronchitis, pneumonia, interstitial lung disease, infectious lung disease, granulomatous lung disease, autoimmune lung disease, mesothelioma, pneumoconiosis, asbestosis, bronchiolitis obliterans organizing pneumonia (BOOP), ventilator associated lung damage, hyperoxia lung damage, pleural effusion, pleuritis, fibrotic lung disease, high altitude pulmonary edema, Idiopathic interstitial pneumonias, lymphoid interstitial pneumonia (LIP), idiopathic interstitial pneumonia: unspecified, Hyper-eosinophilic lung diseases, Allergic bronchopulmonary aspergillosis (ABPA), Pulmonary vasculitis, Alveolar hemorrhage syndromes, Pulmonary lymphangioleiomyomatosis (LAM), Alveolar proteinosis, Pulmonary amyloidosis, Pulmonary Langerhans cell histiocytosis (histiocytosis x), Primary pulmonary lymphoma, Primary ciliary dyskinesia (without or with situs inversus), Rare cause of hypersensitivity pneumonitis (all causes other than farmer's lung disease and pigeon breeder's lung disease), Pulmonary arteriovenous malformations in hereditary hemorrhagic telangiectasia (HHT), Pulmonary manifestations of gastro-intestinal disorders, Interstitial lung disease in connective tissue diseases, Bronchiolitis obliterans in non-transplanted patients, Sarcoidosis, Chronic beryllium disease, Waterproofing spray pneumonitis, Vaping related pneumonitis, Combined pulmonary fibrosis and emphysema, and/or Alpha-1-antitrypsin deficiency emphysema.

In other aspects, the health condition is an infectious disease comprising a viral, bacterial, parasitic, and/or fungal infectious disease, wherein the viral infectious disease comprises a disease caused by SARS-CoV, SARS-CoV-2, MERS-CoV, Hepatitis A, Hepatitis B, Hepatitis C, Hepatitis D, Hepatitis E, Hepatitis F, HIV, Ebola, Herpes, Influenza, Roseola, Variola, rubella, measles, chikungunya, Cytomegalovirus, Respiratory syncytial virus, Epstein bar virus, Adeno virus, Coxsackie A, or Coxsackie B, Marburg, Rabies, Hanta, Dengue, and/or Rota, and wherein the bacterial infectious disease comprises disease caused by Escherichia coli, Klebsiella pneumonia, Enterococcus faecalis, Enterococcus faecium, Vibrio cholera, Staphylococcus aureus, Staphylococcus epidermidis, Bacteroides, Neisseria gonorrhea, Neisseria meningitis, Proteus Mirablis, Hemophilus influenza, Haemophilus ducreyi, Campylobacter jejuni, Salmonella, Citrobacter ferundii, Streptococcus pyogenes, Group A streptococcus, Group B streptococcus, Chlamydia psittaci, Francisella tularensis, Coxiella burnetii, Chlamydia pneumoniae, Mycoplasma pneumoniae, Legionella, Chlamydia trachomatis, Salmonella, Shigella, Clostridium perfringens, Borrelia burgdorferi, Rickettsia, and/or mycobacteria, and wherein the parasitic infectious disease comprises a disease caused by giardia and/or Entamoeba histolytica, and wherein the fungal infectious disease comprises a disease caused by cryptococcosis, Aspergillosis, Coccidioidomycosis, Histoplasmosis, Blastomycosis, Pneumocystis jirovecii, candida, dermatophytes, Malassezia globosa, Malassezia furfur, Tinea Infections, onychomycosis, Tinea Versicolor, and/or cutaneous abscess. In other aspects, the infectious disease comprises bacteremia, sepsis, urosepsis, severe sepsis, septic shock, and systemic inflammatory response syndrome (SIRS), and where in the infectious disease comprises cutaneous abscess, furuncles, carbuncles, Bartholin abscess, sterile abscess, perirectal abscess, perianal abscess, vulvar abscess, perineal abscess, perioral abscess, Mycobacterium abscess, epidermoid abscess and wherein the infectious disease comprises a cellulitis disease caused by Staphylococcus epidermidis, Staphylococcus aureus, MRSA, Pasteurella multocida and Capnocytophaga canimorsus, Aeromonas hydrophila, Vibrio vulnificus, Pseudomonas aeruginosa, Group B Streptococcus, Clostridium species (i.e., Clostridial myonecrosis), Erysipelothrix rhusiopathiae, S. pneumoniae, Cryptococcus neoformans, Streptococcus iniae, and/or Helicobacter cinaedi.

In other aspects, the health condition is a renal disease comprising chronic kidney disease (CKD), end stage kidney disease ESRD, nephrocalcinosis, diabetic nephropathy, hypertensive nephropathy, acute renal failure, acute tubular necrosis, nephrotic syndrome, glomerular nephritis, aldosterone-induced renal tubular cell injury, nephrotoxic renal injury, microalbuminuria, proteinuria, renal amyloidosis, amyloid nephropathy, immunoglobulin A nephropathy, polycystic kidney disease urinary, hepatorenal syndrome, interstitial cystitis, urinary tract infection, pyelonephritis, interstitial cystitis (IC), Ulcerative IC and/or non-ulcerative IC.

In other aspects, the health condition is a gastrointestinal (GI) disease comprising peptic ulcer disease (PUD), gastroesophageal reflux disease (GERD), esophagitis, Barrette's esophagitis, gastric dysmotility disease, small intestinal bacterial over growth (SIB), malabsorption, pancreatitis, pancreatic pseudocyst, cholecystitis, cholelithiasis, cirrhosis, ascites, NALD, hepato-steatosis, steatohepatitis, inflammatory bowel disease, hepatitis, cholestatic liver injury, ischemic-perfusion liver disease, liver cirrhosis, inflammatory bowel disease, ulcerative colitis, microscopic colitis, C-diff colitis (pseudomembranous colitis), and/or GI dysbiosis.

In other aspects, the health condition is a metabolic disease comprising prediabetes, diabetes, obesity, steatohepatitis, hepato-steatosis, non-alcoholic steatohepatitis (NASH), pancreatitis, pancreatic pseudocyst, Chronic Fatigue Syndrome, Fibromyalgia, dyslipidemia, and/or hypercholesterolemia.

In other aspects, the health condition is an autoimmune or inflammatory disease comprising systemic lupus erythematosus (SLE), Wegener's granulomatosis, Sjogren's syndrome, vasculitis, anti-phospholipid syndrome, rheumatoid arthritis, osteoarthritis, myositis, dermatomyositis, psoriasis, spondylo-negative arthritis, autoimmune hepatitis, good pastures, vasculitis, mixed connective tissue disease, Inflammatory arthritis, Ankylosing spondylitis, Gout, pseudogout, Lyme's disease, sarcoid arthritis, rheumatoid arthritis, Psoriatic skin disease, Psoriatic arthritis, Osteoarthritis, Multiple Sclerosis (MS), and/or autoimmune encephalitis.

In other aspects, the health condition is a genetic or rare disease comprising Mediterranean fever, Bechet disease, Muckle-Wells syndrome (MWS), and/or Amyloidosis.

In other aspects, the health condition is an acute or chronic pain comprising Neuropathic pain, trigeminal neuralgia, post herpetic neuralgia, cancer pain, chronic neuropathic pain, bone fracture related pain, bone metastasis related pain, pleuritis related pain, pericarditis related pain, Migraine, Glossodynia, oral dysesthesia, glossopyrosis, glossalgia, stomatodynia, stomatopyrosis, chronic pain syndrome, and/or fibromyalgia.

In other aspects, the health condition is an eye disease comprising Retinopathy, non-proliferative or proliferative diabetic retinopathy, Retinal detachment, Retinitis Pigmentosa, Age related Macular degeneration (AMD), wet AMD, uveitis, iritis, cataracts, dry eyes, blepharitis, dry eye blepharitis syndrome (DEBS), glaucoma, conjunctivitis, chalazion, and/or a sty.

In other aspects, the health condition is a neurologic disorder comprising neurodegenerative disease, dementia, senile dementia, Alzheimer's dementia, Parkinson's dementia, dementia of Lewy body, amyloidosis, mild cognitive impairment, frontotemporal dementia, Huntington's chorea dementia, vascular dementia, Creutzfeldt-Jakob disease, other prion disease dementia, mixed dementia, Dementia in HIV/AIDS, dementia associated with Traumatic Brain Injury, Wernicke-Korsakoff Syndrome, alcohol induced dementia, normal pressure hydrocephalus, amyotrophic lateral sclerosis, Alzheimer's disease (AD), Parkinson's disease, Prion disease, Motor neuron, diseases (MND), Huntington's disease (HD), Spinocerebellar ataxia (SCA), Spinal muscular atrophy (SMA), wolfram syndrome (WFS1, WFS2), memory loss, traumatic brain injury, post-traumatic stress disease (PTSD)), Meningitis, Epilepsy, tonic-clinic or convulsive seizures, absence seizures, atonic seizures, tonic seizures, clonic seizures, myoclonic seizures, jacksonian seizure, partial focal seizures and/or Migraine.

In other aspects, the health condition is a skin disease comprising skin ulcers, non-healing ulcers, decubitus ulcers, pressure ulcers, aphthous ulcers, venous stasis ulcers, Stasis ulcers, neuropathic skin ulcers, ischemic arterial ulcers, buruli ulcers, infectious ulcers, pyoderma gangrenosum, vasculitis ulcers, cryoglobulinemic ulcers, diabetic ulcers, warfarin induced skin ulcers, stab wound ulcers, fungal ulcers, bacterial ulcers, viral skin ulcers, herpes ulcers, protozoal skin infections, Rickettsial skin ulcers, epidermal parasitic skin ulcers, renal induced skin ulcers, vesicular skin lesions, candida oral lesions, angular cheilitis, glossitis, hairy tongue, lichen planus, recurrent aphthous stomatitis, herpes labialis, herpes genitalis, erythema migrans, hairy tongue, herpetiform ulcers, recurrent aphthous stomatitis, major and minor aphthous ulcers, gingivitis, pharyngitis, rosacea, acne, Acne vulgaris, Hidradenitis suppurativa/acne inversa (HS), alopecia, androgenic alopecia, alopecia areata, telogen effluvium, anagen effluvium, traction alopecia, trichotillomania, and/or achromotrichia

In other aspects, the health condition is aging and/or age-related diseases comprising diabetes, diabetes type II, obesity, insulin resistance, inflammatory disease, cardiovascular disease, atherosclerosis, thromboembolism, neurodegenerative disease, Alzheimer's disease, Parkinson's disease, Pick's disease, dementia, Lewy body dementias, renal disease, cancer, osteoarthritis, osteopenia, osteoporosis, periodontal bone loss, sarcopenia, periodontitis, rheumatoid arthritis, maculopathy, chronic obstructive pulmonary disease, hair loss, Hutchinson-Gilford progeria, cellular senescence, Mitochondrial stress, mitochondrial disease, and/or dyslipidemia.

In other aspects, the health condition is a mood disorder comprising Bipolar I Disorder, Bipolar II Disorder, Cyclothymic Disorder, Major Depressive Disorder, Persistent Depressive Disorder, Premenstrual Dysphoric Disorder, Anxiety, post-traumatic stress disorder, and/or an addiction disorder.

In other aspects, the health condition is a cancer comprising lung cancer, gastrointestinal cancer, salivary cancer, oropharyngeal cancer, tongue cancer, salivary cancer, esophageal cancer, hepatoma, liver cancer, gastric cancer, pancreatic cancer, gallbladder cancer, colorectal cancer, renal cell carcinoma, bladder cancer, prostate cancer, multiple endocrine neoplasia, cervical cancer, ovarian cancer, endometrial cancer, uterine cancer, vulvar cancer, breast cancer, Paget's disease, skin cancers, basal cell carcinoma, squamous cell carcinoma, melanoma, head and neck cancers, lymphoma, Hodgkin's lymphoma, non-Hodgkin's lymphoma, multiple myeloma, smoldering myeloma, Waldenstrom's macroglobulinemia, lymphoma, leukemia, glioma, glioblastoma multiforme, retinal cancer, meningeal cancer, sarcoma, osteosarcoma, bone cancer, parathyroid cancer, adrenal cancer, squamous cell cancer, pheochromocytoma, adenocarcinoma, mucinous cancer, Fibrolamellar carcinoma, Gastric adenocarcinoma and proximal polyposis of the stomach, hereditary diffuse gastric cancer, SDH-deficient gastrointestinal stromal tumor, rare endocrine tumors, adrenocortical carcinoma, anaplastic thyroid cancer, medullary thyroid cancer, and/or paraganglioma.

In another embodiment, a method is provided for detecting the presence of at least one SNP in a subject to assess a risk of having or developing a health condition related to inflammasome NLRP3 dysregulation, RAAS dysregulation, KKS dysregulation, protease dysregulation, and/or cytokine dysregulation, comprising the steps of:
i) collecting a sample from the subject;
ii) detecting the presence of at least one SNP within a gene for a modulator of inflammasome NLRP3 dysregulation, RAAS dysregulation, KKS dysregulation, protease dysregulation, and/or cytokine dysregulation, wherein the gene is comprising ACE, ACE2, ADAM17, ADAM9, ADAMTS13, ADORA2A, AGT, AGTR, AGTR1, AGTR2, AIM2, ANAPC1, ANO6, BDKRB1, BDKRB2, BID, BIRC2, BIRC3, BIRC5, BIRC6, BRCC3, BTK, BTK, CAMK2A, CAMP, CAPN1, CAPN2, CASP1, CASP10, CASP11, CASP12, CASP14, CASP2, CASP3, CASP4, CASP5, CASP6, CASP7, CASP8, CASP9, CAST, CAT, CBL, CCL2, CD209, CFL2, CFLAR, CFLAR-AS1, CHMP4A, CHMP4B, CHMP4C, CHUK, CLEC4M, CSTA, CTSB, CTSC, CTSD, CTSF, CTSG, CTSH, CTSK, CTSL, CTSO, CTSS, CTSV, CTSX, CTSZ, CXCL10, CXCL2, CXCL3, CYLD, DDX15, DDX19, DDX5, DNMT1, DPP4, FADD, FURIN, GSDMA, GSDMB, GSDMC, GSDMD, GSDME, GSPT1, GSPT2, H1-1, HIF1A, HIF1AN, HIF3A, HMGB1, HSP90, HSP90AA1, HSP90AB4P, HSP90B1, HSP90B2P, IFIT1, IFNA1, IFNAR1, IFNAR2, IFNLR1, IKBKB, IKBKE, IKBKG, IKK, IL10, IL10RA, IL10RB, IL15, IL17, IL17A, IL18, IL18BP, IL1B, IL1R1, IL1RL1, IL21, IL9, IRF3, IRF7, KEAP, KL, KLKB1, KNG1, LRRK2, LTB4R, MAP3K7, MAPK1, MAPK3, MAPKAPK2, MAS1, MAVS, MIR20A, MIR20B, MIR21, MIR223, MIR30C, MIR30C1, MIR30C5, MIR495, MIR9, MLKL, MME, NAMPT, NEK6, NEK7, NEK9, NFATC3, NFE2L2, NFKB, NLRC3, NLRC4, NLRC5, NLRP1, NLRP10, NLRP11, NLRP12, NLRP13, NLRP14, NLRP2, NLRP3, NLRP4, NLRP5, NLRP6, NLRP7, NLRP8, NLRP9, NLRX1, NMNAT1, NOX, NOX2, NOX4, NR1H4, P2RX1, P2RX2, P2RX2, P2RX3, P2RX3, P2RX4, P2RX5, P2RX5-TAX1BP3, P2RX6, P2RX6P, P2RX7, P2RY1, P2RY10, P2RY11, P2RY12, P2RY13, P2RY14, P2RY2, P2RY4, P2RY6, P2RY8, PADI4, PARP, PARP1, PELI1, PELI2, PELI3, PEP, PRCP, PYCARD, RIPK1, RIPK2, RIPK3, RNF31, SENP6, SENP7, SERPINA1, SERPINA4, SESN2, SIRT1, SIRT2, SLC44A2, SOCS1, SOD, SOD1, SUGT1, TAB1, TAB3, TBK1, TBKBP1, TF, TFP1, TFPI, THOP1, TICAM1, TMPRSS4, TMRPSS2, TNF, TNFAIP1, TNFAIP2, TNFAIP3, TRADD, TRAF2, TRIB3, TRIM31, TRPA4, TRPM2, TRPV1, TRPV4, USP18, VDAC1, VDR, XDH, and/or ZBP1, and combinations thereof in the sample;
   wherein the presence of the SNP indicates a risk of having or developing a health condition related to inflammasome NLRP3 dysregulation, RAAS dysregulation, KKS dysregulation, protease dysregulation, and/or cytokine dysregulation in the subject.

In some aspects, the health condition is SARS-CoV-2 infection and/or COVID-19.

In some aspects, the SNP is selected from the group consisting of: 84R polymorphism of TRIB3, Q705k, rs4612666, rs35829419, rs10754558 polymorphism in NLRP3, rs2043211 polymorphism in PYCARD, 489C>T as a gain-of-function polymorphism of the P2X7R, and combinations thereof.

In some embodiments, a method is provided for selectively treating a subject with a higher risk for having or developing a health condition related to inflammasome NLRP3 dysregulation, RAAS dysregulation, KKS dysregulation, protease dysregulation, and/or cytokine dysregulation comprising the steps of:
a) detecting at least one SNP associated with a higher risk for having or developing a health condition related to inflammasome NLRP3 dysregulation, RAAS dysregulation, KKS dysregulation, protease dysregulation, and/or cytokine dysregulation in the subject by a method comprising the steps of:
   i) obtaining a sample from a subject;
   ii) detecting the presence of at least one SNP in a gene comprising: ACE, ACE2, ADAM17, ADAM9, ADAMTS13, ADORA2A, AGT, AGTR, AGTR1, AGTR2, AIM2, ANAPC1, ANO6, BDKRB1, BDKRB2, BID, BIRC2, BIRC3, BIRC5, BIRC6, BRCC3, BTK, BTK, CAMK2A, CAMP, CAPN1, CAPN2, CASP1, CASP10, CASP11, CASP12, CASP14, CASP2, CASP3, CASP4, CASP5, CASP6, CASP7, CASP8, CASP9, CAST, CAT, CBL, CCL2, CD209, CFL2, CFLAR, CFLAR-AS1, CHMP4A, CHMP4B, CHMP4C, CHUK, CLEC4M, CSTA, CTSB, CTSC, CTSD, CTSF, CTSG, CTSH, CTSK, CTSL, CTSO, CTSS, CTSV, CTSX, CTSZ, CXCL10, CXCL2, CXCL3, CYLD, DDX15, DDX19, DDX5, DNMT1, DPP4, FADD, FURIN, GSDMA, GSDMB, GSDMC, GSDMD, GSDME, GSPT1, GSPT2, H1-1, HIF1A, HIF1AN, HIF3A, HMGB1, HSP90, HSP90AA1, HSP90AB4P, HSP90B1, HSP90B2P, IFIT1, IFNA1, IFNAR1, IFNAR2, IFNLR1, IKBKB, IKBKE, IKBKG, IKK, IL10, IL10RA, IL10RB, IL15, IL17, IL17A, IL18, IL18BP, IL1B, IL1R1, IL1RL1, IL21, IL9, IRF3, IRF7, KEAP, KL, KLKB1, KNG1, LRRK2, LTB4R, MAP3K7, MAPK1, MAPK3, MAPKAPK2, MAS1, MAVS, MIR20A, MIR20B, MIR21, MIR223, MIR30C, MIR30C1, MIR30C5, MIR495, MIR9, MLKL, MME, NAMPT, NEK6, NEK7, NEK9, NFATC3, NFE2L2, NFKB, NLRC3, NLRC4, NLRC5, NLRP1, NLRP10, NLRP11, NLRP12, NLRP13, NLRP14, NLRP2, NLRP3, NLRP4, NLRP5, NLRP6, NLRP7, NLRP8, NLRP9, NLRX1, NMNAT1, NOX, NOX2, NOX4, NR1H4, P2RX1, P2RX2, P2RX2, P2RX3, P2RX3, P2RX4, P2RX5, P2RX5-TAX1BP3, P2RX6, P2RX6P, P2RX7, P2RY1, P2RY10, P2RY11, P2RY12, P2RY13, P2RY14, P2RY2, P2RY4, P2RY6, P2RY8, PADI4, PARP, PARP1, PELI1, PELI2, PELI3, PEP, PRCP, PYCARD, RIPK1, RIPK2, RIPK3, RNF31, SENP6, SENP7, SERPINA1, SERPINA4, SESN2, SIRT1, SIRT2, SLC44A2, SOCS1, SOD, SOD1, SUGT1, TAB1, TAB3, TBK1, TBKBP1, TF, TFP1, TFPI, THOP1, TICAM1, TMPRSS4, TMRPSS2, TNF, TNFAIP1, TNFAIP2, TNFAIP3, TRADD, TRAF2, TRIB3, TRIM31, TRPA4, TRPM2, TRPV1, TRPV4, USP18, VDAC1, VDR, XDH, and/or ZBP1, and combinations thereof in the sample, wherein the presence of the at least one SNP confirms a higher risk for having or developing a health condition related to inflammasome NLRP3 dysregulation, RAAS dysregulation, KKS dysregulation, protease dysregulation, and/or cytokine dysregulation; and
b) treating the subject with a therapy for the treatment of a health condition related to inflammasome NLRP3 dysregulation, RAAS dysregulation, KKS dysregulation, protease dysregulation, and/or cytokine dysregulation, wherein the treatment comprises personalized pharmacogenomics and/or nutrigenomics therapies comprising administration of any compound listed in Tables 1, 2, 3, or 4 in FIGS. 1, 2, 3, and 4.

In some aspects, the health condition is SARS-CoV-2 infection and/or COVID-19.

### Packaged Kits

In another embodiment, a packaged kit is provided for use in the treatment of a health condition related to inflammasome NLRP3 dysregulation, RAAS dysregulation, KKS dysregulation, protease dysregulation, and/or cytokine dysregulation in a subject in need thereof comprising:
a) any of the pharmaceutical compositions disclosed herein;
b) a container housing the pharmaceutical composition during storage and prior to administration; and
c) instructions for carrying out administration of the pharmaceutical composition in a manner effective to treat the health condition, including any of the methods disclosed herein.

The instructions will typically be written instructions on a package insert and/or on a label. Depending on the type of formulation and the intended mode of administration, the kit may also include a device for administering the formulation. The formulation may be any suitable formulation as described herein. For example, the formulation may be an oral dosage form containing a unit dosage of a selected active agent. The kit may contain multiple formulations of different dosages of the same agent. The kit may also contain multiple formulations of different active agents.

### Definitions

It is to be understood that the present disclosure is not limited to the particular compositions, or uses or methodologies or process described, as these may vary for each subject. It is also to be understood that the terminology used in the description is for the purpose of describing the particular aspects or embodiments only and is not intended to limit the scope of the present invention. It is also to be understood that disclosures from one embodiment may be incorporated into other embodiments, and disclosures noted in a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, and processes have not been shown in detail in order not to unnecessarily obscure the invention. It is intended that no part of this specification be interpreted to affect a denial of any part of the full scope of the invention. Thus, the following descriptions and disclosures are intended to only exemplify some particular aspects of the disclosure, and not to exhaustively specify all combinations, permutations, and variations thereof. Additionally, unless defined otherwise in the invention, terms are to be understood according to conventional usage by those of ordinary skill in the relevant art. The terminology used in the description of the disclosure herein is for the purpose of describing particular embodiments only and is not intended to be limiting of the disclosure.

Following long-standing patent law convention, the terms “a,” “an,” and “the” refer to “one or more” when used in this application, including the claims. Thus, for example, reference to “a subject” includes a plurality of subjects, unless the context clearly is to the contrary (i.e., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, i.e., whole integers, including fractions thereof, subsumed within that range (for example, the recitation of 1 to 5 includes 1, 2, 3, 4, and 5, as well as fractions thereof, i.e., 1.5, 2.25, 3.75, 4.1, and the like) and any range within that range.

Active Agents—The terms “agent,” “active agent,” and “pharmacologically active agent” are used interchangeably herein to refer to a chemical compound that induces a desired effect. Furthermore, reference to “an active agent” or “a pharmacologically active agent” includes a single active agent as well as two or more different active agents in combination.

Medical/Technical Terminology—unless otherwise defined in the invention, medical, scientific and/or technical terms used in connection with the present invention shall have the meanings and definitions that are commonly understood by those of ordinary skill in the art. Additionally, nomenclatures utilized in connection with medical disease, medicinal and pharmaceutical chemistry, nutraceuticals and their chemistry described herein are those well-known and commonly used in the art. Standard techniques are used for chemical syntheses, chemical analyses, pharmaceutical preparation, nutraceutical preparation, formulation, compounding, delivery, and administration to patients as treatments and administration in a clinical trial.

Organism—as used in the invention the term “organism” refers to and describes an individual animal, plant, single cell life form.

Mammal—as used in the invention the term “mammal” refers to any animal classified as a mammal, including human, domestic and farm animals, nonhuman primates, zoo, sports, or pet animals comprising dogs, cats, horses, cows, etc.

Bats—as used in the invention the term “Bats” or “Chiroptera” are used interchangeably and refer to or describe mammals of the order Chiroptera which are the only mammals capable of sustained flight. Additionally, bats can harbor deadly viruses (i.e., SARS, Ebola, Marburg, etc.) without severe adverse health consequences.

Artificial or Induced biological mimicry—as used in the invention the term “biological mimicry” refers to or describes an evolved resemblance between an organism of one species with another organisms of another species and/or with an object wherein this evolved resemblance can be chemical, visual, electrical, acoustic, tactile, or electric, or combinations of these modalities. Thus, as used in the invention the term “artificial or induced biological mimicry” refers to or describes the methods or use of compositions to mimic a beneficial advantage present in one organism into other organisms particularly a mammal.

Mimic—as used in the invention the term “mimic” refers to the verb to imitate. For example, in one embodiment of the invention the term “mimic” refers to or describes a methods and compositions to induce the select immune advantageous of a bat in another mammal.

SARS-CoV-2—as used in the invention the term “SARS-CoV-2” or “SARS-COV2” are used interchangeably and refer to Severe acute respiratory syndrome coronavirus 2 which is a strain of a corona virus which is a pathogen and can infect certain mammals.

COVID-19—as used in the invention the term “Covid-19”, or “COVID19”, or Covid19″ are used interchangeably and refer to or describe the physiological condition in mammals that is typically characterized by infection with SARS-CoV-2. COVID-19 has symptoms and signs comprising: cough, anosmia, congestion, sore throat, myalgias, headache, dyspnea, conjunctivitis, hypoxemia, acute lung injury, acute respiratory distress syndrome (ARDS), L phenotype and H phenotype lung injury, lung edema, lung fibrosis, thromboembolic disease, microvascular and/or macrovascular injury, vasculitis, myocardial infarction, stroke, myocarditis, pericarditis, cardiac tamponade, arrythmia, Kawasaki like syndrome (i.e., strawberry tongue, lymphadenopathy, vasculitis), renal disease, gastrointestinal disease, liver injury (i.e., transaminitis, hepatitis), neurologic disease including encephalitis, retinitis, optical neuritis, retinal hemorrhage, cotton wool spots in the retina, conjunctivitis, skin ulcers, vascular skin lesions, rash, urticarial lesions and other clinical presentations.

Host—as used in the invention the term “host” refers to an organism infected with a pathogen and can include a mammal particularly a human host.

Subject—as used in the invention the term “subject” or “human subject” or “patient” or “patient subject” are used interchangeably and as used in the invention refer to a mammalian patient, such as a human or a veterinary subject who is confirmed to have a disease or who has risk factor(s) for a particular disease as determined by various methods comprising family history, environmental exposure history, genetic/epigenetic/metagenomic history or as determined by testing results, medical history, epidemiological factors. Particularly, in some embodiments the term “patient” refers to a human who is suspected of having SARS corona virus-2 (SARS-CoV-2) infection or who has confirmed SARS-CoV-2 infection via methods comprising testing, clinical presentation, or who has probable or confirmed exposure to an inanimate or animate object including a mammal infected or exposed to SARS-CoV-2.

SARS Patient—as used in the invention the term “SARS patient” or “patient having SARS,” “patient infected with SARS,” “patient with SARS,” “patients suffering from SARS,” “SARS-Host,” or “COVID-19 patient” are used interchangeably and refer to or describe a mammal who is infected or is suspected of being infected with a SARS coronavirus pathogen (i.e., SARS-CoV-2, SARS-CoV, MERS-CoV). Additionally, a SARS patient is one who is diagnosed via testing positive for SARS infection or one who is suspected of having SARS infection based on epidemiological factors, or one who is at an imminent risk of contracting SARS (i.e., one who has been exposed or will likely be exposed to SARS in the near future).

Illness—as used in the invention, the term “illness,” or “disease,” or “adverse health sequala” “disease phenotype”, “clinical disease phenotypes”, “medical condition,” or “health condition”, or “abnormal health condition” are used interchangeably and refer to or describe the physiological condition in a mammal that is typically characterized by a disorder in a structure and/or function in a mammal.

Disorder—as used in the present invention, the term “disorder” is any health condition or an illness, or a disease, and/or a disease risk in a mammal, that is resultant from a multi-system dysregulation comprising: dysregulation in PRR particularly a dysregulation in inflammasome priming and/or activation, a dysregulation in RAAS-KKS, a dysregulation in proteases, and a dysregulation in cytokine and wherein the dysregulation responds positively to a compositions administered and used as a treatment as disclosed in the invention wherein the composition is a modulator and the method and use is modulation comprising: modulation of dysregulated inflammasome priming and/or activation, and/or modulation of RAAS-KSS systems, and/or modulation proteases, and/or modulation of cytokines in accordance with the compositions and methods and uses of the present invention. As used in the invention a disorder can be acute and/or chronic, a risk factor and/or a predisposition to a disease and/or a pathological condition which predispose a mammal to the disorder.

Treatment—as used in the invention the term “treatment” is to include preventive, and prophylactic and/or therapeutic and/or restorative treatments and measures. Subjects in need of treatment include those who already have the disorder as well as those in which the disorder is to be prevented.

Therapeutically effective amount—as used in the invention the phrase “therapeutically effective amount,” refers to an amount of a composition (pharmaceutical and/or nutraceutical) to be administered to a mammal (preferably human) in a single dose or as part of a series of doses to render “a therapeutic effect” which is defined herein to generate at a minimum in the treated subject/patient a clinical response comprising: a reduction in the clinical signs, a reduction in the symptoms, and a reduction and/or improvement of the clinical impact of a disease. Furthermore, therapeutically effective amount also refers to compositions that induce a detectable therapeutic effect without undue adverse side effects (such as allergic reactions, toxicity, irritability, adverse health sequalae) and/or which is commensurate with a reasonable benefit versus risk ratio when used in the manner of the invention and the clinical response may range from a minimal decrease in pathogenic burden to prevention of a disease. For example as per one embodiment of the invention (though not limited to this example) “therapeutically effective amount” in a subject with COVID-19 refers to a reduction and/or improvements comprising: in viral burden, viral induced cytokine storm, viral induced immune dysregulation, viral-induced pyroptosis, viral induced tissue damage (i.e., acute lung injury hypoxemia and/or arterial and/or thromboembolism in systemic and/or pulmonary vasculature), reduction in clinical symptoms (i.e. dyspnea, cough, headaches, fatigue), reduction in clinical signs (i.e. fever, tachypnea, tachycardia, hypotension, hypoxemia, rash, weakness), a minimal decrease in pathogen burden from SARS-COV2, or prevention of SARS-COV2 infection. Ideally, the treated subject will not exhibit the more serious clinical manifestations of the disease (i.e., COVID19). The effective amount and dosage of the compositions of the invention can vary depending upon unique conditions of the subject comprising: type of subject, age, weight, height, body mass index, state of health, the presence of other disease, chronic disease risks, the presence of chronic disease, comorbidities, allergies, liver function, kidney function, single nucleotide polymorphisms of metabolizing enzyme genes, genetic risks, environmental exposure, epigenetic status, metagenomics, microbiome status, nature and severity of the condition to be treated, the method of administration, the duration of treatment, the nature of concomitant therapy (if any), drug-drug interactions, the specific formulations employed, and the like. Hence, it is not possible to specify an exact effective amount, dose, frequency, and duration in advance. However, the effective amount for a given situation can be determined in routine clinical trials and/or otherwise by means known to those skilled in the art. For example, a therapeutically effective amount of an active agent includes a dose that is in the range of from about 1 ng to about 10,000 mg, typically in the range of from about 100 ng to about 5,000 mg, and/or greater than about 1 ng, about 5 ng, about 10 ng, about 20 ng, about 30 ng, about 40 ng, about 50 ng, about 100 ng, about 200 ng, about 300 ng, about 400 ng, about 500 ng, about 1 μg, about 5 μg, about 10 μg, about 20 μg, about 30 μg, about 40 μg, about 50 μg, about 100 μg, about 200 μg, about 300 μg, about 400 μg, about 500 μg, about 1 mg, about 5 mg, about 10 mg, about 20 mg, about 30 mg, about 40 mg, about 50 mg, about 100 mg, about 200 mg, about 300 mg, about 400 mg, about 500 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, or about 9,500 mg. In another example, a therapeutically effective amount of an active agent also includes a dose that is in the range of from about 1 fg to about 1 mg, typically in the range of from about 100 fg to about 1 ng, and/or greater than about 1 fg, about 5 fg, about 10 fg, about 20 fg, about 30 fg, about 40 fg, about 50 fg, about 100 fg, about 200 fg, about 300 fg, about 400 fg, about 500 fg, about 1 pg, about 5 pg, about 10 pg, about 20 pg, about 30 pg, about 40 pg, about 50 pg, about 100 pg, about 200 pg, about 300 pg, about 400 pg, about 500 pg, about 1 ng, about 5 ng, about 10 ng, about 20 ng, about 30 ng, about 40 ng, about 50 ng, about 100 ng, about 200 ng, about 300 ng, about 400 ng, about 500 ng, about 1 μg, about 5 μg, about 10 μg, about 20 μg, about 30 μg, about 40 μg, about 50 μg, about 100 μg, about 200 μg, about 300 μg, about 400 μg, or about 500 μg.

Administer a therapeutically effective amount—as used in the invention the term “administer a therapeutically effective amount” of a composition means that the patient subject is treated with the compound(s) in an amount, concentration, frequency and duration which is sufficient to induce or mediate a sustained improvement over baseline in at least one indicator that reflects the severity of the disorder. The degree of improvement is determined based on signs or symptoms and can be determined in by the patient's treating physician and/or treating team via methods comprising conducting a health assessment, history, physical examination, testing, administering questionnaire to the subject. administered to the patient.

Biologically active—as used in the invention the term “biologically active” refers to or describes the ability to modify the physiological system of a mammal. A molecule can be biologically active through its own functionalities or may be biologically active based on its ability to activate or inhibit molecules having their own biological activity.

Receptor—as used in the invention the term “receptor” is a chemical structure composed of protein within the body of the mammal particularly inside a cell, or on the cell membrane that a modulator or a ligand binds and transduces a signal.

Modulator—as used in the invention the term “modulator” refers to or describes an agent, or a compounds or a composition, or a molecule, or a an electron, or a proton, or an ion that modulates comprising: the activity of a molecule, a biochemical pathway, a signal transduction pathway, a receptor, gene transcription, gene activity, gene methylation, gene phosphorylation, the activity of an organelle. For example, a modulator can bind to a receptor to change the receptors response to a stimulus. This modulator is termed an allosteric modulator. Modulators are positive, negative, or neutral.

Ligand—as used in the invention the term “ligand” is a composition or a compound or a molecule, or an ion that binds to a receptor or another molecule and produces a signal. Modulators of receptors are called ligands.

Agonist—as used in the invention the term “agonist”, “receptor agonist”, or “activator” are used interchangeably and refer to or describes a compound, a composition that binds to a receptor and which activates the receptor or signal transduction pathway. The activation is partial or complete.

Mimetic—as used in the invention the term “mimetic” refers to or describes a molecule or a composition such as a peptide that biologically mimics the action or activity of another protein in the mammalian body.

Antagonist—as used in the invention the term “antagonist”, “receptor antagonist” or “inhibitor” are used interchangeably and is a chemical compound that binds to a receptor and inhibits the receptor or signal transduction pathway. The inhibition is partial or complete.

Biochemical reaction—as used in the invention the term “biochemical reaction” is a process in living organism which one set of chemical compositions are transformed to another set of chemical substances. The change involves the position of electrons in breaking and forming a chemical bond and does not involve the nucleus.

Signal transduction pathways—as used in the invention the term “signal transduction pathways” refers to a process in an organism by which a physical or a chemical signal is transmitted through a cell as a set of chemical reactions and/or a cascade of reactions which is started by a ligand binding to a receptor and either activating (partial or full) or inhibiting (partial or full) the receptor or remaining neutral. Signaling pathways can interact with each other and form networks and allow coordinated cellular responses to changes within the cell and changes outside the cell.

Enzyme—as used in the invention the term “enzyme” is a chemical in living organisms that facilitates as a catalyst to accelerate and increase the reaction rate of biochemical reactions. Enzymes are proteins that act as biological catalysts. Ribozymes are RNA molecule catalysts. Different enzymes that catalyze the same chemical reaction are called isozymes. Enzymes comprise oxidoreductase, transferases, hydrolases, lyases, isomerases and ligases. Almost all biochemical reactions in an organism requires enzymes.

Substrate—as used in the invention the term “substrate” refers to a molecule upon which the enzyme acts and the enzyme converts the substrate into a different product.

Nutraceutical—as used in the invention the term “nutraceutical” refers to a chemical that is found in nature and present in organisms (i.e., plants, organism, bacteria, fungi etc.) which has not been intentionally modified in the laboratory. Examples would be peptides, polyphenols, lignans, etc.

Pharmaceutical—as used in the invention the term “pharmaceutical”, “pharmaceuticals”, “pharmaceutical agents”, “pharmaceutical compositions”, “pharmaceutical compounds”, “drugs”, “medications”, are used interchangeably and refers to a composition comprising: a molecule, a compound, an ion, an element, a drug, a medication” which can induce a therapeutic effect when properly administered to a subject and its uses comprise: to diagnose, to test, to prevent, to mitigate, to treat, to restore, to optimize, to vaccinate and/or cure a disease.

Pharmaceutically acceptable—as used in the invention the term “pharmaceutically acceptable” refers to compounds and compositions which are suitable to be administered to mammals including humans and animals without unwarranted adverse side effects or harmful reactions such as toxicity, allergic response, irritability, immune and inflammatory response and other side effects commensurate with a reasonable benefit to risk ratio in the subject.

Active Pharmaceutical Ingredient (API)—as used in the invention the term “active pharmaceutical ingredient”, or “active compound” are used interchangeably and refer to and describe as defined by FDA Guidance—Manufacturing, Processing, or Holding Active Pharmaceutical Ingredients (FDA-2018-D-1176), which states: “Any substance or mixture of substances intended to be used in the manufacture of a drug (medicinal) product and that, when used in the production of a drug, becomes an active ingredient of the drug product. Such substances are intended to furnish pharmacological activity or other direct effect in the diagnosis, cure, mitigation, treatment, or prevention of disease or to affect the structure or function of the body.”

Pharmaceutically acceptable salt and/or hydrate and/or cocrystal, ester, prodrug, organic acid, inorganic acid, amines and the like—as used in the invention the term pharmaceutically acceptable salt and/or hydrate and/or cocrystal and/or ester and/or pro drug or organic and/or inorganic acid, or amine forms refer to or describe the alternative forms that each composition, and/or pharmaceutical and/or nutraceutical and/or formulation in the invention can have and it is not limited to what is disclosed herewith and which can be used as a treatment in a mammal in need thereof and are materials generally known to those skilled in the art. Examples of salts comprise: hydrochloride, hydrobromide, bisulfate, phosphate, nitrate, acetate, valerate, oleate, palmitate, sulfate, stearate, laurate, lactate, phosphate, benzoate, maleate, fumarate, citrate, glutamic, salicylic, sulfanilic, tosylate, succinate, tartarate, toluenesulfonic, methanesulfonic, ethane disulfonic, oxalic, isothionic naphtylate, mesylate, lactobionate, and the likes.

Pharmaceutical carrier—as used in the invention the term “pharmaceutical carrier” or “pharmaceutically acceptable carrier” is used interchangeably and refer to or describe a compositions that do not produce and adverse reaction (i.e., toxicity, irritation, and/or an allergic response) in the subject (i.e. a mammal, particularly human). The pharmaceutical carrier is a pharmaceutically acceptable vehicle, a composition and/or a molecular entity comprising: nanostructure, solvent, suspending agent for delivering the compositions (pharmaceuticals and/or nutraceuticals) of the present invention to the subject. The carrier may be liquid or solid or vaporized and is selected with the planned manner of administration in mind. Examples of pharmaceutically acceptable carriers that may be utilized in accordance with the present invention comprise but are not limited to: excipients, diluents, liposomes, solvents, surfactants, suspending agents, buffering agents, lubricating agents, thickeners, thixotropic agents, penetration agents, adjuvants, aqueous buffers, oils, vehicles, emulsifiers, absorbents, dispersion media, coatings, stabilizers, protective colloids, adhesives, thickeners, sequestering agents, isotonic, nanoparticles, microspheres, microencapsulation's and absorption delaying agents and the likes that do not affect the activity of compounds of the compositions (i.e. active pharmaceutical ingredient, pharmaceutical, nutraceuticals) as disclosed in the invention. Examples of carriers comprising (but not limited to: DMSO, Ringer's solution, a buffered solution, hyaluronan, Ethanol, starch, cellulose, glucose, lactose, sucrose, gelatin, malt, rice, flour, chalk, silica gel, Poly ethylene glycol, magnesium stearate, sodium stearate, glycerol monostearate, sodium chloride, water. The compositions of this invention comprise a therapeutically effective amount of at least one of the compositions described herein in combination with a pharmaceutically acceptable carrier.

Administration routes—as used in the invention, the term “administration routes”, or “routes of administration” are used interchangeably and refer to the means wherein the compounds of the compositions of the present invention can be administered to a subject by any method known in the art, including but not limited to oral, intramuscular, subcutaneous, intradermal, parenteral, intraperitoneal, intranasal, inhalational, nebulized, intramucosal, sublingual, rectal, topical, transdermal, intravitreal, conjunctival, intra ocular, mucosal, intratracheal, with Intravenous Bags, intra-urethral, vaginal, labial, intra auris, and intravenous routes, including both local and systemic applications. The route of administration is also determined by the medical condition being treated and organ and/or tissue whereupon the composition with be acting as well the compositions bioavailability, pharmacology of the composition, subject's state of health, subject's state of consciousness, subject's ability and functionality, subject being on a ventilator, the availability of compounding and/or manufacturing, price, and other like factors. Furthermore, the compositions and compounds of the present invention may be designed to provide (but not limited to) immediate release, delayed, controlled or sustained release using formulation techniques which are well known in the art. Compositions of the invention can be administered in formulations comprising (but not limited to): solids and/or liquids and/or gaseous, vaporized, syrup, tablets, gel capsules, vegetarian gel capsules, pastilles, dry powder sachets, emulsions, pellets, inhalation solution, drops, ampule, gummy bears, effervescent tablets, inhalational, nebulized, nasal sprays, disks, mucosal preparation, rectal preparations, creams, lotions, gels, serums, colloidal preparations, liposomal preparations, topical preparations and transdermal preparations and the likes via techniques which are well known in the art.

Stabilizers—as used in the invention the term “stabilizers” or “excipients”, or “filler” are used interchangeably and refer to or describe compounds and/or molecules that are non-toxic and added to the active pharmaceutical ingredient to maintain the desirable properties of the composition until it is consumed by a subject and comprise (but not limited to): preservatives, thickeners, suspensions, emulsions, vehicle, flavor, binders, granules, gliders and the likes.

Adjuvants—as used in the invention the term “adjuvant” refers to an immunostimulatory substance which is designed to enhance some of the peptide compositions disclosed herewith and can reduce inflammation comprising: an aluminum phosphate, aluminum based mineral salt, calcium based mineral salts, zinc based salts, and dexamethasone and the likes.

Modified and truncated compositions—the compositions and compounds of the invention as disclosed herein can be modified or truncated and continue to retain the desired modulatory effect as one with ordinary skill in the art will appreciate.

Compounding—as used in the invention “compounding” refers to or describes creation of a particular pharmaceutical product to personalize the pharmaceutical and nor nutraceutical treatment to match the unique medical needs of the subject.

Sequential therapy—as used in the invention the term “sequential therapy” refers to or describes a treatment regimen or treatment schedule whereupon a first composition as disclosed by the invention is administered to a subject in need thereof as a treatment followed by a second or third or nth composition of the invention.

Simultaneous therapy—as used in the invention the term “simultaneous therapy” refers to or describes a treatment regimen or treatment schedule whereupon several compositions of the invention are administered simultaneously to a subject in need thereof.

Concurrent therapy—as used in the invention the term “concurrent therapy” is used interchangeably with “other therapies”, “additional therapy”, “combination therapy” and “adjunctive therapy” and refers to a subject who is administered a single or multiple composition of the present invention as a treatment and also receives one or several medications or nutraceuticals which are separate from the compositions of the invention either simultaneously and/or sequentially. An example comprises a subject who is treated with a vaccine and simultaneously receives compositions as disclosed in the invention as treatments.

Cellular stress—as described in the invention “cellular stress” refers to or describes a physiological condition wherein cells are continuously subjected to various endogenous and/or exogenous stress comprising DNA damage, mitochondrial DNA damage, mitochondrial dysfunction, hypoxia, radiation damage, pathogen invasion, extremes of temperature, PH, osmolarity, oxidative stress and free radical damage wherein the stress perturbs cellular homeostasis with resultant macromolecular damage comprising damage to proteins, RNA, DNA, and lipids, gene expression changes, epigenetic changes to DNA.

Cellular stress response—as described in the invention “cellular stress response” refers to or describes a physiological condition of a cell response provided via varied molecular changes towards cellular stress in an attempt to main cell homeostasis.

Damage associated molecular patters (DAMPs)—as described in the invention “DAMPs” and “Alarmins” are used interchangeably and refers to intracellular signals that signify cellular stress and dysregulation and which can activate pattern recognition receptors (PRRs) (i.e., NLRP3). DAMPs comprise toxins, excess glucose, ceramides, fibrillar or oligomeric peptide, fibrillar α-synuclein, neurotoxic prion peptide, uric acid crystals, calcium crystals, calcium phosphate crystals, cholesterol crystals, Endoplasmic (ER stress), Unfolded protein response (UPR), mitochondrial stress (mitochondrial DAMPs), mitochondrial bioenergetic changes, mitochondrial DNA inside a cell cytoplasm, ATP loss pathways via dysregulation of various components of the mitochondrial complex, damaged and/or oxidized mitochondrial DNA, mitochondrial reactive oxygen species (mtROS), xanthine oxidase generated ROS, disruption of Trans Golgi network (TGN), lysosomal rupture, Potassium Efflux (K efflux), Chloride influx, high mobility group box 1 (HMGB1), heat-shock proteins hsp70 and hsp90, cardiolipin release, cathepsin release, and Nlrp3 deubiquitination.

Mitochondrial DAMPS—as described in the invention mitochondrial “DAMPs” refer to DAMPS associated and released by mitochondria.

Pathogen associated molecular patterns (PAMPs)—as described in the invention, “PAMPs” comprise viral DNA, single or doubles stranded RNA (i.e., SARS-CoV-2), nucleic acids, lipopolysaccharides (LPS), fungal, protozoal, pathogen nucleic acids, amyloid B, and extracellular ATP which can sense PRRs and activate these PRRs.

Pattern recognition Receptors (PRRs)—as used in the invention the term “PRRs” refer to mammalian cell receptors which are located inside a cell and/or on a cell's, membrane called pattern recognition receptors (PRRs) which detect endogenous and/or exogenous stress which can for example signify to a cell that it is being damaged and/or invaded. The cell stress comprises of pathogen invasion (i.e. bacterial, viral, fungal, parasitic), misfolded proteins, oxidative damage, damaged proteins, damaged, DNA, damaged mitochondria, energy imbalance in the cell, accumulation of crystals in the cell and other damage and pathogen signals. PRRs respond to DAMPs and/or PAMPS or other signals and are activated and induce a signal transduction pathway.

PRR types—as used in the invention PRRs are subdivided into two major classes based on their subcellular localization: 1) Toll-like receptors (TLRs) and C-type lectin receptors (CLRs) are transmembrane proteins found in the plasma membrane and endosomes, where they can survey PAMPs and DAMPS in the extracellular space and/or inside an endosome; and 2) a second class of PRRs resides in intracellularly in the cytoplasm or in a compartment such as endosomes or nucleus and comprise: absent-in-melanoma 2 (AIM2) like receptor (ALR), retinoic acid-inducible gene I (RIG-I) like receptor (RLR), and nucleotide-binding oligomerization domain (NOD), leucine-rich repeat (LRR)-containing proteins (NLR) proteins. There are approximately 22 cytosolic NOD-like receptors NLRs. However, only select ones are able to oligomerize and form multimeric protein complexes called an inflammasome.

PPRs are activated by PAMPs and/or DAMPs and when a PRR is activated, it can trigger downstream inflammatory signal transduction pathways to eliminate microbial infection and repair damaged tissues. However, dysregulation in the PRRs activation can lead to dysregulated immunity and cytokine storm.

RAAS-KKS—as used in the invention RAAS-KKS refers to the Renin aldosterone angiotensin system (RAAS) and the Kallikrein-Kinin (KKS) system.

RAAS/ACE Renin aldosterone angiotensin system (RAAS)—as used in the invention RAAS is a system of enzymes, peptides/ligands and receptors that involve multiple organs, it has 2 opposing arms ACE/AngII and its ligands and ACE2/Ang (1-7)/Mas receptor and their respective ligands. RAAS, as used in the invention, is involved with hemostasis components common to venous and arterial diseases.
Angiotensin converting enzyme (ACE; kinase II, dipeptidyl carboxypeptidase)—as used in the invention, ACE is a zinc metalloprotease and responsible for the conversion of angiotensin I generated by the Enzyme Renin acting on angiotensinogen, to angiotensin II (AngII). ACE inactivates other proteins comprising bradykinin, Lys-bradykinin. ACE is located mainly in capillaries of lungs and can be found in endothelial and kidney epithelial cells and other organs. ACE has several ligands and can cleave these ligand peptides comprising angiotensin I, bradykinin, Lys-Bradykinin, Angiotensin I, and N-acetyl SDKP. ACE is a membrane bound enzyme and it can be cleaved and shed through ADAM9 and becomes secretory ACE. ACE is related to hypo-fibrinolysis by both increasing the PAI-1 expression and decreasing t-PA production.
ACE and its ligands impact thrombogenesis comprising: Angi-I can impact vascular clotting by inducing Tissue factor and PAI-1, rendering endothelial thrombogenic. Ang-II can be prothrombotic through adhesion and aggregation of platelets and stimulation of PAI-1 and PAI-2 Ang III which also acts via AT1/AT2 can be prothrombotic through adhesion and aggregation of platelets and stimulation of PAI-1 and PAI-2. Ang-IV acting through AT4 (TRAP or c-Met receptors) can n endothelial cells and VSMC (vascular smooth muscle cells), Ang-IV up-regulates plasminogen activator inhibitor-1 expression and could participate in thrombus formation. prothrombotic potential through adhesion and aggregation of platelets and stimulation of PAI-1 and PAI-2. AngIV-AT4R signaling has a key role in fibrinolysis and the subsequent formation of arterial thrombosis after vascular injury. Aldosterone a hormone involved in blood pressure control via sodium and water retention is part of the RAAS system.
ACE via kinin receptor has an inhibitory role on IFN responsive gene inhibition.
Angiotensin II (Ang II)—as used in the invention, Angiotensin II is the major bioactive peptide of the renin-angiotensin system (RAS), plays a major role in the regulation of vascular function and structure. It is a multifunctional vasoactive peptide produced systemically and locally within the vascular wall. Ang II is a potent vasoconstrictor that also has mitogenic, proinflammatory, and profibrotic actions. Angiotensin converting-enzyme affects hemostasis by decreasing fibrinolysis. Ang-II causes alveolar epithelial apoptosis via AT1R.
Ang II exerts its diverse actions via two G-protein-coupled receptors, Ang II type 1 (AT1R) and type 2 (AT2R) receptors. The AT1R mediates most of the known actions of Ang II. The AT2R is associated with antiproliferative, proapoptotic, and vasodilatory actions of Ang II and tends to counteract AT1R vasoconstrictive and prothrombotic effects. AngII activates several pathways and mediates its effects via superoxide anion and hydrogen peroxide (H2O2).
Ang-II also activates vascular NADPH Oxidase (NOX). Ang-II can activate both NADPH and NADH oxidases with p22phox or CyBA which causes oxidative damage. Cytb245 is the redox element of the NADPH oxidase complex. Cytb245 is composed of two subunits: 1) p22phox (also called CYBA, the alpha/small subunit/light chain of cytb) and 2) gp91phox (renamed NOX2) (the beta/heavy chain/large subunit.) P22phos uses NADH or NADPH as the electron donor for the reduction of oxygen into superoxide anion and its derivatives including peroxynitrite (OONO2), hydrogen peroxide (H2O2) and hydroxyl radical (OH.). ROS regulated pro-inflammatory cytokine production and can be toxic if produced in excess. This superoxide production is important in phagocytic cells and lysosomes to destroy pathogens. p22phox is also expressed in endothelial and vascular smooth muscle cells. Specific polymorphisms of the CYBA gene are associated with a decreased risk of coronary artery disease. Ang II can increase vascular NOX mediated ROS production and mediate endothelial dysfunction. Ang-II can impact mitochondrial NOX.
Ang II also triggers NLRP3 inflammasome activation in a dose- and time-dependent manner, mediated via AT1 receptor rather than AT2 receptor.
Ang II stimulates generation of mitochondrial Nox4 and the overproduction of superoxide and hydrogen peroxide.
Ang II damages mitochondrial function comprising increased mitochondrial ROS, mitochondrial membrane potential decline. Mitochondrial dysfunction mediates Ang II-induced NLRP3 inflammasome activation. NLRP3 inflammasome activation is involved in Ang II-induced kidney damage via mitochondrial dysfunction.
Ang II also inhibits antioxidants, MnSOD, Cu/ZnSOD and catalase and increase in ROS induces xanthine oxidase.
Chymase—as used in the invention, chymase can produce AngII via an ACE independent mechanism primarily in vasculature and heart. Chymase is produced by Mast Cells. Thus, mast cells are the source of the vascular ACE-independent pathway, and the antihypertensive benefit of combining ACE inhibitor therapy with AT1 receptor antagonist therapy is most likely due to negation of chymase-catalyzed Ang II generation.
Genetic polymorphisms comprising ACE DD genotype—As used in the invention, genetic polymorphisms comprising ACE DD genotype represents a susceptibility marker of thrombosis in subjects apparently without predisposing factors and the DD genotype increases risk of venous thromboembolism in subjects in whom a thrombogenic condition occurs and AGT M235T single nucleotide polymorphism. Angiotensinogen (AGT) produced by the liver is cleaved to angiotensin I by renin in response to low blood pressure. Angiotensinogen increases proportionally to body mass index and RAS is more activated in Obesity. Single nucleotide polymorphisms in AGT gene such as M235T may be a risk factor for myocardial infarctions, especially in Asian population.
ACE Inhibitors and AT1R—as used in the invention, ACE Inhibitors and AT1R are used to treat hypertension and prevent cardiovascular events, chronic kidney disease protection.
NADPH oxidase enzyme—as used in the invention, NADPH oxidase enzyme is the primary source of superoxide production in inflammatory cells (i.e., macrophages, neutrophils, alveolar macrophages). The NADPH oxidase enzyme comprises of a number of protein subunits including the catalytic subunit NOX2, Rac1 (in endothelial cells it is Rac2).
NADPH Oxidase 2 (NOX2)—as used in the invention, NOX2 (also called phagocytic oxide, NADPH oxidase 2, NADPH oxidase components gp91phox) is encoded by CYBB gene and its protein enzyme is a superoxide generating enzyme generating ROS. NOX2 is found in plasma membranes including plasma membranes of endosome, phagosomes, endothelial cells, macrophages, neutrophils and other cells. It is membrane bound and It is inactive until it binds to P22phox. It is a terminal component of respiratory chain that creates superoxide molecule inside the endosome which is necessary to inactivate the pathogens by phagocytosis. It also functions as a voltage-gated proton channel that mediates the H (+) currents of resting phagocytes and participates in the regulation of cellular pH in endosomes. NOX2/CYBB also functions as a voltage-gated proton channel that mediates the H (+) currents of resting phagocytes and participates in the regulation of cellular pH in endosomes. Nox2 regulates PH in the phagosome, lysosome which is also required for efficient antigen cross-presentation by dendritic cells. NOX2 controls phagosome proteolysis in dendritic cells also via redox modulation of local cysteine cathepsins Nox2 activity participates in the regulation of the phagosome and endosomal pH in dendritic cells, and is required for efficient antigen cross-presentation. In the absence of active NOX2, the phagosomal and endosomal pH decreased. Nox2 deficiency from mutations in this gene results in chronic granulomatous disease, where neutrophils are able to phagocytize bacteria but cannot kill them in the phagocytic vacuoles as a result of not being able to deliver activated oxygen into the phagocytic vacuole to create reactive oxygen species. NOX2 also controls phagosomal proteolysis in DCs through redox modulation of local cysteine cathepsins. In viral infections the generation of H2O2 can inactivate TLR7 and possibly inhibit the viral response. Additionally, NOX2 was noted to activate NLRP3 inflammasome in the brain. Nox2 NADPH oxidase is dispensable for platelet ROS generation as well as platelet activation, adhesion, secretion, and aggregation.
Susceptibility to carotid artery thrombosis in a photochemical injury model was similar in wild-type and Nox2-deficient male or female mice. Our findings indicate that Nox2 NADPH oxidase is not an essential source of platelet ROS or a mediator of platelet activation or arterial thrombosis in large vessels, such as the carotid artery.
ACE-2 its ligands, Ang (1-7), Mas receptors and its ligands—as used in the invention ACE-2 its ligands, Ang (1-7), Mas receptors and its ligands are described as follows:
The RAAS system primarily has two axes mediated by the its two major enzymes comprising ACE its AT1R, AT2R and ligands and ACE2, Mas receptor, Ang (1-7) and its other ligands. These two axes primarily work in opposition. A dysregulation in these two axes can lead dysregulation in the lung cardiovascular, thromboembolic neurologic, renal, metabolic, immune, gastrointestinal and others.
ACE2 is membrane-bound. Most of the physiological function of ACE2 is exerted through its role in opposing the harmful effects of ACE and the Ang II-AT1R axis by activating Mas receptors and AT2R signaling, leading to the protective anti-oxidant, anti-inflammation, antifibrotic, antithrombotic, anti-hypertrophic and vasodilatory effects.
ACE2 can be secreted into circulation as an enzymatically active ectodomain ACE2 (sACE2). Soluble ACE2 is detected in human bronchoalveolar lavage fluid (BALF), in apical secretions of polarized epithelial cells.
ACE2 was mainly expressed in lung type 2 pneumocytes, liver cholangiocyte, colon colonocytes, esophagus keratinocytes, ileum ECs, rectum ECs, stomach epithelial cells, and kidney proximal tubules. It is also seen in vascular endothelial cells and cardiomyocytes.
There are no differences found in ACE2 activity among different age groups in bronchoalveolar lavage fluid, or in expression profiles of various ethnic groups. Genetic variants in ACE2 were not linked to susceptibility to COVID-19 infection. Missense ACE2 alleles may account for asymptomatic COVID-19 carriers.
Co-expression of ACE2 and TMPRSS2 was found in human nasal and respiratory sinuses, bronchial epithelium (in particular glandular epithelium), and alveolar epithelial type II cells, in the gastrointestinal system (esophagus, stomach, ileum, and colon), cardiomyocytes, in blood vessels (endothelial cells and vascular smooth muscle cells) and some other tissues.
Healthy hearts and kidneys are characterized by high levels of ACE2 mRNA and protein expression, with moderate expression of ACE.
ACE2 cleaves pyr-apelin-13 in a negative feedback loop. Apelin peptides upregulate ACE2 expression in heart and vasculature.
ACE2 knockout hypertensive mice show increased levels of proinflammatory cytokines IL1-b, IL-6, TNF-alpha. AT1R blockers induce ACE2 expression and reduced inflammatory cytokines and induction of anti-inflammatory cytokine IL-10.
Diabetes which has cardiovascular dysfunction has dysregulation of RASA toward the ACE/Ang-II proinflammatory arm. Increase of ACE2 through ACE inhibitors is protective in nephropathy and retinopathy.
ACE2 is expressed in gastrointestinal system at a highest rate and are involved in gut dysbiosis. ACE2 gain mutant improve leaky gut. Lungs can be affected through gut-lung axis, leading to pulmonary hypertension.
ACE2 has a RAAS-independent function regulating intestinal amino acid homeostasis and gut microbiome.
ACE2 in lungs is expressed in differentiated epithelial cells. ACE2 knockout mice have very severe ARDS, which could be partially rescued by additional ACE knockout. Injection of rhACE2 improved lung injury and inflammation in LPS (lipopolysaccharide)-induced acute lung injury. Oral feeding of bioencapsulated form of ACE2 protects and arrests the progression of pulmonary hypertension.
There is a sex difference between ACE-2 expression in males versus females being higher in males.
Many compounds can modulate the expression of ACE2. For example, hypoxia and decrease the expression of ACE2.
ACE2 gene also resides on the X chromosome. Women have x chromosome inactivation which can result in some females having less ACE2 gene expression
ACE2 SNPs can have an impact in various diseases comprising rs200745906, rs142984500, rs370187012.\
ADAM-17, is a sheddase and cleaves ACE2 into secretory ACE2 (sACE2) which is secreted into extracellular space. Loss of ACE2 activity causes overactivation of RAS and subsequent upregulation of ADAM-17, which leads to further loss of ACE-2 in a positive feedback loop. ADAM17 also cleaves its primary substrate releasing soluble TNF-α.
Ang (1-7) is a 7 amino-acid peptide that is cleaved by ACE2 from Angiotensin II. Ang (1-7) can also be produced by ACE from Ang (1-9) and Neprolysin converts Angiotensin I to Ang (1-7). Ang (1-7) counteracts effects of AngII. Ang (1-7) acts via Mas1 receptor and can increase NO release via activation of PI3K/Akt/eNOS pathway, thereby acting as a vasodilator and improving vascular endothelial function. These enzymes acting on Ang (1-7) comprise of: ACE2, ACE, Neprilysin (NEP), Prolyl endopeptidase (PEP), Prolyl-carboxypeptidase (PRCP), thimet oligopeptidase (THOP1). Ang (1-7) binds to Mas receptor and less to AT2R, AT1r, Neprilysin (NEP), Prolyl endopeptidase (PEP), Prolyl carboxypeptidase (PRCP), thimet oligopeptidase (THOP1).
The genes enzymes that produce Ang (1-7) comprise PEP (prolyl endopeptidase): PEP gene; NEP (neprilysin): MME gene; PRCP (Prolyl carboxypeptidase): PRCP gene; and THOP (thimet oligopeptidase): THOP1 gene.
Ang (1-7) actions include:
a) acting on the vasculature endothelium comprising antithrombotic, vasodilation, nitric oxide release through AKT-eNOS of FOXO1 activation, counter regulation of ANG II signaling, inhibiting c-Src, ERK1/2, NAD(P)H oxidase and VCAM, an Anti-thrombogenic effects through NO and prostacyclin release.
b) acting on vascular smooth muscle cells (VSMC): antiproliferation, decreases of ERK1/2 activation through prostacyclin release, reduction of neointimal thickness and area, retardation of osteogenic transition.
c) acting via MAS receptor, the receptor for Ang (1-7) which is angio-protective, antithrombotic. Animals knockout for Mas receptors have a shorter bleeding time and increased size of thrombi.
d) Ang (1-7) can attenuate the classical RAAS through a mitogen activated protein kinase.
Ang (1-9) is also cleaved by ACE2 or carboxypeptidase A or cathepsin A and acts on ATR2. Ang (1-9) ay be an endogenous ACE inhibitor.
Pyr-appelin-13 and dynorphin are cleaved by ACE2. ACE2 is a negative regulator of Pyr-appelin. NEP inactivates Pyr-appelin-13. Pyr-appelin-13 acts via AAPJ. Loss of Pyr-appelin-13 can increase platelet aggregation. directly Pyr-appelin-13 directly interferes with thrombin-mediated signaling pathways and platelet activation, secretion, and aggregation.
Alamandin also is cleaved by ACE2 and act via Mas-related G protein-coupled receptor member D (MrgD) receptor exerts vasodilatory and antihypertensive actions, likely induces NO production via increasing eNOS activity. Mice with MrgD receptor deletion develop dilated cardiomyopathy.
Currently there are no approved drug treatments for ACE2.

Kallikrein-Kinin (KKS) system—as used in the invention the KKS system comprises:

A high molecular weight Kininogen, Factor12 and Pre-kallikrein is produced by the liver enter endothelial cell gap junctions upon endothelial cell (EC) injury and immune activation of EC. Factor XII (Hageman factor) is activated by collagen (i.e., during type 1 or type 2 activation of endothelium by immune cells can reveal collagen from the media). Collagen activates Factor XII to Factor XIIa which then activates pre-kallikrein to Kallikrein which transforms High and Low molecular weight Kininogens (HMWK, LMWK) respectively to Bradykinin (a nonapeptide) and Lys-Bradykinin (kallidin, a decapeptide) respectively. Kininogen 1 is encoded by KNG1. Kininogen 1 is the precursor of HMWK, LMWK and bradykinine. Kallidin can also be converted into bradykinin by aminopeptidase B. Bradykinin and Kallidin are broken down to other metabolically active molecules by several enzymes including ACE, Neprolysin and others. The KKS re-enhances type type I and type II activation of endothelial cells by mast cells via histamine release.

Kinin peptides have a broad spectrum of activities that include the regulation of blood vessel tone, renal function, and protection from ischemia reperfusion injury. Kinins also participate in inflammation, producing vasodilatation, increased vascular permeability, neutrophil chemotaxis and pain. Bradykinin and Lys-bradykinin—are pro-inflammatory proliferative peptide hormones inducing vasodilation, vascular permeability, edema, increase the type 1 activation of endothelial cells and can cause angioedema.

The kallikrein system comprises of tissue kallikrein and 14 kallikrein-related peptidases (KLKs). These peptidases are subgroups of secreted trypsin or chymotrypsin like serine proteases. KLKs are expressed in many cell types. They regulate important physiological activities such as immune response, neural development, blood pressure, skin desquamation, tooth enamel formation, semen liquefaction.

Kallikrein is made from pre-kallikrein which is encoded by KLKB1 gene. Factor XII (Hageman factor) upon activation cleaves pre-kallikrein to kallikrein.

Plasma kallikrein is produced in the pancreas and circulates in the blood and cleaves HMWK to kinin peptides (bradykinin and lys-bradykinin).

Tissue kallikrein is expressed in most cells and abundant in pancreas, kidney, urine and saliva. It is also present in epithelial or secretory cells of various ducts, (i.e., salivary, sweat, pancreatic, prostatic and intestinal ducts, distal nephron). Tissue kallikrein is also present in neutrophils, colonic mucous cells, the trachea, nasal mucosa, and anterior pituitary. Tissue kallikrein cleaves LMW to Kinin peptides (bradykinin or Lys-bradykinin. The tissue kallikreins consist of 15 proteases.

All kallikreins are single polypeptides, called kallikrein-related proteases and named KLK1-KLK15. Kallikreins share 40% identity with each other.

KLK12 is distributed in many tissues with highest levels found in lungs. KLK12 has an angiogenic function and degrades several members of the Cysteine-rich angiogenic protein (CCN) which can modulate vascular endothelial growth factor and this proangiogenic activity of KLK12 is not kinin dependent activation of its receptor, the B2 receptor (B2R).

In respiratory disease KLK1 is consistently elevated in the bronchial alveolar lavage fluid of subjects with asthma or chronic bronchitis, where it is responsible for releasing kinins from HMWK which activates B2R which induces bronchoconstriction and hypersecretion of mucus and may contribute to metaplasia. KLK1 is also elevated in renal and cardiovascular disease. ROS upregulate KLK1 activity which induces degradation of the hyaluronic acid lining epithelial cells of lung airways. Additionally, dysregulated increased activity of KLK1 results in proteolytic cleavage of pro-EGF into mature EGF which activates EGFR.

As used in the invention, there are two types of kinin receptors comprising type 1 (B1) and type 2 (B2) receptor. The B2 receptor normally predominates, whereas B1 receptors are induced by tissue injury and are involved in inflammation. Bradykinin and kallidin are more potent on the B2 receptors. For example, in crystal induced arthropathy it was shown that B2 receptor antagonism decreased monocyte and neutrophil infiltration.

Decreased leukocyte infiltration was associated with reduced monocyte (CCL2) chemokine levels. MSU crystal-induced damage to the surrounding visceral membrane was also attenuated in the presence of B2 receptor antagonism. Together, these data show that bradykinin receptor 2 plays a role in maintaining MSU crystal-induced leukocyte infiltration and membrane permeability and identify the B2 receptor as a potential therapeutic target for managing inflammation in gout.

As used in the invention, NF-kappa-B, p38 and JNK are important for IL-1beta induced stimulation of B1 receptors, and NF-kappa-B and p38 are important for B2 receptor. Also, p38 and JNK were important for TNF-alpha induced stimulation of B1 receptors, whereas NF-KB, p38 and JNK are involved in TNF-alpha induced expression of B2 receptors. IL-1beta and TNF-alpha upregulate B1 and B2.

As used in the invention, Bradykinin and desArg-bradykinin stimulate macrophages to release TNF and IL-1.

Bradykinin, as used in the invention, acts on Br and BZ receptors, being much more potent at B2 receptors.

DesArg-bradykinin, as used in the invention, is a selective agonist for the Bi receptor.

Bradykinin receptors, as used in the invention, are present on macrophages that stimulate and kinins can stimulate TNF and IL-1 release in inflammatory lesions.

D2R, as used in the invention, activation counteracts the pro-oxidative, pro-inflammatory, and pro-apoptotic effects induced through B2R and modulated the expression of apoptosis markers, such as Bcl-2, Bcl-xL, Bax, and caspase 3/7 activity.

Bradykinin, as used in the invention, can be broken down by several enzymes at specific amino acids. Bradykinin has 10 amino acids. Bradykinin is cleaved by ACE 75% to (BK1-7), 21.5% by APP to (BK2-9), and 3.5% by kininase I to (Des-arg BK, BK1-8) which acts on BK1 receptor which is then broken down (half-life 8 minutes) by enzymes comprising: APP to BK (2-8) 65% and via ACE to BK (1-5).

As used in the invention, at low levels of Bradykinin there is affinity more for enzymes that have a low Michaelis constant (i.e., ACE). At higher bradykinin levels there is more affinity for enzymes that have a high Michaelis Constant (i.e., Neprilysin). For example, in the lung, cardiac and renal bush border membranes, and urine, Neprilysin was more contributory to BK than ACE. ACE and Neprilysin are 2 enzymes that cleave bradykinin. ACE and Neprilysin inhibitors increase bradykinin levels and can cause angioedema.

As used in the invention, the cleaving enzymes of Kinins comprise:

1. ACE (Kininase I) cleaves bradykinin (which acts on B1 receptor) at amino acid 7-8 which yield an 8 amino acid peptide (bradykinin (1-8) which acts on B2 receptors. ACE can also cleave bradykinin between 5-6 amino acid. ACE can form Bradykinin (1-5) which in studies have shown not to increase blood flow, nor does it increase tissue plasminogen activator (tPA) (which bradykinin increased tPA.) Both bradykinin and Bradykinin (1-5) inhibited alpha and gamma induced platelet aggregation.

2. Carboxypeptidase M or N (kininase II), as used in the invention, can breakdown bradykinin between 7-8 or 8-9 amino acid. The peptide cleaved between 8-9 is active on B1 receptors. Carboxypeptidase N (CPN) (Kininase I) is a membrane bound enzyme in plasma and present in diverse tissues comprising blood vessels, lung, kidney and placenta. The carboxypeptidase in endothelial cells and cultured fibroblasts is carboxypeptidase M, which also cleaves basic C-terminal amino acids. CPN has many actions including inactivating Bradykinin by removing and arginine to make Bradykinin (1-8) and removing a lysin from Lys-bradykinin to make bradykinin (1-9) which are bioactive and more potent on B1 receptors.

3. ACE2 can cleave the BK (1-8) formed by CPN to BK (1-7) which then can be cleaved by ACE to BK (1-5).

4. Neprilysin (neutral peptidase), as used in the invention, can breakdown bradykinin between 4-5 and 7-8 amino acids. Neprilysin gene knockout in mice causes increased basal vascular permeability, hypotension and reduced heart weight/body weight ratio.

5. NEP2 can breakdown bradykinin between 4-5 and 5-6 amino acids.

6. Aminopeptidase p, as used in the invention, can breakdown bradykinin between 1-2 amino acids. It also coverts kallidin to bradykinin.

7. DPP-IV can take BK2-9 (from APP cleavage) and form BK (4-9).

8. Prolyl peptidase can breakdown bradykinin between 3-4 and 7-8 amino acid.

9. Endothelin-1 (PH optimum: 6.8) can breakdown bradykinin between 7-8 amino acids.

10. Endothelin-1 (PH optimum: 5.5) can breakdown bradykinin between 7-8 amino acids.

11. Endopeptidase 24.15 can breakdown bradykinin between 5-6 amino acids.

12. DPPIV.

Kallistatin, as used in the invention, is a kallikrein binding protein and binds with kallikrein rendering it inactive. At high oxygen concentration Kallistatin is inhibited. At low oxygen levels Kallistatin is enhanced. SERPINA4 is the gene for Kallistatin. SERPINA1 is the gene for alpha-antitrypsin inhibitor which is protective in the lungs. Kallistatin can inhibit VEGH, Transforming growth factor beta, Vascular endothelial growth factor (VEGF), HMGB1, TNF-alpha. Kallistatin matches shares 44-46% sequence identity with human alpha 1-antitrypsin, alpha 1-antichymotrypsin, corticosteroid-binding globulin, protein C inhibitor, thyroxin-binding globulin, and rat kallikrein-binding protein.

Clotting, Kinins, Complement system, as used in the invention describe that upon tissue injury or infection PAMPS and or DAMPs such as S100 and the high mobility group box 1 (HBGM1) proteins, defensins, lectins, mitochondrial cardiolipin, cellular DNA can activate Kallikrein-Kinin system (KKS) in which HMWK becomes a cofactor for activated factor 12 to initiate the intrinsic clotting system.

Caspase (Casp)—as used in the invention “Casp” are a family of proteases enzymes playing a role in inflammation and/or cell death comprising apoptosis, pyroptosis, and necroptosis. Caspases directly process pro-inflammatory cytokines comprising pro-IL-1β, and/or pro-Il-18 to IL-1B and IL-18 respectively.

Cytokines—as used in the invention “cytokines” are small proteins (peptides) and cannot cross the lipid bilayer of cells to enter cells and are important in cell signaling wherein they are immune modulators comprising: proinflammatory, anti-inflammatory. Cytokines comprise chemokines, interferons, interleukins, lymphokines and tumor necrosis factor. They are produced by immune cells as well as other cells (i.e. endothelial cells, epithelial cells, fibroblasts). As used in the invention Cytokines are further described herein comprising:
1) Interferons (IFNs)—as used in the invention, are anti-viral compounds released by viral infected cells as a cellular response to detection of PAMPs. An IFN response is one of the responses of the innate immune system IFNs interfere with viral replication in host cells, they can activate other immune cells, they can lead to fever response. IFNs have direct antiviral effects in epithelial cells. Interferons comprise of: types I, II, and III. Type I IFNs comprises of IFN-α, -b u, -k. In humans IFN-α genes are composed of more than 13 subfamily genes. There is a single IFN-b gene. Type I IFNs promotes the polarization of macrophages to an inflammatory “M1” phenotype and increases the production of nitric oxide. Type I IFNs can also cause fever, myalgias and are inflammatory. Type II IFNs are referred to as gamma interferon (IFNγ). There is a single IFNγ gene. IFNγ is induced in immune cells such as T cells or natural killer (NK) cells. Type III is Interferon lambda (IFN k). Type I and Type III interferons are stimulated by the same PAMPs. Type III interferons preferentially act on epithelial cells and provide antiviral protection. Type III IFNs are less inflammatory than type I IFNs. IFNs are produced as a result of multiple signaling pathways within the cell which are activated upon invasion with a viral pathogen which lead to interferon production. The IFN activation signaling can occur through PRRs comprising TLR3, TLR7, TLR7/8, RIG-I and melanoma differentiation-associated protein 5 (MDA5), cGAS which activate other mediators such as TRIF which converge on NF-KB activation and Interferon regulatory factor 3 (IRF3) and Interferon regulatory factor 7 (IRF7) transcription factors which induce INF production.
2) PRRs—as used in the invention, single-stranded RNA (ssRNA) viruses which will be recognized by PRRs comprise TLR7 and RIG-I, double-stranded RNA (dsRNA) viruses will activate TLR3, RIG-I, MDA5. Signaling through PRRs will ultimately result in the translocation of NFκB, IRF3 and IRF7 transcription factors to the nucleus. NFκB transcription factor will result in induction of proinflammatory cytokines. The IRF3, IRF7 transcription factors will induce production if IFNs.
3) IRF3—as used in the invention IRF3 is expressed constitutively and exist ubiquitously as an inactive monomer in the cytosol. IRF3 regulates expression of IFN-beta gene. IRF3 also regulates IFN lambda 1 gene expression and only weakly induces IFN lambda 2 and 3. IFNβ gene is regulated by NF-KB, MAPK activated transcription factors and IRFs particularly IRF3 which is activated by virus induced phosphorylation of serine residues in the c terminal of IRF3 by serine-threonine kinases (TBK1 and IKKε) and form homodimers of IRF3 which translocates from the cytosol into the nucleus and binds to responsive elements on the DNA for IFNβ gene transcription leading to an antiviral response. IFNβ and IFN-lamda1 can be activated by RIG-I/Mays, TLR3/TRIF and TLR7/8/MyD88 pathways.
4) IRF7—as used in the invention, IRF7 is an IFN-stimulated gene and often induced at later phases of virus infection. IFN alpha genes are regulated by IRF7 which is induced by type I IFNs produced at early times of virus infection. IRF7 activates both late phase of IFN alpha and IFN B genes expression. IRF7 provides a positive feedback loop interferon production and has major role in IFN induction, even if IRF3 is absent. IRF3 mediated IFN production is minimal if IRF7 is low. IFN lambda, particularly IFN lambda 2 and 3 and IFN alpha have similar promoters and more restricted to positive regulation by IRF7. IFN lambda 2 and 3 genes are regulated by NF-KB, IRF7, which are the main regulators controlling the expression of these genes. IFN alpha and IFN-lambda 3 were activated by MyD88 via IRF7 and to a lesser extent by IRF1. Type I IFN induction is mainly regulated by IRF3 and IRF7. IFN lambda 1 and IFN beta are regulated by IRF3 and IRF7.
5) Interferon production—as used in the invention, interferon production is a signal transduction that is initiated by PRRs recognizing viral proteins or nucleic acids and comprising: RIGI/MAVS/IRF3: Retinoic acid inducible gene I (RIG-I) is ubiquitously available in all cell types including epithelial cells. The ligand for RIG-I is dsRNA and viral 5′ phosphorylated ssRNA. RIG-I is a unique molecule that can distinguish between self and non-self RNA. RIG-I upon activation with its ligand, transmits the signal via caspase recruitment domain to a mitochondrial protein cardif also known as MAVS which leads to the activation of IRF3 via IKKe/TBK1 and NF-KB via the classical IKK pathway and induces IFNs I beta and IFNs III Lambda-1.
6) TLR 3—as used in the invention, TLR3 recognizes viral dsRNA occur which occurs during viral replication. TLR3 has Toll/IL-1R domain containing adapter-inducing IFN-beta (TRIF) as an adapter molecule which activates IkB Kinase (IKK) E and TANK-binding kinase 1 (TBK1) which are virally induced and regulate phosphorylation and activation of IRF3 and IRF7 and interferon induce IFNs alpha beta production.
7) TLR7—as used in the invention, TLR7 plays an important role in inducing antiviral response against ssRNA. The signal transduction is via MyD88, IL-1 associated Kinase 1 and 4 (IRAK1, IRAK4) which activates IRF7 and NF-KB. This pathway is active in all cells including dendritic cells which produced high levels of IFN-alpha and IFN lambda 2 and 3. Thus, viral ssRNA antiviral response is mediated via TLR7/MyD88/IRAK1,4, IRF7/NF-KB and interferon alpha and lambda 2 and 3.
8) IFNs I and III—as used in the invention, IFNs I and III both exhibit early and late phase of IFN induction. IFN lambda 2/3 genes are functionally closer to IFN alpha and TLR7/8 or TLR9 MYD88 IRF activating pathway are important in regulating them. IFN alpha and IFN Lambda 3 are activated by the MyD88 dependent signal transduction via IRF7 and minimally by IRF1. IFN lambda 2 and 3 have 96% homology with each other, IFN lambda 1 has 81% homology with IFN lambda 2 and 3. The promoter of IFN lambda 2 and 3 geneses are identical.
9) Prolyl isomerase Pin 1—as used in the invention, Prolyl isomerase Pin 1 is able to terminate IRF3 dependent transcription by interacting with phosphorylated IRF3. Pin 2 triggers polyubiquitination and proteasome dependent degradation of IRF3. Pin1 also inhibits MyD88 dependent signal transduction pathway resulting in the activation of IRF7 and IRF1.
10) Viruses and IFN response—as used in the invention, some viruses can evade host immune system such as +ssRNA viruses and dysregulate the IFN system of the host causing diminished antiviral response and or a prolonged increase in interferon response leading to immune dysregulation and tissue damage in the host. SARS-CoV and MERS-CoV were found to inhibit interferon (IFN)-β production.
11) Interferon III Lambda—as used in the invention, there are 4 IFN Lambda genes in humans comprising IFNL1 (IL29), IFNL2 (IL28A), IFNL3 (IL28B), and IFNL4. Previously, IFN lambda was named as interleukin-28 (IL-28) and IL-29 and classified into the IL-10 family genes since they signal through the common IL-10 receptor subunit 2 (IL-10R2). IFNλ1-3 are similar in sequence, while IFNλ4 is more divergent and only functional in 40% of Caucasians (frameshift mutation allele in rest), IFNλ1 has the highest affinity to receptors, IFNλ3 the lowest. IFNλ are preferentially induced over IFN type I in epithelial cells. IFNλ is produced by epithelial cells, hepatocytes and some immune cells (macrophages, Natural killer (NK) cells and DCs), blood brain barrier, urogenital system. IFNλ3 can induce the proliferation and maturation of NK cells these cells exhibit strong antiviral activity during the early stage of infection (i.e., influenza virus). Type 2 myeloid DCs are the main producers of IFNλs. IFNλ is much less inflammatory than IFN type I, while IFNλ has similar antiviral effect and that of Type I IFNs. Single nucleotide polymorphisms (SNPs) in IFNλs and their receptors are associated with different susceptibility to viral infections and modulate the vaccination response. SNPs mostly affect expression level of these genes. IFNλs are not associated with autoimmune diseases, rather they seem to be protective in autoimmune mouse models of asthma, colitis and arthritis
12) Interferon lambda receptors—As used in the invention, there are 2 Interferon lambda receptors which interferon lambdas signals through consisting of IL10RB also called IL10R2 or CRF2-4) and IFNLR1 (formerly called IL28RA, CRF2-12). subunit IL10R2 is ubiquitously expressed. IFNLR1 limited to epithelial cells, subsets of myeloid and neuronal cells. While they have potent antiviral effects on viruses including, they are less inflammatory than IFN-1-Beta. SARS CoV (+ssRNA Coronaviridae). The strength of IFNλ response is modulated by different affinities of the IFNλs to their receptor, by different levels of expression of receptors in various cells and negative feedback loop. Also, soluble secreted IFNLR1 might sequester IFNλ. IFNλ via their receptors activate JAK-STAT pathway is important for controlling viral infections at epithelial barrier comprising lungs, blood-brain barrier, gastrointestinal tract. There may be novel signaling pathway of IFNλs regulating endothelial cells as they restrict endothelial cell junctions in blood-brain barrier independent of STAT1, in vitro study.
13) IFNλs—as used in the invention, IFNλs act on murine neutrophils (that exacerbate disease severity in respiratory virus infections) via non transcriptional/translational way, (i.e., via ROS release). IFNλs polarize the response toward Th1 phenotype and suppress Th2 and B cell responses. Some respiratory viruses have evolved the mechanisms to suppress IFNλ function or signaling, which underscores the importance of IFNλ for respiratory immunity. Interferon 1 receptor may mediate more immune dysregulation in viral infections (i.e., SARS-CoV, SARS-CoV-2). It has been noted that SARS-CoV induced a delayed IFN-I signaling immune dysregulation. The responses of IFN I and II are balanced and if the host is exposed to high virus load, has higher expression of virus receptors, or a genetic predisposition to producing more IFN I, or if virus inactivates IFNλ, the responses may be skewed toward IFN type I, inducing an inflammation. It has been shown that MERS-CoV potently suppresses IFNλ production from human epithelial cells through the expression of Non-structural protein 4a(NS4a) and NS4b with little effect on type I IFNs.
14) Natural killer (NK) cells—as used in the invention, NK cells are part of the innate immune system and have antiviral and antitumor activities. NK cells can be induced by INFλ as well as by IL-12, IL-15 and IL-21. IL-15 also increases cytotoxic potential of NK cell. IL-15 also induces Interferon II gamma and not IFN I and IFN-γ can, in turn, stimulate or prime monocytes/macrophages to secrete IL-12 and TNF-α which can further activate NK cells. IL-18 also increased IL-15 levels.
15) Chemokine C-X-C ligand 10 (CXCL10)—as used in the invention, Chemokine C-X-C ligand 10 (CXCL10) is also known as interferon-γ-inducible protein 10 (IP-10), exerts biological function mainly through binding to its specific receptor, CXCR3. Epithelial cells, endothelial cells, fibroblasts, thyrocytes, keratinocytes, infiltrating inflammatory cells (i.e., neutrophils, monocytes) and they can express CXCL10 and CXCR3 under inflammatory conditions

Inflammasome and Pyroptosis—as used in the invention “inflammasome and pyroptosis” is described as follows. The innate immune system is the first line of host defense when infected by pathogens (i.e., viruses, bacteria, fungi). Inflammasomes are part of the innate system. They are intracellular protein complexes, are in the cytosol and/or in subcellular structures (i.e. endosomes, nucleus). Inflammasomes upon activation with DAMPs and/or PAMPs or other activators form multiprotein oligomers which activate highly proinflammatory cytokines including and interleukin-1β (IL-1β) and IL-18 as well as pyroptosis (a highly inflammatory cell death). There are five members of PRRs that have been confirmed to form inflammasomes: the (NLR) family members NLRP1, NLRP3, and NLRC4, AIM2 and pyrin. In addition, other members of PRRs, such as NLRP2, NLRP6, NLRP7, NLRP12, and IFI16, are also reported to form inflammasomes.

Canonical and Non-Canonical Activation of Inflammasomes, Pyroptosis and Necroptosis—Canonical inflammasome activation is described as follows. An inflammasome is defined by its sensor protein (a PRR). An inflammasome is a multimeric complex comprising: a sensor (a PPR), a mediator (i.e. ASC) and an effector (i.e., Casp1 in canonical pathway inflammasome activation or Casp11, Casp4, Casp5 in non-canonical inflammasome activation). Casp3 can also trigger pyroptosis (for example chemotherapy drugs) or Tumor Necrosis Factor (TNF) via the TNF1 receptor can activate Casp3, which activates gasdermin and leads to pyroptosis.

An NLR inflammasome (i.e., NLRP3, NLRC4, NLRP1) is further described as follows. An inflammasome sensor molecule (a PRR, i.e., an NLR) connects to Casp-1 via ASC which is an adaptor protein encoded by PYCARD that is common to all inflammasomes. The sensor, a PRR (i.e., an NLR) begins to oligomerize in response to extracellular PAMPs and/or intracellular DAMPs wherein it forms a biological motor which comprises the NLR attaching to a bipartite adaptor protein called apoptosis-associated speck-like protein containing a caspase activation and recruitment domain (CARD; ASC) and an effector termed Caspase-1 (Casp-1). Caspase-1 is activated via proximity-induced autocatalytic activation upon recruitment to an inflammasome. Active Casp-1 cleaves the cytokines pro-interleukin-13 (pro-IL-13) and pro-IL-18 into their mature and biologically active forms, IL-1β and IL-18 respectively.

Interleukin 1 beta (IL-1β)—as described in the invention IL-1B is used interchangeably with IL-1β and is described as follows. IL-1B is a cytokine and induces the expression of genes that control fever (thus it is pyrogenic), pain threshold, vasodilatation, and hypotension, and can lead to an endothelial cell response which mediates infiltration of immune cells to infected or damaged tissues. IL-1β also promotes adaptive T helper 1 (Th1), Th17, and humoral immunity. While most cytokines are secreted through the classical endoplasmic reticulum-Golgi route as active cytokines, IL-1β is produced as a biologically inactive precursor protein that is cleaved (i.e., with Casp1) prior to its secretion as a bioactive cytokine. Expression of pro-IL-1β is regulated by nuclear factor-κB (NF-kB) mediated transcription and is induced upon activation of NF-κB which can in turn be activated by many signals including other PRRs such as TLRs.

Interleukin-18 (IL-18)—as described in the invention IL-18 is described as follows. IL-18 is a cytokine and is necessary for interferon-gamma (IFN-γ) production and it is a co-stimulatory cytokine that mediates adaptive immunity. IL-18 is important for IL-17 expression by Th17 cells and may polarize T cells toward Th1 or Th2 profiles in combination with other cytokines. While most cytokines are secreted through the classical endoplasmic reticulum-Golgi route as active cytokines, IL-18 is produced as a biologically inactive precursor protein that is cleaved (i.e., with Casp1) prior to its secretion as a bioactive cytokine. Pro-IL-18 is expressed constitutively in macrophages.

Gasdermin D (GSDMD)—as described in the invention GSDMD is described as follows. Active Casp-1 cleaves GSDMD, which allows the N-terminal domain of GSDMD to form pores in the plasma membrane, wherein GSDMD triggers a highly pro-inflammatory form of cell death, termed pyroptosis.

Pyroptosis—as described in the invention the term “pyroptosis” refers to a highly lytic and inflammatory cell death wherein GSDMD activation by inflammasomes mediates membrane pore formation, cell swelling, plasma membrane rupture, and cell bursting. Pyroptosis is highly inflammatory and a non-homeostatic that requires the enzymatic activity of caspase-1 or Casp1. Pyroptosis causes severe immune dysregulation. Pyroptosis of infected cells removes the protective, intracellular replicative niche of the pathogen, enabling the innate immune effector cells to kill the pathogen. Pyroptosis can spread viruses and triggers a severe immune response, cytokine release, and the generation of DAMPs to further prepare the immune system to respond to infection. When this process is dysregulated and upgraded it can lead to severe immune dysregulation and cytokine storm.

Canonical pathways for inflammasome activation—as used in the invention, canonical pathways for inflammasome activation involve ASC, Casp-1 which lead to secretory and active IL-1β, Il-18, GSDMD, and pyroptosis.

Non-canonical inflammasome activation—as used in the invention, non-canonical inflammasome activation involve Casp-11 in mammals and Casp 4 and casp5 in humans. Casp4, Casp5 or Casp11 is activated with DAMPs and/or PAMPs and/or other pathways and can cause a lytic cell death which is between necroptosis and pyroptosis (i.e., gram-negative bacteria can activate casp-11). Both Casp1 and Casp 11 can lead to pyroptosis, but only caspase1 processes IL-1β and IL-18. IL-1B can be activated via IL-21.

Necroptosis—as used in the invention is a caspase-independent form of cell death. Necroptosis is a cellular process which is characterized by RIP1/RIP3 phosphorylation. The phosphorylated RIP1/RIP3 complex then interacts with a downstream molecule mixed lineage kinase domain-like protein (MLKL), which often results in a lytic cell death. MLKL is similar to GSDMD. RIPK3 phosphorylation of MLKL will shift inert cytoplasmic monomeric MLKL to membrane-associated MLKL oligomers. MLKL and GSDMD can bind to cardiolipin which is found in bacterial cells as well as in Mitochondria, thus MLKL and GSDMD can damage organelles (i.e. mitochondria, lysosomes). Necroptosis can intensify inflammation by activating inflammasomes. Mitochondrial ROS and JNK pathway activation can lead to necroptosis. Necroptosis is activated when apoptosis is prevented such as in a viral infection. In the absence of caspase activity, death receptors (i.e., TNF receptor 1 (TNFR1), TLR3, and/or TLR4), can activate RIPK3 and MLKL to cause necroptotic cell death. Following TLR or TNFR1 signaling, RIPK3 oligomerizes and becomes activated through RIP homotypic interaction motif (RHIM), which interacts with adaptor proteins comprising RIPK1 and TRIF; and is termed the necrosome, which triggers RIPK3 induced phosphorylation of the activation loop in MLKL's pseudo-kinase domain which then translocates MLKL to cellular and/or subcellular membranes and cause disruption in the membrane. Activators of necroptosis comprising: caspase inhibitors, receptor-interacting protein 1 (RIP1 or RIPK1 are used interchangeably), RIP3 or RIPK3 used interchangeably, ubiquitin E3 ligases, deubiquitylating enzymes, glycogenolysis, glutaminolysis, mROS, ROS generated by NADPH oxidase 1, pro-apoptotic B cell lymphoma 2 (BCL-2) family members, and poly(ADP-ribose) polymerase (PARP). RIPK1 also acts with RIPK3. In absence of Caspase 8 (Casp8), necroptosis is activated and in the presence of CASP8 and Fas-interacting protein (FADD) apoptosis can be activated as opposed to necroptosis. Casp8 also activates NF-KB where the inactive Casp8 remains intact. It appears for apoptosis the inactive casp8 is cleaved into subunits to assemble and form a mature active casp8. CASP8 and FADD like apoptosis regulator also termed FLICE-like inhibitory protein (cFLIPL) is a protein encoded by CFLAR gene and regulates CASP8 and FADD. cFLIR has 3 isosoms and regulates apoptosis (programmed cell death), necroptosis and has a role in inflammasome activation and pyroptosis. cFLIPl, and caspase-8 interact with the NLRP3 inflammasome, and hemizygous deletion of cFLIPL inhibited canonical NLRP3 inflammasome activation. c-FLIP is required for the full NLRP3 inflammasome assembly and NLRP3 mitochondrial localization. c-FLIP downregulation reduced AIM2 inflammasome activation as well. PARP-1 can also activate RIPK3-RIPK1 and activate necroptosis.

NLRP3 inflammasome as used in the invention is part of the innate immune system and is a cytosolic pattern recognition receptor. NLRP3 is found in all mammalian cells, including: immune cells, epithelial cells, vascular cells and endothelial cells. NLRP3 is encoded by NLRP3 gene which is on the long arm of chromosome 1. NLRP3 is also known as NALP3. NLRP3 is a tripartite protein that consists of an amino-terminal domain (PYD), a central nucleotide-binding and oligomerization domain (NOD or NACHT), and a C-terminal leucine-rich repeat (LRR) domain and this entire structure is the sensor. The NACHT domain of NLRP3 has ATPase activity and has a role in the oligomerization of the NLRP3 proteins. The LRR domain is not involved in the autoinhibitory of NLRP3, has a regulatory function and is involved in interaction with ligands. NLRP3 is kept in an inactive state in the cytoplasm by complexing with heat shock protein 90 (HSP90) and SGT1 encoded by Suppressor of G2 allele of SKP1 homolog (SUGT1) gene. When NLRP3 detects danger signals it is released from HSP90 and SGT1, is activated, oligomerizes and recruit ASC protein and caspase-1 to the inflammasome complex.

NLRP3 senses the extracellular matrix, tissue environment and intracellular space for danger signals that arise from pathogen invasion, or signals from cell death (i.e., apoptosis, necroptosis, pyroptosis), or elevated levels of metabolites from cell metabolic dysregulation, or aggregate or crystalline material, or cellular stress, mitochondrial stress, organelle stress, cellular or subcellular damage, or by molecules which are physiological constituents of functioning cells, but have been displaced from actual location, or when they aggregate.

NLRP3 can be activated via a two-signal process of priming by TLRs (i.e., TLR4, MyD88 and NF-KB signal transduction pathways) that can also increase transcriptionally NLRP3, its mediator PYCARD (ASC) and its effector Casp 1 which is activated and cleaves inactive IL-1B and IL-18 into their active forms and also activates GSDMD. DAMPs can prime and/or activate NLRP3. NLRP3 can be activated non transcriptionally as well. While the mechanism of NLRP3 activation by diverse stimuli was unknown, in 2018 it was found that Trans Golgi Complex causes activation of NLRP3 and that different NLRP3 stimuli and DAMPs lead to disassembly of the trans-Golgi network (TGN). NLRP3 polybasic region binds to the negatively charged phosphatidylinositol-4-phosphate (PtdIns4P) which becomes visible when the trans Golgi is disrupted. This becomes a scaffold for NLRP3 aggregation into multiple puncta which leads polymerization of the adaptor protein ASC which is on the mitochondrial surface and transported by Dynein on the mitochondria towards the dTGC. The colocalization of NLRP3 and ASC mediates the activation of NLRP3 which leads to recruitment of CASP-1 and its activation.

Upon activation with a stress ligand, NLRP3 can oligomerize, recruit a mediator, ASC, which can activate Casp-1. NLRP3 can become activated by numerous ligands comprising: DAMPs, mitochondrial DAMPs, Nlrp3 deubiquitination, toxins, excess glucose, ceramides, fibrillar or oligomeric Aβ peptide, fibrillar α-synuclein, neurotoxic prion peptide, uric acid crystals, calcium crystals, calcium phosphate crystals, cholesterol crystals, potassium (K) efflux (movement of potassium from inside the cell to outside), endoplasmic (ER) stress, unfolded protein response (UPR) in the ER, Golgi, Mitochondria, mitochondrial stress and change in mitochondrial bioenergetics, ATP loss pathways (via dysregulation of various components of the mitochondrial complex), damaged and/or oxidized mitochondrial DNA, mitochondrial reactive oxygen species (mROS), XA generated ROS, disruption of Trans Golgi network (TGN), potassium (K) efflux, chloride influx, ER calcium depletion into the cytoplasm, high mobility group box 1 (HMGB1), heat-shock proteins hsp70 and hsp90, cardiolipin release, cathepsin release) and extracellular pathogen associated molecular patters (PAMPS) comprising lipopolysaccharides (LPS), double stranded DNA, single stranded RNA, double stranded RNA, fungal, protozoal, other bacterial and viral nucleic acid, extracellular ATP, RhoGTPase, and amyloid beta protein.

Protease—as used in the invention the term “protease” or “peptidase” or “proteinase” is an enzyme that catalyzes (increases the rate of) proteolysis which is the breakdown of proteins or polypeptides, or peptides into smaller polypeptides or single amino acids. Examples of proteases comprise of: TMPRSS2, TMPRSS4, Cathepsin, DPP4 and Furin.

Serine protease—as used in the invention the term “Serine proteases” refer to or describe enzymes that are endopeptidases and which cleave peptide bonds and serine serves the electron donor of the serine protease enzyme's active site. Serine proteases are involved in diverse functions comprising digestion, immune response, blood coagulation, wound healing, digestion, tumor invasion, embryonic invasion, viral fusion and entry. There are approximately 176 human serine proteases. Type II transmembrane serine proteases (TTSPs) are a subset of serine proteases, anchored in the plasma membrane which share a common protein structures comprising a transmembrane domain at the N-terminus which anchors a canonical serine protein domain into the plasma membrane. There are approximately 17 known TTSP comprising: TMPRSS2, TMPRSS4, TMPRSS11 and others. DPP4 is another serine protease.

Transmembrane Protease, Serine 2 (TMPRSS2)—as used in the invention “TMPRSS2” is a protease encoded by the TMPRSS2 gene. It is a type II transmembrane serine protease (TTSP) and interacts with a variety protein comprising cell surface proteins, soluble proteins, matrix components, and proteins on adjacent cells. TMPRSS2 is androgen induced and activates several substrates such as ACE2, matriptase (ST14), pro-hepatocyte growth factor (HGF), protease activated receptor 2 (F2R11), trigeminal neurons, the spike glycoprotein of several viruses comprising SARS-CoV, SARS-COV2, HCoV-229E, HCoV-ECM, Sendai virus, HPIC, Influenza A virus, HMPV. As the expression of TMPRSS2 is regulated by androgen, it is highly expressed in normal and neoplastic prostate epithelium and it forms the oncogenic TMPRSS2-ERG gene fusion. TMPRSS2 is upregulated by androgens in prostate cancels cells and downregulated in androgen independent prostate cancer cell. TMPRSS2 is involved in serine proteases prostate-specific antigen and human kallikrein K2. Select cancer cells can localize TMPRSS2 into their surface. TMPRSS2 has a role in nociception and neurogenic inflammation and involved in cancer related pain. TMPRSS2 activates protease activated receptor-2 (PAR2). TMPRSS2 is elevated in head and neck cancers. TMPRSS2 facilitates SARS-CoV and SARS-CoV infections via two independent mechanisms, cleavage of ACE2 and cleavage of viral spike glycoprotein, which activates the S protein. TMPRSS2 is highly expressed in epithelial tissues. The spike protein S of corona viruses has two domains, S1 which binds to ACE2, and S2, which enables the fusion of cell-host and virus membranes. Downstream of the fusion peptide there are heptad repeat regions (HR1 and HR2). The TMPSS2 protein primes the virus S protein by cleaving it between the two domains, which results in conformational changes and allows the fusion. TMPRSS2 has 2 independent mechanisms in which it proteolytically cleaves ACE2 which allows binding of SARS-COV2 and it cleaves the coronavirus spike glycoprotein which activates the S protein and enables membrane fusion. For Other proteases comprising lysosomal proteases (i.e., cathepsin, B, cathepsin L), HAT, Furin may also be involved in SARS corona viral binding, fusion and entry. SARS-CoV-2 uses Spike protein (S) for entry into cells. Spike protein S is thought to be cleaved into S1 and S2 peptides by serine protease TMPRSS2 or Cat/L. Blocking TMPRSS2 with the inhibitor camote mesylate left some residual entry of viruses into host cells in lung epithelial cell lines in SARS-CoV. TMPRSS4 and TMPRSS11, a/d/e have also been found to cleave SARS-CoV, SARS-COV2 and MERS-CoV. TMPRSS11d, also known as human airway trypsin-like protease (HAT), is unable to activate SARS-CoV S protein but can activate MERS-CoV. Corona virus has developed the flexibility to enable various proteases to activate it. Lipid rafts on the membrane have cholesterol and ACE2 is possibly a raft protein. Cholesterol and high intracellular calcium can impact viral binding and fusion of some viruses comprising SARS Corona viruses.

Transmembrane Protease, Serine 4 (TMPRSS4)—as used in this invention the term “TMPRSS4” refers to is a serine endopeptidase which is encoded by TMPRSS4 gene. Genetic polymorphism including single nucleotide polymorphisms (SNPs) in the TMPRSS2 gene have been shown to be related to severity of some infections (i.e., rs2070788 GG genotype and rs383510), erg fashion status (i.e., SNPs in rs6698333, rs1889877, rs3798999, rs10215144, rs3818136, rs9380660 and rs1792695). As am example, the rs2070788 GG genotype, correlated with higher TMPRSS2 expression in human lung tissues and was found in one study to create a greater than 2-fold higher risk of severe A(H1N1) influenza.

Cathepsins—as used in this invention the term “Cathepsins” refer to lysosomal cysteine proteinase that play a major role in intracellular protein catabolism. Cathepsins comprise of Cathepsins A, L (L1), B, C, F, H, K, O, V (L2), X, S, and W. Cathepsin L and cathepsin B become active in the late and early endosome, respectively. Cathepsin S may participate in degradation of antigenic proteins to peptides for presentation to the MHC Class II and is present in antigen presenting cells comprising macrophages, dendritic cells, B lymphocytes, microglia. It is also found in epithelial cells, keratinocytes, Cathepsin S also acts as an elastase cleaves extra cellular matrix and can cause vessel permeability and angiogenesis. It is activated by proinflammatory cytokines including tumors or senescent cells. Cathepsin S does not require a low PH unlike other cathepsins and can function in a range of PH. Cathepsin L acts on substrates comprising collagen, elastin, alpha-1 protease inhibitor, a major controlling element of neutrophil elastase activity. It's implicated in several pathologic processes, including myofibril necrosis in myopathies, in myocardial ischemia, in the renal tubular response to proteinuria. Cathepsin B can enhance other protease activities comprising including matrix metalloproteinases, urokinase. It's involved and autophagy and cancers. Acidic PH activates Cathepsins L and B. Cathepsin B/L appear to be involved SARS-COV viral fusion. It acts in lysosomes (virus moving from endosomes to late endosomes to lysosomes). They may be involved in SARS-CoV-2 entry. Cathepsin S is encoded by CTSS gene and is a powerful elastase. Cathepsin S is a lysosomal is important for immune signaling and subsequent activation of CD4⁺ T cell mediated immune responses. Cathepsin D is encoded by CTSD gene. Cathepsin D is a lysosomal aspartic proteinase involved in degradation of proteins, in apoptotic processes induced by oxidative stress, cytokines and aging. Cathepsin D is involved in depredation of polypeptide hormone and growth factor, activation of enzymatic precursors, it is also found in extracellular space. It can also show activity in neutral PH. It can also activate vascular endothelial growth factors. Cathepsin D can reduce thiredoxin-1 (Trx) and increase ROS. Optimal PH for cathepsin D is 4.5. Cathepsin A or Cystatin A are used synonymously in this invention and is encoded by gene CTSA. Cathepsin A cleaves peptide bonds optimally at acid pH, but esters and amide bonds of C-terminal amino acids at a neutral pH. It can inhibit Cathepsin B gene (CTSB). Additionally, it can increase Ang (1-9) fro RAAS-KKS. Cathepsin A is present in high concentrations in cells and or tissue comprising: macrophages, endothelial cells, kidney, urine, placenta, fibroblasts, platelets and brain. It is also found in the heart where it can convert AngI to Ang (1-9). Ang(1-9) and ang (1-7) can augment BK activity on its B2 receptor. Cathepsin A can inactivate various peptides including bradykinin.

DPP4—as used in the invention Dipeptidyl peptidase-4 (DPP4), adenosine deaminase complexing protein 2 or CD26 are used interchangeably. DPP4 is encoded by DPP4 gene and is an enzyme expressed on the surface of most cell types. DPP4 is associated with signal transduction, immune regulation, and programmed cell death (apoptosis). DPP-4 cleaves a broad range of substrates including growth factors, chemokines, neuropeptides, and vasoactive peptides and some viral spike proteins (i.e. MERS Coronavirus).

Furin—as used in this invention the term “Furin” refer to or describe an enzyme encoded by the Furin gene. It belongs to the subtilisin-like proprotein convertase family. It usually works in Golgi networks to cleaves off its substrate, an inactive newly synthesized proteins to its active form. It is ubiquitously expressed. Furin is found in many human tissues including the lungs, liver and small intestines, which means that the virus has the potential to attack multiple organs. Some of Furin's substrates are: pro-parathyroid hormone, transforming growth factor beta 1 precursor, pro-albumin. In T-cells Furin is required for maintenance of peripheral immune tolerance. Furin also enhances the secretion of IFNγ, which in turn activates the FUR promoter. Furin can move to plasma membrane via endosome and can be shed into the extracellular space as an active enzyme. Furin may be involved in the binding/fusion of certain viruses (i.e., HIV, -1, Influenza). In some coronaviruses Furin cleavage site between S1 and S2 is not absolutely required for infection, although lack of cleavage can lower S-mediated cell-cell fusion. It is not clear whether Furin is required for SARS-CoV 2 viral binding/fusion. SARS-CoV-2 has potential cleavage site for Furin protease in S spike protein, between S1 and S2 domains. Furin may facilitate SARS-CoV-2 to enter some types of cells (particularly those with low expressions of TMPRSS2 and/or lysosomal cathepsins; although Furin's role not fully realized. Furin may potentially cleaves SARS-COV2 spike protein before entry to host cell, priming it for TMPRSS2 cleavage possibly leading to more pathogenicity. Mutation of SARS-CoV-2 without Furin motif had moderately affected entry into host cells.

Bat Immune Advantages—as used in the invention the term “bat immune advantages” refers to or describes that bats have a unique immune system which is different than other mammals. Bats can harbor deadly viruses (i.e., SARS corona viruses, Ebola, others) and do not succumb to these viral infections which have detrimental health sequalae and which can be deadly to human patients. Further, bats have longevity. Bats are considered the natural reservoir for several highly pathogenic zoonotic viruses (i.e. Ebola, rabies, SARS-CoV, MERS-CoV, SARS-Cov-2). Bats can host these viruses and these viruses have minimal to no pathologic effects in bats. There are several immune response differences that occur in bats when infected with these deadly viruses which are not observed in other mammals (i.e., humans). For example, in bats while they mount a strong initial proinflammatory response, they simultaneously mount an anti-inflammatory response via Il-10 gene induction and upregulation

NLRP3 in bats—as used in the invention the term “NLRP3 in bats” refers to or describes that there is an overall dampening of NLRP3-mediated inflammasome activation in bat primary immune cells, as opposed to human cells with respect to RNA viral infections. For example, a bat's response to a coronavirus such as MES-CoV, as opposed to a human subject's response, is a dampened host inflammatory response without affecting viral load, which enables the bat to withstand the pathogenic MES-CoV. There is a dampened transcriptional priming specific for NLRP3 following different TLR stimulation in bat immune cells. Bats have a robust induction of IL-1β, IL-6 and TNF which suggests functional NF-KB signaling in bat immune cells. When bat immune cells are infected, such as with MERS-CoV, they have a dampened NLRP3 response, can have a high transient phase of high viral load, and tolerate the viral infection. For instance, in bats, a high-dose of Ebola virus and MERS-CoV causes no clinical disease and limited pathology, despite high viral titers detected in tissues or sera. Bats have improved innate immune tolerance rather than an enhanced antiviral defense system.

The immune advantages in bats when infected with deadly viruses comprise:

1) Bats produce low levels of NLRP3 in their baseline uninfected state. Bats have low NLRP3 inflammasome priming and/or activation in the face of viral infections and resultantly low levels of Casp-1, IL-1β, Il-18, GasderminD (GSDMD), low pyroptosis and cytokine storm.

2) During the initial phase of viral infections, bats produce a rapid and potent surge and secretion of antiviral interferon response (interferon1α/β) with a quick decline whereas in humans can mount a dysregulated antiviral response during viral infections with these deadly viruses. For example, bats mount a rapid antiviral (interferon β, INF-β) and proinflammatory response (interleukin-1β, Il-1β and tumor necrosis factor, TNF) which is rapid and then declines but in human subjects it appears to be prolonged and can lead to cytokine storm.

3) Bats minimize chronic inflammation by concomitantly producing anti-inflammatory cytokines such as IL-10 which diminish cytokine storm, while in humans these deadly viruses can result in cytokine storm.

4) Bats do not produce high levels of nitric oxide when infected with viruses while other mammals do, which can contribute to oxidative tissue damage.

Activators of Inflammasome(s)—as described in the invention the term “Activators of Inflammasome(s)” refers to or describes that there are various activators of inflammasome priming and/or activation which when dysregulated can lead to immune dysfunction. As used in the invention several activators of NLRP3 inflammasome activation in a mammalian subject are briefly described. Inflammasomes have many activators, including but not limited to:

1) Mitochondrial stress—as used in the invention “mitochondrial stress” activates inflammasomes via mitochondrial reactive oxygen species (mtROS) dependent and/or mtROS independent pathways comprising: decrease in mitochondrial membrane potential (Δψm), generation of mROS, decrease in ATP and NAD generation, mitochondrial outer membrane permeabilization (MOMP), dysregulation of electron transport chain and complex 1-IV, mitochondrial dysfunction resulting in reduction in NAD+ and microtubule αtubulin acetylation which can colocalize ASC and NLRP3, mtROS induced mitochondrial DNA (mtDNA) and the inner mitochondrial membrane, lipid cardiolipin to translocate from inner mitochondrial membrane to the outer mitochondrial membrane, mtROS activated mitochondrial antiviral signaling protein (MAVS) on the outer membrane which can associate with NLRP3, mitofusin 2 (Mfn2).

2) Oxidoreductase—as used in the invention, “oxidoreductase” induces mitochondrial ROS via the PI3K-AKT-mTOR pathway. which initiates a signaling cascade that changes cellular metabolism, apoptosis, autophagy and inflammatory responses. The formation of ROS and oxygen superoxide by phagocytes is critical for pathogen killing, but it can also activate NLRP3 inflammasome. Multiple intracellular sources of ROS exist comprising: NOX complexes, oxidase form of XOR.

3) Xanthine oxidoreductase (XOR)—as used in the invention, XOR is an enzyme complex, constitutively expressed enzyme widely distributed through various organs comprising: liver, liver, lung, kidney, and plasma. XOR is involved in purine degradation wherein it catalyzes the conversion of hypoxanthine or xanthine to uric acid (UA). XOR participates in a variety of biochemical reactions for example hydroxylation of various pterins, purines, aromatic heterocycles, as well as aliphatic and aromatic aldehydes. XOR can colocalize to mitochondria or cytoplasm. XOR is a rate limiting enzyme of purine metabolism, and catalyzes the conversion of hypoxanthine, xanthine into xanthine and uric acid, respectively. Furthermore:
a) XOR is synthesized as xanthine dehydrogenase (XDH), but it can be converted to xanthine oxidase (XO) during cellular oxidative stress. Both XDH and XO can generate UA, but the XO form also produces superoxide and hydrogen peroxide.
b) XOR produces large amount of Hydrogen peroxide (H2O2) and superoxide (O2⁻) which is a reactive oxygen species (ROS).
c) XOR can transform nitrates into nitrites and nitric oxide (NO). XOR also catalyzes the reaction of NO with O2- to form highly reactive peroxynitrites.
d) XDH has very low reactivity toward molecular oxygen but high reactivity toward nicotinamide adenine dinucleotide (NAD⁺). XO does not interact with NAD⁺ but is very efficient in producing superoxide anion (O2.) and H2O2. NAD can exist in its oxidized or reduced forms (NAD+ and NADH) respectively.
e) Both XO and XDH can oxidize (remove an electron) NADH, with the concomitant formation of reactive oxygen species.
f) XOR mediates NLRP3 inflammasome activation via mtROS production as well. XO can be activated during hypoxia (low oxygen states).
g) The endothelial xanthine oxidoreductase protein expression is regulated by hydrogen peroxide and calcium. Circulating XO binds to glycosaminoglycans on the surface of endothelial cells to produce oxidants, which can trigger organ injury, (i.e. endothelial dysfunction, lung injury).
h) The XO form of XOR is the major source of ROS in macrophages that mediates NLRP3 inflammasome activation. XO inhibitors do not inhibit NLRP3 inflammasome priming but inhibit NLRP3 inflammasome activation.
i) XOR protein is localized to both the cytoplasm and mitochondria and participates in ROS generation.

4) Uric Acid (UA)—as used in the invention, increased intracellular uric acid crystals can lead to NLRP3 inflammasome activation. Uric acid is formed by xanthine oxido reductase break down of purines. UA crystals are potent inflammasome activators, though not soluble urate. Though hyperuricemia from the UA released from dying cells can potentially form uric acid crystals and activate inflammasomes via XOR activation. High urate levels can be pro-inflammatory to the endothelium and renal tubules most likely secondary to NLRP3 inflammasome activation. Calcium crystals are also activators of inflammasomes;

5) Cellular autophagy—as used in the invention, “cellular autophagy” is a catabolic process responsible for turnover of macromolecules and organelles. Mitophagy involves autophagy of mitochondria. Autophagy enables turnover of macromolecules, removes unwanted, toxic, harmful materials and structures; it also replenishes metabolic intermediates. Additionally, some of the recycled intermediate breakdown products supply antimicrobial peptides and antigenic fragments for presentation by innate immune cells. In mammals, autophagy is regulated by the mammalian target of rapamycin (mTOR) and is carried out by autophagy-related (ATG) proteins. Plasmacytoid dendritic cells (pDCs) are crucial mediators for the establishment of immunity against most viruses, given their extraordinary capacity to produce large quantities of type I interferons. pDCs are able to detect viruses in acidified endosomes with the help of TLRs. Autophagy is necessary for production of IFN-α by pDCs.

6) Autophagy—as used in the invention, “autophagy” is negatively regulated by the AKT pathway and inhibition of autophagy can activate NLRP3 inflammasome. XOR dependent ROS generation also activates the AKT-PI3K-mTOR pathway in macrophages. ROS inactivate the regulatory subunit PTEN (phosphatase and tensin homologue deleted on chromosome). PTEN enables downstream activation of AKT, and the mTOR complex.

7) Nuclear factor kB (NF-kB)—as used in the invention, NF-kB is a family of transcription factors that regulates the expression of a large number of genes involved in innate and adaptive immunity, the inflammatory responses, inflammation, cell survival, proliferation and cell differentiation. NF-kB transcription factors are activated in response to various stimuli, PAMPs and DAMPs including cytokines, infectious agents and cellular stress. Dysregulation of NF-KB is implicated in chronic inflammation, autoimmunity and other condition. In mammals, the NF-kB family consists of five proteins that share a highly conserved Rel homology domain. These proteins comprise c-Rel, RelB, p65 (RelA), p105 (NF-kB1) and p100 (NF-kB2). C-Rel, RelB and P65 contain C-terminal transactivation domains, while the others share a long C-terminal domain with multiple copies of ankyrin repeats, which inhibit their activation. p105 and p100 can also form DNA-binding proteins p50 and p52 via proteolysis, respectively. The highly conserved Rel homology domain is responsible for DNA binding, dimerization, nuclear translocation and interaction with the inhibitor of Nuclear Factor Kappa β (IkBα).

8) The IκB kinase (IKK)—as used in the invention, IKK is an intracellular enzyme complex composed of the catalytic subunits IKKa and IKKb and the regulatory subunit IKKg, also known as NF-kB essential modulator (NEMO) (NF-kB essential modulator)/IKKAP1 (IKK associated protein 1). The IKK enzyme complex keeps NF-kB in an inactive state in the cytoplasm by members of the IkB, containing ankyrin repeats which binds to NF-kB's ankarin repeats and blocks its nuclear translocation in resting state NFKβ is bound to its inhibitor IkBα.

9) Mechanism of NF-KB activation—as used in the invention, there are several mechanisms of NF-KB activation and translocation to the nucleus. The canonical or classical NFKβ activation pathway comprises of a ligand (i.e., PAMPs) binding to a receptor comprising T or B-cell receptors or the Toll-like receptor (TLR), Il-1 or tumor necrosis factor (TNF) type 1/2 receptors (TNFR1/2). This binding actives IKK which phosphorylates IkBα protein at specific sites equivalent to Ser32 and Ser36 of IkBα. The phosphorylation triggers polyubiquitination at sites equivalent to Lys21 and Lys22 of IkBα and its degradation by the 26S proteasome which results in IkBα dissociating from NFKβ and NFKβ dislocating to the nucleus for binding to the promoters of genes containing specific NF-k B-binding sequences, which are involved in immune related pathways comprising encoding chemokines, cytokines, interferons, and adhesion molecules. NF-KB activation can also induce the first priming signal for NLRP3 inflammasome activation. NF-KB activation can increase NLRP3, Casp1, GSDMD, pro-IL-1β and proIL-18 gene transcription. ELKS is an essential scaffolding component in the activation of NF-k B in response to IL-1 and TNF and is involved in recruitment of IkB to the IKK complex. Increased phosphorylation of the p65 subunit of NF-κB can activate NLRP3.

10) The alternative NF-KB activation pathway—as used in the invention, NF-KB is activated by other receptors (i.e., lymphotoxin-b receptor, CD40, and CD30, RANKL, B-cell-activating factor belonging to the TNF family receptor (BAFF-R)) which causes NF-kB2/p100 to phosphorylate by NF-KB Inducing kinase (NIK) at two C-terminal sites by the IKKa homodimer. IKKa is then ubiquitinated and sent for proteasomal degradation, producing p52. Then p52 dimerizes with RelB. RelB-p52 DNA translocate to nucleus as a transcription factor.

11) JAK2-STAT 3 pathway—as used in the invention, JAK2-STAT 3 is a signal transduction pathway. Janus Kinases (JAKs) are intracellular tyrosine kinases and transduce cytokine mediated signals through JAK-SAT pathways. Signal transducer and activator of transcription proteins (STATs) are intracellular transcription factors. There are 4 types of JAKs and 7 STATs proteins. JAK-SAT pathways communicates extracellular signals to the nucleus for gene transcription. Proteins that are overexpressed by STAT3 activation comprise: Mcl-1, Bcl-2, Bcl-XL. STAT 3 downregulates expression of proteins comprising: P53, interferon β, BAX, Fas and its ligand. Erythropoietin is an endogenous glycoprotein and a hormone produced by the kidney in response to anemia and hypoxia to increase red blood cell synthesis. It binds to a cell surface receptor, Epo Receptor (EPOR), in the bone marrow. Epo binds EPOR and activates JAK2 through phosphorylation and dimerization which results in phosphorylation and dimerization of STATs which are substrates of JAK. Phosphorylated STAT3 inhibits NF-KB P65 phosphorylation and nuclear translocation and suppression of NLRP3 priming.

12) Toll Like Receptors (TLR)—as used in the invention, TLRs comprise: TLRs 7, 9, 4 and 3 containing leucine-rich repeat motifs in their extracellular domain, which recognize distinct PAMPs. There are approximately 13 subfamilies of TLRs. TLRs have both an extracellular domain and intracellular domain (TIR domain). The intracellular domain is an interleukin-I (IL-1) receptor. All TLRs have adaptor protein MyD88 except TLR3 which has the adaptor protein TIR-domain-containing adapter-inducing interferon-β (TRIF). TLR4 has both TRIF and MyD88 adaptor proteins. TRIF dependent pathways activate TIR-domain-containing adapter-inducing interferon-β (IRF3) and NFKβ. TLRs with MyD88 adapter protein activate Interleukin-1 Receptor-Associated Kinase 4 (IRAK4) which activates NF-KB and MAPK downstream of MyD88.

13) MicroRNAs (miRNA)—as used in the invention, the terms “miRNA” or “MIR” are used interchangeably to refer to a small non-coding RNA molecule (containing about 22 nucleotides) which functions as RNA silencing and affects post-transcriptional regulation of gene expression. As used in the invention MIRs comprise: MIR9, MIR20A, MIR21, MIR223, MIR30C, MIR30C1, MIR30C5, MIR495, MI, miR and in particular miR-223 control Nlrp3 expression and NLRP3 inflammasome activation in human monocytes and in Dendritic Cells. TLR4 activation can activate NLRP3 with 1 signal and miR-233 may play a role.

14) Histone deacetylases—as used in the invention, the NF-kB pathway is also regulated by histone acetyltransferases and deacetylases (HDACs), histone acetylation is an epigenetic mechanism which can ultimately control gene expression. Histone acetylation causes the unwinding of the chromatin structure and allows transcription factor access to promoter sites of the gene. HDACs remove acetyl groups and inhibit gene transcription. HDACs comprise HDAC1, HDAC2, and HDAC type 3 (Sirt1). The inhibition of HDAC function also increases acetylation of histones and activates gene transcription and expression.

15) Unfolded Protein Response and organelle stress—as used in the invention, mitochondrial stress or endoplasmic reticulum stress, or Golgi stress, or oxidative stress are referred to as organelle stress. In addition, oxidative stress and pathogen invasion related stress can result in increased protein misfolding, disrupted TGC, and can signal mitochondrial unfolded protein response (UPR^{MT}) or ER UPR, respectively, which can activate inflammasomes. The cellular changes resulting from these stress responses can become DAMPS that activate the innate immune response including NLRP3 inflammasome. ER or Golgi stress can lead to increase in cytosolic calcium mediated by depletion of ER or Golgi-stored calcium which can activate NLRP3.

16) Nuclear receptor subfamily 1, group H, member 4 (NR1H4)—as used in the invention NR1H4 is used interchangeably with bile acid receptor (BAR) or Farnesoid X receptor (FXR). NR1H4 is encoded by NR1H4 gene and it is a nuclear receptor. NR1H4 inhibits ER stress-mediated protein kinase RNA-like endoplasmic reticulum kinase (PERK) activation which can inhibit NLRP3 inflammasome activation. NR1H4 can also inhibit TXNIP.

17) Thioredoxin proteins—as used in the invention, thioredoxin proteins can act as antioxidants and lower oxidative stress. Thioredoxin-interacting protein (TXNIP) regulates thioredoxin enzymes by binding to the catalytic active center of reduced Thioredoxins (TRX) and inhibits its expression and activity. TXNIP belongs to the α-arrestin family and can also regulate kinase 1 (ASK1). Furthermore:
a) TXNIP during oxidative stress can activate NLRP3 inflammasome by binding to the LRR region of NLRP3.
b) Tissue plasminogen activator activates NLRP3 via TXNIP.
c) Ion channels comprising Transient receptor potential cation channel, subfamily M, member 2 and member 7 TMPR7 are proteins encoded by the TRPM2 and TRPM7 gene respectively. TMPR2 increases mRNA expression of TXNIP. Hyperglycemia in the range of 10-30 millimoles can increase TRPM2. TRPM2 is a is a non-selective calcium-permeable cation channel which mediates influx of Ca++ ion influx into cell upon activation with activators comprising hyperglycemic stress, hyperosmotic stress, oxidative stress, ADP-ribose, NAD+, PARP. TRPM7 is another cation channel that is upregulated by Angiotensin II.

18) Nuclear factor erythroid 2-related factor 2 (Nrf2)—as used in the invention Nrf2 is an antioxidant gene. Upon extracellular stimulation, NRF2 translocates to the nucleus and induces the transcription of several antioxidant genes. Vitamin D receptor can activate translocation of NRF2 to the nucleus. Activation-induced NQO1 expression inhibits NLR family, pyrin domain containing 3 (NLRP3) inflammasome activation. Nrf2 also activates the Trx1 gene, prompting the formation of a compound consisting of Trx1 and TXNIP, thereby inhibiting TXNIP activation of the NLRP3 inflammasome.

19) Klotho—as used in the invention, Klotho is an enzyme encoded by the KL gene, and can suppress TTXNIP expression and NLRP3 activation. Klotho as 3 subfamilies: α-klotho, β-klotho, and γ-klotho. Klotho is membrane bound as well has secreted and circulating form. Klotho is involved in insulin sensitivity and aging.

20) Annexin A1 (ANXA1)—as used in the invention ANXA1 is a protein encoded by the ANXA1 gene and it is involved in priming and activation of NLRP3. ANXA1 is involved in transcriptional upregulation of NLRP3, IL-1B and it directly binds to NLRP3 and activates NLRP3 independent of its anti-inflammatory properties. Annexin A1 is involved in suppressing phospholipase A2 and inhibiting eicosanoid production which lowers inflammation as well. Annexin1 is the main mechanism by which glucocorticoids achieve immune suppression.

21) High mobility group box 1 protein (HMGB1)—as used in the invention HMGB1 can be secreted extracellularly when a cell undergoes necroptosis via non-canonical inflammasome activation wherein HMGB1 binds to the Receptor of advanced glycation end product (RAGE) enters the cell's lysosomes and induces cathepsin B (Cat B) activation and release from the ruptured lysosomes which activates NLRP3 inflammasome. Additionally, N4-acetylcytidine (ac4C) which is present on tRNA and rRNA can activate HMGB1 and activate NLRP3. Gene expression profile of older individuals who have worse chronic disease and higher mortality shown to have higher N4-acetylcytidine gene expression than elderly with less pronounced chronic disease.

22) Necroptosis—as used in the invention, non-canonical inflammasome activation is mediated via caspase-11 and human homologs casp 4 and 5 are intracellular proteases activated by TRIF signaling by both TLR4 and TLR3 during innate immune response. PAMPs binding and activation of TLR4 leads to Casp 11/Casp4/Csp5 activation via to TRIF, MyD882/Trif2 activate Casp 5, Casp 5, Casp 11 initiate cell death but do not release mature IL-1β or IL-18. Casp 11/Casp 4/casp 5 release ATP, HMGB1, IL-1 alpha (IL-1a). Caspase-11 acts upstream of NLRP3. The lytic cell death resembles necroptosis than pyroptosis. Another difference between canonical and noncanonical inflammasomes is in the release of IL-la and the danger signal high-mobility group box 1 (HMGB1). When HMGB1 is released extracellularly, it can bind to RAGE and activate NLRP3. Resting macrophages or dendritic cells (DCs) express very low levels of pro-caspase-11. The pro-caspase-11 gene promoter contains NF-kB and STAT binding sites, and is inducible upon NF-KB activation, TNF-alpha, and Interferons.

23) Microtubules—as used in the invention microtubules are made of tubulin polymers that form the cytoskeleton of the cell and enable subcellular transport including transport of ASC and NLRP3. Microtubule acetylation promotes the accessibility of microtubule-associated proteins to microtubules. Microtubule motor protein dynein facilitates movement of cargo along microtubules. Dynein motor attaches to the acetylated tubulin portions of the microtubule and move cargo by moving across mitochondria moving toward the negative sense, towards the nucleus. Acetylation of microtubules mediates the transport of ASC which has colocalized to the mitochondria upon activation with DAMPs, PAMPs, TLRs and other signals to NLR which resides on dTGC or ER which is necessary for NLRP3 inflammasome activation. Furthermore:
a) Microtubules are involved in NLRP3 activation and not NLRC4 or AIM2.
b) Under resting conditions ASC is located in the nucleus, cytoplasm, mitochondria, and NLRP3 mainly in the ER. With PAMPs and/or DAMPs signaling which results in priming and/or activation of NLRP3 approaches TGC at the perinuclear region which mediates colocalization of ASC on mitochondria and NLRP3 on dTGC and activates NLRP3 inflammasome. Motor proteins such as Dynein enable intracellular transport. Dynein slides along microtubules towards its minus end, towards the perinuclear region of the cell. Dynein binds microtubules along the acetylated alpha tubulin.

24) Sirtuins—as used in the invention, Silent information regulator-1 and -3 (SIRT1 and SIRT3) are cellular proteins that regulate cellular health and homeostasis. Sirtuins are NAD+-dependent enzymes and are conserved from bacteria to humans and all mammals contain sirtuins. Mammals contain seven sirtuin enzymes (SIRT1-7). Furthermore:
a) Sirtuin 1 (SIRT1) is an NAD-dependent deacetylase and is a member of the sirtuin family. SIRT-1 acts as an intracellular regulatory protein and is involved in processes comprising: mitochondrial biogenesis, mitochondrial proliferation, oxidative phosphorylation and energy production, energy homeostasis, development, cell survival, and lifespan. SIRT-1 interacts with peroxisome proliferator-activated receptors γ (PPARγ) coactivator 1α (PGC-1α activates the peroxisome proliferator activated receptor γ co-activator 1α (PGC-1α) which mediates transcription of nuclear and mitochondrial genes which encode proteins that promote mitochondrial proliferation, oxidative phosphorylation and energy production.
b) Sirtuin2 (SIRT2) deacetylates alpha tubulins. For example, inhibition of SIRT2 shows accumulation of acetylated alpha tubulin around the perinuclear region and transport of mitochondrial to the minus side close to perinuclear region. Coenzyme NAD+ is required for SIRT2 activation. NAD+ functions as a cofactor for numerous metabolic enzymes, and is a co-substrate for the sirtuin family. DAMPs can lower intracellular NAD+. Activators of NLRP3 inflammasome lower SIRT2 by lowering NAD+ to caused accumulation of acetylated alpha tubulin to promote NLRP3 inflammasome activation. ATP and lysosomal rupture can cause mitochondrial damage, low membrane potential, mitochondrial damage or stress can also lower NAD+. Overactivity of PARP can lead to NAD+ depletion as well.
c) SIRT3 directly acts as an activator of proteins and is important in energy production (i.e., oxidative phosphorylation, tricarboxylic acid (TCA) cycle and fatty-acid oxidation and activation of PGC-1α and AMP-activated protein kinase (AMPK). SIRT3 boosts mitochondrial oxidative metabolism in response to nutrient stress. Sirtuin 3 also acts to scavenge reactive oxygen species (ROS).
d) SIRT3 balances ROS. SIRT3 also modulates complex I and complex II of the mitochondrial oxidative phosphorylation proteins.
e) Mitochondrial sirtuins SIRT3-5 function as sensors of many distinct stress inputs and integrate this information in a coordinated response to maintain cellular homeostasis.

25) Diminished cellular NAD+ levels—as used in the invention, because NAD+ is required for sirtuin activity, low cellular NAD+ can inhibit Sirtuin function and activate inflammasome.

26) NEK7—as used in the invention, NEK7 is member of the family of mammalian NIMA-related kinases (NEK proteins). NEK7 is activated as a result of K efflux which promotes binding of NEK7 to NLRP3 which enables its oligomerization and binding to ASC upon stimulation and mediates NLRP3 activation.

27) c-Jun N-terminal kinases (JNKs)—as used in the invention JNKs are part of the mitogen-activated protein kinase (MAPK) family. They respond to cellular stress stimuli comprising ROS, cytokines, heat or osmotic shock. JNKs are kinases that bind and phosphorylate c-Jun on Ser-63 and Ser-73 within its transcriptional activation domain. JNK can promote mitochondrial apoptosis through the phosphorylation of Bcl-2 family proteins, such as Bcl-2, Bcl-xL and Bax and Bcl-2 regulatory proteins. JNK, as used in the invention, can also promote activation of NLRP3 and AIM2 inflammasomes through the regulation of ASC Oligomerization. JNK induces the phosphorylation of ASC which is required for the oligomerization of ASC.

28) Heat shock proteins (Hsps)—as used in the invention, Hsps are chaperones and assist in proper folding of newly synthetized proteins. Hsps repair misfolded proteins and can also prevent harmful protein aggregation. Hsps 27 and 70 are inducible proteins. Hsp60, Hsc70, and Hsp90 are continuously expressed in mammalian cell. HSP90 guards NLRP3. When it is separated from NLRP3 it enables NLRP3 autophagy. In response to stress stimuli, Hsp90 is released, and NLRP3 can be activated to promote inflammation. If NLRP3 is translated after the priming signal, it is removed from the cell unless protected by a HSP90.

29) Suppressor of the G2 allele of Skp1 (SGT1)—as used in the invention, SGT1 is encoded by Suppressor of G2 allele of SKP1 homolog (SUGT1) gene along with HSP90 which bind to the NLRP3 and prevent its activation. SGT1 is a highly conserved protein found in yeasts, plants, and mammals' functions in multiple biological processes through interaction with different multiprotein complexes.

30) M1 Macrophages—as used in the invention, inflammasomes primarily reside in monocytes and macrophages. Macrophages are polarized to M1, proinflammatory and M2 anti-inflammatory. Given a PAMP (i.e., bacterial pathogen) NLPR3 activation, Casp1 and IL-1β is higher in M1 macrophages then M2.

31) Phospholipase A2 (PLA2)—as used in the invention, PLA2 is an enzyme and produces a variety of biologically active metabolites through the lipoxygenase (LOX), cyclooxygenase (COX), and cytochrome P450 (CYP450) pathways. LOX-1 is a Type II membrane protein found in endothelial cells and immune cells (i.e., macrophages). It can be cleaved and released as soluble form of LOX-1. LOX-1 is the receptor for oxidized LDL. LOX is involved in leukotriene metabolism.

32) Chemokines—as used in the invention, chemokines are small cytokines, signaling proteins secreted by cells. Chemokines enable cells to move through the chemokine's gradient towards higher gradient. Chemokines have four subgroups comprising CXC, CC, CX3C, and C chemokines). These chemokines are recognized by the positioning of the conserved cysteines near the amino-terminus. The CXC subgroup are involved in angiogenesis in both physiologic and pathologic settings. The CXC chemokine family is defined as ELR positive (i.e., CXCL1, CXCL2, CXCL3, CXC16, CXCL7, CXCL8) or ELR negative (i.e., CXCL4, CXCL4L1, CXCL9, CXCL10, CXCL11, CXCL12, CXCL14). ELR defines the presence or absence of a three amino acid sequence, glutamic acid-leucine-arginine (called the “ELR” motif) proximal to the CXC sequence respectively. The ELR” positive CXCs are potent promoters of angiogenesis, while the (interferon inducible), ELR negative CXCs are potent inhibitors of angiogenesis. Chemokines are involved in development, angiogenesis, immune surveillance. Additionally, some chemokines are involved in the inflammatory response which include but are not limited to CCL2, CCL3, CCl5, CXCL1, CXCL2, and CXCL8. Inflammatory chemokines are secreted by immune cells, infected or damaged cells.

33) CXCL1 and CXCL2—as used in the invention, CXCL1 and CXCL2 activate NLRP3 inflammasome via their receptor CXCR2 and via PKCm-ILK axis activating NLRP3 and CASP1. Chemokines are also involved in T cell polarization from T2 to T1. CX3C called fractalkine acts as a chemoattractant and is involved in cell adhesion. Select polymorphisms in Fractalkine have been involved in a higher risk or lower risk of Myocardial infarction (i.e., V249I and T280M polymorphisms, the 1249 allele displays a low level of T cell inflammation and a reduced risk of developing vulnerable plaques and they have a decreased incidence of acute events.) CCL2 is involved as a chemoattractant for monocytes. Genetic polymorphisms int the CCL2 G-2518A and MBL codon 54 variant have a significantly cumulative effect on increased risk of SARS-CoV infection. No studies on SARSCOV2 have been performed yet.

34) Interleukin 17 (IL-17)—as used in the invention, IL-17 is produced by T helper-17 which has a receptor for IL-1β and upon stimulation generates IL-17A, IL-17F, IL-21 and IL-22. When NLRP3 is dysregulated and its activity enhanced it leads to increased production of IL-17, 11-22. In turn IL-17 and IL-22 feedback and further activate NLRP3 inflammasome via ROS generation via NF-KB pathway. IL-17 and IL-22 and (to a smaller extent) IFN-γ appear to induce an increase in NLRP3, the active form of caspase-1, and mature IL-1β.

35) Osmotic stress—as used in the invention, osmotic stress can activate NLRC4 and NLRP3 inflammasomes via increased mROS and the inflammasome converts the osmotic stress (i.e., hyperosmolar conditions) to an immune response which can also cause autoimmunity.

36) Purinergic receptors—as used in the invention, purinergic receptors are a family of plasma membrane molecules found in all mammalian cells and involved in cell processes comprising: apoptosis, cytokine secretion, proliferation, migration and vascular reactivity. Initially they were classified as P2 receptors (which mediated relaxation of GI smooth muscles in response to ATP) and Adenosine P1 receptors. There are 3 classes of purinergic receptors P1, P2Y, P2x. P2x is a ligand gated ion channel, whereas P1 and P2Y are G protein could receptors. P2x family are ion channels gated by extracellular ATP present in many cells and tissues. Some P2X receptors (i.e. P2x2, P2x4, P2x7) when activated by prolonged exposure to high concentrations of ATP, undergo the formation of a pore that allows passage of large cations which can trigger ATP-induced NALP3/NLRP3 inflammasome activation and consequently caspase-1 maturation, pro-interleukin (IL)-1β cleavage and IL-1β release by immune cells. ATP at millimolar concentrations are capable of activating inflammasomes. P1 receptors is preferentially activated by adenosine and P2y receptors are preferentially activated by ATP. Purinoreceptors are involved in NLRP3 activation. Furthermore:
a) P2x2 and P2x7 receptors also interact with β-tubulin.
b) P2x4 receptor is activated in response to hydrostatic pressure and activates NLRP3.
c) P2x5 receptor activates NLRP3. It is expressed in higher amounts in multinucleated cells.
d) P2x7R is a protein encoded by P2X7R gene. It is found in a variety of cells and organs comprising macrophages, epithelial cells, lung, atrial cardiomyocytes and cardiac microvascular endothelial cells, central and peripheral nervous system, microglia, uterine endometrium and retina. In healthy cells and tissue, ATP release into the extracellular space is tightly regulated and its concentration is kept low by extracellular ATP/ADPases. In damaged tissues, ATP is released from injured cells. Transient increase in extracellular ATP is ubiquitously used for cell to cell communication in various organs comprising nervous, vascular, and immune systems. P2RX7 is a pattern recognition receptor for extracellular ATP. It is a ligand gated ion channel and is stimulated in the presence of High ATP concentration and upon activation leads to K⁺ and calcium²⁺ efflux and cell depolarization which leads to NLRP3 activation. It is upregulated following injury or chronic inflammatory situations. For example, ATP is released via the P2X7 receptor-activated pannexin-1 (Panx1) hemichannel in injured lung cells, leading to sustained P2X7 receptor activation on alveolar macrophages, which then activates NLRP3 inflammasome. P2RX7 reaches its peak at day 4 following injury stimulus. Injured tissue will release ATP which activates P2RX7. PRRX7 gene is highly polymorphic including SNPs and insertion/deletion mutations which can confer risks of developing various diseases (i.e., crescentic glomerular nephritis).
e) TLR2/TLR4 can activate P2x4/P2x7 and activate NLRPs inflammasome in response to extracellular matrix biglycan during tissue injury and tissue stress.
f) K efflux via P2RX7 receptor activates NLRP3 inflammasome.
e) P2YR primes NLRP3 in response to hyperosmolar states (i.e. high salt (NACL) in the extracellular space).

37) High Na—Cl (Hyperosmolar state)—as used in the invention, a hyperosmolar state induces expression of the NFAT gene. This signaling involves a pannexin-dependent release of ATP and P2Yi receptor activation. Activation of P2Yi receptors may trigger a PLC- and IP₃-mediated calcium mobilization from internal stores and activation of PKC; inhibition of these intracellular pathways decreased the NaCl-induced expression of the NLRP3 gene. Significant effects of high extracellular NaCl on the expression of the NLRP3 gene have been found when more than 10 mM NaCl were added to the culture medium. It is generally accepted that the highest pathological blood osmolarity in human subjects is around 360 mOsm/kg which can be achieved by addition of 40 mM NaCl to the culture medium. However, less well appreciated is that the local extracellular NaCl concentration in the interstitium may be considerably higher (160-250 mM) than the plasma concentration of NaCl (˜140 mM). These results may have relevance for in-vivo conditions. High NACL induces priming of NLRP3.

38) ELAVL1—as used in the invention, the Inhibition of MicroRNA 9 increases ELAV-like protein 1 (ELAVL1) under hyperglycemic conditions can activate NLRP3 inflammasome.

39) Angiotensin II (AngII)—as used in the invention AngII mediates NLPR3 activation via Micro-RNA 23.

40) Amyloid B proteins—as used in the invention, Amyloid B proteins activate NLRP3 inflammasome via Purinoreceptors and other mechanisms.

41) Cathepsins—as used in the invention, cathepsins contribute to priming step of NLRP3 activation by increasing IL-1B transcriptionally and activation of NLRP3 inflammasome. Cathepsins comprise B, C, D, L, S and X.

42) Lysosomal instability—as used in the invention, upon rupturing, lysosomes can activate NLRP3 inflammasome.

43) Casp 8 and its adapter protein Fas-associated protein with death domain (FADD)—as used in the invention, Casp8 and FADD activate NLPR3 inflammasome. Casp8 is involved in apoptosis. Activation of death receptors comprising: CD95, TRAIL receptor, and TNFR1, result in recruitment of casp8 and its adaptor protein FADD to initiate an apoptosis inducing caspase cascade. Casp8 promotes apoptosis induction in response to canonical inflammasome stimuli when the induction of inflammasome dependent pyroptosis is prevented. Casp8 interacts with ASC of the inflammasome. FADD and caspase-8 promote NF-kB dependent transcriptional upregulation of pro-IL-1β. thus priming NLRP3 inflammasome.

44) Thrombin receptor PAR4—as used in the invention, thrombin receptor PAR4 drives canonical NLRP3 inflammasome.

45) Deubiquitinase enzyme (DUB)—as used in the invention, DUB can activate NLRP inflammasome.

46) CXCR2-PKCm-ILK—as used in the invention, the CXCR2-PKCm-ILK axis activates NLRP3 inflammasomes. 47) Type I interferons—as used in the invention, Type I interferons can activate inflammasome activation.

48) RIPK3/RIK1/ZBP1/MLKL/necroptosis—as used in the invention this signal transduction pathway leads to K efflux and NLRP3 activation.

49) Flightless I (Fli-I)—as used in the invention, Fli-I is a member of the gelsolin family of actin-remodeling proteins, has two functional protein family domains: An N-terminal leucine rich repeat (LRR) domain and a C-terminal gelsolin-like domain. Fli-I is an inhibitor of NLRP3 activation by inhibiting casp-1 as it contains a caspase-1 pseudo substrate site, which inhibits casp1 activity.

50) Leucine-rich repeat Flightless-1-interacting protein 2 li (LRRFIP2)—as used in the invention, LRRFIP2 is widely expressed in many tissues including lung, liver, brain and muscle. Its encoded by LRRFIP2 gene. LRRFIP2 and MYD88, bind to the cytosolic tail of toll-like receptor 4 (TLR4), induce activation of NF-KB signaling. The ubiquitin-like protein FAT10 prevents the interaction. LRRFIP2 downregulates NLRP3 inflammasome by recruiting the caspase-1 inhibitor Flightless-I to the inflammasome complex. LRRFIP2 interacts with NLRP3 via its N-terminal motif in response to NLRP3 inflammasome stimulations and well as the coil motifs of LRRFIP2 inhibit NLRP3 activation by promoting interaction of leucine rich repeat domain of Fli-I with CASP1.

51) Adapter B-cell adaptor for PI3K (BCAP)—as used in the invention, BCAP limits signaling by activating PI3K, which provides an inhibitory pathway limiting TLR responses. BCAP links TLR signaling to PI3K activation. BCAP bind sPI3K, is constitutively phosphorylated and associated with the p85 subunit of PI3K thus BCAP limits cellular responses to TLR stimulation and IL-1β. BCAP interacted with Flightless-1 and its binding partner leucine-rich repeat Flightless-1-interacting protein 2 (LRRFIP2), which promoted an association between BCAP and the inflammasome component NLRP3. BCAP reduced the abundance of active caspase-1.

52) Cellular inhibitors of apoptosis proteins (cIAPs)—as used in the invention cIAPs are essential regulators of cell death and immunity. cIAPs inhibit RIPK3 dependent necroptosis and inhibit Casp1 of NLRP3 inflammasome.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing subject matter has been described in some detail by way of illustration and example for purposes of clarity of understanding, it will be understood by those skilled in the art that certain changes and modifications can be practiced within the scope of the appended claims.

The following examples are offered by way of illustration and not by way of limitation.

### EXAMPLES

The examples provided herein are not meant to be limiting. All reagents and/or active agents, unless otherwise indicated, are obtained commercially.

### Example 1

Four groups of subjects will be enrolled with Mild, Moderate, Severe, and Critical Covid-19 with matching controls.

Blood will be drawn from each subject.

Gene transcriptomics and/or proteomics studies will be performed in different cell lines (i.e. platelet, macrophages, neutrophils) and on available tissues obtained from the subjects.

Biomarkers will be checked from peripheral blood from subjects: comprising Il-18, Il-1b, Il-6, CRP, Troponin, PT, PTT, Complete blood count, Complete metabolic panel, ESR, D-Dimer, TNF alpha, microparticles, and others.

Ten to thirty compositions will be selected from Tables 1 to 4 shown in FIGS. 1 to 4 and will be administered in combination to the subjects from the following categories:

1) Two or more inhibitors of NLRP3 priming and/or activation signal, comprising:
a) An inhibitor of cGAS-STING;
b) An inhibitor of NLRP3 mediated CASP1 activation mediated Calpastatin cleave induced filamin cleavage mediated procoagulant microparticles expressing decrypted Tissue factor with high activity in different cells lines (endothelial cells, alveolar epithelial cells, macrophages, platelets, neutorphils) formation leading to thrombosis comprising:
   i) An activator of SARS-CoV-2 induced CAST downregulation;
   ii) An inhibitor of TXNIP mediated NLRP3 activation;
   iii) An inhibitor of P2RX7 mediated NLRP3 inflammasome hyperactivation via increasing extracellular ATP release;
   iv) An inhibitor of NLRP3 hyperactivation induced GSDMD mediated Pyroptosis or MLKL mediated Necroptosis, or NLRP3 inflammasome mediated panoptosis;
   v) An inhibitor of NLRP3 mediated GSDMD induced NETosis; or
   vi) An inhibitor of NLRP3 mediated pyroptosis in Platelets including BTK inhibitor; and
c) An inhibitor of AIM1 inflammasome (i.e., methylene blue).

2) A modulator of RAAS dysregulation comprising an inhibitor of the ACE/Angiotensin II arm of RAAS, and/or an activator of the ACE2/Ang(1-7)/Mas1 ARM of RAAS;

3) A modulator of KKS dysregulation;

4) An inhibitor of Proteases, comprising TMRPSS2, TMPRSS4, Cathepsin B, Cathepsin L, and/or Furin (preferably administered together);

5) A cytokine modulator, including a cytokine modulator that activates Interferon I and Interferon III 0 to 48 hours post infection and/or inhibits Interferon I and Interferon III more than 48 hours post infection, a cytokine modulator that activates IL-10 and others.

Group 5 subjects providing composition from Table 1-4 shown in FIGS. 1-4 to prevent SARSCOV2 infections.

Monitor subjects for symptoms of COVID-19.

### Example 2

In one example, a composition is administered to a mammal, preferably a human, in need thereof to induce an artificial biological mimicry wherein the immune advantages of a bat's immune system is mimicked and induced in the mammal as a prophylaxis and/or a treatment of SARS-CoV-2 and or COVID-19. Where the mammal is a human subject, the human subject has clinical signs and symptoms including but not limited to thromboembolism, stroke, myocardial infarction, lung injury, acute respiratory distress syndrome (ARDS), anti-phospholipid like syndrome, Kawasaki like syndrome, and/or vasculitis.

The composition is a modulator of inflammasome priming and/or activation preferably NLRP3 inflammasome signaling pathways comprising: NRLP3 inflammasome, its downstream mediator (ASC/PYCARD), its effector Caspaise-1, cytokines (IL-1beta, IL-18), GSDMD and pyroptosis. The modulator is an inhibitor of a single or multiple activators of NLRP3 priming and/or activation including but not limited to:
1) An inhibitor of inflammasome priming and or activation (i.e NLRP3) comprising but not limited to compositions listed in Table 1,
2) an inhibitor of Endoplasmic Reticulum stress and ER associated UPR comprising of:
   a) sodium phenyl butyrate, Tauro-urso-deoxycholic acid (TUDCA) Silybin, curcumin
   b) an inhibitor of IRE1a and eIF2a, JNK-dependent IRS-1 serine phosphorylation activation (indicative of ER stress (i.e. TUDCA, Silybin, 4-phenylbutyrate),
   c) an activator of NR1H4 comprising: TUDCA, genistein, angelicin, montelucast, felodipine, and catechin gallate, tretinoin, yangonin, ivermectin
3) an inhibitor of microtubule function comprising of:
   a) an inhibitor of microtubule assembly (i.e. colchicine, demecyclin)
   b) an inhibitor of acetylation of alpha tubulin of microtubules which allows dynein to move along microtubules (i.e. NAD+, Nicotinamide Riboside, NMN, Silybin)
   c) an inhibitor of tubulin assembly into microtubules (i.e. berberine)
   d) an inhibitor of NAD+ depletion (i.e. NAD+, silybin, NMN) comprising of:
      i) an activator of SIRT2 and or an activator of SIRT2 gene expression and or activity (i.e. Metformin, Niacin, Resveratrol, Valproic Acid)
         (1) an activator of NAMPT (i.e. Copper Sulfate, Cyclosporine, Demecolcine, Dexamethasone, Estriol, Fenofibrate, gossypol acetic acid, GSK-J4, Levofloxacin, Metformin, NAD, Progesterone, Resveratrol, Sunitinib, Testosterone, Troglitazone, Zinc
         (2) an activator of NMNAT1 (i.e. AZM551248, Cyclosporine, Ethinyl Estradiol, pirinixic acid, Valproic Acid, vinclozolin, Zinc)
         (3) an inhibitor of PARP/PARP1 (i.e. Acetylcysteine, Clobetasol, Curcumin, Cyclosporine, delphinidin, Doxorubicin, Dronabinol, fisetin, flavone, Flavonoids, gossypetin, Isoproterenol, NAD, Niacinamide, Niclosamide, Nicotine, Nitroglycerin, pirinixic acid, Polyphenols, Quercetin, Silymarin, vinclozolin, Zinc, Zinc Sulfate,
         (4) an agonist of NAD+
         (5) a mimetic of NAD+
4) an inhibitor of mitochondrial stress (nonlimited examples include decrease in mitochondrial membrane potential (Δψm), mitochondrial ROS, decrease in ATP and NAD+ generation, decrease in mitochondrial outer membrane permeabilization (MOMP), increase mitochondrial UPR, damaged mitochondrial DNA, MAVP) comprising:
   a) an inhibitor of mtROS comprising of:
      i) MB, MitoTempo, MitoQ, Mito-C, SKQ1, SS-31, N-Acetyl Cysteine (NAC), vitamin D, methylene blue, ubiquinol, N-acetyl cysteine, alpha lipoic Acid, Tempol, Vitamin C, Apocynin, MnP and others
      ii) an activator of NFE2L2 gene expression or activity comprising: curcumin)
      iii) an inhibitor of NADPH oxidase comprising: NAC, apocynin)
      iv) an activator and or a mimetic of SOD (i.e. Tempol)
      v) an activator and or a mimetic catalase mimetic (i.e. EUK134, nanoceria)
      vi) an activator of autophagy comprising of: ambroxol, bromhexine, MB, hydroxychloroquine mtROS, chloroquine, epigallocatechin gallate EGCG, and daidzein
   b) an inhibitor of diminished mitophagy comprising of:
      i) an activator of mitophagy—(i.e. EGCG)
   c) an inhibitor of mtDNA damage
      i) an inhibitor of mtROS (i.e. NAC)
      ii) an inhibitor of LOX-1 comprising: quinacrine, Boswellia Serratia, and pycnogenol
   d) an inhibitor of diminished mitochondrial membrane potential (Δψm) (i.e. MB)
   e) an inhibitor of mitochondrial fission comprising: mitoQ
   f) an inhibitor of mitochondrial apoptosis comprising:
      i) an inhibitor of MAPK, P38 (P38 delta), JNK pathway comprising: curcumin, resveratrol, oridonin
   g) an activator of NAD+ salvage pathway enzymes comprising: NMAT1, NAMPT
5) an inhibitor of an oxidoreductase enzyme comprising of:
   a) an inhibitor of xanthine oxidoreductase (XOR) (which include xanthine oxidase (XO)) (XO induces mitochondrial ROS via the PI3K-AKT-mTOR and can prime and or activate NLRP3) comprising of:
      i) a purine analogue comprising: allopurinol, oxypurinol, and tisopurine
      ii) a non-purine analogue of XOR comprising: febuxostat, topiroxostat, and inositols (i.e. phytic acid inositol hexaphosphate (IP6) and myo-inositol)
      iii) an electrophilic nitrated fatty acid derivative, (i.e. nitro-oleic acid)
      iv) a SOD mimetic
      v) a Daidzein derivative i.e. X-11-5-27 which activates autophagy as well
6) an inhibitor of TXNIP comprising: metformin, gl1-agonist, resveratrol, melatonin, TUDCA, silybin, linarin
7) an inhibitor of lysosomal rupture comprising of:
   a) an inhibitor of NADPH oxidase comprising of:
      i) NAC, Apocynin (4′-hydroxy-3′-methoxyacetophenone)
      ii) an inhibitor of MAPK, P38 (P38 delta), JNK pathway comprising: curcumin, resveratrol, oridonin
8) an inhibitor of Lysosomal membrane permeabilization and or cathepsins comprising of cathepsins B, C, F, H, K, L, O, S, V, and Z (also known as X). Some examples of cathepsin inhibitorscomprise: an inhibitor cathepsin B and/or Cathepsin L comprising: E-64, Miraziridine A, Tokaramide, Guttiferone A, β-Ursolic acid, Schinol, Flavonoid F5, Allicin, Aurantiamide acetate, Quercetin, and bioflavonoids agathisflavone isolated from the leaves of Ouratea nigroviolacea, tetrahydrorobustaflavone isolated from the fruits of Schinus terebenthifolius; an inhibitor of cathepsin Z comprising: buspirone, cyclosproin, dexamethasone, genistein, prinixic acid, sunitinib, valproic acid; an inhibitor of cathepsin C comprising: acetaminophen, copper sulfate, cupric chloride, disulfiram, fenofibrate, ibuprofen, metformin, mifepristone, testosterone, progestins, valproic acid, and vancomycin; an inhibitor of cathepsin S comprising: Acetaminophen, Acetylcarnitine, anagliptin, astaxanthine, Buspirone, Cadmium Chloride, Calcium, Cisplatin, Cisplatin, coenzyme Q10, Copper, Curcumin, Dehydroepiandrosterone, Doxorubicin, Erianin, Estradiol, Fenretinide, Flutamide, (+)-JQ1 compound, Lycopene, Methotrexate, Mifepristone, nimesulide, Phenobarbital, pirinixic acid, PP242, Pregnenolone Carbonitrile, Progesterone, tempol, Thioctic Acid, Valproic Acid, Valsartan, vanadyl sulfate, and Vancomycin, an inhibitor of cathepsin C comprising: Acetaminophen, adefovir dipivoxil, Cadmium Chloride, cobaltous chloride, Copper Sulfate, cupric chloride, Cyclosporine, Disulfiram, Fenofibrate, Fenretinide, Flavonoids, Flutamide, Ibuprofen, K 7174, Metformin, Mifepristone, Niclosamide, Oxycodone, Oxygen, Phenobarbital, Phenylephrine, pirinixic acid, Progestins, Sunitinib, Tamoxifen, Testosterone, Tretinoin, Valproic Acid, Vancomycin, XL147; an inhibitor of cathepsin G comprising: Acetaminophen, Ethinyl Estradiol, honokiol, Methotrexate, sulforafan, and Tretinoin,
9) an inhibitor of DPP4 comprising: Acetaminophen, Adenosine, Adenosine, Adenosine, alogliptin, anagliptin, Azathioprine, beta-hydroxy simvastatin acid, Cadmium Chloride, ciprofibrate, Clofibrate, cobaltous chloride, Copper Sulfate, cupric chloride, Cyclosporine, Doxorubicin, Fenretinide, Flutamide, (+)-JQ1 compound, K 7174, Linagliptin, MDV 3100 (enzalutamide androgen rec antagonist), MK0626, MK0626, Phenobarbital, pirinixic acid, PT-100 dipeptide, Quercetin, Rimonabant, saxagliptin, saxagliptin, saxagliptin, Sitagliptin Phosphate, Ursodeoxycholic Acid, Valproic Acid, Vildagliptin, voglibose
10) an inhibitor of HMGB1/RAGE (i.e. lysosomal disruption leads to cathepsin B release and HMGB1-RAGE signaling pathway activates NLRP3) comprising: Glycyrrhizin, EGCG
11) an inhibitor of oxidative stress, reactive oxygen species (ROS) and superoxide comprising of:
   a) an inhibitor of NADPH Oxidase (NOX: i.e. NOX2) comprising of:
      i) apocynin, celastrol, methylene blue, acetyl-1-carnitine, Tempol, and phycocyanobilin
      ii) an inhibitor NOX2 assembly comprising: acetyl cysteine, glutathione
      iii) an inhibitor of NOX2 gene expression comprising: astaxanthin, Genistein, Resveratrol
   b) Methylene blue
   c) a SOD mimetic (i.e. tempol)
   d) an activator of NFE2L2 gene expression or activity (i.e. curcumin)
   e) an antioxidant comprising: vitamin C, NAC, Melatonin, alpha and gamma tocopherols, MB, alpha lipoic Acid)
   f) an activator of autophagy comprising: ambroxol, bromhexine, MB, Hydroxychloroquine, chloroquine, epigallocatechin gallate EGCG, daidzein
12) an inhibitor of NF-KB comprising of:
   a) an inhibitor of NF-KB phosphorylation comprising; beta hydroxybutyrate, and ketone body
   b) an inhibitor of STAT3 upregulation of Histone 3 and 4 acetylation and & H3K4 trimethylation comprising: BHB, ketone bodies
   c) an inhibitor of MAPK, JNK, P38 pathways to downregulate NF-KB pathways and Toll like receptor pathways (i.e. TLR 7, 9, 4, 3) which can transcriptionally prime inflammasomes (NLRP3 inflammasome) comprising: curcumin, resveratrol, oridonin
   d) an inhibitor of NF-KB activation comprising:
      i) curcumin, methylene blue, melatonin, Cichorium intybus L. (chicory, chicoric acid), quinacrine
      ii) an inhibitor of inducible isoform of nitric oxide synthase comprising: melatonin, methylene blue
   e) an inhibitor of IKK (inhibits NF-KB) (i.e. wedelolactone) which inhibits NF-KB and also decrease Casp11)
   f) an activator of SIRT1 (i.e. melatonin)
13) an inhibitor of HSP90 (i.e. curcumin, gelendamycin, withaferin A, 17-DMAG)
14) an inhibitor of SUGT1 (i.e., tretinoin, phlorhizine, cobaltous chloride)
15) an inhibitor of NEK7 (to prevent NEK7-NLRP3 Interaction) (i.e. Oridonin)
16) an inhibitor of intracellular calcium increase comprising of:
   a) an inhibitor of ER mediated calcium release (i.e. calcium channel blockers)
   b) an inhibitor of voltage dependent calcium channels (i.e. verapamil, magnesium) (ER depletion of calcium into the cytoplasm can prime and or activate NLRP3)
17) an inhibitor of Angiotensin II which activates NLRP3 inflammasome
   a) an inhibitor of ACE comprising: lisinopril, captopril, enalapril, ramipril, fosinopril, perindopril
   b) an inhibitor of AT1R comprising: telmisartan, losartan, valsartan
   c) an inhibitor of MIR21 gene transcription comprising: arctigenin, curcumin resveratrol, quercetin, valproic acid, copper)
   d) an activator of ACE2
   e) an activator of MAS receptor comprising of:
      i) Ang (1-7) mimetic
   f) an activator of neprolysin and or MME gene expression and or activity
18) an inhibitor of intracellular rise of particulate/crystals (i.e. uric acid crystals, calcium phosphate crystals) which activate NLRP3 inflammasome comprising of:
   a) an inhibitor of xanthine oxidase comprising of:
      i) a purine analogue comprising: allopurinol, oxypurinol, and tisopurine)
      ii) a non-purine analogue of XOR comprising: febuxostat, topiroxostat, and inositols (phytic acid inositol hexaphosphate (IP6) and myo-inositol)
      iii) an electrophilic nitrated fatty acid derivative (i.e. nitro-oleic acid)
      iv) a SOD mimetic
19) an inhibitor of necroptosis which activates NLRP3 inflammasome comprising:
   a) an inhibitor RIP1/RIP3 comprising: methylene blue, kongensinA, debrafinib, and GSK872
   b) an inhibitor of RIPK3 gene transcription and activity and or methylation comprising: Copper, Ketamine, Valproic Acid, GSK-J4, DHT, ICG 00
   c) an inhibitor of MLKL gene expression, and or activity or phosphorylation
   d) an inhibitor of necroptosis related gene comprising RIPK3, RIPK1, MLKL, and ZBP1 wherein the inhibition comprises: an inhibitor of mRNA expression, an inhibitor of gene protein activity, stability, reaction, function resulting in downregulation of the gene and or its protein
   e) a modulator of PARP1 comprising inhibitor of PARP1 gene expression, PARP1 protein activity and or post translational modification
   f) an activator of Casp8 gene expression and or activity and cleavage of Casp8
20) an inhibitor of cFLIP1 comprising: acetylcysteine, bortezomib, cafetol, cardanol, clofibrate, curcumin, quercetin, pentoxifylline, prinixic acid, paclitacel, resveratrol, rosiglitazone, steviol, stevioside, sulindac, tretinoin, vorinostat, zerumbone
21) an inhibitor of a gene transcription and or a gene activity comprising of:
   a) an inhibitor of NLRP3 gene transcription and/or activity (i.e. valproic acid, quercetin, minocycline, estradiol, progesterone, simvastatin, rhodioloside, MC-950) (vinpocetine in pigmented retinal epithelium)
   b) an inhibitor of Il-1b gene transcription and activity
   c) an inhibitor of Il-18 gene transcription and activity
   d) an inhibitor of PYCARD gene transcription and activity
   e) an inhibitor of Casp-1 gene transcription and activity
   f) an inhibitor of caspases (i.e. methylene blue)
   g) an inhibitor of GSDMD gene transcription and activity
   h) an inhibitor of NLRP3 oligomerization (i.e. Tranilast)
   i) an inhibitor of ASC oligomerization (i.e. MCC950, Methylene blue)
   j) an inhibitor of NF-KB gene transcription and activity
   k) an inhibitor of Angiotensinogen gene transcription and activity
   l) an inhibitor of ACE gene transcription and activity
   m) an inhibitor of ADAM9 gene transcription and activity
   n) an inhibitor of cathepsin genes activity and or transcription comprising: CSTB, CSTL, CSTS, CSTG, and CSTX genes
   o) an inhibitor of NOX gene transcription and activity
   p) an inhibitor of HMGB1 gene transcription and activity
   q) an inhibitor of MAVS gene transcription and activity and or methylation (Copper, Ketamine, Valproic Acid, GSK-J4, DHT, ICG 001)
22) an inhibitor of Chloride channels (i.e. Flufenamic acid)
23) an inhibitor of ATP sensitive K channels (i.e. glybenclamide)
24) an inhibitor of ASC oligomerization (i.e. orodonin)
25) an inhibitor of DUBS (deubiquitinase enzyme) (DUB via BRCC3 mediates deubuitination of NLRP3 which primes and activates NLRP3) comprising: Bortezomib, Catechin, Copper Sulfate, Cyclosporine, Ethinyl Estradiol, Flavonoids, Piroxicam, Plant Extracts, Polyphenols, Promethazine, Resveratrol, and Sunitinib,
   a) an inhibitor of BRCC3 (BRCC3 (BRCA1-BRCA2-containing complex subunit 3)
   b) a Vitamin D receptor agonist (i.e. calcitriol) which binds to NLRP3 and prevents NLRP3s binding to BRCC3
26) An inhibitor of NLRP3 desumoylation comprising an inhibitor SENP6, SENP7
   a) An inhibitor of SENP7 comprising: Acetaminophen, Cisplatin, Doxorubicin, entinostat, jinfukang, Ketamine, pentanal, Quercetin, Resveratrol, schizandrin B, Topotecan, Troglitazone, Valproic Acid, and Zinc,
   b) An inhibitor of SENP6 comprising: Acetaminophen, Copper Sulfate, Cyclosporine, Irinotecan, jinfukang, pentanal, Topotecan, Valproic Acid, and Vorinostat,
27) an inhibitor of non-canonical inflammasome activation in which activated casp11, 5, 4 activates proptosis and secretes II-1 alpha, HMGB1, ATP into the extracellular space which activates NLRP3 inflammasome comprising of:
   a) an inhibitor of Casp 11, Casp 4 & 5 (in humans) comprising of:
      i) methylene blue, Wedelolactone (7-methoxy-5,11,12-trihydroxy-coumestan)
   b) an inhibitor of IKK (inhibits NF-KB) (i.e. wedelolactone) which inhibits NF-KB and Casp 11)
   c) an inhibitor of Mast cell cytokine and protease release (i.e. Resveratrol, a mast cell stabilizer comprising: Azelastine, Cromoglicic acid, Ketotifen, Olopatadine, Rupatadine, Mepolizumab, Omalizumab, Pemirolast, Quercetin, Nedocromil, Tranilast, Palmitoylethanolamide, b-adrenergic agonists and Vitamin D3)
28) an inhibitor of CXCR2-PKCm-ILK axis
29) an inhibitor of IL-17 (i.e an inhibitor of IL17a comprising: Chloroquine, Cyclophosphamide, Diclofenac, Estradiol, ferulic acid, ferulic acid, (+)-JQ1 compound, Methotrexate, Methylene Blue, methyl salicylate, Metyrapone, morin, Prednisolone, Resveratrol, saikosaponin, Simvastatin, tetrandrine, trikatu, tripterine (celastrol), U 0126, and Wortmannin,
30) an inhibitor of IL-21 comprising: Cyclophosphamide, (+)-JQ1 compound, Simvastatin, Triclosan,
31) an inhibitor of P2X5 comprising: Cadmium Chloride, Cisplatin, Cyclosporine, Demecolcine, Endosulfan, Estradiol, jinfukang, (+)-JQ1 compound, Sunitinib, Tamoxifen, Tretinoin, Valproic Acid, Vanadates, and Vincristine,
32) an inhibitor of PRXS (i.e., berberine)

### Example 3

In one example, a composition is administered to a mammal, preferably a human, in need thereof to induce an artificial biological mimicry wherein the immune advantages of a bat's immune system is mimicked and induced in the mammal as a prophylaxis and/or a treatment of SARS-CoV-2 and or COVID-19. Where the mammal is a human subject, the human subject has clinical signs and symptoms including but not limited to thromboembolism, stroke, myocardial infarction, lung injury, acute respiratory distress syndrome (ARDS), anti-phospholipid like syndrome, Kawasaki like syndrome, and/or vasculitis.

The composition is a modulator of inflammasome priming and/or activation preferably NLRP3 inflammasome signaling pathways comprising: NRLP3 inflammasome, its downstream mediator (ASC/PYCARD), its effector Caspaise-1, cytokines (IL-1beta, IL-18), GSDMD and pyroptosis. The modulator is an inhibitor of a single or multiple activators of NLRP3 priming and/or activation including but not limited to:
1) an activator of cellular autophagy comprising:
   a) ambroxol, bromhexine, MB, hydroxychloroquine, chloroquine, epigallocatechin gallate EGCG, quinacrine
      i) an inhibitor of phosphorylation of proteins in in PI3K/AKT/mTOR signaling pathways which enhances autophagy mediation ROS reduction (i.e. daidzein, a Daidzein derivative i.e. X-11-5-27)
2) an activator of mitophagy (nonlimiting examples: mitoQ, methylene blue)
3) an activator of purine salvage pathways (nonlimiting examples: febuxostat, ribose)
4) an activator to restore Mitochondrial Cellular Bioenergetics comprising: febuxostat, ubiquinol, coenzyme q10, MB
5) an activator of Nrf2 gene expression and activity comprising of
   a) an inhibitor of ampk/gsk3b)
   b) curcumin, linarin, xanthohuman, mangiferrin, melatonin, curcumin,
6) an activator of Sirt1 comprising: Melatonin, MB, resveratrol, buteine, piceatannol, soliquiritigenin (4,2 0,4 0 trihydroxychalcone), Fisetin (3,7,3 0,4 0 tetrahydroxyflavone, Quercetin (3,5,7,3 0,4 0 pentahydroxyflavone), Piceatannol, (3,5,3 0,4 0-tetrandroxytransstilbene
7) an activator ACE2/MAS/ANG (1-7) comprising of
   a) a mimetic of Ang (1-7, ang (1-7)
   b) an activator of ACE2
   c) an activator of MAS receptor
8) an activator of NLRP3 ubiquitination and degradation comprising of:
   a) an activator dopamine (DA)-DRD1 receptor signaling comprising: bromocriptine, cabergoline
9) an activator of NAD+ (i.e. silybin, NAD+, NMN) Nicotinamide Riboside
   a) an activator of nicotinamide salvage pathways
   b) a precursor of NAD+ (i.e. NMN)
   c) an agonist of NAD+
   d) an NAD+ mimetic
   e) an inhibitor of ROS and oxidative stress
   f) an inhibitor of PARP1 comprising: Acetylcysteine, Clobetasol, Curcumin, Cyclosporine, delphinidin, Doxorubicin, Dronabinol, fisetin, flavone, Flavonoids, gossypetin, Isoproterenol, AD, Niacinamide, Niclosamide, Nicotine, Nitroglycerin, pirinixic acid, Polyphenols, Quercetin, Silymarin, vinclozolin, Zinc, Zinc Sulfate
   g) an activator of gene expression comprising of NMAT, NMAT3, NAMPT
   h) an activator of activity of enzymes comprising of NMAT, NMAT3, NAMPT
10) an activator of NAD+ dependent SIRT2 (i.e. silybin)
11) activator of mitochondrial uncoupling
12) an activator of NFE2L2 gene expression and or activity comprising:
13) an activator of mitochondrial biogenesis
14) an activator of transcription and or activity of genes comprising of:
   a) an activator NAD+ comprising: NM, NAD, and methylene blue)
   b) an activator of SIRT1 comprising of methylene blue (MB), resveratrol, buteine, piceatannol, soliguirtigenin, fisetin, and quercetin
   c) an Activator of SIRT2 comprising: resveratrol, niacin, metformin and valproic acid
   d) Activator of NAMPT gene (i.e. metformin, resveratrol, progesterone, troglitazone, zinc)
   e) An activator of NMNAT1 gene comprising: AZM551248, Cyclosporine, Ethinyl Estradiol, pirinixic acid, Valproic Acid, vinclozolin, and Zinc,
   f) an activator of MicroRNAs comprising:
      (1) an activator of MIR9 comprising: resveratrol, Epigallocatechin gallate
      (2) an activator of MicroRNA30c-5p comprising: resveratrol, cupric chloride, and tretinoin,
      (3) an activator of MicroRNA 20A comprising: genistein, acetaminophen, tamoxifen and zinc,
      (4) an activator of MIR223 comprising: cisplatin, curcumin, elaginc acid, and zinc
      (5) an activator of MIR495 comprising: phenobarbital, testosterone
   g) an activator of CASP8 which inhibits necroptosis
15) an activator of NLRP3 sumoylation (i.e activator of MAPL)
16) an activator of NLRP3 ubuitination
17) an activator of Farnesoid receptor x stress and NR1H4 comprising: taurodeoxycholic acid, genistein, angelicin, montelucast, felodipine, and catechin gallate
18) an activator of M2 macrophage polarization from M1 (i.e. progesterone)

### Example 4

In one example, a composition is administered to a mammal, preferably a human, in need thereof to induce an artificial biological mimicry wherein the immune advantages of a bat's immune system is mimicked and induced in the mammal as a prophylaxis and/or a treatment of SARS-CoV-2 and or COVID-19. Where the mammal is a human subject, the human subject has clinical signs and symptoms including but not limited to thromboembolism, stroke, myocardial infarction, lung injury, acute respiratory distress syndrome (ARDS), anti-phospholipid like syndrome, Kawasaki like syndrome, and/or vasculitis.

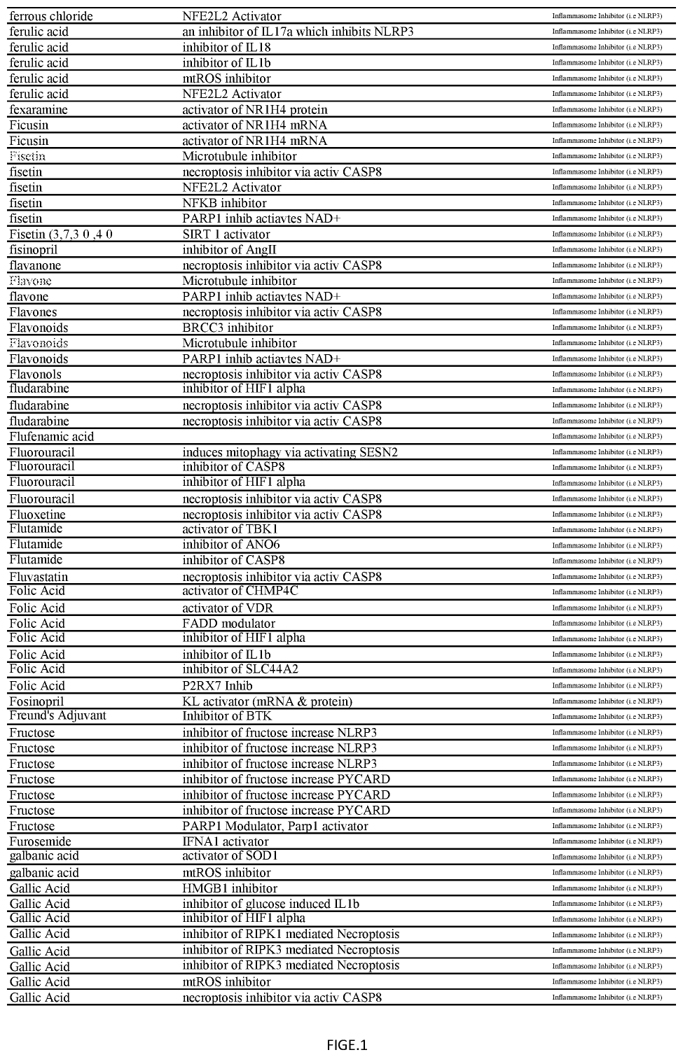

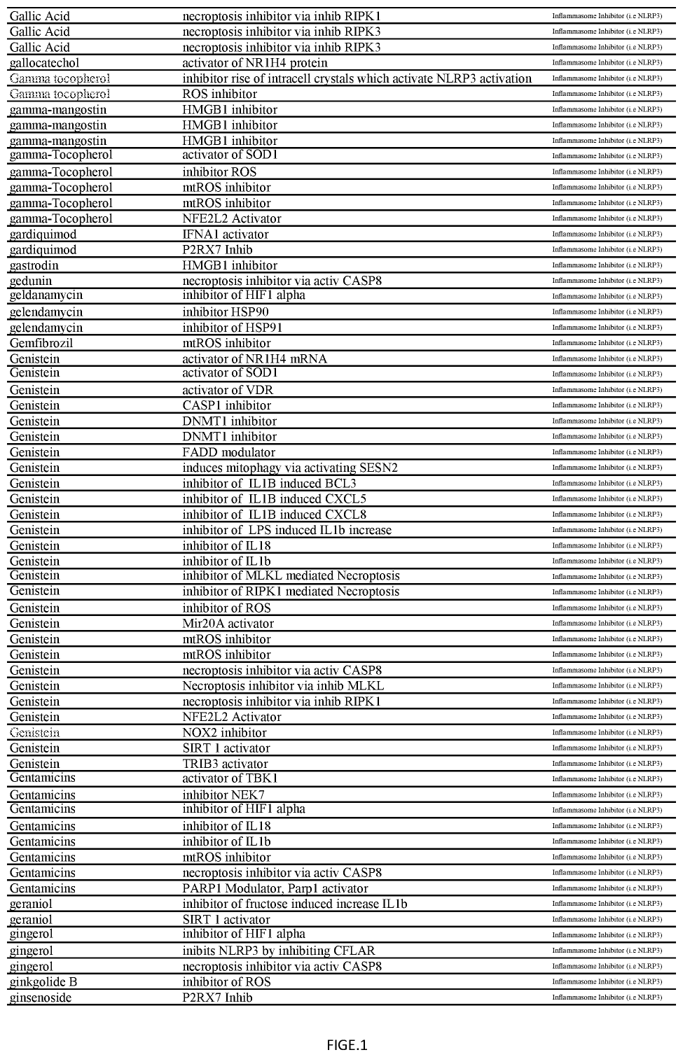

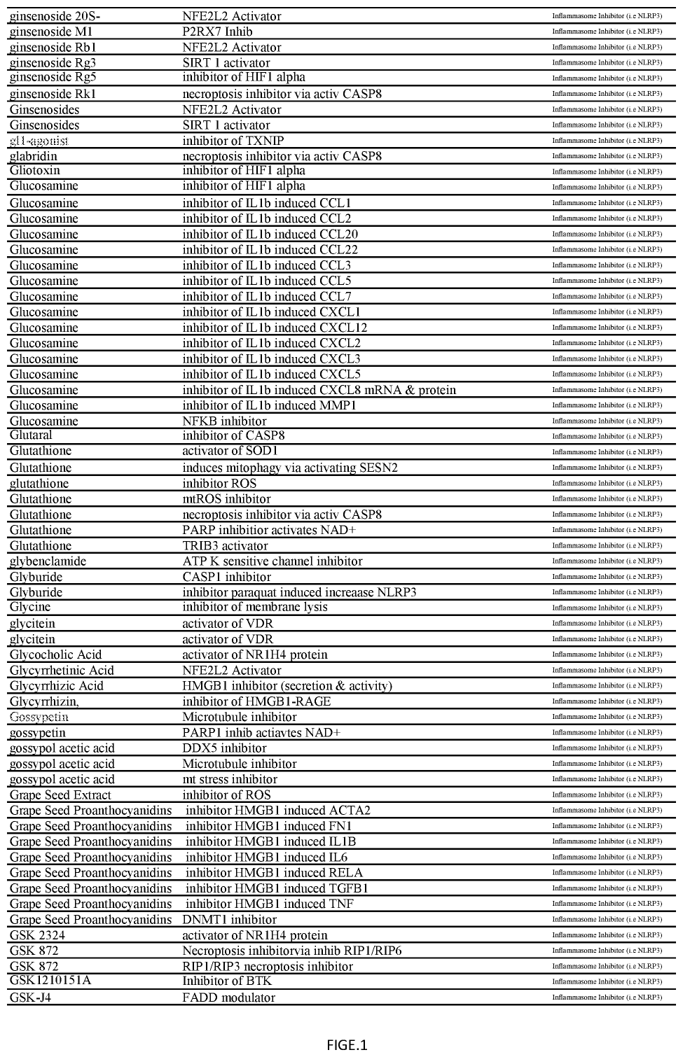

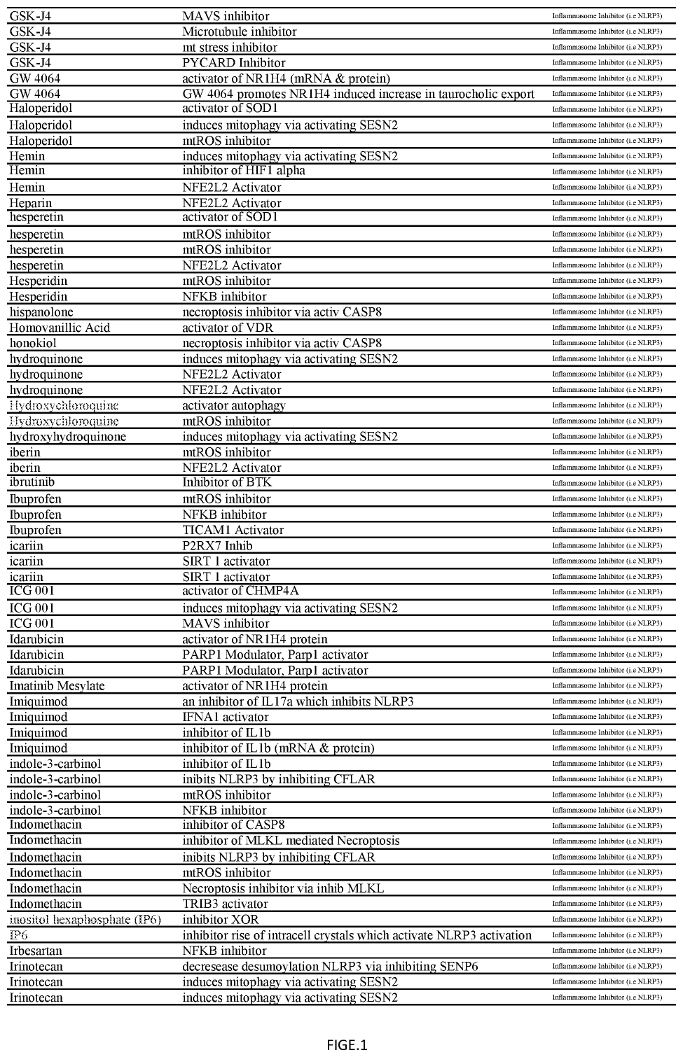

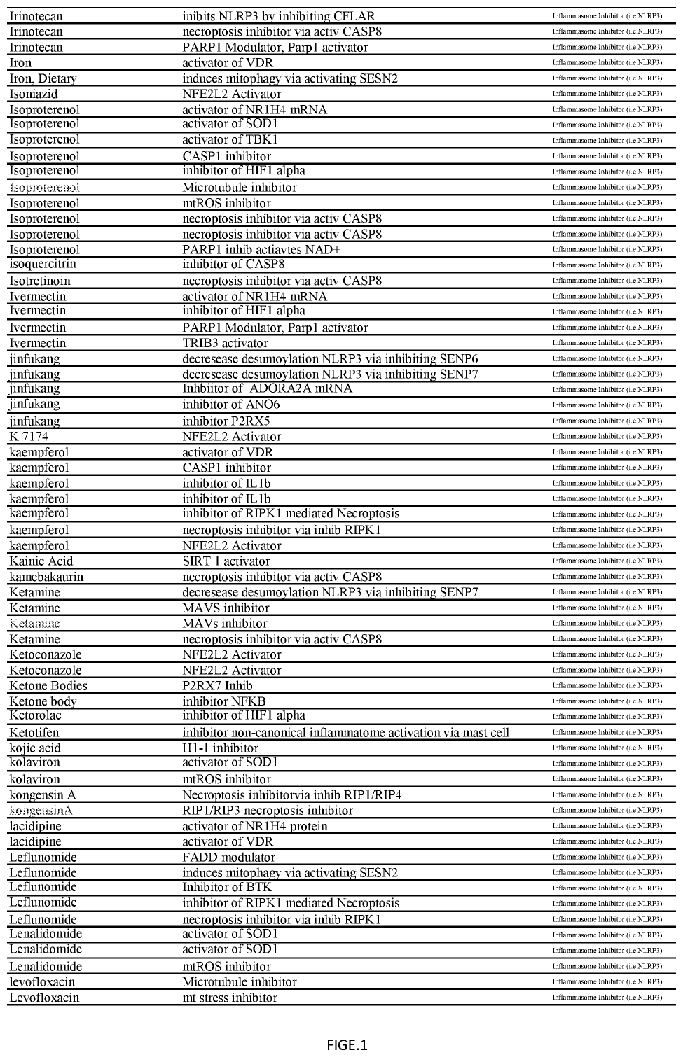

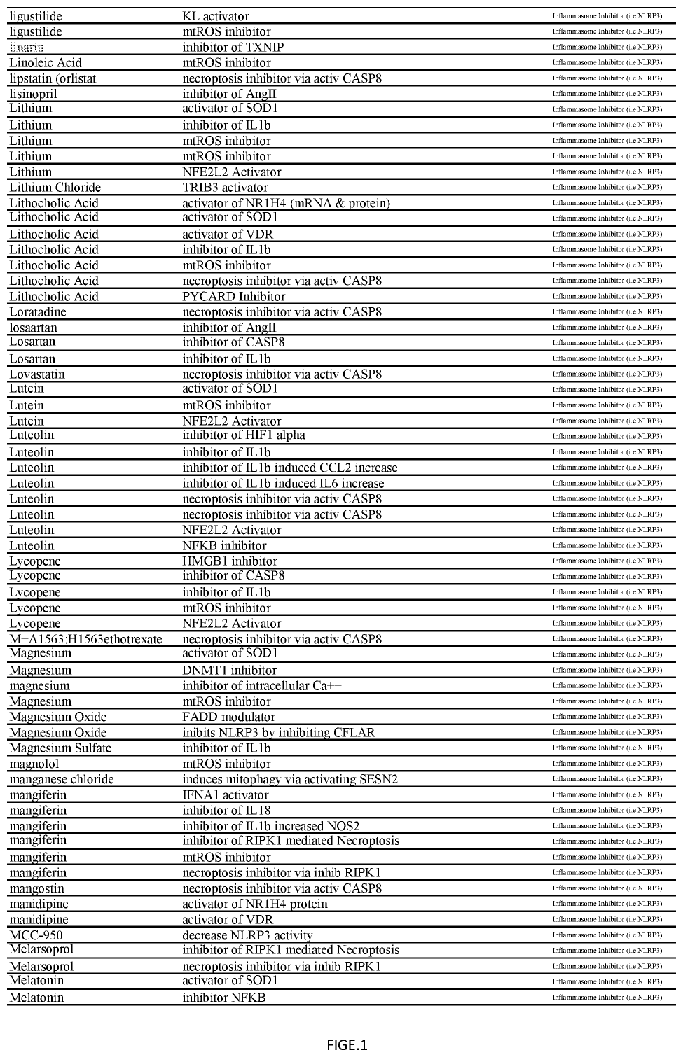

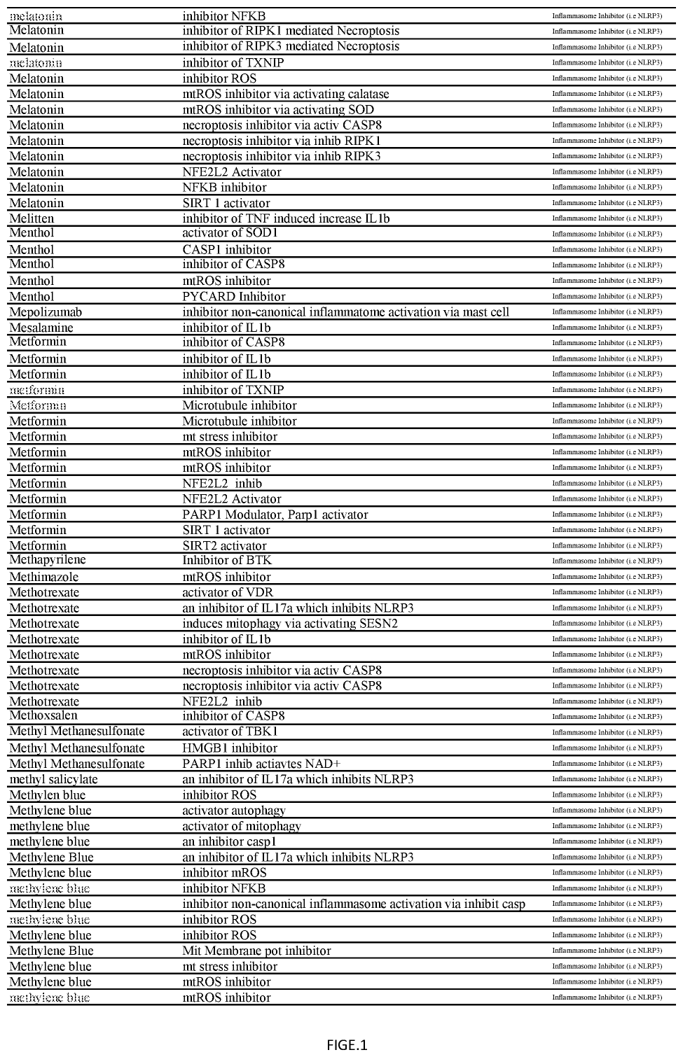

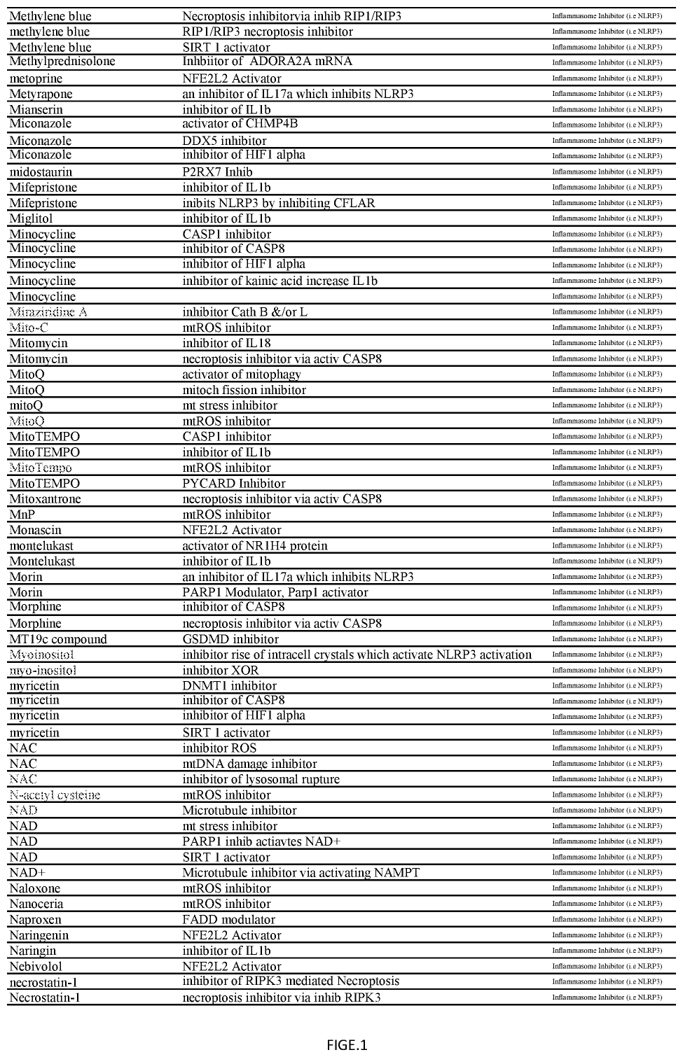

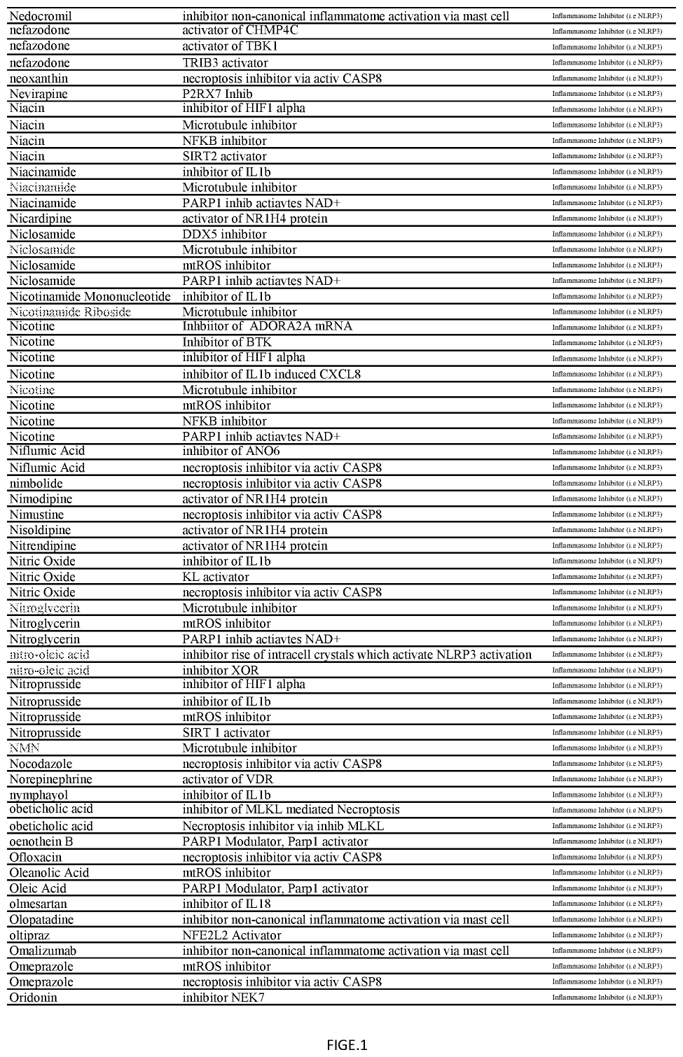

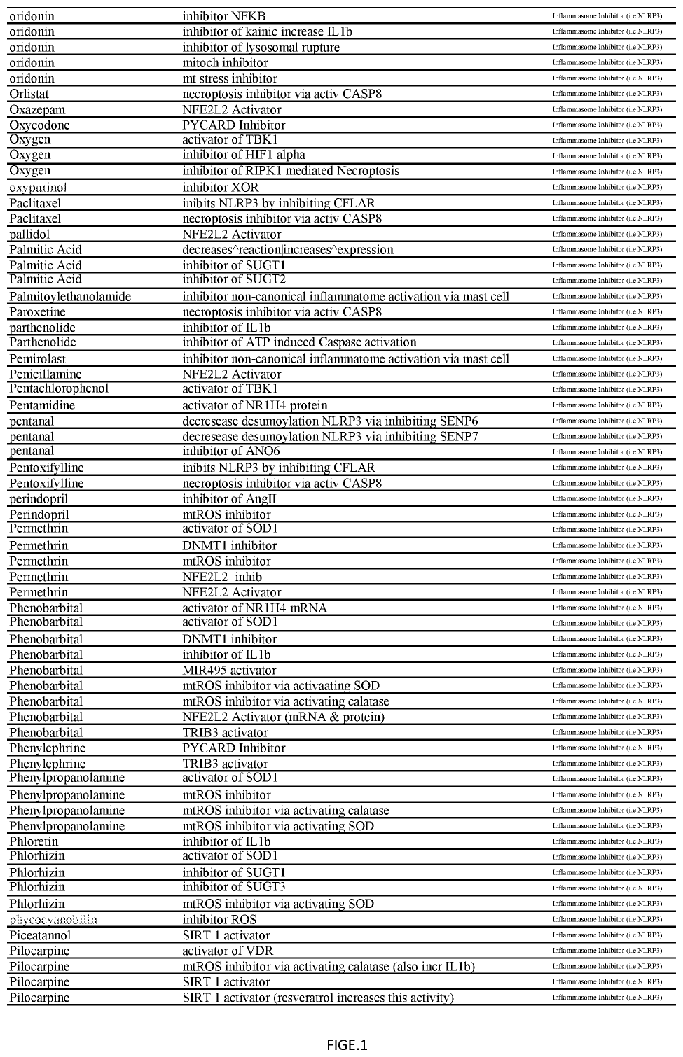

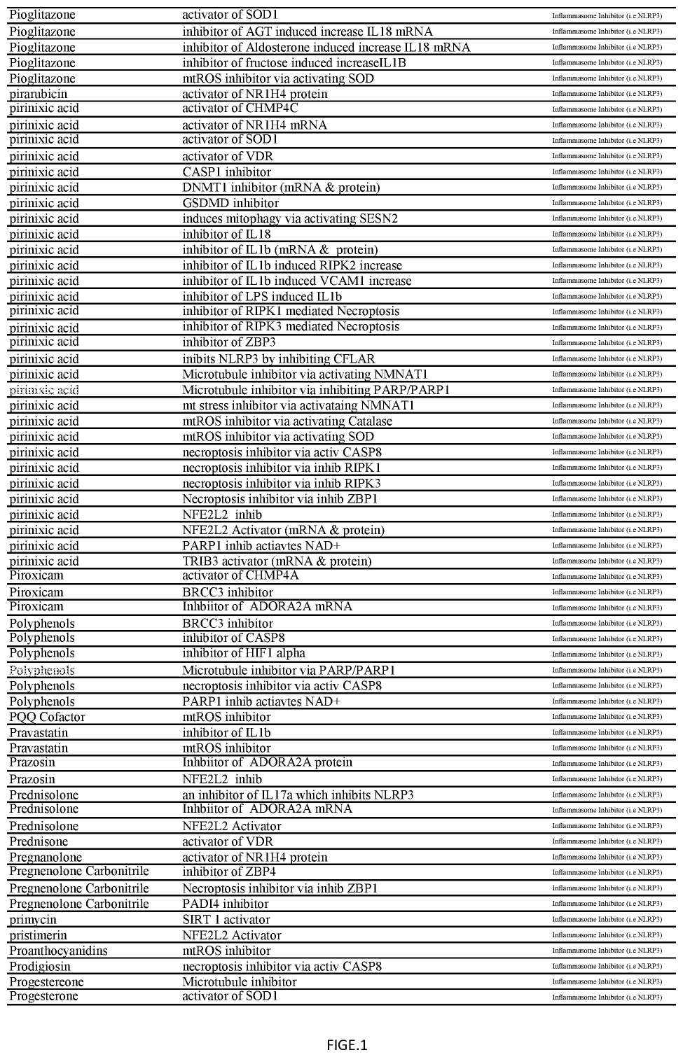

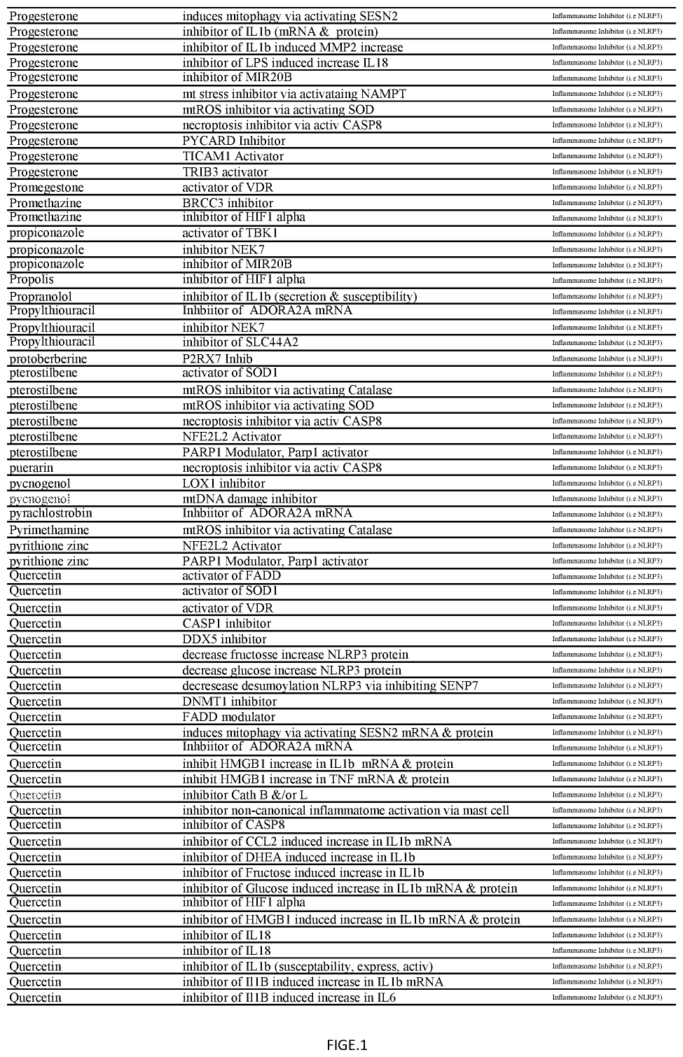

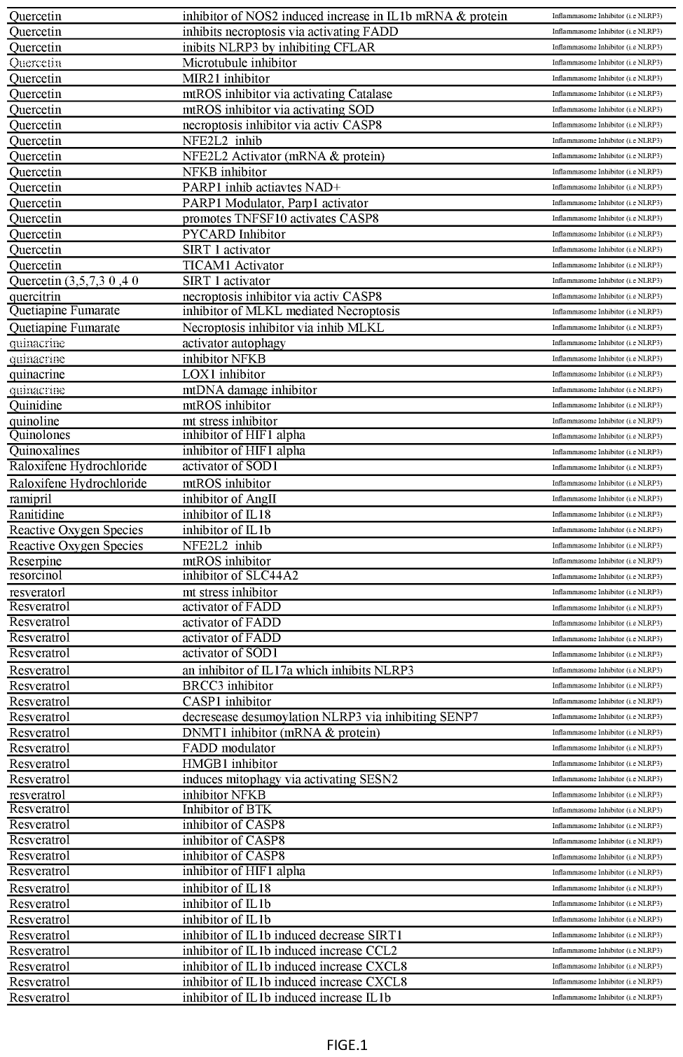

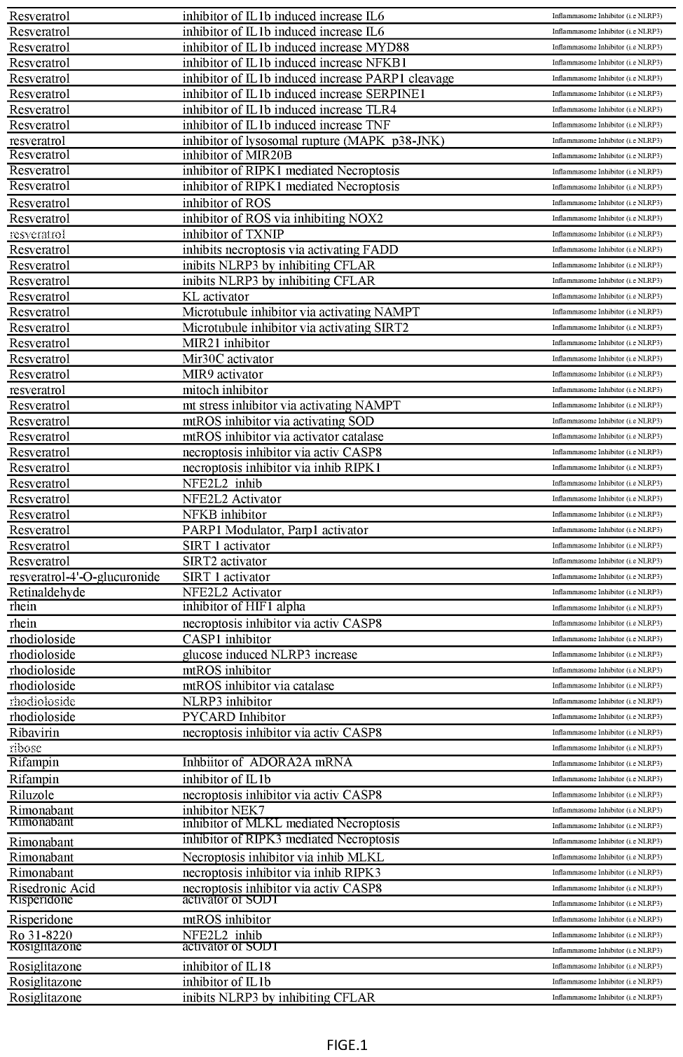

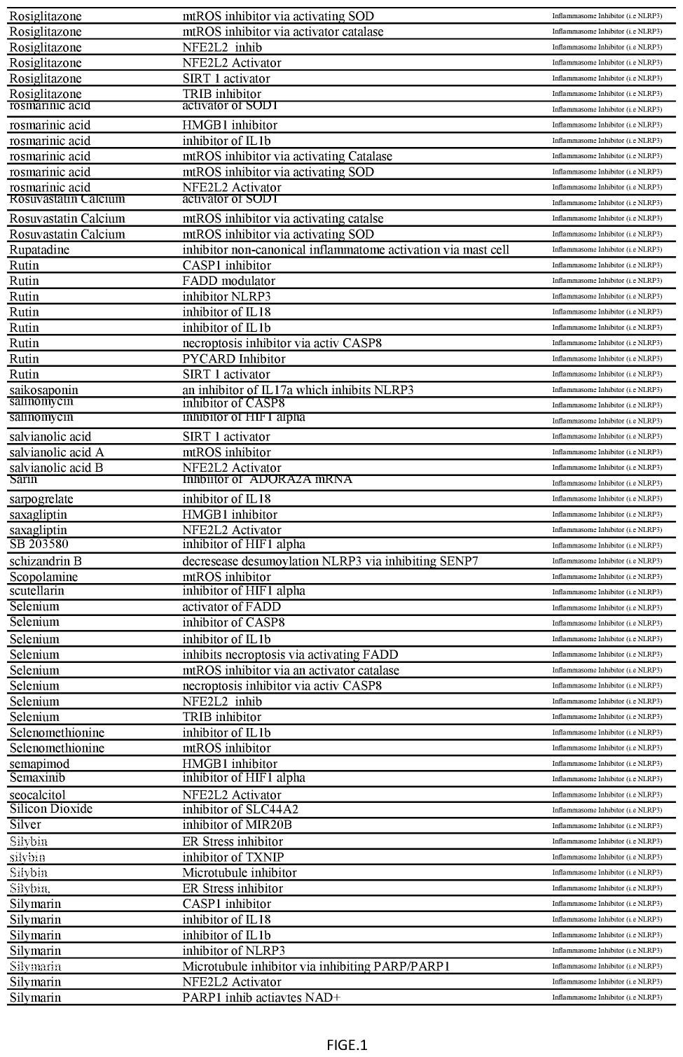

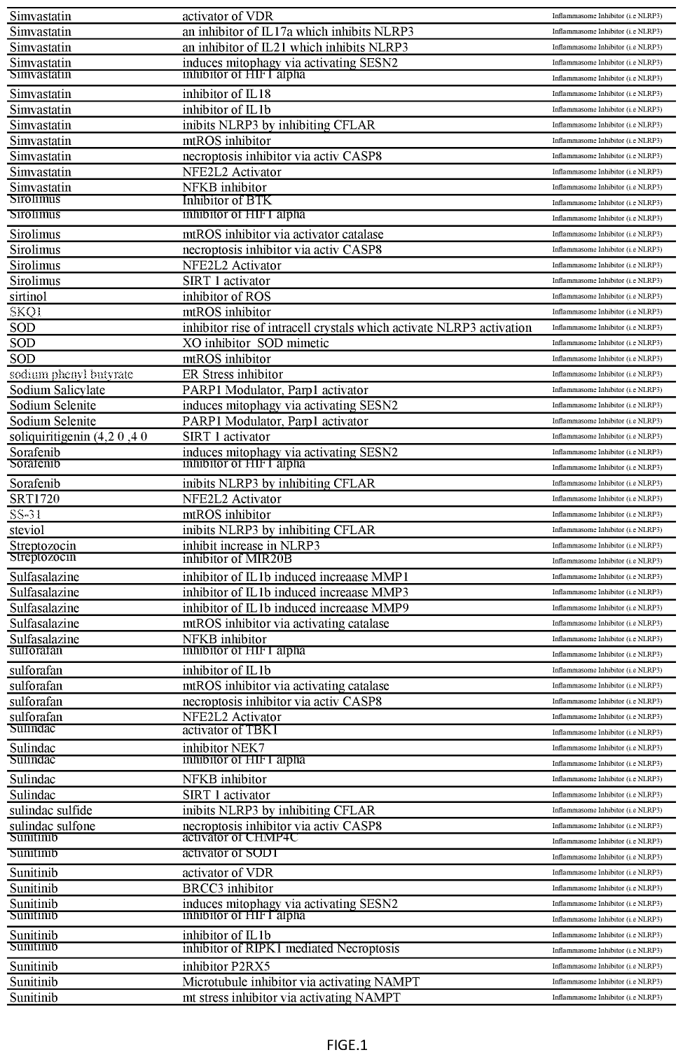

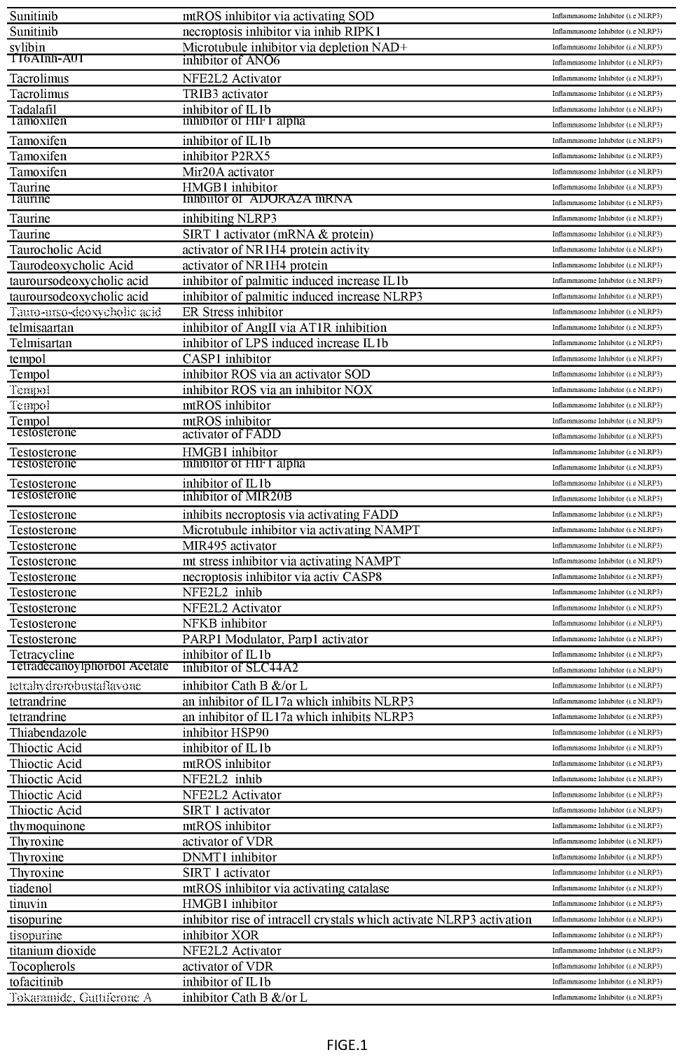

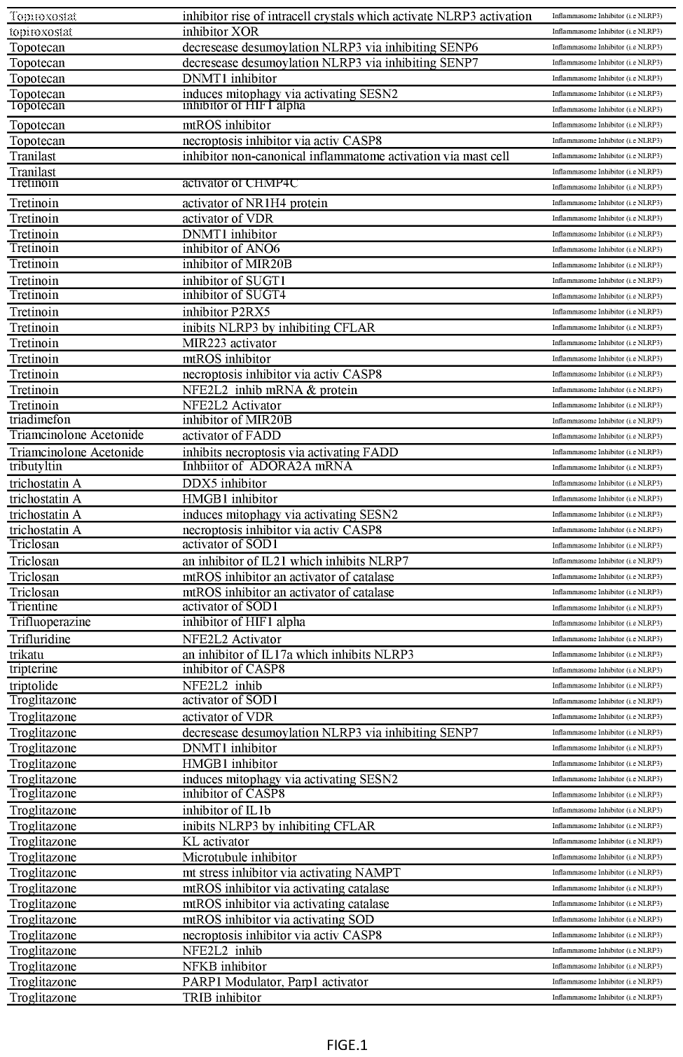

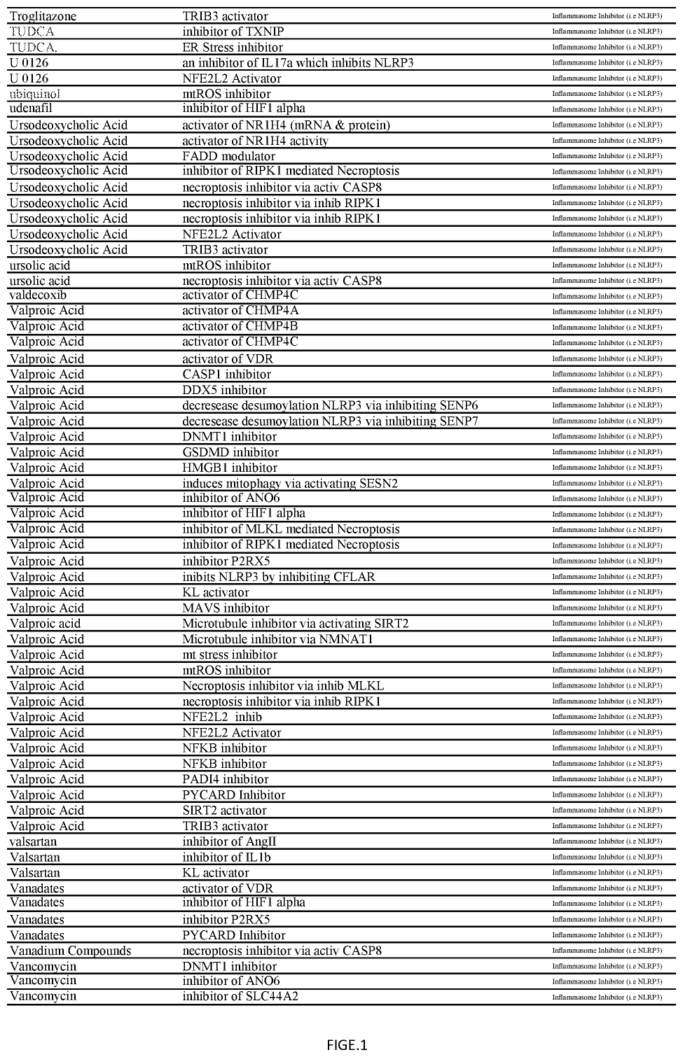

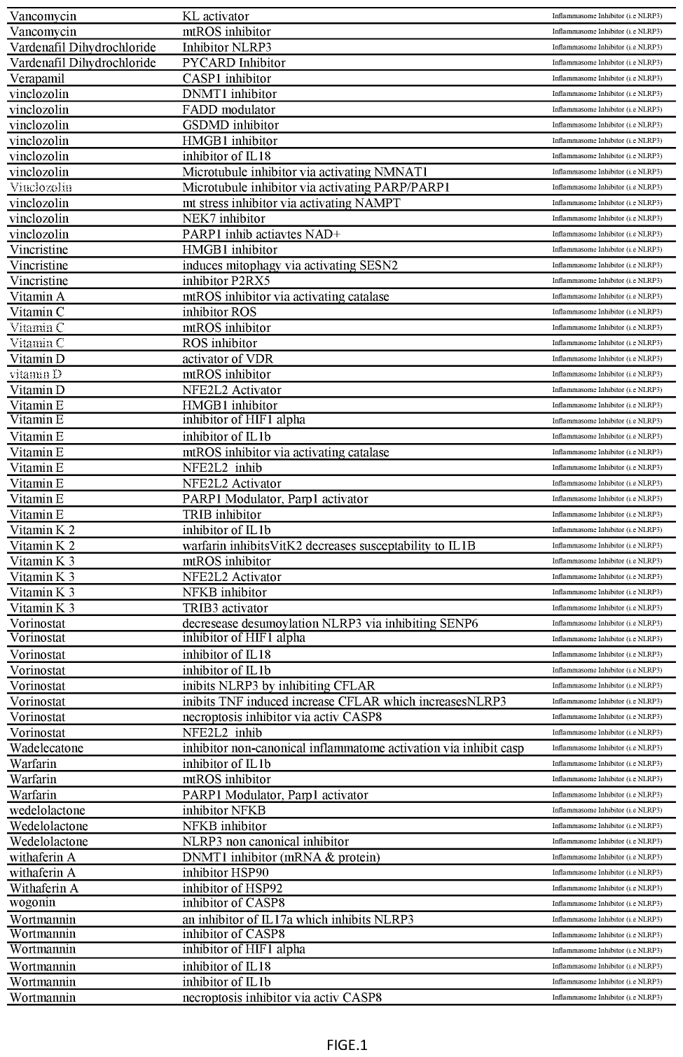

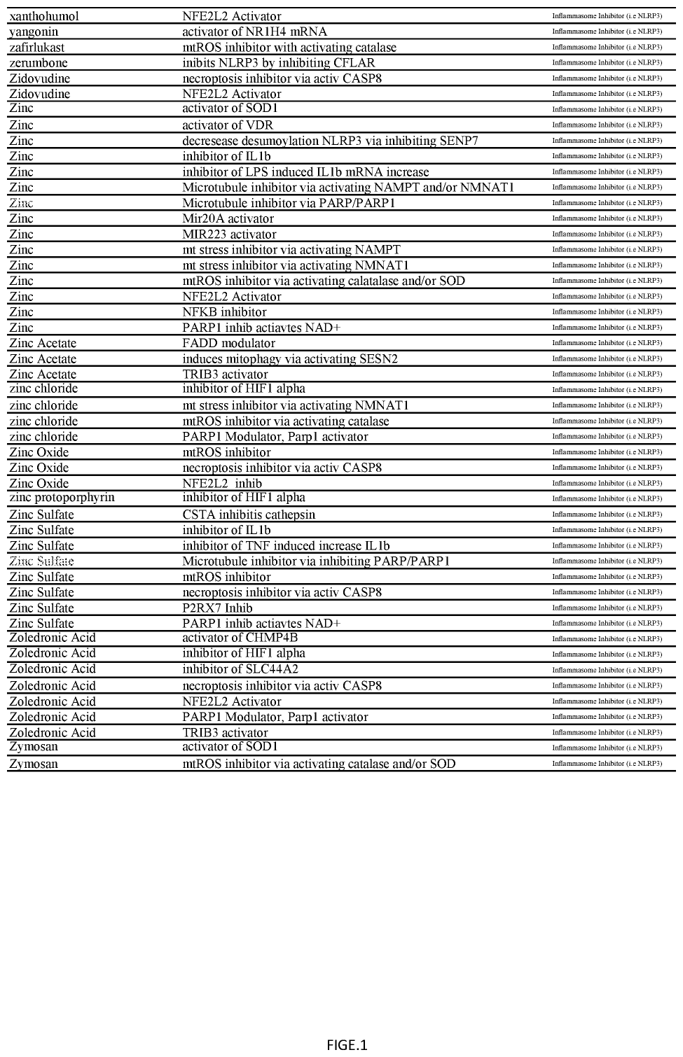

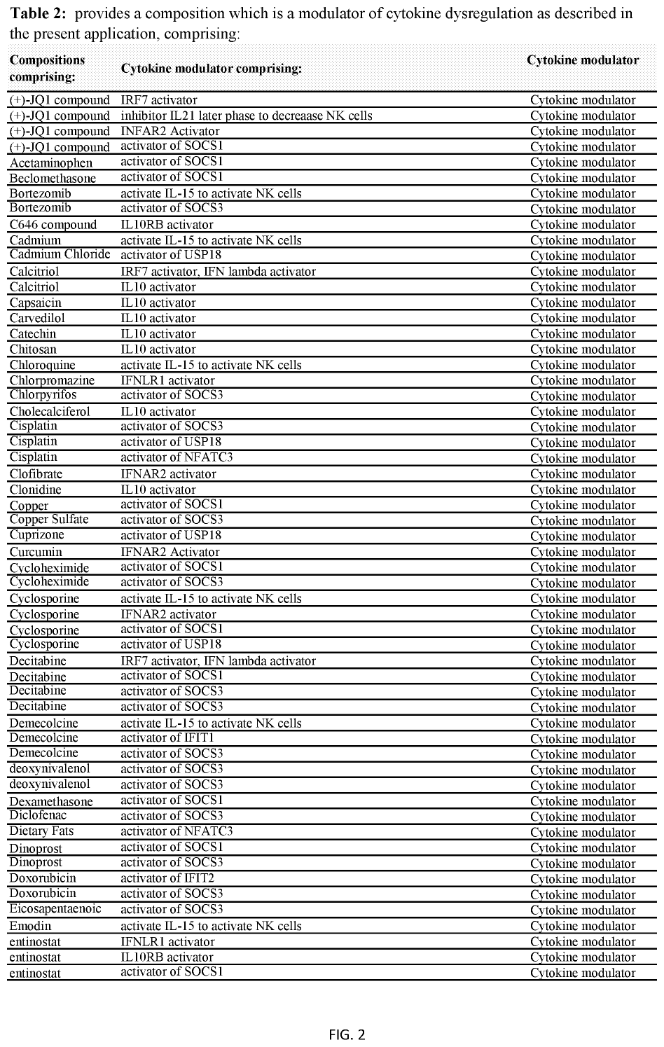

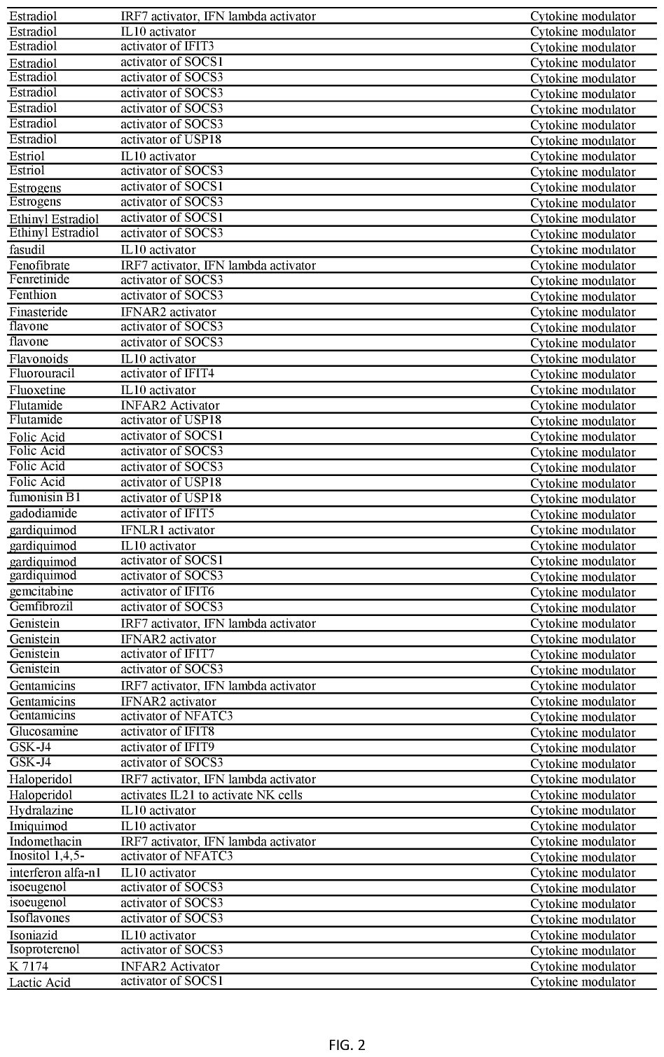

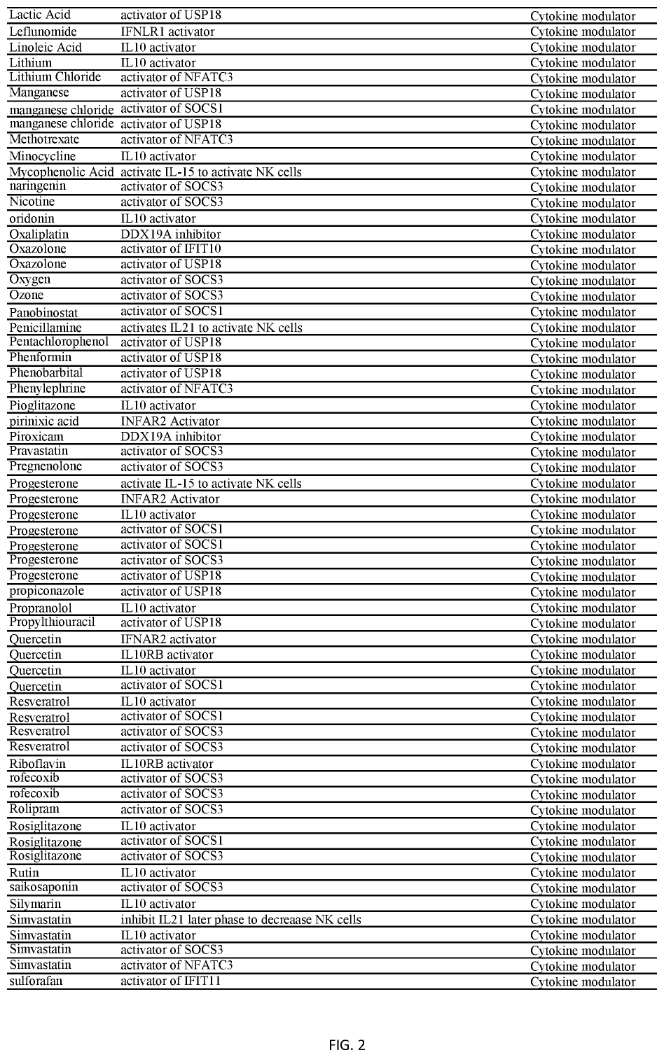

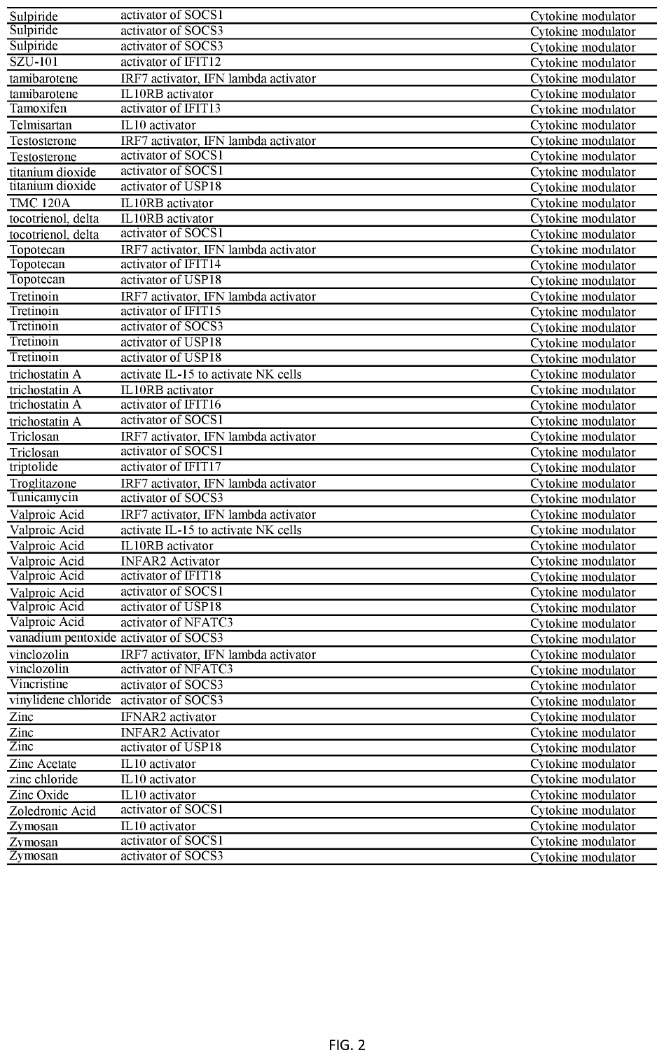

The composition is a modulator of a single or many activators of SARS-CoV-2 associated inflammasome (i.e. NLRP3) priming and/or activation to be administered alone or in combination with another composition to be used in steps where in the composition is a modulator of Sars-CoV-2 associated cytokine dysregulation wherein the said compositions and steps of methods and/or use comprise:
i) a modulator of inflammasome induced cytokine dysregulation comprising but not limited to compositions listed in Table 2 shown in FIG. 2,
ii) an inhibitor of single or multiple activators of NLRP3 inflammasome comprising: methylene blue, colchicine, SPB, TUDCA, allopurinol, resveratrol, NAC, quercetin, Mitotempo, mitoQ, EGCG, alpha lipoic acid, silybin, genistein, daidzein, berberine, ambroxol, curcumin, ginsenoside M1 and zinc sulfate,
iii) An activator/stimulator of Interferon III lambda during early infection comprising:
   a. Genistein Calcitriol, Estradiol, Fenofibrate, Genistein, Gentamicin, Indomethacin, (+)-JQ1 compound, tamibarotene, Testosterone, Topotecan, Triclosan, Troglitazone, Valproic Acid,
   b. Flavonoids comprising: Epigallocatechin gallate, Quercetin, and Liquiritigenin
   c. Flavonoid glycosides comprising: Centaurein, centaureidin, and Epimedin C,
   d. pentacyclic triterpenoid comprising: Glycyrrhizin or glycyrrhizic acid or glycyrrhizinic acid,
   e. Allicin, licorice, astralagus, echinacea, eleuthero, vitamin C
   f. Followed by an inhibitor of Interferon Lambda receptor INFLR1, IFR3, IRF7 and IL10RB,
iv) a pegylated-interferon-lambda,
v) an agonist of Interferon Lambda comprising:
   a. an activator IRF3 gene expression and or activity comprising: copper, selenium, quercetin, gentamicines, maduramicin, vinclozine)
   b. an activator of IRF7 gene expression and or activity comprising: Calcitriol, Decitabine, Decitabine, Estradiol, Fenofibrate, Genistein, Gentamicin, Indomethacin, (+)-JQ1 compound, tamibarotene, Testosterone, Topotecan, Triclosan, Troglitazone, and Valproic Acid,
   c. an activator of Interferon Lambda receptor INFLR1 gene expression and or activity (i.e. genistein, Catechin, Chlorpromazine, Choline, epigallocatechin gallate, Folic Acid, gardiquimod, Grape Seed Proanthocyanidins, Methionine, Phenobarbital)
   d. an activator of Interferon Lambda receptor IL10RB gene expression and or activity comprising: C646 compound entinostat, Quercetin, Riboflavin, tamibarotene, tocotrienol, delta, trichostatin A, and Valproic Acid
vi) a modulator of the SARS-associated host cell antiviral and cytotoxic response wherein the modulator is an activator of IL-15 comprising:
   a. Lactoferrin
   b. an activator of IL-15 gene mRNA expression and or activity comprising of: Bortezomib, Chloroquine, Cyclosporine, Demecolcine, Emodin, Mycophenolic Acid, Oxygen, Progesterone, trichostatin A, and Valproic Acid
   c. an activator of Interferon lambda
   d. an activator of IL-12 gene expression and or activity (i.e. SZU-101)
   e. an activator of IL-21 gene expression and or activity comprising: penicillamine, Low dose naltrexone,
vii) an activator of Interferon I comprising:
   a. an activator of Interferon) beta comprising: Stinging Nettle Lectin, Urtica Dioica agglutinin (UDA))
   b. a pegylated-interferon-1beta.
   c. an Interferon Lambda mimetic
   d. an activator of IRF3 gene expression and or activity comprising: copper, selenium, quercetin, gentamicines, maduramicin, and vinclozine,
   e. an activator of interferon 1 receptor genes (IFNAR1/IFNAR2) expression and or activity comprising: Acetaminophen, Amantadine, Curcumin, Gentamicins, progesterone, Valproic Acid, and vinclozolin)
viii) a modulator of IFNAR1/IFNAR2 comprising:
   a. inhibitor of IFNAR1 gene expression and or activity comprising: clofibrate, Cyclosporine, Finasteride, Genistein, Gentamicins, Quercetin, and Zinc,
   b. an inhibitor of IFNAR2 gene expression and or activity comprising of gardiquimod, (+)-JQ1 compound, schizandrin B, Sunitinib
ix) an activator of IL-10 comprising:
   a. An inhibitor of AT1R (i.e. losartan, valsartan, telmisartan)
   b. An activator of Heat Shock Factor 1 (i.e. quercetin, triploide, cactharidin, fisetin, stresgenein B, rocaglamide, 2,4-Bis (4-hydroxy benzyl) Phenol, CL-43)
   c. Low dose naltrexone
   d. Hypericum triquetrifolium
      where in the compositions and uses of this embodiment can be administered to a human subject as sequential therapies and or the compositions and uses of this embodiment can be administered to a human subject with other drugs and or medication and or supplements. (i.e. a vaccine, Remdesivir and others) as concurrent therapies.

### Example 5

In one example, a composition is administered to a mammal, preferably a human, in need thereof to induce an artificial biological mimicry wherein the immune advantages of a bat's immune system is mimicked and induced in the mammal as a prophylaxis and/or a treatment of SARS-CoV-2 and or COVID-19. Where the mammal is a human subject, the human subject has clinical signs and symptoms including but not limited to thromboembolism, stroke, myocardial infarction, lung injury, acute respiratory distress syndrome (ARDS), anti-phospholipid like syndrome, Kawasaki like syndrome, and/or vasculitis.

The composition is a modulator of a single or many activators of SARS-CoV-2 associated inflammasome (i.e. NLRP3) priming and/or activation to be administered alone or in combination with another composition to be used in steps where in the composition is a modulator of Sars-CoV-2 associated cytokine dysregulation wherein the said compositions and steps of methods and/or use comprise:
i) a modulator of inflammasome induced cytokine dysregulation comprising but not limited to compositions listed in Table 2 shown in FIG. 2,
ii) an inhibitor of single or multiple activators of NLRP3 inflammasome comprising: methylene blue, colchicine, SPB, TUDCA, allopurinol, resveratrol, NAC, quercetin, Mitotempo, mitoQ, EGCG, alpha lipoic acid, silybin, genistein, daidzein, berberine, ambroxol, curcumin, ginsenoside M1 and zinc sulfate,
iii) An activator/stimulator of Interferon III lambda during early infection comprising:
   a. Genistein Calcitriol, Estradiol, Fenofibrate, Genistein, Gentamicin, Indomethacin, (+)-JQ1 compound, tamibarotene, Testosterone, Topotecan, Triclosan, Troglitazone, Valproic Acid,
   b. Flavonoids comprising: Epigallocatechin gallate, Quercetin, and Liquiritigenin
   c. Flavonoid glycosides comprising: Centaurein, centaureidin, and Epimedin C,
   d. pentacyclic triterpenoid comprising: Glycyrrhizin or glycyrrhizic acid or glycyrrhizinic acid,
   e. Allicin, licorice, astralagus, echinacea, eleuthero, vitamin C
   f. Followed by an inhibitor of Interferon Lambda receptor INFLR1, IFR3, IRF7 and IL10RB,
iv) a pegylated-interferon-lambda,
v) an agonist of Interferon Lambda comprising:
   a. an activator IRF3 gene expression and or activity comprising: copper, selenium, quercetin, gentamicines, maduramicin, vinclozine)
   b. an activator of IRF7 gene expression and or activity comprising: Calcitriol, Decitabine, Decitabine, Estradiol, Fenofibrate, Genistein, Gentamicin, Indomethacin, (+)-JQ1 compound, tamibarotene, Testosterone, Topotecan, Triclosan, Troglitazone, and Valproic Acid,
   c. an activator of Interferon Lambda receptor INFLR1 gene expression and or activity (i.e. genistein, Catechin, Chlorpromazine, Choline, epigallocatechin gallate, Folic Acid, gardiquimod, Grape Seed Proanthocyanidins, Methionine, Phenobarbital)
   d. an activator of Interferon Lambda receptor IL10RB gene expression and or activity comprising: C646 compound entinostat, Quercetin, Riboflavin, tamibarotene, tocotrienol, delta, trichostatin A, and Valproic Acid
vi) a modulator of the SARS-associated host cell antiviral and cytotoxic response wherein the modulator is an activator of IL-15 comprising:
   a. Lactoferrin
   b. an activator of IL-15 gene mRNA expression and or activity comprising of: Bortezomib, Chloroquine, Cyclosporine, Demecolcine, Emodin, Mycophenolic Acid, Oxygen, Progesterone, trichostatin A, and Valproic Acid
   c. an activator of Interferon lambda
   d. an activator of IL-12 gene expression and or activity (i.e. SZU-101)
   e. an activator of IL-21 gene expression and or activity comprising: penicillamine, Low dose naltrexone,
vii) an activator of Interferon I comprising:
   a. an activator of Interferon) beta comprising: Stinging Nettle Lectin, Urtica Dioica agglutinin (UDA))
   b. a pegylated-interferon-1beta.
   c. an Interferon Lambda mimetic
   d. an activator of IRF3 gene expression and or activity comprising: copper, selenium, quercetin, gentamicines, maduramicin, and vinclozine,
   e. an activator of interferon 1 receptor genes (IFNAR1/IFNAR2) expression and or activity comprising: Acetaminophen, Amantadine, Curcumin, Gentamicins, progesterone, Valproic Acid, and vinclozolin)
viii) a modulator of IFNAR1/IFNAR2 comprising:
   a. inhibitor of IFNAR1 gene expression and or activity comprising: clofibrate, Cyclosporine, Finasteride, Genistein, Gentamicins, Quercetin, and Zinc,
   b. an inhibitor of IFNAR2 gene expression and or activity comprising of gardiquimod, (+)-JQ1 compound, schizandrin B, Sunitinib
ix) an activator of IL-10 comprising:
   a. An inhibitor of AT1R (i.e. losartan, valsartan, telmisartan)
   b. An activator of Heat Shock Factor 1 (i.e. quercetin, triploide, cactharidin, fisetin, stresgenein B, rocaglamide, 2,4-Bis (4-hydroxy benzyl) Phenol, CL-43)
   c. Low dose naltrexone
   d. Hypericum triquetrifolium
      where in the compositions and uses of this embodiment can be administered to a human subject as sequential therapies and or the compositions and uses of this embodiment can be administered to a human subject with other drugs and or medication and or supplements. (i.e. a vaccine, Remdesivir and others) as concurrent therapies.

### Example 6

In one example, a composition is administered to a mammal, preferably a human, in need thereof to induce an artificial biological mimicry wherein the immune advantages of a bat's immune system is mimicked and induced in the mammal as a prophylaxis and/or a treatment of SARS-CoV-2 and or COVID-19. Where the mammal is a human subject, the human subject has clinical signs and symptoms including but not limited to thromboembolism, stroke, myocardial infarction, lung injury, acute respiratory distress syndrome (ARDS), anti-phospholipid like syndrome, Kawasaki like syndrome, and/or vasculitis.

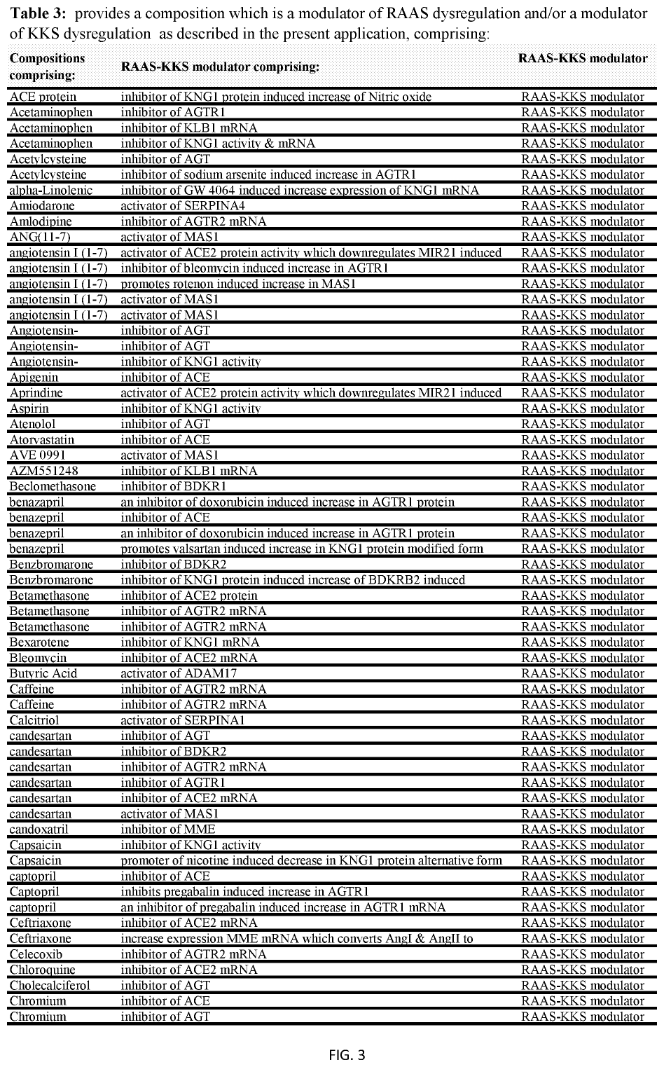

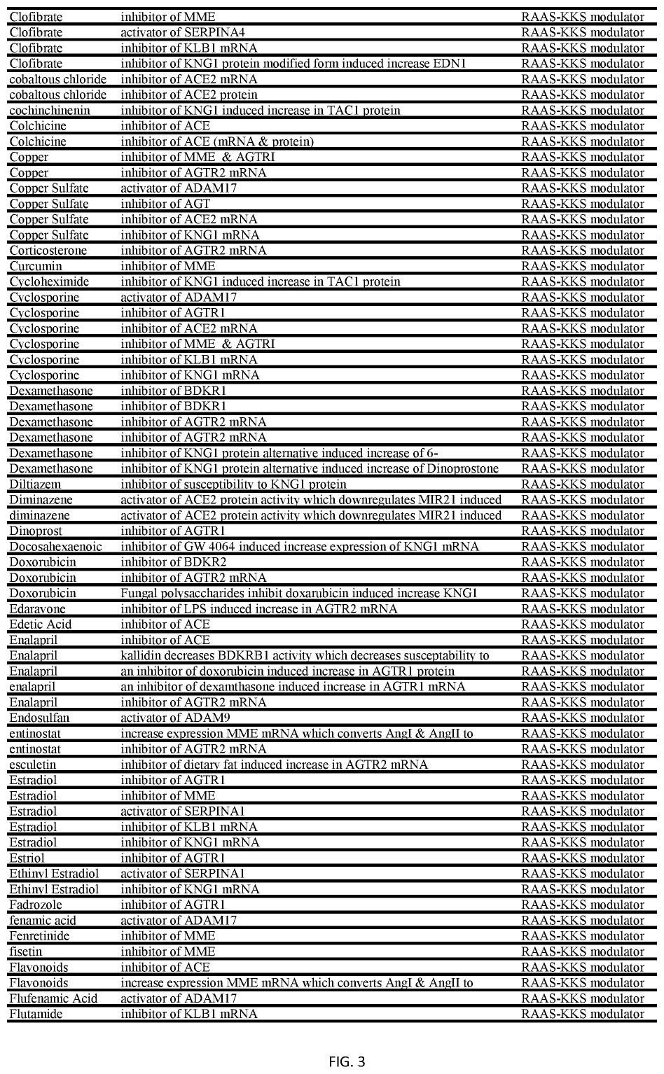

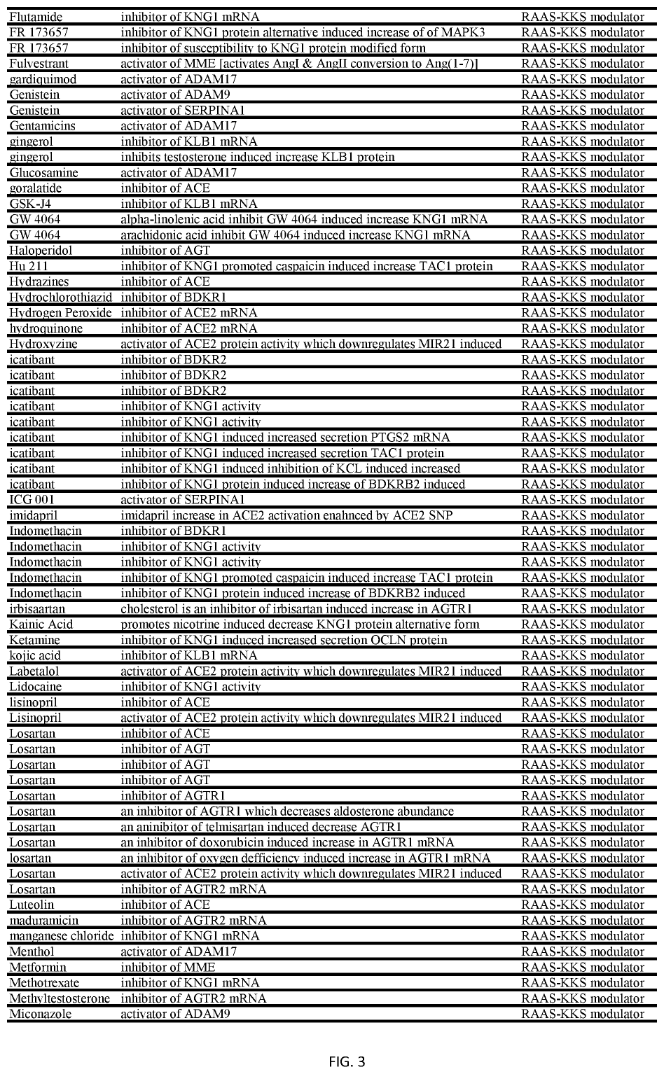

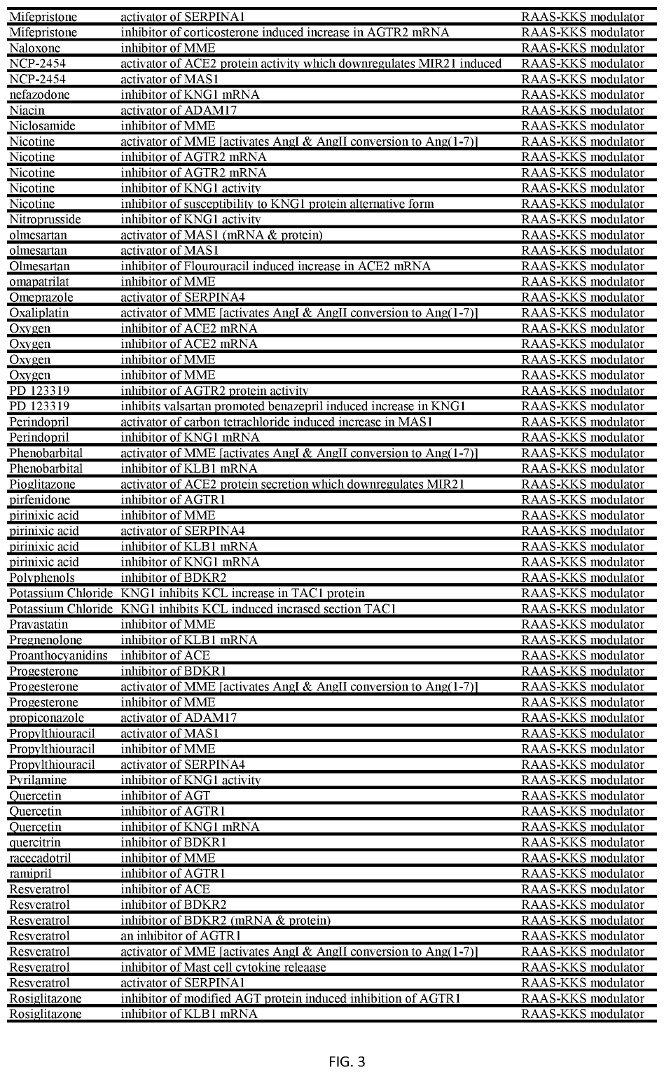

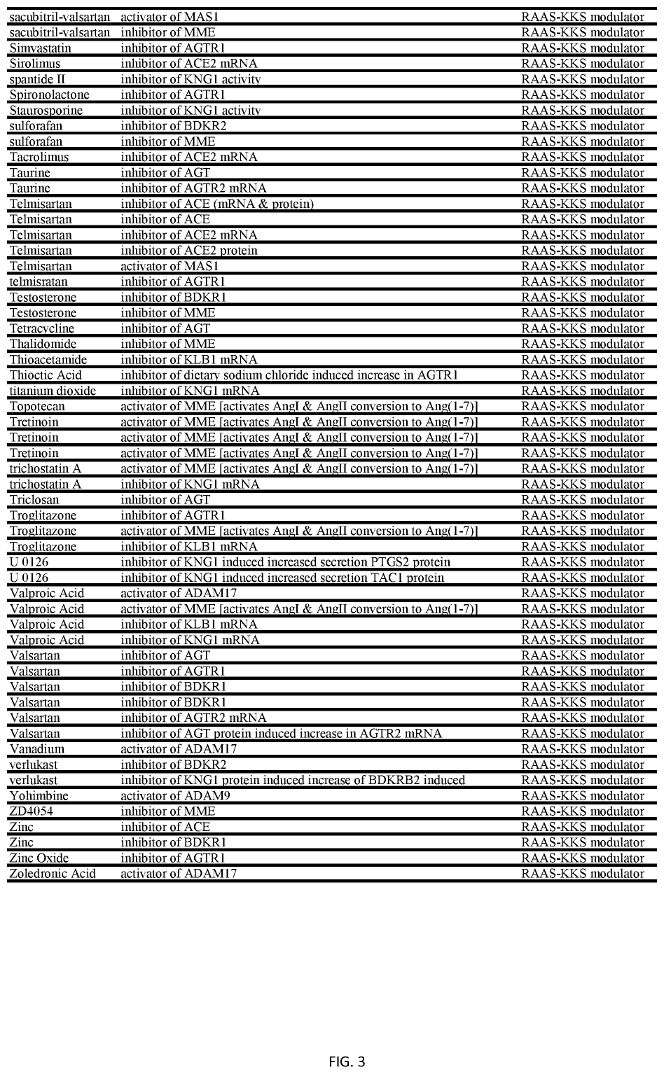

The composition is a modulator of Sars-CoV-2 associated RAAS/KKS dysregulation comprising of RAAS Axis I upregulation (ANGII/ACE), RAAS Axis II downregulation (ACE2/Mas/Ang(1-7) and KKS upregulation wherein the said compositions and methods and or use in steps comprising:
i) a modulator of inflammasome induced RAAS-KKS dysregulation comprising but not limited to compositions listed in Table 3 shown in FIG. 3,
ii) an inhibitor of single or multiple activators of NLRP3 inflammasome comprising: methylene blue, colchicine, SPB, TUDCA, allopurinol, resveratrol, NAC, quercetin, Mitotempo, mitoQ, EGCG, alpha lipoic acid, silybin, genistein, apocynin, apigenin, fisetin, daidzein, berberine, ambroxol, curcumin, ginsenoside M1 and zinc sulfate,
iii) an inhibitor of Angiotensin II comprising of:
   a. an inhibitor of angiotensin converting enzyme comprising of lisinopril, Ramipril, captopril, Eosinophil, enalapril
   b. an inhibitor of AT1R (Angiotensin 1 Receptor blockers) comprising of Losartan, Telmisartan, valsartan and olmisartan,
   c. an inhibitor of ACE gene transcription comprising: Atorvastatin, Flavonoids, goralatide, Hydrazines, Losartan, Resveratrol, Telmisartan, ACEI, and ARBs,
   d. an inhibitor of ACE gene activity: Apigenin, benazepril, Chromium, Colchicine, Edetic Acid Enalapril, Luteolin, Zinc Proanthocyanidins,
   e. an inhibitor of Renin comprising of 3rd generation renin inhibitors (i.e. Aliskiren)
   f. an inhibitor of Chymase comprising of: Soybean Bowman-Birk protease inhibitor. Benzoxazinon)
   g. a mast cell stabilizer (i.e. nedocromil),
   h. an inhibitor of histamine: comprising amlexanox, Benadryl, hydroxyzine, loratadine, cetirizine,
iv) an activator of Ang (1-7) and Mas receptor comprising of:
   a. an activator of ACE2 enzyme activity comprising of
      1. (i.e. D Diminazene aceturate (DIZE; 4-(2-(4-carbamimidoylphenyl) iminohy-drazinyl) benzenecarboximidamide
      2. an inhibitor of ACE
      3. an inhibitor of AT1R (i.e. ARBs comprising) telmisartan, losartan, valsartan
      4. an inhibitor of AGTR1 gene expression and or activity comprising: Acetaminophen, Acetylcysteine, Aldosterone, angiotensin, I (1-7), benazepril, candesartan cilexetil, Captopril, Cyclosporine, Dexamethasone, Dinoprost, Doxorubicin, Enalapril, Estradiol, Estriol, Fadrozole, Irbesartan, Losartan, Oxygen, pirfenidone, Pregabalin, Quercetin, Resveratrol, Rosiglitazone, Simvastatin, Spironolactone, Telmisartan, Thioctic Acid, Troglitazone, Zinc Oxide
      5. a modulator of neprolysin comprising an activator of MME comprising: Ceftriaxone, entinostat, Flavonoids, Fulvestrant, Nicotine, Oxaliplatin, Phenobarbital, Progesterone, Resveratrol, Topotecan, Tretinoin, Tretinoin, Tretinoin, trichostatin A, Troglitazone, and Valproic Acid.
      6. an inhibitor of MME gene expression comprising: Estradiol, Fenretinide, fisetin, Metformin, Niclosamide, omapatrilat, Progesterone, Propylthiouracil, Testosterone, Thalidomide, ZD4054
      7. a combined At1R-Nep inhibitor (ARNI) (i.e. Sacubitril/valsartan)
   b. an Activator of ADAM17 gene activity and or transcription comprising: Copper Sulfate, Cyclosporine, Glucosamine, Niacin, Valproic Acid, and Vanadium, Zoledronic Acid
   c. an activator of MAS receptor comprising of:
      1. Mas receptor agonist
      2. Ang (1-7) mimetics (i.e. AVE 0991, Angiotensin-(1-5)
      3. An activator of Mas gene transcription or activity (ARBs (i.e. Olmesartan, telmisartan), Propylthiouracil
   d. An activator of Ang (1-7)
      i. an activator of ACE2 comprising: angiotensin I (1-7), Aprindine, Diminazene, diminazene aceturate, Hydroxyzine, Labetalol, Lisinopril, and Losartan
      ii. An agonist or mimetic Ang (1-7) (i.e. AVE 0991, ang (1-7))
   e. an AT2R agonist (i.e. Compound 21 (N-butyloxycarbonyl-3-(4-imidazol-1-ylmethylphenyl)-5-isobutylthiophene-2-sulphonamide)
v) a modulator of KKS comprising:
   a. a modulator of Neprilysin (i.e. Thiorphan, Phosphoramidon, Candoxatrilat,
   b. an inhibitor of Atlr and Neprilysin (i.e. Valsartan & candoxatril)
   c. an inhibitor of ACE and Neprilysin comprising: Ecadotril and Lisinopril
   d. an inhibitor of Bradykinin B2 receptor comprising: benzbromarone, candesartan, doxorubicin, icatibant, resveratrol, sulforafan, and verlukast
   e. An inhibitor of Bradykinin B1 receptor comprising: beclomethasone, dexamethasone, hydrochlorothiazide, indomethacin, progesterone, quercetin, testosterone and zinc,
   f. an inhibitor histamine comprising: Benadryl, hydroxyzine
   g. a mimetic of Kallistatin
   h. an activator of SERPIN4a comprising: Amiodarone, Benzbromarone, Clofibrate, Omeprazole, pirinixic acid, Propylthiouracil
   i. an activator of SERPINA1 comprising: Calcitriol, Estradiol, Ethinyl Estradiol, Genistein, ICG 001, Mifepristone, Resveratrol,
   j. a modulator of Cathepsin A (Cystatin A)
   k. an inhibitor of KNG1, HMWK and or LMWK comprising: acetaminophen, alphalinolenic acid, aspirin, nicotine, ketamine, lidocaine, nitroprusside, perindopril, PD 123319, quercetin, spantide II, trichostatin A, verlukast
   l. an inhibitor of KLKB1 comprising: clofibrate, cyclosporin, estradiol, gingerol, GSK-J4, kojic acid, pregnenolone, rosiglitazone, thioacetamide, troglitazone, acetaminophen, valproic acid, phenobarbital
   m. an inhibitor of gene expression, activity and or posttranslational modification of genes comprising: KLKB1, KNG1, BDKRB1, BDKRB2, ACE
   n. an activator of genes comprising: SERPINA4, CTSA, SERPINA1
      where in the compositions and uses of this embodiment can be administered to a human subject as sequential therapies and or the compositions and uses of this embodiment can be administered to a human subject with other drugs and or medication and or supplements. (i.e. a vaccine, Remdesivir, anticoagulants comprising: heparin, warfarin, and apixaban) as concurrent therapies.

### Example 7

In one example, a composition is administered to a mammal, preferably a human, in need thereof to induce an artificial biological mimicry wherein the immune advantages of a bat's immune system is mimicked and induced in the mammal as a prophylaxis and/or a treatment of SARS-CoV-2 and or COVID-19. Where the mammal is a human subject, the human subject has clinical signs and symptoms including but not limited to thromboembolism, stroke, myocardial infarction, lung injury, acute respiratory distress syndrome (ARDS), anti-phospholipid like syndrome, Kawasaki like syndrome, and/or vasculitis.

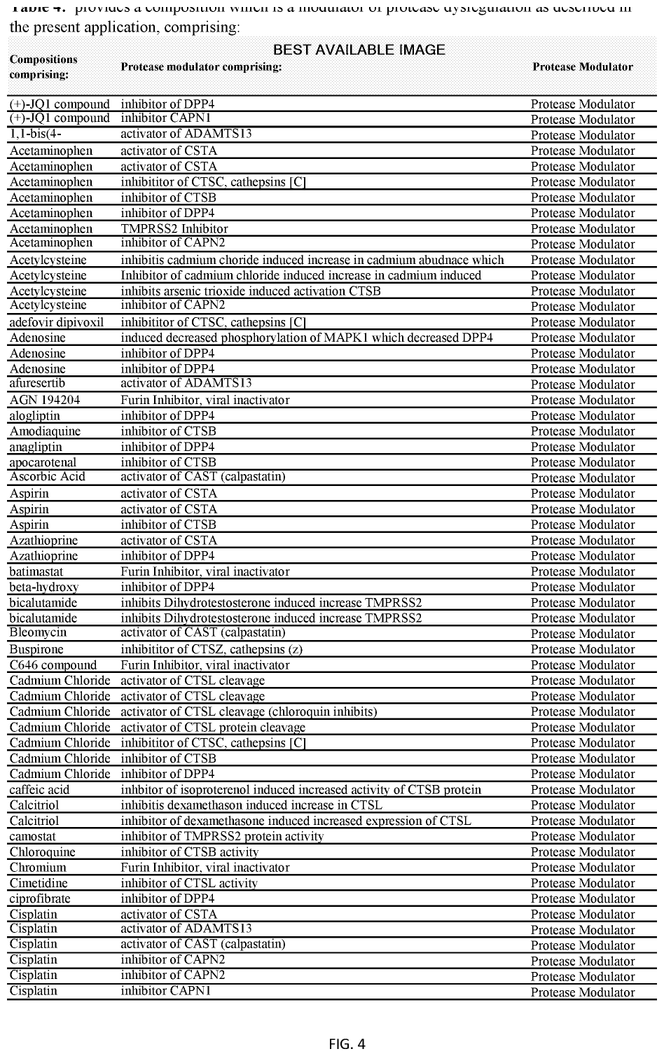

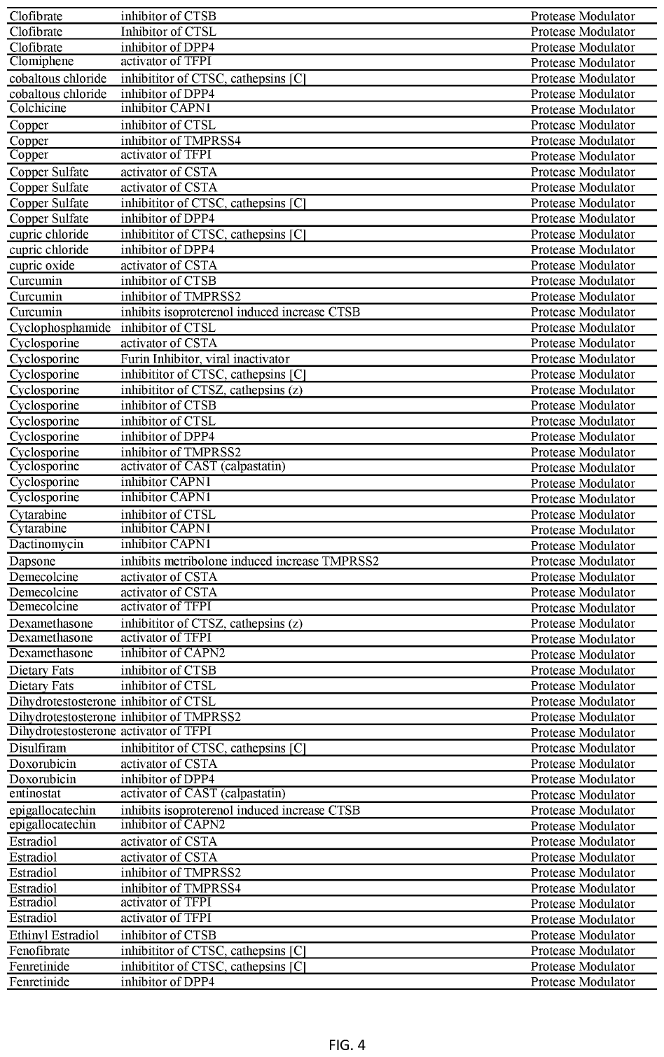

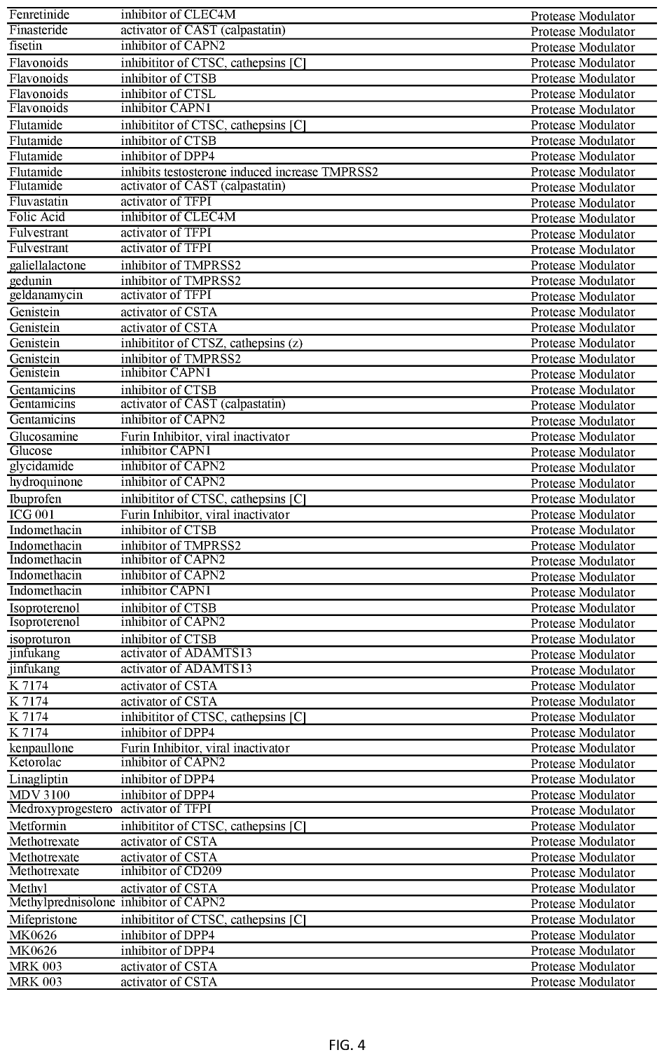

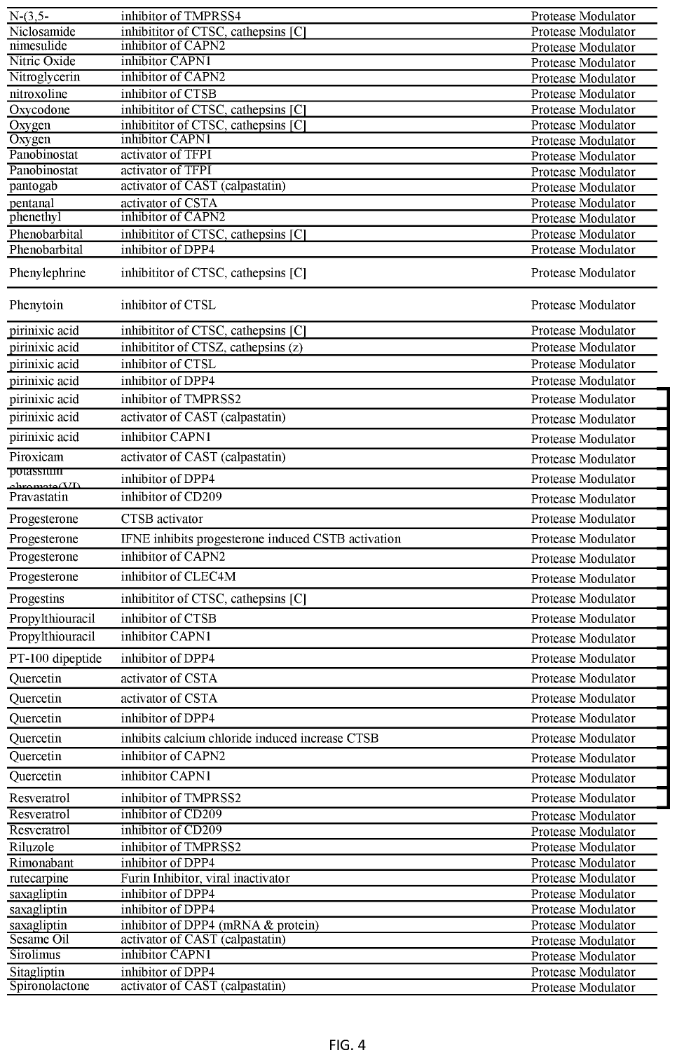

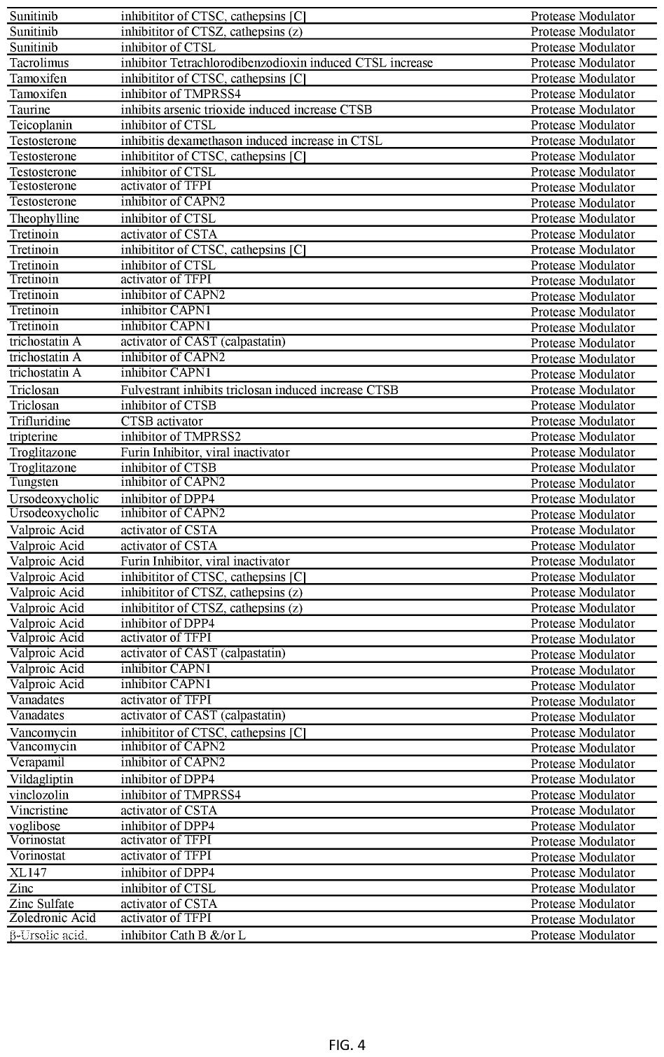

The composition is a modulator of Sars-CoV-2 associated protease dysregulation and or SARS-CoV-2 associated RAAS/KKS dysregulation wherein the said compositions and methods and or use in steps comprising:
i) a modulator of protease dysregulation comprising but not limited to compositions listed in Table 4 shown in FIG. 4
ii) an inhibitor of TMPRSS2 comprising: galiellalactone, celastrol, resveratrol, gedunin, curcumin, genistein, glyzorrhizic acid
iii) an inhibitor of Cathepsin B comprising: Guttiferone A, β-Ursolic acid, Flavonoid F5, E-64, Miraziridine A, and Tokaramide, quercetin, bioflavanoid
iv) an inhibitor of CTSB gene expression and or activity and or post translational modification comprising: Acetylcysteine, Aspirin, Chloroquine, Curcumin, Cyclosporine, epigallocatechin gallate, ethinyl Estradiol, and Flavonoids
v) an inhibitor of Cathepsin L comprising: Schinol, β-Ursolic acid Chloroquine, Hydroxychloroquine, Z-FY(t-Bu)-DMK, teicoplanin, SID 26681509, N-Acetyl-L-leucyl-L-leucyl-L-methional, quercetin, SSAA09E1, E 64d, Bafilomycin A1, Flavonoid F5, Nicolaioidesin C, E-64, aretemisia annua, 3-epiursolic acid (3), 3-(hydroxyimino) oleanolic acid), and 3-(hydroxyimino) masticadienoic acid)
vi) an inhibitor of CTSL gene expression and or activity comprising: calcitriol, copper, cyclosporine, Copper, tacrolimus, zinc
vii) an activator of cystatins (i.e cystatin A) comprising: Acetaminophen, Aspirin, Azathioprine, Cisplatin, Copper Sulfate, cupric oxide, Cyclosporine, Demecolcine, Doxorubicin, Estradiol, Genistein, K 7174, Methotrexate, MRK 003, pentanal, Quercetin, Tretinoin, Valproic Acid, Vincristine, and Zinc Sulfate,
viii) an inhibitor of Furin comprising: Calcium, Pirfenidone, capric acid, and phenylacetyl-Arg-Val-Arg-4amidinobenzylamide, oroxylum indicum (comprising: flavonoids, baicalein, chrysin, oroxylin-A)
ix) an inhibitor of cathepsin S & Z
x) an inhibitor of Furin gene expression comprising: AGN 194204, C646 compound, Chromium, Cyclosporine, ICG 001, kenpaullone, rutecarpine, Troglitazone, and Valproic Acid
xi) an inhibitor of Furin activity (i.e. glucosamine, batimastat)
xii) an inhibitor of DPP4 (i.e., Clofibrate, Copper Sulfate, pirinixic acid, Quercetin, Sitagliptin Phosphate, Ursodeoxycholic Acid, voglibose)
xiii) an activator of ACE2 gene activity comprising: angiotensin I (1-7), Aprindine, Diminazene, diminazene aceturate, DIZE, Hydroxyzine, Labetalol, Lisinopril, and Losartan
xiv) an ACE inhibitor
xv) an activator of ADAM17 comprising: Copper Sulfate, Cyclosporine, Glucosamine, Niacin, Valproic Acid, Vanadium, Zoledronic Acid
xvi) a mimetic of Kallistatin
xvii) an activator of SERPIN4a comprising: Amiodarone, Benzbromarone, Clofibrate, Omeprazole, pirinixic acid, Propylthiouracil
xviii) an activator of SERPIN1a comprising: Calcitriol, Estradiol, Ethinyl Estradiol, Genistein, ICG 001, Mifepristone, Resveratrol

### Example 8

Further embodiments are provided as examples and are not to limit the scope of the invention as there are many combinations and permutations of the invention.
1. Cardiovascular disease: as used in the invention the term “cardiovascular disease” refer to or describe the physiological condition in mammals that is typically characterized by endothelial dysfunction, atherosclerosis, vascular inflammation and injury, cardiac systolic or diastolic, inflammation of heart and vasculature. Examples of cardiovascular disease comprise of (but are not limited to) cerebrovascular accident, transient ischemic attack, stroke, vascular dementia, peripheral arterial disease (PAD), venous disease, myocardial infarction, dilated cardiomyopathy, hypertrophic cardiomyopathy, congestive heart failure, pericarditis, pericardial effusion, myocarditis, dyslipidemia, hypercholesterolemia, hypertension, abdominal aortic aneurysm, peripheral vascular disease, vasculitis (comprising of good pastures, Kawasaki, giant cell arteritis and others). NLRP3 inflammasome activation, dysregulated KKS (for example but not limited to KB1 receptor activation), dysregulated RAAS (for example but not limited to increased Ang-II, lower ACE2 activation; Cathepsins (Cath) (for example but not limited to cathepsin B upregulation (Cathepsin B breaks down Prorenin, Cathepsin K down regulation cathepsin S upregulation) dysregulated cytokines (i.e., for example but not limited CXCL10 (and its receptor CXCR3), CXCL5 and CCL2, fractalkine) play a role in cardiovascular disease.
   In one embodiment of the invention a composition and its use as disclosed in aspect 1 and aspect 2 of the invention is used as a treatment administered to a human subject for a cardiovascular disease where in the composition is a modulator comprising: an inhibitor of multiple activators of inflammasome (i.e. NLRP3, AIM2, NLRC4, NLRP12) priming and or activation given alone and or with a modulator of an inflammasome-induced dysregulation in a downstream pathway comprising:
   i) a modulator of RAAS-KKS dysregulation comprising:
      a. an inhibitor of ACE
      b. an inhibiter of chymase
      c. an activator of ACE2
      d. an activator of Mas1
      e. an inhibitor of KB1 receptor, an inhibitor KBRB1 gene expression and or activity,
      f. an activator of Kallistatin
      g. an activator of SerpinA4
   ii) a modulator of proteases dysregulation (i.e. cathepsin B, K, S)
   iii) a modulator of dysregulated cytokines comprising: (an inhibitor of CCL2/CCR2, CXCL10/CXCR3, CXCL5/CXCR2, fractalkine).

In the treatment of thromboembolism, particularly that of SARS-COV2 micro thrombosis, SARS-COV2 can induce procoagulant microparticle formation. It is hypothesized that sustained NLRP3 inflammasome activation can induce procoagulant microparticles in varied cell lines that are directly infected by SARS-COV2.

Accordingly, the following combination leads to an increased thrombosis particular in micro vessels: 1) SARS-COV2 induced hyperactivation of NLRP3 induced pyroptosis, and/or necroptosis can lead to tissue damage and generation of clotting cascade; 2) NLRP3 inflammasome mediated CASP1 activation can lead to generation of procoagulant microparticles with Tissue Factor embedded in its plasma membrane which is released from many cells lines (endothelial cells, alveolar epithelial cells, platelets, macrophages, neturophils) causing thrombosis particularly in alveolar micro vessels and fibrin deposition in alveoli; 3) SARSCOV 2 induced NLRP3 activation in platelets activates platelet inducing thrombosis; 4) SARS-COV2 induced NLRP3 inflammasome hyperactivation induces GSDMD mediated NETosis in neturophils which also contributes to platelet aggregation and thrombosis, 5) SARS-COV2 induced ACE2 downregulation induces angiotensin II upregulation which is prothrombotic. The combination.

In one aspect, compositions and methods are provided comprising an inhibitor of SARS-COV2 induced progracoagulant TF bearing microparticle formation from varied cell lines (endothelial cells, alveolar epithelial cells, platelets, macrophages, neturophils) via inhibiting NLRP3 inflammasome hyperactivation, inhibiting its neutrophils adaptor and effector (CASP1 & GSDMD) and pyroptosis comprising:
a. Inhibiting P2RX7 induced NLRP3 activation
b. Inhibiting ADOR2A
c. Inhibit TXNIP
d. Inhibit mtROS
e. Inhibitor NEK7
f. Inhibitor NEK9
g. Inhibitor HSP90
h. Inhibitor of CASP1
i. Inhibit CASP1 mediated Calpastatin Cleavage
Activate CAST (calpastatin) Gene activity and/or expression
k. Activate calpastatin
l. Inhibitor of CASP8
m. Inhibitor of CASP9
n. Inhibit GSDMD mediated pyroptosis
o. Inhibit GSDMD mediated NETosis in neutrophils
p. Inhibit GSDMD mediated pyroptosis in Platelets
q. Inhibit GSDMD mediated pyroptosis in varied cell lines
r. Inhibit TF gene activity and/or expression
s. Activate TFP1 gene activity and/or expression
t. Inhibit BID
u. Inhibit CASP 9
v. Inhibit RIPK1-RIPK3 dependent Necroptosis and/or apoptosis
   i. Inhibit RIPK1, RIPK2, MLKL
   ii. Inhibitor FADD
   iii. Inhibit CASP8
   iv. Activate BIRC2
   v. Activate BIRC3
   vi. Inhibit CYCLd
   vii. Inhibit TAK1
   viii. Activator of CFLAR gene or CFLIP
w. Inhibitor of RAAS ACE/angiotensin II
x. Activator of Ang (1-7)
y. Activator of ANG (1-7)'s receptor MAS1
z. Modulators of all gene's expression and/or activity for all proteins noted above
2. Pulmonary disease: as used in the invention the term “pulmonary disease” refer to or describe the physiological condition in mammals that is typically characterized by injury and inflammation in the airways, alveolus, lung parenchyma, pleura etc. Examples of pulmonary disease comprising acute lung injury, acute respiratory distress syndrome (ARDS), neonatal ARDS, chronic obstructive pulmonary disease, emphysema, anti-alpha trypsin deficiency, asthma, bronchitis, pneumonia, interstitial lung disease, infectious lung disease, granulomatous lung disease (comprising: Wegener's, sarcoid, mycobacterial Tuberculosis), autoimmune lung disease (comprising: SLE, good pastures), mesothelioma, pneumoconiosis, asbestosis, bronchiolitis obliterans organizing pneumonia (BOOP), ventilator associated lung damage, hyperoxia lung damage, pleural effusion, pleuritis, fibrotic lung disease, high altitude pulmonary edema, Idiopathic interstitial pneumonias (examples comprising: idiopathic pulmonary fibrosis, idiopathic interstitial pneumonia: unspecified, desquamative interstitial pneumonia (DIP), respiratory bronchiolitis interstitial lung disease (RBILD), acute interstitial pneumonia (AIP), nonspecific interstitial pneumonia (NSIP), cryptogenic organizing pneumonia (COP (idiopathic BOOP)), lymphoid interstitial pneumonia (LIP), idiopathic interstitial pneumonia: unspecified, Hyper-eosinophilic lung diseases (examples comprising: chronic idiopathic eosinophilic pneumonia, acute idiopathic eosinophilic pneumonia, idiopathic hyper-eosinophilic syndrome with pulmonary manifestations, hyper-eosinophilic lung disease), Allergic bronchopulmonary aspergillosis (ABPA), Pulmonary vasculitis (examples comprising: Wegener's granulomatosis, microscopic polyangiitis, Churg-Strauss syndrome, pulmonary vasculitis: unspecified), Alveolar hemorrhage syndromes (examples comprising: Goodpasture syndrome, idiopathic pulmonary hemosiderosis, alveolar hemorrhage syndrome of undetermined origin, alveolar hemorrhage syndrome of determined origin), Pulmonary lymphangioleiomyomatosis (LAM) (examples comprising: Sporadic pulmonary lymphangioleiomyomatosis (S-LAM), Pulmonary lymphangioleiomyomatosis in tuberous sclerosis (TSC-LAM)), Alveolar proteinosis, Pulmonary amyloidosis, Pulmonary Langerhans cell histiocytosis (histiocytosis x), Primary pulmonary lymphoma, Primary ciliary dyskinesia (without or with situs inversus), Rare cause of hypersensitivity pneumonitis (all causes other than farmer's lung disease and pigeon breeder's lung disease), Pulmonary arteriovenous malformations in hereditary hemorrhagic telangiectasia (HHT), Pulmonary manifestations of gastro-intestinal disorders (examples comprising: pulmonary manifestations in inflammatory bowel diseases, severe hepatopulmonary syndrome (pa02<55 mmHg)), Interstitial lung disease in connective tissue diseases (examples comprising: interstitial lung disease in systemic sclerosis, interstitial lung disease in rheumatoid arthritis, interstitial lung disease in idiopathic inflammatory myopathies, polymyositis, dermatomyositis, anti-synthetase syndrome, interstitial lung disease in Sjogren syndrome, interstitial lung disease in mixed connective tissue disease (MCTD), interstitial lung disease in overlap syndromes, interstitial lung disease in undifferentiated connective tissue disease), Bronchiolitis obliterans (in non-transplanted patients), Sarcoidosis, Chronic beryllium disease, Waterproofing spray pneumonitis, Vaping related pneumonitis, Combined pulmonary fibrosis and emphysema (examples comprising combined pulmonary fibrosis and emphysema without associated connective tissue disease, combined pulmonary fibrosis and emphysema with connective tissue disease), and Alpha-1-antitrypsin deficiency emphysema. NLRP3 inflammasome activation, dysregulated RAAS-KKS (i.e., ACE activation, ACE2 inhibition, Chymase activation), Cathepsin (i.e., Cathepsin G activation), cytokine dysregulation plays a role in pulmonary disease. Polymorphisms in cytokines can impact susceptibility to a certain disease for example CCL2 genetic polymorphisms can impact susceptibility to allergic asthma, pulmonary fibrosis, bronchiolitis obliterans syndrome.

In one embodiment of the invention a composition and its use as disclosed in aspect 1 and aspect 2 of the invention is used as a treatment administered to a human subject for a pulmonary disease where in the composition is a modulator comprising:
i) an inhibitor of multiple activators of inflammasome (i.e. NLRP3) priming and or activation given or with a modulator of an inflammasome-induced dysregulation in a downstream pathway comprising:
   i) a modulator of RAAS-KKS dysregulation comprising:
      a. an inhibitor ACE/Ang-II upregulation comprising:
         i. an inhibitor of ACE
         ii. an inhibitor of Ang-II (i.e., AT1R inhibitor)
         iii. an inhibitor of chymase
      b. an activator (ACE2/Mas/Ang(1-7
         i. an agonist of ACE2,
      c. an inhibitor of KB1 receptor
      d. an inhibitor of KBRB1, KBRB2 gene expression and or gene activity
   ii) a modulator of proteases dysregulation (i.e. an inhibitor of cathepsin G)
   iii) a modulator of dysregulated cytokines comprising:
      a. an inhibitor of CCL2/CCR2, IL-18, IL-17a, 11-2, IL-lb, CXCL10/CXCR3, CXCL5/CXCR2, fractalkine/CXC3L1)
         and the compositions and uses as disclosed in this embodiment of the invention can be administered as concurrent therapies of thrombolytics and or anticoagulants and anti-lipid medications as determined by the patient's health care provider.
3. Thromboembolic disease: as used in the invention the term “thromboembolism” or “thromboembolic disease” refer to or describe the physiological condition in mammals that is typically characterized by endothelial injury, activation of the immune system, kallikrein-kinin system, clotting system, platelets. Examples of thromboembolic disease comprise of (but are not limited to) to arterial or venous thromboembolism, small or large vessel thromboembolism, sepsis associated thromboembolism, stroke, myocardial infarction, mesenteric embolism, antiphospholipid syndrome, vasculitis, Kawasaki disease, deep venous thrombosis, and pulmonary embolism. NLRP3 inflammasome activation, dysregulated RAAS-KK, cytokine dysregulation, protease dysregulation plays a role in thromboembolic disease. For example, neutrophil extracellular traps (NETs) is associated with an atheroma which is rich in cathepsin G. Cathepsin G cleaves IL-1 alpha to its active form.

In one embodiment of the invention a composition as disclosed in aspect 1 of the invention is used as a treatment administered to a human subject for thromboembolic disease (i.e. stroke, a myocardial infarction, a pulmonary embolism and the likes), where in the composition is a modulator comprising:
ii) an inhibitor of multiple activators of inflammasome (i.e. NLRP3) priming and or activation given alone (i.e. MB, S-PB, Allopurinol, Mitotempo, NAC, Resveratrol, NMN, Quercetin, Zinc, apigenin, apocynin, ECGC, melatonin, tempol, metformin) and or with
iii) a modulator of an inflammasome-induced dysregulation in a downstream pathway comprising:
   a. a modulator of RAAS-KKS dysregulation comprising:
      i. an inhibitor AngII upregulation comprising:
         1. an inhibitor of ACE and or chymase, and
         2. an inhibitor of AT1R,
      ii. an activator (ACE2/Mas/Ang (1-7 down regulation comprising:
         1. an agonist of ACE2,
         2. an agonist of Mas
      iii. an inhibitor of KB1 receptor, an inhibitor KBRB1 gene expression and or activity,
   b. A modulator of proteases dysregulation (i.e. an inhibitor of cathepsins B and or G)
   c. A modulator of dysregulated cytokines comprising: (an inhibitor of CCL2/CCR2, CXCL10/CXCR3, CXCL5/CXCR2, fractalkine)
      and the compositions and uses as disclosed in this embodiment of the invention can be administered as concurrent therapies of thrombolytics and or anticoagulants and anti-lipid medications as determined by the patient's health care provider.
4. Metabolic disease: as used in the invention the term “metabolic disease” refer to or describe a physiological condition in a mammal that is typically characterized by dysregulation in cellular metabolic functions comprising of dysfunction in mitochondria, dysfunction in cellular metabolism and/or energy production, immune dysregulation and inflammation. Examples of metabolic disease comprise of (but are not limited to) prediabetes, diabetes, obesity, transaminitis, steatohepatitis, hepato-steatosis, non-alcoholic steatohepatitis (NASH), pancreatitis, pancreatic pseudocyst, Chronic Fatigue Syndrome, Fibromyalgia (or “Myalgic encephalomyelitis/chronic fatigue syndrome (ME/CFS)” are used interchangeably), dyslipidemia, hypercholesterolemia. NLRP3 inflammasome activation, dysregulated RAAS-KKS (downregulation ACE2, upregulation ACE, upregulation KB1), Cathepsins (i.e., upregulation of cathepsin K, S), cytokine dysregulation play (i.e., increase IFN gamma, fractalkine (i.e. in diabetes), CXCL10 (i.e. in NASH)) a role in metabolic disease.

In one embodiment of the invention a composition as disclosed in aspect 1 of the invention is used as a treatment administered to a human subject for Metabolic Disease (i.e. NASH, DMII, Obesity, and the likes), where in the composition is a modulator comprising:
i) an inhibitor of multiple activators of inflammasome (i.e. NLRP3) priming and or activation given alone (i.e. MB, S-PB, Allopurinol, Silbene, Mitotempo, NAC, Resveratrol, NMN, Quercetin, Zinc, apigenin, apocynin, ECGC, tempol, metformin) and or with
ii) a modulator of an inflammasome-induced dysregulation in a downstream pathway comprising:
   a. a modulator of RAAS-KKS dysregulation comprising:
      i. an inhibitor AngII upregulation comprising:
         1. an inhibitor of ACE and or chymase, and
         2. an inhibitor of AT1R,
      ii. an activator (ACE2/Mas/Ang (1-7 down regulation comprising:
         1. an agonist of ACE2,
         2. an agonist of Mas
      iii. an inhibitor of KB1 receptor, an inhibitor KBRB1 gene expression and or activity,
   b. a modulator of proteases dysregulation (i.e. an inhibitor of cathepsins K and or S)
   c. a modulator of dysregulated cytokines comprising: (an inhibitor of CXCL10/CXCR3, CXCL5/CXCR2, CCL2/CCR2, fractalkine/CX3CR1, CCL2/CCR2)
      and the compositions and uses as disclosed in this embodiment of the invention can be administered as concurrent therapies of other medications (i.e. oral hypoglycemic medications) as determined by the patient's health care provider.
5. Amyloidosis: as used in the invention the term “amyloidosis” refer to or describe the physiological condition in mammals that is typically characterized by buildup of amyloid in tissue inflammation which can affect organs comprising of: heart, liver, kidney, spleen, adrenal glands, thyroid gland, blood vessels, tongue, skin, peripheral nerves. NLRP3 inflammasome activation, dysregulated RAAS-KKS (downregulation ACE2, upregulation ACE, upregulation KB1), Cathepsins (i.e., upregulation of cathepsin b, d), cytokine dysregulation play (i.e., increase IFN gamma) a role in metabolic disease.

In one embodiment of the invention a composition as disclosed in aspect 1 of the invention is used as a treatment administered to a human subject for Amyloidosis comprising:
I) an inhibitor of multiple activators of inflammasome (i.e. NLRP3) priming and or activation given alone or with II) a modulator of an inflammasome-induced dysregulation in a downstream pathway comprising:
i) a modulator of RAAS-KKS dysregulation comprising:
   an inhibitor AngII upregulation comprising:
   an inhibitor comprising: of ACE and or chymase, and AT1R,
   an activator (ACE2/Mas/Ang (1-7) down regulation comprising:
   an agonist of ACE2, Mas
   an inhibitor of KB1 receptor, an inhibitor KBRB1 gene expression and or activity,
   an activator of KB2 receptor, an agonist KBRB2 gene expression and or activity,
ii) a modulator of proteases dysregulation (i.e. an inhibitor of cathepsins B, and or D)
iii) a modulator of dysregulated cytokines comprising: (an inhibitor of IFN II, CCL2/CCR2
6. Autoimmune disease: as used in the invention the term “autoimmune disease” refer to or describe the physiological condition in mammals that is typically characterized by the immune system of the mammal mistakenly attacking self-antigens which are molecules or chemical that the self-immune system attacks via antibodies mistakenly as foreign antigen. Antibodies to self-antigens are comprised of but not limited to anticardiolipin antibodies, antineutrophil cytoplasmic antibodies (peri-nuclear (P-ANCA, cytoplasmic (C-ANCA), antibodies to thyroid peroxidase, anti-nuclear antibodies (ANA), anti-SSA, anti-ssb, anti-double stranded DNA antibody, anti-smith antibodies, anti-Jo-1 antibodies, anti-rheumatoid factors (RF), anti-perinuclear factor (APF), anti-keratin antibodies (AKA), anti-filaggrin antibodies (AFA), anti-phosphatidyl choline. Non limiting examples of autoimmune disease comprising: systemic lupus erythematosus (SLE), Wegener's granulomatosis, Sjogren's syndrome, vasculitis, anti-phospholipid syndrome, rheumatoid arthritis, osteoarthritis, myositis, dermatomyositis, psoriasis, spondylo-negative arthritis, autoimmune hepatitis, good pastures, vasculitis, mixed connective tissue disease, Inflammatory arthritis (examples of Inflammatory arthritis comprise of Ankylosing spondylitis, Gout, pseudogout, Lyme Psoriatic arthritis, Lupus, sarcoid arthritis, rheumatoid arthritis, osteoarthritis), Psoriatic skin disease, Psoriatic arthritis, Osteoarthritis, Multiple Sclerosis (MS), Autoimmune encephalitis. Multi-level cell dysfunction generating DAMPS and inflammasome production produce a role (i.e., NLRC4, NLRP3) inflammasome activation, dysregulated RAAS-KKS (downregulation ACE2, upregulation ACE, upregulation KB1), Cathepsins (i.e., upregulation of cathepsin s), cytokine dysregulation play (i.e., increase CCL2, CCL5) a role in metabolic disease.

In one embodiment of the invention a composition as disclosed in aspect 1 of the invention is used as a treatment administered to a human subject who has an Autoimmune disease as described herein comprising:
I) an inhibitor of multiple activators of an inflammasome (i.e. NLRP3, NLRPC4) priming and or activation given alone or with II) a modulator of an inflammasome-induced dysregulation in a downstream pathway comprising:
i) a modulator of RAAS-KKS dysregulation comprising:
   an inhibitor AngII upregulation comprising:
   an inhibitor comprising: of ACE and or chymase, and AT1R,
   an activator (ACE2/Mas/Ang (1-7) down regulation comprising:
   an agonist of ACE2, Mas and
   an inhibitor of KB1 receptor, an inhibitor KBRB1 gene expression and or activity,
ii) a modulator of proteases dysregulation comprising: an inhibitor of cathepsins G, S, C
iii) a modulator of dysregulated cytokines comprising: an inhibitor of IFN II, CCL2/CCR2 and the compositions and uses as disclosed in this embodiment of the invention can be administered as concurrent therapies with other medications (i.e. corticosteroids) as determined by the patient's health care provider.
7. Osteoarthritis: as used in the invention the term “osteoarthritis” refers to or describe the physiological condition in mammals that is typically characterized by destruction of cartilage and joints. Multi-level cell dysfunction generating DAMPs and inflammasome activation (i.e., NLRP3 inflammasome), dysregulation in RAAS-KKS (i.e., activation of Bradykinin (BK) and BK B2 receptor), dysregulation in cathepsins (i.e., cathepsin K, D), dysregulation in Cytokines (i.e., activation of interferon regulatory factor I (IRF1) plays a role in osteoarthritis.

In one embodiment of the invention a composition as disclosed in aspect 1 of the invention is used as a treatment administered to a human subject for Osteoarthritis comprising:
I) an inhibitor of multiple activators of an inflammasome (i.e. NLRP3, NLRPC4) priming and or activation given alone or with II) a modulator of an inflammasome-induced dysregulation in a downstream pathway comprising:
i) a modulator of RAAS-KKS dysregulation comprising:
   an inhibitor AngII upregulation comprising:
   an inhibitor comprising: of ACE and or chymase, and AT1R,
   an activator (ACE2/Mas/Ang (1-7) down regulation comprising:
   an agonist of ACE2, Mas, and
   an inhibitor of KB2 receptor,
   an inhibitor of bradykinin
ii) a modulator of proteases dysregulation comprising: inhibitor of cathepsins K, D
iii) a modulator of dysregulated cytokines comprising: an inhibitor comprised: IRF1, CCL2/CCR2, CXCL10/CXCR3
and the compositions and uses as disclosed in this embodiment of the invention can be administered as concurrent therapies with other medications (i.e. NSAIDs) as determined by the patient's health care provider.
8. Familial Mediterranean fever: as used in the invention the term “Familial Mediterranean fever” refers to or describes a physiological condition in mammals that is typically characterized by recurrent attacks of fever, inflammation of the lining of the abdomen (peritonitis) and lining of the lungs (pleuritis), joint edema and rash. Inflammasome dysregulation (i.e., activation of NLRP3 inflammasome), dysregulation in the RAAS/KKS (i.e., ACE gene insertion/deletion variant), dysregulation of cathepsins (i.e., cathepsin B) dysregulation of Cytokines (i.e., activation of IFN gamma and TH1 polarization) plays a role in Mediterranean fever.

In one embodiment of the invention a composition as disclosed in aspect 1 of the invention is used as a treatment administered to a human subject for Familial Mediterranean Fever comprising:
I) an inhibitor of single or multiple activators of an inflammasome (i.e. NLRP3, AIM2) priming and or activation given alone or with II) a modulator of an inflammasome-induced dysregulation in a downstream pathway comprising:
i) a modulator of RAAS-KKS dysregulation comprising:
   an inhibitor comprising: of ACE and AT1R
   an activator (ACE2/Mas/Ang (1-7) down regulation comprising:
   an agonist of ACE2, Mas, and
   an inhibitor of KB2 receptor,
   an inhibitor of bradykinin
ii) a modulator of proteases dysregulation comprising: inhibitor of cathepsins B
iii) a modulator of dysregulated cytokines comprising: an activator of Interferon lambda, activating TH2 polarization
and the compositions and uses as disclosed in this embodiment of the invention can be administered as concurrent therapies with other medications as determined by the patient's health care provider.
9. Bechet disease: (BD) as used in the invention the term “Bechet disease” refer to or describe the physiological condition in mammals that is typically characterized by blood vessel inflammation and symptoms of oral and genital ulcers, skin rash, arthritis, and uveitis. Inflammasome dysregulation (i.e., activation of NLRP3 inflammasome), dysregulation in the RAAS/KKS (i.e., DD genotype of ACE gene I/D polymorphism), Dysregulation of cathepsins (i.e., cathepsin B, G) dysregulation of Cytokines (i.e., low IL-10, lower MHC class II trans-activator (CIITA) which is a n NLR) play a role in BD.

In one embodiment of the invention a composition as disclosed in aspect 1 of the invention is used as a treatment administered to a human subject for Bechet's Disease comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of RAAS-KKS dysregulation comprising:
   an inhibitor comprising: of ACE and AT1R
   an activator (ACE2/Mas/Ang (1-7) down regulation comprising:
   an agonist of ACE2, Mas, and
   an inhibitor of KB2 receptor,
ii) a modulator of proteases dysregulation comprising: inhibitor of cathepsins B, G
iii) a modulator of dysregulated cytokines comprising: an activator of IL-10, inhibiting CIITA gene expression and or activity
and the compositions and uses as disclosed in this embodiment of the invention can be administered as concurrent therapies with other medications as determined by the patient's health care provider.
10. Atopic dermatitis: as used in the invention the term “Atopic dermatitis” or “skin eczema”, “severely dry skin” which are used interchangeably refer to or describe the physiological condition in mammals that is typically characterized by loss of surface of skin, with erythema, scaling and pruritis. Inflammation dysregulation (i.e., activation of NLRP3 inflammasome), dysregulation in the RAAS/KKS (i.e., Ang-II elevation via chymase, Kinin are involved as co-mitogens in cellular proliferation and propagating pain and inflammation), Dysregulation of cathepsins (i.e., activation of cathepsin S) dysregulation of Cytokines (i.e., IFN alpha and gamma down regulation) play a role in Atopic dermatitis, and eczema.

In one embodiment of the invention a composition as disclosed in aspect 1 of the invention is used as a treatment administered to a human subject for Atopic dermatitis comprising:
I) an inhibitor of single or multiple activators of an inflammasome (i.e. NLRP3) priming and or activation given alone or with II) a modulator of an inflammasome-induced dysregulation in a downstream pathway comprising:
   i) a modulator of RAAS-KKS dysregulation comprising:
      an inhibitor of AngII
      an inhibition chymase
      an inhibitor of KB1R
   ii) a modulator of proteases dysregulation comprising: inhibitor of cathepsins s
   iii) a modulator of dysregulated cytokines comprising: inhibitors of 11-18, CCL2, CCL5)
and the compositions and uses as disclosed in this embodiment of the invention can be administered as concurrent therapies with other medications as determined by the patient's health care provider.
11. Inflammatory arthritis: as used in the invention the term “inflammatory arthritis” refer to or describe the physiological condition in mammals that is typically characterized by immune activation (i.e., recruitment of neutrophils) in many joints of the body which results in joint edema, calor, pain, stiffness, erythema (in some joints) and decreased ROM and joint destruction. Examples of Inflammatory arthritis comprise of Ankylosing spondylitis, Gout, pseudogout, Lyme Psoriatic arthritis, Lupus, sarcoid arthritis, rheumatoid arthritis, osteoarthritis. Inflammation dysregulation (i.e., NLRP3 overactivation by DAMPs (i.e., excess intracellular crystal levels, xanthine oxidase, Bradykinin and Kinin B1 B2 receptor (antagonism)), dysregulation of RAAS-Kinin (Ang-II overactivity), Cathepsin dysregulation (i.e., and cathepsin B and C (in gout) B & L in RA/Gout/psoriatic arthritis, Cathepsin D), dysregulation in cytokines (upregulation of CXCL8 recruits neutrophils to the joint, Il28a (Interferon lambda2 (IL-28A) acting as agonist IFNLR1 inhibits CXCL8) play a role in inflammatory arthritis.

In one embodiment of the invention a composition as disclosed in aspect 1 of the invention is used as a treatment administered to a human subject for Inflammatory arthritis comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of RAAS-KKS dysregulation comprising:
   an inhibitor of AngII
   an inhibitor of chymase
   an inhibitor of KB1R
ii) a modulator of proteases dysregulation comprising of an inhibitor of cathepsins: B, C, D,
iii) a modulator of dysregulated cytokines comprising: an inhibitor of CXCL8 via agonist of IFNLR1, an inhibitor of CCL2, inhibitors of 11-18, CCL2, CCL5)
and the compositions and uses as disclosed in this embodiment of the invention can be administered as concurrent therapies with other medications as determined by the patient's health care provider.
12. Liver Cirrhosis—as used in the invention the term “liver cirrhosis” refer to or describe the physiological condition in mammals that is typically characterized by scarring (fibrosis) of the liver. Examples of liver cirrhosis comprise of but not limited to hepatitis (i.e., chronic viral hepatitis (hep B and C), primary biliary cirrhosis, and primary sclerosing cholangitis, cirrhosis in autoimmune hepatitis, cirrhosis from alcoholic liver disease. Dysregulation of inflammasome (i.e., NLRP3 activation), dysregulation in RAAS-KKS (i.e., upregulation ACE/Ang-II, downregulation of ACE2/Ang(1-7), Mas receptor, Kallikrein kinin activity is altered in renal hemodynamics of severe liver disease), dysregulation of cathepsins (B, L, D are prognostic indicators of malignant transformation in liver cirrhosis to hepatocellular carcinoma, TMRSS2 can activate hepatitis C infection), dysregulation in cytokines (i.e., low IL-10, high IFN-lambda levels predisposed to spontaneous resolution of HCV infection, CCL2-CCR2, CX3CL1-CX3CR1) play roles in liver cirrhosis.

In one embodiment of the invention a composition as disclosed in aspect 1 of the invention is used as a treatment administered to a human subject for Liver Cirrhosis comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of RAAS-KKS dysregulation comprising:
   an inhibitor of AngII
   an inhibitor of ACE
   an inhibitor of chymase
   an inhibitor of KB1R
   activator of ACE2/Mas/Ang (1-7)
ii) a modulator of proteases dysregulation comprising of an inhibitor of cathepsins: (i.e. B, L, D), an inhibitor of TMPRSS2
iii) a modulator of dysregulated cytokines comprising: an inhibitor of CCL2-CCR2, an inhibitor of CX3CL1-CX3CR1, activator IL-10, inhibitor IFN lambda, and an inhibitor of 11-18)
and the compositions and uses as disclosed in this embodiment of the invention can be administered as concurrent therapies with other medications as determined by the patient's health care provider.
13. Neuropathic pain: as used in the invention the term “neuropathic pain” refer to or describe the physiological condition in mammals that is typically characterized by caused by disease affecting the somatosensory nervous system and can be associated with abnormal sensations (dysesthesia) or pain from non-painful stimuli (allodynia). It may be continuous or episodic. Examples comprise of but not limited to trigeminal neuralgia, post herpetic neuralgia, cancer pain, chronic neuropathic pain, pain with bone fractures, pain with bone metastasis, pain from pleuritis, pain from pericarditis. Dysregulation of inflammasome (i.e., NLRP3 activation), dysregulation in proteases (i.e., TMPRSS2 activate protease activated receptor-2 (PAR2)), dysregulation of cathepsins (i.e., cathepsin E (is involved in nociceptive pain), autophagy and Cathepsin L are in involved in antinociception, cathepsin S upregulated in CSF in patient with neuropathic pain), dysregulated RAAS-KKS (i.e., ang-II via AT1R leading to hyperexcitability of neurons, Kinin B1 involved in hyperalgesia), dysregulated cytokines (i.e., upregulation of IFN gamma, CCL2, CCL3, and fractalkine) play roles in neuropathic pain.

In one embodiment of the invention a composition as disclosed in aspect 1 of the invention is used as a treatment administered to a human subject for Neuropathic Pain comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of RAAS-KKS dysregulation comprising:
   an inhibitor of AT2R (angiotensin 2 Receptor)
   an inhibitor of KB1R
   an activator of Mas
ii) a modulator of proteases dysregulation comprising of an inhibitor of cathepsins: (i.e. E, L, S), an inhibitor of TMPRSS2
iii) an inhibitor of dysregulated cytokines comprising: IFN gamma, CCL2, CCR2, CCR4, CCL3, and CX3CL1/CX3CR1
and the compositions and uses as disclosed in this embodiment of the invention can be administered as concurrent therapies with other medications as determined by the patient's health care provider.
14. Migraine: as used in the invention the term “migraine” refers to or describe the physiological condition in mammals that is typically characterized by caused by recurrent throbbing headaches with or without sensory disturbance (aura), nausea, vomiting, sensitivity to light or sound. Examples of migraine comprise of but not limited to migraine variant, optical migraine, migraine with or without aura, vestibular migraine, retinal migraine, migraine with brainstem aura. Dysregulation in inflammasome (i.e., NLRP3 activation, mitochondrial dysfunction, Mitochondrial DNA polymorphism (i.e., 16519C→T highly associated with migraine), dysregulation of RAAS-KKS (i.e., Angiotensin II, ACE), dysregulation cathepsin (i.e., cathepsin G), dysregulation of cytokines (i.e., upregulation of CXCL8, CCL3) have roles in migraine.

In one embodiment of the invention a composition as disclosed in aspect 1 of the invention is used as a treatment administered to a human subject for Migraine comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of RAAS-KKS dysregulation comprising:
   an inhibitor of AT2R (angiotensin 2 Receptor)
   an inhibitor of ACE
ii) a modulator of proteases dysregulation comprising of an inhibitor of cathepsins (i.e. G)
iii) an inhibitor of dysregulated cytokines comprising: CXCL8, CCL3 and the compositions and uses as disclosed in this embodiment of the invention can be administered as concurrent therapies with other medications as determined by the patient's health care provider.
15. Fibromyalgia: as used in the invention the term “fibromyalgia” refers to or describe the physiological condition in mammals that is typically characterized by extensive musculoskeletal pain, localized tender points, fatigue. Dysregulation of inflammasome (i.e., mutations in cytochrome B, mitochondrial dysfunction acting as DAMPs for NLRP3 activation), dysregulated RAAS-KKS (i.e., Kinin, ACE conversion of Kinin to bradykinin and K1B and K2B), dysregulation of cathepsins (i.e., elevated cathepsin S, C), dysregulation of cytokines (i.e., IL-8/CXCL8 increase) have roles in fibromyalgia.

In one embodiment of the invention a composition as disclosed in aspect 1 of the invention is used as a treatment administered to a human subject for Fibromyalgia comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of RAAS-KKS dysregulation comprising:
   an inhibitor of ACE
   an inhibitor of K1BR and or K2BR
ii) a modulator of proteases dysregulation comprising of an inhibitor of cathepsins (i.e. S, C)
iii) an inhibitor of dysregulated cytokines comprising: CXCL8, Il-18, IL-4, Il-5, IL-13 and the compositions and uses as disclosed in this embodiment of the invention can be administered as concurrent therapies with other medications as determined by the patient's health care provider.
16. Muckle-Wells syndrome (MWS): as used in the invention the term “MWS” refer to or describe the physiological condition in mammals that is typically characterized by episodic ever, rash and joint pain. MWS is an autoinflammatory disease characterized by excessive interleukin-1 (IL-1) release, resulting in recurrent fevers, sensorineural hearing loss, and amyloidosis. Dysregulation of NLRP3 inflammasome (i.e., mutations in NLRP3 gene), dysregulation of cathepsins (i.e., cathepsin B), dysregulated cytokines (i.e., Il-1beta), play roles in MWS.

In one embodiment of the invention a composition as disclosed in aspect 1 of the invention is used as a treatment administered to a human subject for MWS comprising: an inhibitor of single or multiple activators of an inflammasome (i.e. NLRP3) priming and or activation given alone or with an inhibitor of Cathepsin B and the compositions and uses as disclosed in this embodiment of the invention can be administered as concurrent therapies with other medications as determined by the patient's health care provider.
17. Multiple Sclerosis (MS): as used in the invention the term “MS” refer to or describe the physiological condition in mammals that is typically characterized by immune attack on the protective sheath (myelin) that covers nerve fibers causing a debilitating disease in the brain and spinal cord. Autoimmune encephalitis is similar to MS. Dysregulation in inflammasomes (i.e., NLRP3 involved in astrocytes and perivascular macrophages in active demyelinating lesions of MS, increase DAMPs cell free mitochondrial DNA), dysregulated cathepsins (i.e., increase cathepsins B, A, D, DD), dysregulation in RAAS-KKS (i.e., genotype of ACE gene might contribute to a higher risk of developing MS in men. Kinin is involved via Kb 1 receptor, angiotensinogen produced in astrocytes converted to angiogenic II and via AT1 maintain blood brain barrier, ACE/ACE2 dysregulation in CSF higher ACE and lower ACE2), dysregulated cytokines (i.e., upregulation of IFN gamma (elevated IL-18 can increase IFN gamma), downregulation of IL-10) play roles in MS and Autoimmune encephalitis.

In one embodiment of the invention a composition as disclosed in aspect 1 of the invention is used as a treatment administered to a human subject for MS comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of RAAS-KKS dysregulation comprising:
   an inhibitor of ACE
   activator of ACE2
   an inhibitor of K1BR
ii) a modulator of proteases dysregulation comprising of an inhibitor of cathepsins (i.e. B, A, D, DD)
iii) an inhibitor of dysregulated cytokines comprising: activator of an IL-10, an inhibitor of IFN gamma, an inhibitor of CCL2/CCLR2, an inhibitor of IL-18
and the compositions and uses as disclosed in this embodiment of the invention can be administered as concurrent therapies with other medications as determined by the patient's health care provider.
18. Retinopathy: as used in the invention the term “retinopathy” refers to or describe the physiological condition in mammals that is typically characterized disease of the retina. Examples comprised of non-proliferative or proliferative diabetic retinopathy. Dysregulation of inflammasome (i.e., NLRP3 activation DAMPS: i.e., HMGB1, via RIP3 dependent Necrosis), dysregulation of cathepsins (i.e., cathepsin D), dysregulation of RAAS-KKS (i.e., systemic and or local RAS system is involved), dysregulation of cytokines (i.e., CCL2) are involved in retinopathy.

In one embodiment of the invention a composition as disclosed in aspect 1 of the invention is used as a treatment administered to a human subject for Retinopathy comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. D, L)
iii) an inhibitor of dysregulated cytokines comprising: an inhibitor of CCL2, CCLR2, an inhibitor of IL-18
and the compositions and uses as disclosed in this embodiment of the invention can be administered as concurrent therapies with other medications as determined by the patient's health care provider.
19. Retinal detachment as used in the invention the term “retinal detachment” refer to or describe the physiological condition in mammals that is typically characterized by operation of retina from its underneath layer. NLRP3 inflammasome activation, cathepsin A in subretinal fluid and cathepsin D has a role. Systemic and or local RAS system is involved and dysregulation of cytokine (i.e., IL-10, IL-13).

In one embodiment of the invention a composition as disclosed in aspect 1 of the invention is used as a treatment administered to a human subject for Retinal detachment comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
ii) a modulator of proteases dysregulation comprising of an inhibitor of cathepsins (i.e. A, D)
iii) an inhibitor of dysregulated cytokines comprising: an inhibitor of IL-10, an inhibitor of IL-13)
and the compositions and uses as disclosed in this embodiment of the invention can be administered as concurrent therapies with other medications as determined by the patient's health care provider.
20. Neurologic disease: dementia & neurodegenerative disease: as used in the invention the terms “dementia” and “neurologic disease: dementia & neurodegenerative disease” refer to or describe the physiological condition in mammals that is typically characterized by diminished cognitive function that is severe enough to affect daily function. Dementia impacts language, memory and decision making and diseases that affect the neurons. Dysregulation of inflammasome (i.e., NLRP3 inflammasome activation, pyroptosis, dysregulation RAAS-KKS (i.e., ACE, Ang II hat cause a long-term and often gradual decrease in the ability to think and remember that is severe enough to affect daily functioning and AT1, diminished ACE2, ACE protein is encoded for by ACE gene which can contain a functional Insertion deletion variant in which the odds of dementia were 2-fold higher among those with ACE Insertion/Insertion genotype. Upregulated B1R and B2R is constitutive and can act a neuroprotective factor. The KKS system is found surrounding blood vessels, in neurons and glial cells. Kinins can mediate NO release, increase vascular permeability, inflammation, increase brain excitotoxicity. NO can also mediate disruption of blood brain barrier which plays a role in neurodegenerative disease. Cathepsins E, D, Cathepsin dipeptidyl aminopeptidase II, (i.e., elevated cathepsin H, D in Huntington's disease. Cathepsins have a role (i.e., Cath B, L, D). Interferon beta and IFNAR can have a protective role and promote neurite growth, branching, autophagy flux, aα-synuclein degradation in neurons, prevention of dopaminergic neuron loss in a familial Parkinson's disease model. Examples of dementia comprise of: senile dementia, Alzheimer's, Parkinson's, dementia of Lewy body, amyloidosis, mild cognitive impairment, frontotemporal dementia, Huntington's chorea dementia, vascular dementia, Creutzfeldt-Jakob disease, other prion disease dementia, mixed dementia, Dementia in HIV/AIDS, dementia associated with Traumatic Brain Injury, Wernicke-Korsakoff Syndrome (Includes dementia from alcohol abuse). Normal pressure hydrocephalus. Examples of neurodegenerative disease comprise of: dementias as discussed herein, amyotrophic lateral sclerosis, Alzheimer's disease (AD), Parkinson's disease, Prion disease, Motor neuron, diseases (MND), Huntington's disease (HD), Spinocerebellar ataxia (SCA), Spinal muscular atrophy (SMA), wolfram syndrome (WFS1, WFS2), memory loss, traumatic brain injury, post-traumatic stress disease (PTSD)), Meningitis (Meningitis comprise of aseptic meningitis (i.e., viral, fungal, chemical), bacterial meningitis), Epilepsy (examples comprising epilepsy comprise of: tonic-clinic or convulsive seizures, absence seizures, atonic seizures, tonic seizures, clonic seizures, myoclonic seizures, jacksonian seizure, partial focal seizures) and Migraine.

In one embodiment of the invention a composition as disclosed in aspect 1 of the invention is used as a treatment administered to a human subject for Neurologic disease comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an AngII inhibitor
   inhibitor of ACE
   an inhibitor of AT1R
   an inhibitor of Kinin B receptors
ii) a modulator of proteases dysregulation comprising of an inhibitor of cathepsins (i.e. B, E, D, H, L)
iii) an inhibitor of dysregulated cytokines comprising: an activator of IFN beta/IFNAR, an inhibitor of CCL/CCR2, an inhibitor of IL-18, an inhibitor of CXCL10)
and the compositions and uses as disclosed in this embodiment of the invention can be administered as concurrent therapies with other medications as determined by the patient's health care provider.
21. Epilepsy: as used in the invention the terms “epilepsy refer to or describe the physiological condition in mammals that is typically characterized by. A pattern of repeated discharge of high electrical activity by neurons (up to 4 times normal) leading to electrical storm in the brain. Dysregulation in ACE and KKS have role in seizures, NLRP3 activation (i.e., via hyperosmolar stress) plays a rule, cathepsins (i.e., B, D, E, F) play a role, cytokines including Interferon gamma have a role in epilepsy. Examples of epilepsy comprise of: tonic-clinic or convulsive seizures, absence seizures, atonic seizures, tonic seizures, clonic seizures, myoclonic seizures, jacksonian seizure, partial focal seizures.

In one embodiment of the invention a composition as disclosed in aspect 1 of the invention is used as a treatment administered to a human subject for Epilepsy comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. B, D, E, F)
iii) a modulator of cytokines (i.e. IFN Gamma)
and the compositions and uses as disclosed in this embodiment of the invention can be administered as concurrent therapies with other medications as determined by the patient's health care provider.
22. Inflammatory bowel disease (IBD) as used in the invention “IBD” refer to or describe the physiological condition in mammals that is typically characterized by inflammation and ulcers in the in the digestive system. Some examples of IBD comprise of: Ulcerative colitis: as used in the invention the terms “ulcerative colitis” refer to or describe the physiological condition in mammals that is typically characterized by inflammation and ulcers in the colon and rectum referred to as pancolitis. Crohn's disease: as used in the invention the term “crohn's disease” refer to or describe the physiological condition in mammals that is typically characterized by inflammation of different portions of the digestive tract, it can involve the entire cross section area of the digestive tract creating fistulas and there are presence of granulomas. NLRP3 and AIM 2 inflammasomes are involved in IBD. of RAS (ACE is reduced, ACE2 has anti-inflammatory action). Cathepsins are involved IBD (i.e., Cath G, B, L, D), Dysregulation of KKS system plays a role in IBD (i.e., Kinin B1 receptor upregulation).

In one embodiment of the invention a composition as disclosed in aspect 1 of the invention is used as a treatment administered to a human subject for IBD comprising:
I) an inhibitor of single or multiple activators of an inflammasome (i.e. NLRP3, AIM2) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   activator of ACE2/Ang (1-7)
   activator of MasR
   an inhibitor of KB1R
ii) a modulator of proteases dysregulation comprising of an inhibitor of cathepsins (i.e. G, B, L, D)
iii) a modulator of dysregulated cytokines (i.e. IL-18)
and the compositions and uses as disclosed in this embodiment of the invention can be administered as concurrent therapies with other medications as determined by the patient's health care provider.
23. Dysbiosis: as used in the invention “Dysbiosis” refer to or describe the physiological condition in mammals that is typically characterized by imbalance in microbiome bacterial species. Examples of where dysbiosis can occur: Urinary Microbiota, GI Microbiota, Genital microbiota, Skin Microbiota, Lung microbiota. For example, GI microbiota can lead to leaky gut and inflammation. Dysregulated inflammasomes activation (i.e., NLRP3, AIM2 NLRP6), dysregulated RAAS-KKS and cathepsins (i.e., s renin-angiotensin, dysregulation of cathepsin S by changes in Bacteroidetes species) play a role in dysbiosis.

In one embodiment of the invention a composition as disclosed in aspect 1 of the invention is used as a treatment administered to a human subject for GI Dysbiosis comprising: I) an inhibitor of single or multiple activators of an inflammasome (i.e. NLRP3, AIM2 NLRP6) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. S)
iii) a modulator of Cytokine dysregulation (i.e. IL-8, CXCL10, IL-33)
and the compositions and uses as disclosed in this embodiment of the invention can be administered as concurrent therapies with other medications as determined by the patient's health care provider.
24. Achromotrichia: as used in the invention “achromotrichia” refer to or describe the physiological condition in mammals that is typically characterized by graying of the hair. T Cell polarization can lead to graying of hair. Dysregulated inflammasomes, cytokines, RAAS-KSS, Cathepsin play a role in achromotrichia (i.e., cathepsins B, Cathepsin L″. IL1B from inflammasome activation reduces melanogenesis associated transcription factor (MITF-M) gene which can lead to achromotrichia

In one embodiment of the invention a composition as disclosed in aspect 1 of the invention is used as a treatment administered to a human subject for achromotrichia comprising:
I) an inhibitor of single or multiple activators of an inflammasome (i.e. NLRP3) priming and or activation given alone or with II) a modulator of an inflammasome-induced dysregulation in a downstream pathway comprising:

i) a modulator of local RAAS-KKS dysregulation comprising:
ii) a modulator of proteases dysregulation comprising of an inhibitor of cathepsins (i.e. B, L)
iii) a modulator of dysregulated cytokines comprising: (IFN, IRF-1, activator of MITF-M gene expression and or activity)
and the compositions and uses as disclosed in this embodiment of the invention can be administered as concurrent therapies with other medications as determined by the patient's health care provider.
25. Skin ulcers: as used in the invention “skin abscess” refer to or describe the physiological condition in mammals that is typically characterized by open wound that develops as a lesion that erodes the skin or mucus membrane and can erupt on skin overlying different organs including the foot. NLRP3 inflammasome activation (i.e., NLRP3, AIM2), dysregulated RAAS-KKS (i.e., Ang(1-7)/Mas), cathepsins (i.e., Cath B, L), cytokine dysregulation (i.e., IL-17, IL-22 IL18, Il-1), IL-8 (CXCL8) play a role in skin abscess. Examples of skin ulcers comprise of (but not limited to): non healing ulcers, decubitus ulcers, pressure ulcers, aphthous ulcers, venous stasis ulcers. Stasis ulcers, neuropathic skin ulcers, ischemic arterial ulcers, buruli ulcer, infectious ulcers, pyoderma gangrenosum, vasculitis ulcers, cryoglobulinemic ulcers, diabetic ulcers, warfarin induced skin ulcers, stab wound ulcers, fungal ulcers, bacterial ulcers, viral skin ulcers (i.e., herpes zoster), protozoal skin infections (i.e., Loa, acanthamoeba), rickettsial skin ulcers, epidermal parasitic skin ulcers, renal induces skin ulcers (i.e., secondary to calciphylaxis), vesicular skin lesions, candida oral lesions, angular cheilitis, glossitis, hairy tongue, lichen planus, recurrent aphthous stomatitis, herpes labialis, herpes genitalis, erythema migrans, hairy tongue, herpetiform ulcers, recurrent aphthous stomatitis, major and minor aphthous ulcers, gingivitis, pharyngitis, rosacea, acne (examples comprising Acne vulgaris, Hidradenitis suppurativa/acne inversa (HS)),

In one embodiment of the invention a composition as disclosed in aspect 1 of the invention is used as a treatment administered to a human subject for Skin Ulcers comprising:
I) an inhibitor of single or multiple activators of an inflammasome (i.e. NLRP3, AIM2) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   activator of ACE2/Ang (1-7)
   activator of MasR
ii) a modulator of proteases dysregulation comprising of an inhibitor of cathepsins (i.e. B, L)
iii) a modulator of dysregulated cytokines comprising: IL-17, IL-22 IL18, Il-1), IL-8 (CXCL8 and the compositions and uses as disclosed in this embodiment of the invention can be administered as concurrent therapies with other medications as determined by the patient's health care provider.
26. Cutaneous abscess: as used in the invention “cutaneous abscess” refer to or describe the physiological condition in mammals that is typically characterized by a well circumscribed collection of pus under the skin and can occur anywhere on the body. They can result secondary to infections comprising of Staphylococcus aureus, MRSA, Staphylococcus epidermidis, streptococcus species, Actinomyces bovis, Pasteurella pneumotropica, Neisseria gonorrhea, gram negative bacilli, anaerobes, nontuberculous mycobacteria, blastomycosis, nocardiosis, and cryptococcosis. They can be sterile and result from causes comprising of irritants, injected drugs, injected oils, autoimmune disease. NLRP3 inflammasome activation (i.e., NLRP3, AIM2), dysregulated RAAS-KKS (i.e., Ang(1-7)/Mas), cathepsins (i.e., Cath B, L), cytokine dysregulation (i.e., IL-17, IL-22 IL18, Il-1), IL-8 (CXCL8) play a role in cutaneous abscess. Examples of a cutaneous abscess comprise of but not limited to cutaneous abscess comprise of but not limited to furuncles, carbuncles, Bartholin abscess, sterile abscess, perirectal abscess, perianal abscess, vulvar abscess, perineal abscess, perioral abscess, Mycobacterium abscess, bacterial abscess, viral abscess, and autoimmune mediated abscess,

In one embodiment of the invention a composition as disclosed in aspect 1 of the invention is used as a treatment administered to a human subject for Cutaneous Abscess comprising:
I) an inhibitor of single or multiple activators of an inflammasome (i.e. NLRP3, AIM2) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   activator of ACE2/Ang (1-7)
   activator of MasR
ii) a modulator of proteases dysregulation comprising of an inhibitor of cathepsins (i.e. B, L)
iii) a modulator of dysregulated cytokines comprising: IL-17, IL-22 IL18, Il-1), IL-8 (CXCL8 (CXCL8 and the compositions and uses as disclosed in this embodiment of the invention can be administered as concurrent therapies with other medications as determined by the patient's health care provider.
27. Cellulitis: as used in the invention “cellulitis”, “erysipelas” are used interchangeably refer to or describe the physiological condition in mammals that is typically characterized by a bacterial infection in the upper dermis which can extend to subcutaneous or lymphatic vessels and can occur anywhere on the body. Staphylococcus epidermidis, Staphylococcus aureus, MRSA, Pasteurella multocida and Capnocytophaga canimorsus, Aeromonas hydrophila, Vibrio vulnificus, Pseudomonas aeruginosa, Group B Streptococcus, Clostridium species (i.e., Clostridial myonecrosis), Erysipelothrix rhusiopathiae (i.e., Erysipelothrix), S. pneumoniae (i.e., in Orbital cellulitis), Cryptococcus neoformans, Streptococcus iniae, Helicobacter cinaedi, Mycobacterium. NLRP3 inflammasome activation (i.e., NLRP3, AIM2), dysregulated RAAS-KKS (i.e., Ang(1-7)/Mas), cathepsins (i.e., Cath B, L), cytokine dysregulation (i.e., IL-17, IL-22 IL18, Il-1), IL-8 (CXCL8) play a role in cellulitis.

In one embodiment of the invention a composition as disclosed in aspect 1 of the invention is used as a treatment administered to a human subject for cellulitis comprising:
I) an inhibitor of single or multiple activators of an inflammasome (i.e. NLRP3, AIM2) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   activator of ACE2/Ang (1-7)
   activator of MasR
ii) a modulator of proteases dysregulation comprising of an inhibitor of cathepsins (i.e. B, L)
iii) a modulator of dysregulated cytokines comprising: IL-17, IL-22 IL18, Il-1), IL-8 (CXCL8 (CXCL8 and the compositions and uses as disclosed in this embodiment of the invention can be administered as concurrent therapies with other medications as determined by the patient's health care provider.
28. Psoriatic skin disease: as used in the invention “psoriatic skin disease” or “psoriasis” are used interchangeably and refer to or describe the physiological condition in mammals that is typically characterized by an autoimmune disease that typical involves the skin and can cause red inflames scaly erythematous, white, silvery rash and or plaques that are pruritic, burning and painful on the different parts of the body including knees, elbows, back, chest, abdomen, scalp and other location. Psoriatic skin disease comprises of plaque, inverse, pustular, guttate, erythrodermic. The disorder causes skin cells to multiply up to 10 times faster than normal. Dysregulated inflammasome activation (i.e., NLRP3 inflammasome autoactivation producing IL-1beta that increases 11-17 which feedback to increase NLRP3 inflammasome activation), dysregulated RAAS-KKS (i.e., upregulation ACE, polymorphism ACE insertion/insertion which increases activity of ACE, upregulation bradykinin), Cathepsins (i.e., L, B, D, S, K (via Th17)) cytokine dysregulation (i.e., upregulation of cytokines comprising of Il-17, 11-20, 11-22, IL-18 (via NLRP3 inflammasome), IFN gamma) play a role in Psoriatic skin disease.

In one embodiment of the invention a composition as disclosed in aspect 1 of the invention is used as a treatment administered to a human subject for Psoriatic skin disease comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. L, B, D, S, K)
iii) a modulator of dysregulated cytokines comprising: Il-17, Il-20, Il-22, IL-18
and the compositions and uses as disclosed in this embodiment of the invention can be administered as concurrent therapies with other medications as determined by the patient's health care provider.
29. Psoriatic arthritis: as used in the invention “psoriatic arthritis” refer to or describe the physiological condition in mammals that is typically characterized by a chronic autoimmune disease which affects distal joints in finger or toes, the lower back, wrists, knees, ankles. There are nail changes (i.e., punctate depressions in the nail (pitting), thickening of nails (onychauxis, detachment of nails from nailbed (onycholysis) Dysregulated inflammasome activation (i.e., NLRP3), dysregulated RAAS-KKS (i.e., upregulation ACE, polymorphism ACE insertion/insertion which increases activity of ACE, upregulation bradykinin), Cathepsins (i.e., L, B, D, S, K (via Th17)) cytokine dysregulation (i.e., upregulation of cytokines comprising of Il-17, Il-20, Il-22, Il-23, IL-18 (via NLRP3 inflammasome), IFN gamma) play a role in Psoriatic skin disease.

In one embodiment of the invention a composition as disclosed in aspect 1 of the invention is used as a treatment administered to a human subject for Psoriatic Arthritis comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. L, B, D, S, K)
iii) a modulator of dysregulated cytokines comprising: 11-17, 11-20, 11-22, IL-18, CCL2, CXCL10
and the compositions and uses as disclosed in this embodiment of the invention can be administered as concurrent therapies with other medications as determined by the patient's health care provider.
30. Lupus: as used in the invention “lupus” refer to or describe the physiological condition in mammals that is typically characterized by an autoimmune disease which impacts many organs including nervous system, heart, kidneys, joints, lungs, liver and skin. Lupus skin rash which can be erythematous rash, or scaly red violaceous rash, pruritic rash, or papular raised scaly red, non-pruritic rash that occurs in subjects who have SLE, drug induced lupus, neonatal comprising of butterfly rash, lupus pernio, discoid rash, rash in mucus membranes, Inflammasome activation (i.e., NLRP3n NEK7), dysregulated RAAS-KKS (i.e., upregulation ACE, polymorphism ACE insertion/deletion, upregulation bradykinin), Cathepsins (i.e., L, B, D, S, K (via Th17)) cytokine dysregulation (i.e., upregulation of cytokines comprising of Il-17, Il-20, Il-22, Il-23, IL-18 (via NLRP3 inflammasome), IFN gamma) play a role in Psoriatic skin disease.

In one embodiment of the invention a composition as disclosed in aspect 1 of the invention is used as a treatment administered to a human subject for Lupus comprising:
I) an inhibitor (i.e. NEK7 inhibitor, MB, TUDCA, S-PB, allopurinol, NAC, Mitotempo, apigenin, resveratrol, NMN) of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. L, B, D, S, K)
iii) a modulator of dysregulated cytokines comprising: 11-17, 11-20, 11-22, 11-23, IL-18, CX3CL1, CCL2
and the compositions and uses as disclosed in this embodiment of the invention can be administered as concurrent therapies with other medications as determined by the patient's health care provider.
31. Age related Macular degeneration (AMD): as used in the invention “AMD” refer to or describe the physiological condition in mammals that is typically characterized by loss of central vision in the macula which can lead to blindness. AMD can eventually progress wet macular degeneration as a result of abnormal blood vessel growth and leaking of fluid into the retina which is termed choroidal neovascularization (CNV). Dysregulated inflammasomes (i.e., NLRP3), dysregulation in RAAS-KKS (i.e., upregulation ACE via AT1R, B2R inhalation smaller role in CNV), dysregulation in cathepsins (i.e., Cathepsin L, B, Z), dysregulation in (i.e., CCL2) play a role in AMD including wet macular degeneration.

In one embodiment of the invention a composition as disclosed in aspect 1 of the invention is used as a treatment administered to a human subject for AMD comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB2R
ii) a modulator of proteases dysregulation comprising of an inhibitor of cathepsins (i.e., L, B, Z)
iii) a modulator of cytokines comprising: (i.e. CCL2, CCLR2, CX3CL1)
and the compositions and uses as disclosed in this embodiment of the invention can be administered as concurrent therapies with other medications as determined by the patient's health care provider.
32. Infectious disease: as used in the invention “infectious disease” refer to or describe the physiological condition in mammals that is typically characterized by entry of a pathogen comprising of a virus, bacteria, fungus, parasite into the body causing damage and dysregulation. Examples of viral infectious disease comprise of but not limited to: SARS-CoV, SARS-CoV-2, MERS-CoV, hepatitis A, B, C, D, E, F, HIV, Ebola, Herpes, Influenza, Roseola, Variola, rubella, measles, chikungunya, Cytomegalovirus, Respiratory syncytial virus, Epstein bar virus, Adeno virus, Coxsackie A or B, Marburg, Rabies, Hanta, Dengue, Rota, Examples of bacterial infections comprise of but not limited to E-coli, Escherichia coli, Klebsiella pneumonia, Enterococcus faecalis, Enterococcus faecium, Vibrio cholera, Staphylococcus aureus, Staphylococcus epidermidis, Bacteroides, Neisseria gonorrhea, Neisseria meningitis, Proteus Mirablis, Hemophilus influenza, Haemophilus ducreyi, Campylobacter jejuni, Salmonella, Citrobacter ferundii, Streptococcus pyogenes, Group A streptococcus, Group B streptococcus, Chlamydia psittaci, Francisella tularensis, Coxiella burnetii, Chlamydia pneumoniae, Mycoplasma pneumoniae, Legionella, Chlamydia trachomatis, Salmonella, Shigella (i.e., S. dysenteriae, S. flexneri, S. boydii), Clostridium perfringens, Borrelia burgdorferi, Rickettsia (i.e., rickettsia Rickettsi, Rickettsia prowazekii, mycobacteria (i.e., MTB, MAI). Examples of parasites comprise of but not limited to giardia, Entamoeba histolytica. Fungal infections comprise of but not limited to cryptococcosis. Aspergillosis. Coccidioidomycosis, Histoplasmosis. Blastomycosis. Pneumocystis jirovecii, candida, dermatophytes, Malassezia globosa, Malassezia furfur, Sepsis: (examples of sepsis comprising bacteremia, sepsis, urosepsis, severe sepsis, septic shock, and systemic inflammatory response syndrome (SIRS)), Tinea Infections (examples comprising tinea pedis, Tinea Manus, Tinea corporis, Tinea faciei, Tinea unguium, Tinea cruris, Tinea capitis), onychomycosis, Tinea Versicolor, cutaneous abscess (examples comprising: cutaneous abscess comprise of but not limited to furuncles, carbuncles, Bartholin abscess, sterile abscess, perirectal abscess, perianal abscess, vulvar abscess, perineal abscess, perioral abscess, Mycobacterium abscess), Cellulitis (examples comprising: Staphylococcus epidermidis, Staphylococcus aureus, MRSA, Pasteurella multocida and Capnocytophaga canimorsus, Aeromonas hydrophila, Vibrio vulnificus, Pseudomonas aeruginosa, Group B Streptococcus, Clostridium species (i.e., Clostridial myonecrosis), Erysipelothrix rhusiopathiae, S. pneumoniae (i.e., in Orbital cellulitis), Cryptococcus neoformans, Streptococcus iniae, and Helicobacter cinaedi). Dysregulation in inflammasome activation (i.e., NLRP3, NLRC4, AIM2), dysregulated RAAS-KKS, dysregulated cathepsins (i.e., cathepsin B, L), dysregulated cytokines (i.e., IFN I, III (Lambda))

In one embodiment of the invention a composition as disclosed in aspect 1 of the invention is used as a treatment administered to a human subject for Infectious Disease comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. B, L), inhibitor of TMPRSS2
iii) a modulator of dysregulated cytokines comprising: CCL2, CCLR2, IL-1B, IL-18, IFN, CXCL10, etc.
and the compositions and uses as disclosed in this embodiment of the invention can be administered as concurrent therapies with other medications as determined by the patient's health care provider.
33. Tinea Infections: as used in the invention “Tinea infections” refer to or describe the physiological condition in mammals that is typically characterized by cutaneious rash which is pruritic red, macular or papular, circular, itchy which can occur on different parts of the body. Dysregulated inflammasomes (i.e., NLRP3, NLRP4, AIM2), dysregulation in RAAS-KKS (i.e., bradykinin), dysregulation in cathepsins (i.e., Cathepsin B, L), dysregulation of cytokines (i.e., CCL2, IL-8, CXCR1, CXCR2) play a role in Tinea Dermatophyte infections. Examples comprising: tinea pedis, Tinea Manus, tinea corporis, Tinea faciei, Tinea unguium, Tinea cruris, Tinea capitis), onychomycosis, and tinea Versicolor

In one embodiment of the invention a composition as disclosed in aspect 1 of the invention is used as a treatment administered to a human subject for Tinea infection comprising:
I) an inhibitor of single or multiple activators of an inflammasome (i.e. NLRP3, NLRP4, AIM2) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. B, L)
iii) a modulator of dysregulated cytokines comprising: CCL2, IL-8, CXCR1, CXCR2 and the compositions and uses as disclosed in this embodiment of the invention can be administered as concurrent therapies with other medications as determined by the patient's health care provider.
34. Onychomycosis: as used in the invention “onychomycosis” refer to or describe the physiological condition in mammals that is typically candida species, Trichophyton, Microsporum, and Epidermophyton genera. Dysregulated inflammasomes (i.e., NLRP3, NLRP4, AIM2), dysregulation in RAAS-KKS (i.e., bradykinin), dysregulation in cathepsins (i.e., Cathepsin B, L), dysregulation of cytokines (i.e., CCL2, IL-8, CXCR1, CXCR2) play a role in onychomycosis.

In one embodiment of the invention a composition as disclosed in aspect 1 of the invention is used as a treatment administered to a human subject for Onychomycosis comprising:
I) an inhibitor of single or multiple activators of an inflammasome (i.e. NLRP3, NLRP4, AIM2) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. B, L)
iii) a modulator of dysregulated cytokines comprising: CCL2, IL-8, CXCR1, CXCR2 and the compositions and uses as disclosed in this embodiment of the invention can be administered as concurrent therapies with other medications as determined by the patient's health care provider.
35. Tinea Versicolor: as used in the invention “Tinea versicolor” refer to or describe the physiological condition in mammals that is typically characterized by cutaneious rash which light, tan or pink colored rash on the skin caused by the fungus Malassezia globosa, or Malassezia furfur. Dysregulated inflammasomes (i.e., NLRP3, NLRP4, AIM2), dysregulation in RAAS-KKS (i.e., bradykinin), dysregulation in cathepsins (i.e., Cathepsin B, L), dysregulation of cytokines (i.e., CCL2, IL-8, CXCR1, CXCR2) play a role in Tinea Versicolor.

In one embodiment of the invention a composition as disclosed in aspect 1 of the invention is used as a treatment administered to a human subject for Tinea versicolor comprising:
I) an inhibitor of single or multiple activators of an inflammasome (i.e. NLRP3, NLRP4, AIM2) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   an activator of ACE2/Ang (1-7)
   an agonist of MasR
ii) a modulator of proteases dysregulation comprising of an inhibitor of cathepsins (i.e. B, L)
iii) a modulator of dysregulated cytokines comprising: CCL2, IL-8, CXCR1, CXCR2 and the compositions and uses as disclosed in this embodiment of the invention can be administered as concurrent therapies with other medications as determined by the patient's health care provider.
36. Rosacea: as used in the invention “Tinea versicolor” refer to or describe the physiological condition in mammals that is typically characterized by chronic inflammatory skin disease characterized by redness, red bumps and dilated capillaries Dysregulated inflammasomes (i.e., NLRP3, NLRP4, AIM2), dysregulation in RAAS-KKS (i.e., bradykinin, kinin (i.e., KLKS), dysregulation in cathepsins (i.e., Cathepsin B, L, G), dysregulation of cytokines (i.e., CCL2, CXCL8, IL-8) play a role in Rosacea.

In one embodiment of the invention a composition as disclosed in aspect 1 of the invention is used as a treatment administered to a human subject for Rosacea comprising:
I) an inhibitor of single or multiple activators of an inflammasome (i.e. NLRP3, NLRP4, AIM2) priming and or activation given alone or with II) a modulator of an inflammasome-induced dysregulation in a downstream pathway comprising:
i) a modulator of local RAAS-KKS dysregulation comprising:
   an inhibitor of KLKS
ii) a modulator of proteases dysregulation comprising of an inhibitor of cathepsins (i.e. B, L, G)
iii) a modulator of dysregulated cytokines comprising: CCL2, CXCL8, IL-8 and the compositions and uses as disclosed in this embodiment of the invention can be administered as concurrent therapies with other medications as determined by the patient's health care provider.
37. Acne: as used in the invention “acne” refer to or describe the physiological condition in mammals that is typically characterized by skin eruptions on the epidermis and hair follicles. Acne can comprise of Acne vulgaris, Hidradenitis suppurativa/acne inversa (HS). Dysregulated inflammasomes (i.e., NLRP3, NLRP4, AIM2), dysregulation in RAAS-KKS (i.e., ACE I/R polymorphism, bradykinin), dysregulation in cathepsins (i.e., Cathepsin B, L), dysregulation of cytokines (i.e., IL-1, IL-8, CXCR1, CXCR2, IL-12) play a role in Acne vulgaris.

In one embodiment of the invention a composition as disclosed in aspect 1 of the invention is used as a treatment administered to a human subject for Acne comprising:
I) an inhibitor of single or multiple activators of an inflammasome (i.e. NLRP3, NLRP4, AIM2) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. B, L)
iii) a modulator of dysregulated cytokines comprising: L-1, IL-8, CXCR1, CXCR2, IL-12 and the compositions and uses as disclosed in this embodiment of the invention can be administered as concurrent therapies with other medications as determined by the patient's health care provider.
38. Alopecia as used in the invention “alopecia” refer to or describe the physiological condition in mammals that is typically characterized by hair loss. Dysregulated inflammasomes (i.e., NLRP3, NLRP4, AIM2), dysregulation in RAAS-KKS (i.e., upregulation of ACE via local RAS), dysregulation in cathepsins (i.e., deficiency of cathepsin L), dysregulation of cytokines (i.e., CX3CL1, L-1, IL-8, CXCR1, CXCR2, CXCL2, CXCL1, CXCL10, Th1 polarization, IL-1) play a role in alopecia. Examples of alopecia comprise of but not limited to androgenic alopecia, alopecia areata, telogen effluvium, anagen effluvium, traction alopecia, trichotillomania,

In one embodiment of the invention a composition as disclosed in aspect 1 of the invention is used as a treatment administered to a human subject for Alopecia comprising:
I) an inhibitor of single or multiple activators of an inflammasome (i.e. NLRP3, NLRP4, AIM2) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. L)
iii) a modulator of dysregulated cytokines comprising: L-1, IL-8, CXCR1, CXCR2, CXCL2, CXCL1, CXCL10, CX3CL1
and the compositions and uses as disclosed in this embodiment of the invention can be administered as concurrent therapies with other medications as determined by the patient's health care provider.
39. Sepsis: as used in the invention “sepsis” refer to or describe the physiological condition in mammals that is typically characterized by an adverse maladaptive life-threatening response provided by the body towards infections in the blood stream. Examples of sepsis comprise of bacteremia, sepsis, urosepsis, severe sepsis, septic shock, systemic inflammatory response syndrome (SIRS). Dysregulated inflammasomes (i.e., NLRP3, NLRP4, AIM2), dysregulation in RAAS-KKS (i.e., upregulation of AngII, bradykinin (give antagonist)), dysregulation in cathepsins (i.e., Cath B, G, L), dysregulation of cytokines (i.e., IL-1, IL-8, IL-18, CL2) play a role in sepsis.
   In one embodiment of the invention a composition as disclosed in aspect 1 of the invention is used as a treatment administered to a human subject for Sepsis comprising:
   I) an inhibitor of single or multiple activators of an inflammasome (i.e. NLRP3) priming and or activation given alone or with II) a modulator of an inflammasome-induced dysregulation in a downstream pathway comprising:
   i) a modulator of local RAAS-KKS dysregulation comprising:
      an inhibitor of Kinins B receptors
   ii) a modulator of proteases dysregulation comprising of an inhibitor of cathepsins (i.e. B, G, L)
   iii) an inhibitor of dysregulated cytokines comprising: an inhibitor of IL-lb inhibitor of IL-18 and the compositions and uses as disclosed in this embodiment of the invention can be administered as concurrent therapies with other medications as determined by the patient's health care provider.
40. Renal disease: as used in the invention “renal disease” refer to or describe the physiological condition in mammals that is typically characterized by disease in renal system comprising of kidneys, ureters, bladder, urethra, prostate. Examples of renal disease comprise of but not limited to chronic kidney disease CKD (stages 1-5), end stage kidney disease ESRD, nephrocalcinosis, diabetic nephropathy, hypertensive nephropathy, acute renal failure, acute tubular necrosis, nephrotic syndrome, glomerular nephritis (i.e., crescentic glomerulonephritis) aldosterone-induced renal tubular cell injury, nephrotoxic renal injury, microalbuminuria, proteinuria, renal amyloidosis, amyloid nephropathy, immunoglobulin A nephropathy, polycystic kidney disease urinary, hepatorenal syndrome, interstitial cystitis, urinary tract infection, pyelonephritis. Dysregulated inflammasome (i.e., NLRP3 activation), dysregulation in RAAS-KKS (i.e., upregulation of ACE, B2R polymorphism (i.e., C181→T: T allele higher in ESRD than C allele, upregulation in urinary kallikrein with more severe renal disease, downregulation of Kinin B2 receptor with renal disease, upregulation of Kinin B1 receptors in renal parenchymal disease (i.e., polycystic kidney disease), dysregulation in cathepsins (i.e., cathepsin D, S, B, L), dysregulation of cytokines (i.e., L-1, IL-8 (i.e., MX1 inactivation induces IL-8), CCL2, CXCL12, CXCR1, CXCR2, CXCL2, CXCL1, CXCL9, CXCL12, fractalkine) play a role in renal disease. Fractalkine gene polymorphism shows increased susceptibility to ESRD in t haplotype+/A/T/I which was more significant in patients and associated with higher risk of ESRD. Further, the haplotype of CX3CR1 (T280M, V249I) gene has shown 3.6-fold higher risk of ESRD in an individual carrying T/I haplotype.

In one embodiment of the invention a composition as disclosed in aspect 1 of the invention is used as a treatment administered to a human subject for Renal disease comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an activator of KB2R
   an inhibitor of KB1R
   activator of ACE2/Ang (1-7)
   activator of MasR
ii) a modulator of proteases dysregulation comprising of an inhibitor of cathepsins (i.e. D, S, B, L)
iii) a modulator of dysregulated cytokines wherein the cytokine comprises: fractalkine, CCL2, CXCL12, CXCR1, CXCR2, CXCL2, CXCL1, CXCL9, CXCL12, IL-1)
   a modulator which is an activator of klotho gene expression and or activity and the compositions and uses as disclosed in this embodiment of the invention can be administered as concurrent therapies with other medications as determined by the patient's health care provider.
41. Interstitial Cystitis (IC): as used in the invention “interstitial cystitis” refer to or describe the physiological condition in mammals that is typically characterized by chronic symptoms comprising of bladder pain, pressure, pelvic pain, urinary frequency, urinary urgency, pain in the bladder. Examples of IC comprise of ulcerative IC, non-ulcerative IC. Dysregulated inflammasome (i.e., NLRP3 activation dysregulation in RAAS-KKS (i.e., Upregulation AngII, kallikrein, Kinin), dysregulation in cathepsins (i.e., cathepsin D), dysregulation of cytokines (i.e., IL-1, CXCR3, CCL1, Il-10) play a role in IC.

In one embodiment of the invention a composition as disclosed in aspect 1 of the invention is used as a treatment administered to a human subject for Interstitial Cystitis comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. D)
iii) a modulator of dysregulated cytokines wherein the modulator is an inhibitor of cytokines comprising: IL-1, CXCR3, CCL1, Il-10
and the compositions and uses as disclosed in this embodiment of the invention can be administered as concurrent therapies with other medications as determined by the patient's health care provider.
42. Gastrointestinal disease (GI): as used in the invention “gastrointestinal disease” refer to or describe the physiological condition in mammals that is typically characterized by disease involving the gastrointestinal tract comprising of mouth, esophagus, stomach, small intestine, large intestine, rectum, pancreases, liver, gallbladder. Examples of GI disease comprising: peptic ulcer disease (PUD), gastroesophageal reflux disease (GERD), esophagitis, Barrette's esophagitis, gastric dysmotility disease, small intestinal bacterial over growth (SIB), malabsorption (i.e., celiac sprue), pancreatitis (i.e., chronic, acute), pancreatic pseudocyst, cholecystitis, cholelithiasis, cirrhosis, ascites, NALD, Hepato-steatosis, steatohepatitis, inflammatory bowel disease (as describe herewith, colitis, Whipple's disease, hemorrhoids, diverticulitis, diverticulosis, esophageal varices Hepatitis examples comprising: hepatitis comprise of but not limited to alcoholic hepatitis, steatohepatitis, autoimmune hepatitis, infectious hepatitis comprising of bacterial, ameboid, fungal, parasitic, viral hepatitis (comprising hepatitis virus A, B, C, D, E), granulomatous hepatitis, obstructive hepatitis, cholestatic liver injury, ischemic-perfusion liver disease. Liver Cirrhosis (examples comprising liver cirrhosis secondary to chronic viral hepatitis (i.e. hep B and C), primary biliary cirrhosis, primary sclerosing cholangitis, autoimmune hepatitis related liver cirrhosis, alcoholic liver disease related liver cirrhosis, Inflammatory bowel disease (examples comprising Crohn's disease, Ulcerative colitis, microscopic colitis (i.e., lymphocytic colitis, collagenous colitis), C-diff colitis (pseudomembranous colitis) and GI Dysbiosis. Dysregulated inflammasomes (i.e., NLRP3, NLRP1, NLRC4, AIM2), dysregulation in RAAS-KKS, dysregulation in cathepsins (i.e., cathepsin B, S, D, L), dysregulation of cytokines (i.e., CXCR3, CXCL10, CXCL11, L-1, IL-8, CXCR1, CXCR2, CXCL2, CXCL1, IL-1, IL-18, IL-22) play a role in hepatitis.

In one embodiment of the invention a composition as disclosed in aspect 1 of the invention is used as a treatment administered to a human subject for a gastrointestinal disease comprising:
I) an inhibitor of single or multiple activators of an inflammasome (i.e. NLRP3, NLRP1, NLRC4, AIM2) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   an activator of ACE2/Ang (1-7)
   activator of MasR
ii) a modulator of proteases dysregulation comprising of an inhibitor of cathepsins (i.e. B, S, D, L)
iii) a modulator of dysregulated cytokines wherein the cytokine comprises: CXCR3, CXCL10, CXCL11, L-1, IL-8, CXCR1, CXCR2, CXCL2, CXCL1, IL-1, IL-18, IL-22
and the compositions and uses as disclosed in this embodiment of the invention can be administered as concurrent therapies with other medications as determined by the patient's health care provider.
43. Hepatitis: as used in the invention “hepatitis” or “liver disease” which are used interchangeably refer to or describe the physiological condition in mammals that is typically characterized by inflammation of the hepatocytes. Examples of hepatitis comprise of but not limited to alcoholic hepatitis, steatohepatitis, autoimmune hepatitis, infectious hepatitis comprising of bacterial, ameboid, fungal, parasitic, viral hepatitis (i.e., hepatitis virus A, B, C, D, E), granulomatous hepatitis, obstructive hepatitis, cholestatic liver injury, ischemic-perfusion liver disease. Dysregulated inflammasomes (i.e., NLRP3, NLRP1, NLRC4, AIM2), dysregulation in RAAS-KKS (i.e., upregulation of ACE, pre-kallikrein, kinin), dysregulation in cathepsins (i.e., cathepsin B, S, D), dysregulation of cytokines (i.e., CXCR3, CXCL10, CXCL11, L-1, IL-8, CXCR1, CXCR2, CXCL2, CXCL1, IL-1, IL-18) play a role in hepatitis.

In one embodiment of the invention a composition as disclosed in aspect 1 of the invention is used as a treatment administered to a human subject for hepatitis comprising:
I) an inhibitor of single or multiple activators of an inflammasome (i.e. NLRP3, NLRP1, NLRC4, AIM2) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of kinin/KB1R
   activator of ACE2/Ang (1-7)
   activator of MasR
ii) a modulator of proteases dysregulation comprising of an inhibitor of cathepsins (i.e. D, L)
iii) a modulator of dysregulated cytokines wherein the cytokine comprises: CXCR3, CXCL10, CXCL11, L-1, IL-8, CXCR1, CXCR2, CXCL2, CXCL1, IL-1, and IL-18
and the compositions and uses as disclosed in this embodiment of the invention can be administered as concurrent therapies with other medications as determined by the patient's health care provider.
44. Aging and age-related diseases as used in the invention “aging and age-related diseases” refer to or describe the physiological condition in mammals that is typically characterized by changes that occur through the body of a mammal as the mammal ages which leads to chronic degenerative diseases. Examples of age-related diseases comprising diabetes, obesity, insulin resistance, inflammatory disease, cardiovascular disease, atherosclerosis, neurodegenerative disease, dementias (i.e., Alzheimer's, Parkinson's disease, renal disease (i.e., chronic kidney disease), cancer, osteoarthritis, osteopenia, osteoporosis, periodontal bone loss, receding gums, sarcopenia, periodontitis, rheumatoid arthritis, maculopathy, chronic obstructive pulmonary disease, hair loss, Hutchinson-Gilford progeria, cellular senescence, Mitochondrial stress, mitochondrial disease (mitochondrial cytopathies” which are used interchangeably), and dyslipidemia. Dysregulated inflammasomes (i.e., NLRP3, NLRP4, AIM2), dysregulation in RAAS-KKS (i.e., upregulation of ACE via local RAS), dysregulation in cathepsins (i.e., upregulation of Cathepsin B, D, E, F, K and cathepsin L downregulated in most tissues), dysregulation of cytokines (i.e., upregulation of IL-1, T cell CC chemokines, CCL19, downregulation of CXCL1, dysregulation CXCL8, decreased sensitivity of immune cells to interferon (i.e., IFN alpha) play a role in age-related diseases.

In one embodiment of the invention a composition as disclosed in aspect 1 of the invention is used as a treatment administered to a human subject for Aging and age-related diseases comprising:
I) an inhibitor of single or multiple activators of an inflammasome (i.e. NLRP3, NLRP4, AIM2) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   activator of ACE2/Ang (1-7)
   activator of MasR
ii) a modulator of proteases dysregulation comprising of an inhibitor of cathepsins (i.e. B, D, E, F, K, L)
iii) a modulator of dysregulated cytokines comprising:
   an inhibitor of IL-1, CCL19,
   an activator of CXCL1,
   a modulator of CXCL8
   a modulator of IFN alpha resistance
and the compositions and uses as disclosed in this embodiment of the invention can be administered as concurrent therapies with other medications as determined by the patient's health care provider.
45. Mood Disorders: as used in the invention “mood disorders” refer to or describe the physiological condition in mammals that is typically characterized by lowering or elevation of a person's mood. Examples of mood disorder comprise of Bipolar I Disorder, Bipolar II Disorder, Cyclothymic Disorder, Major Depressive Disorder, Persistent Depressive Disorder (Dysthymia), Premenstrual Dysphoric Disorder, anxiety disorder, post-traumatic stress disorder, and Addiction disorders (i.e., alcohol addiction). Dysregulated inflammasomes (i.e., NLRP3, NLRP1, NLRC4, AIM2), dysregulation in RAAS-KKS (i.e., polymorphisms in ACE gene ((insertion/deletion), upregulation of ACE, kinin B1 receptor), dysregulation in cathepsins (i.e., cathepsin B, D), dysregulation of cytokines (i.e., Il-1B, IL-10, CCL2) play a role in mood disorders.

In one embodiment of the invention a composition as disclosed in aspect 1 of the invention is used as a treatment administered to a human subject for Mood Disorder comprising:
I) an inhibitor of single or multiple activators of an inflammasome (i.e. NLRP3, NLRP1, NLRC4, AIM23) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of KB1R
   an activator of ACE2/Ang (1-7)
   activator of MasR
ii) a modulator of proteases dysregulation comprising of an inhibitor of cathepsins (i.e. B, D)
iii) a modulator of dysregulated cytokines wherein the cytokine comprises: IL-10, IL-1B, CCL2,
and the compositions and uses as disclosed in this embodiment of the invention can be administered as concurrent therapies with other medications as determined by the patient's health care provider.
46. Cellular senescence: as used in the invention “cellular senescence” or Senescent associated secretory phenotype (SASP) are used interchangeably and “refer to or describe the physiological condition in mammals that is typically characterized by old or damaged cells undergo irreversible cell arrest and exit the cell cycle. These cells undergo other changes comprising of metabolic reprogramming, chromatin rearrangement, or autophagy modulation. Senescent cells also produce chemical (i.e., proinflammatory cytokines) that have adverse impact on cells. Senescent cells producing these chemicals are referred to as SASP. Senescent cell has stabilization of P53, Increased expression of P20, hypo phosphorylation of RB protein. Cellular senescence can involve any organs in the body. Cellular senescence comprises of replicative senescence, stress-induced premature senescence (SIPS), proteasome inhibition-induced premature senescence, (PIIPS). Dysregulated inflammasome activation (i.e., NLRP3), dysregulation in RAAS-KKS (i.e., angiotensin II), ACE2Ang ((1-7)/Mas1 receptor positively impact cell senescence via activating klotho gene. Dysregulation in cathepsins (i.e., cathepsin L, X, B, K), dysregulation of cytokines (i.e., upregulation of CXCR2, CXCL12, its receptor CXCR4) play a role in cellular senescence.

In one embodiment of the invention a composition as disclosed in aspect 1 of the invention is used as a treatment administered to a human subject for Cellular senescence comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   activation of Bradykinin type 2 receptor
   activator of ACE2/Ang (1-7)
   activator of MasR
   activate Klotho gene and or activity
   agonist of klotho
ii) a modulator of proteases dysregulation comprising of an inhibitor of cathepsins (i.e. L, X, B, K)
iii) an inhibitor of dysregulated cytokines comprising: an inhibitor of CXCR2, CXCL12, CXCR4
and the compositions and uses as disclosed in this embodiment of the invention can be administered as concurrent therapies with other medications as determined by the patient's health care provider.
47. Meningitis: as used in the invention “meningitis” refer to or describe the physiological condition in mammals that is typically characterized by inflammation of the leptomeningeal membranes that surround the brain. Meningitis comprise of aseptic meningitis (i.e., viral, fungal, chemical), bacterial meningitis, Dysregulated inflammasomes (i.e., NLRP3), dysregulation in RAAS-KKS (i.e., upregulation of ACE, bradykinin), dysregulation in cathepsins (i.e., cathepsin B, D), dysregulation of cytokines (i.e., CCL2, IL-8 CXCL5, of CXCL10, CXCL11, CXCR3) play a role in meningitis.

In one embodiment of the invention a composition as disclosed in aspect 1 of the invention is used as a treatment administered to a human subject for Meningitis comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT1R
   an inhibitor of bradykinin
   an inhibitor of KB1R
   activator of ACE2/Ang (1-7)
   activator of MasR
ii) a modulator of proteases dysregulation comprising of an inhibitor of cathepsins (i.e. B, D)
iii) a modulator of dysregulated cytokines comprising: CCL2, IL-8 CXCL5, of CXCL10, CXCL11, and CXCR3
and the compositions and uses as disclosed in this embodiment of the invention can be administered as concurrent therapies with other medications as determined by the patient's health care provider.
48. Mitochondrial disease: as used in the invention “Mitochondrial disease” or “mitochondrial cytopathies” which are used interchangeably refer to or describe the physiological condition in mammals that is typically characterized by either inherited or spontaneous mutations in mtDNA or nDNA which lead to altered functions of the proteins or RNA molecules that normally reside and function in mitochondria and can result in varied clinical phenotypes. Dysregulated inflammasomes (i.e., NLRP3), dysregulation in RAAS-KKS (i.e., AngII via upregulation of Ang 2 R activates (vascular) kinin), dysregulation in cathepsins (i.e., Cathepsin B) play a role in mitochondrial disease.

In one embodiment of the invention a composition as disclosed in aspect 1 of the invention is used as a treatment administered to a human subject for Mitochondrial disease comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE
   an inhibitor of AT2R
   an inhibitor of KB1R
   activator of ACE2/Ang (1-7)
   activator of MasR
ii) a modulator of proteases dysregulation comprising of an inhibitor of cathepsins (i.e. B)
iii) a modulator of dysregulated cytokines comprising: an inhibitor of CCL2, CCLR2, CX3CL1, IL-18
and the compositions and uses as disclosed in this embodiment of the invention can be administered as concurrent therapies with other medications as determined by the patient's health care provider.
49. Chronic Fatigue Syndrome: as used in the invention the term “fibromyalgia” or “Myalgic encephalomyelitis/chronic fatigue syndrome (ME/CFS)” are used interchangeably and refer to or describe the physiological condition in mammals that is typically characterized by extensive persistent fatigue. Dysregulation of inflammasome (i.e., mutations in cytochrome B, mitochondrial dysfunction acting as DAMPs for NLRP3 activation), dysregulated RAAS-KKS (i.e., AngII K1B and K2B), dysregulation of cathepsins (i.e., elevated cathepsin S, C, G), dysregulation of cytokines (i.e., upregulation of CCL2, IL-8/CXCL8, Il-1B) have roles in fibromyalgia.

In one embodiment of the invention a composition as disclosed in aspect 1 of the invention is used as a treatment administered to a human subject for Chronic Fatigue Syndrome: comprising:
I) an inhibitor of single or multiple activators of an inflammasome (i.e. NLRP3) priming and or activation given alone or with II) a modulator of an inflammasome-induced dysregulation in a downstream pathway comprising:
   a modulator of local RAAS-KKS dysregulation comprising: an inhibitor of ACE, an inhibitor of AT1R, an inhibitor of K1BR, an inhibitor of K2BR, an activator of ACE2/Ang (1-7) and an activator of MasR
ii) a modulator of proteases dysregulation comprising of an inhibitor of cathepsins (i.e. S.C, G)
iii) a modulator of cytokine dysregulation comprising of an inhibitor of CCL2, IL-8/CXCL8, Il-1B)
and the compositions and uses as disclosed in this embodiment of the invention can be administered as concurrent therapies with other medications as determined by the patient's health care provider.
50. Osteoporosis/Osteopenia: as used in the invention “Osteoporosis/Osteopenia” refer to or describe the physiological condition in mammals that is typically characterized by low bone mass, low bone mineralization, depletion of bone calcium and bone protein. Dysregulated inflammasomes (i.e., NLRP3), dysregulation in RAAS-KKS (i.e., upregulation of AngII, Ang II type 1 receptor, bradykinin receptor (BR)-1, bradykinin, kallidin, ACE VD polymorphism), dysregulation in cathepsins (i.e., cathepsin S, K), dysregulation of cytokines (i.e., Il-1, CCL2, CXCL10, IL-10) play a role in osteoporosis/osteopenia.

In one embodiment of the invention a composition as disclosed in aspect 1 of the invention is used as a treatment administered to a human subject for Osteoporosis, Osteopenia, periodontal bone loss comprising:
I) an inhibitor of single or multiple activators of an inflammasome (i.e. NLRP3) priming and or activation (i.e. inhibitor of P2x5R) given alone or with II) a modulator of an inflammasome-induced dysregulation in a downstream pathway comprising:
iv) a modulator of local RAAS-KKS dysregulation comprising:
   an inhibitor of ACE
   an inhibitor of AT1R
   an inhibitor of KB1R
   an inhibitor of Bradykinin, and kallidin
   an activator of ACE2/Ang(1-7)
   activator of MasR
v) a modulator of proteases dysregulation comprising of an inhibitor of cathepsins (i.e. S.K)
vi) a modulator of dysregulated cytokines wherein the cytokine comprises: Il-1, CCL2, CXCL10, IL-10
and the compositions and uses as disclosed in this embodiment of the invention can be administered as concurrent therapies with other medications as determined by the patient's health care provider.
51. Cancer: as used in the invention the term “cancer” or “cancerous” are used interchangeably and refer to or describe the physiological condition in mammals that is typically characterized by unregulated cell growth which can be localized and or metastatic. Examples of cancer comprising of (but are not limited to): prostate carcinoma lung cancer, gastrointestinal cancer (salivary cancer, oropharyngeal cancer, tongue cancer, esophageal cancer, hepatoma, gastric cancer, pancreatic cancer, gallbladder cancer, colorectal cancer), renal cell carcinoma, bladder cancer, prostate cancer, multiple endocrine neoplasia, thyroid cancer, cervical cancer, ovarian cancer, endometrial cancer, vulvar cancer, breast cancer, head and neck cancer, skin cancers (i.e., basal cell carcinoma, squamous cell carcinoma, melanoma), head and neck cancer, glioblastoma, lymphoma, Hodgkin's lymphoma, non-Hodgkin's lymphoma, leukemia, glioma, glioblastoma multiforme, retinal cancer, meningeal cancer, sarcoma, bone cancer, parathyroid cancer, adrenal cancer, squamous cell cancer, adenocarcinoma, mucinous cancer. Dysregulation in inflammasome activation (i.e., NLRP3), dysregulation in RAAS-KKS (i.e., upregulation of ACE/angiotensin II, kallikrein, kinin, downregulation of ACE2/Mas), dysregulation in cytokines (i.e., CCL2, IL-1B, CCL5, CXC chemokines ELR positive (CXCL1, CXCL2, CXCL3, CXC16, CXCL7, CXCL8) and ELR negative CXCs), dysregulation in cathepsins (i.e., (cathepsin D (i.e., breast cancer), Cathepsin L (i.e., breast cancer, endometrial cancer), cysteine cathepsin (i.e., B (i.e., squamous cell lung cancer, breast ca, colon cancer), C (i.e., squamous cell cancer, colorectal, non-small cell lung cancer), cathepsin D (i.e., breast cancer), cathepsin F (i.e., cervical cancer, gastric cancer), H, K (i.e., breast cancer, skeletal cancer, prostate, ca, bone metastasis), L2 (i.e., colorectal cancer), 0, S (i.e., pancreatic cancer, Z/X (i.e., gastric cancer, prostate cancer), TMPRSS2 (i.e., prostate cancer)) play a role in cancer.

In one embodiment of the invention a composition as disclosed in aspect 1 of the invention is used as a treatment administered to a human subject with cancer comprising:
I) an inhibitor of single or multiple activators of an inflammasome (i.e. NLRP3) priming and or activation given alone or with II) a modulator of an inflammasome-induced dysregulation in a downstream pathway comprising:
i) a modulator of local RAAS-KKS dysregulation comprising:
   an inhibitor of ACE,
   an inhibitor of AngII,
   an inhibitor of AT1R,
   an inhibitor of KB1R,
   an inhibitor of Bradykinin,
   an agonist of Mas1, and
   an activator of ACE2,
ii) a modulator of proteases dysregulation comprising of an inhibitor of cathepsins (i.e. B, C, D, H, K, L, L2, 0, S, Z, X), an inhibitor of TMPRSS2
iii) a modulator of dysregulated cytokines comprising: CCL2, IL-1B, CCL5, CXC chemokines ELR positive (CXCL1, CXCL2, CXCL3, CXC16, CXCL7, CXCL8 and the compositions and uses as disclosed in this embodiment of the invention can be administered as concurrent therapies with other medications as determined by the patient's health care provider.
52. Glossodynia: Glossodynia, oral dysesthesia, glossopyrosis, glossalgia, stomatodynia, stomatopyrosis, are used interchangeably. As used in the invention “Glossodynia” “refer to or describe the physiological condition in mammals that is typically characterized by pain, burning, irritation of the oral cavity comprising of the tongue, lips. Dysregulated inflammasome activation (i.e., NLRP3), dysregulation in proteases (i.e., TMPRSS2 activate protease activated receptor-2 (PAR2)), dysregulation of cathepsins (i.e., upregulation cathepsin E S), dysregulated RAAS-KKS (i.e., ang-II via AT1R, upregulation kallikrein-kinin, Kinin B1 involved in hyperalgesia), dysregulated cytokines (i.e., upregulation of IFN gamma, CCL2, CCL3, and fractalkine) play roles in Glossodynia.

In one embodiment of the invention a composition as disclosed in aspect 1 of the invention is used as a treatment administered to a human subject for Glossodynia comprising:
I) an inhibitor of single or multiple activators of an inflammasome (i.e. NLRP3) priming and or activation given alone or with II) a modulator of an inflammasome-induced dysregulation in a downstream pathway comprising:
iv) a modulator of local RAAS-KKS dysregulation comprising: an inhibitor of AT1R, an inhibitor of KB1R
v) a modulator of proteases dysregulation comprising of an inhibitor of cathepsins (i.e. E, S), an inhibitor of TMPRSS2
vi) a modulator of dysregulated cytokines which is an inhibitor of cytokines comprising: fractalkine, CCL2, CCL3, IFN gamma
and the compositions and uses as disclosed in this embodiment of the invention can be administered as concurrent therapies with other medications as determined by the patient's health care provider.

That which is claimed:

1. A pharmaceutical composition comprising:
a) two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3; and/or
b) one or more additional active agents comprising:
i) one or more modulators of Renin Aldosterone Angiotensin System (RAAS) dysregulation, comprising an inhibitor of the ACE/Angiotensin II arm of RAAS and/or an activator of the ACE2/Ang(1-7)/Mas1 arm of RAAS comprising an inhibitor of ACE, an inhibitor of Angiotensin II activation, an activator of ACE2, an activator of Ang(1-7), an inhibitor of AT1R, an inhibitor of ACE gene expression and/or activity, an inhibitor of Renin, an inhibitor Chymase, a mast cell stabilizer, an inhibitor of histamin, an inhibitor of neprolysin, a combined inhibitor of At1R and Neprilysin, an activator of ADAM17 activity and/or gene expression, an activator of MAS1, an activator of MAS1 gene expression and/or activity, an activator of AT2R, and/or an activator of AT2R;
ii) one or more modulators of Kallikrein-Kinin System (KKS) dysregulation;
iii) one or more modulators of protease dysregulation; and/or
iv) one or more modulators of cytokine dysregulation;
wherein the pharmaceutical composition further comprises a pharmaceutically acceptable carrier;
and wherein the active agents are each present in therapeutically effective amounts to prevent and/or treat a health condition related to inflammasome NLRP3 dysregulation, RAAS dysregulation, KKS dysregulation, protease dysregulation, and/or cytokine dysregulation in a subject in need thereof.

2. The pharmaceutical composition of claim 1, further characterized wherein:
a) the two or more active agents of 1(a) comprise a full or partial inhibitor of NLRP3, NLRC4, NLRP1, NLRP6, IFI16, Pyrin, and/or AIM2;
b) the one or more modulators of KKS dysregulation comprise an inhibitor of KKS activation comprising an inhibitor of Neprilysin, a combined inhibitor of At1R and Neprilysin, an inhibitor of ACE and Neprilysin, an inhibitor of Bradykinin B2, an inhibitor of Bradykinin B1, inhibitor of Kallikrein B1(KLKB1), an inhibitor of Kininogen 1 (KNG1), an inhibitor of High-molecular-weight kininogen (HMWK), an inhibitor of Low-molecular-weight kininogen (LMWK), an activator of Kallistatin, an inhibitor of histamine, an activator of SERPINA1, an activator of SERPINA4, and/or an activator of Cystatin A;
c) the one or more modulators of protease dysregulation comprise an inhibitor of a protease comprising Furin, Cathepsin B, Cathepsin C, Cathepsin D, Cathepsin F, Cathepsin G, Cathepsin K, Cathepsin O, Cathepsin S, Cathepsin L, Cathepsin V, Cathepsin X, Cathepsin Z, TMPRSS2, CLEC4M, CAPN2, CAPN1, CD209, and/or TMPRSS4, and/or the one or more modulators of protease dysregulation comprise an activator of CAST (calpastatin) gene activity and/or gene expression, an activator of Calpastatin, and/or an activator of Cystatin A; and
d) the one or more modulator of cytokine dysregulation comprises an activator of IFN-λ, an activator of Type I interferon, an inhibitor of CXCs, an inhibitor of CCL2, and/or an activator of IL-10.

3. The pharmaceutical composition of claim 2, further wherein the two or more active agents of 1(a) comprise an activator of cellular autophagy, an activator of mitophagy, an activator of purine salvage pathways, an activator of mitochondrial cellular bioenergetics, an activator of NRF2 gene expression and/or activity, an activator of SIRT1, an activator of ACE2/MAS/ANG(1-7), an ANG(1-7) mimetic, an activator of NLRP3 ubiquitination and degradation, an activator of dopamine receptor-1 (DRD1), and/or an activator of NAD+, further wherein the activator of NAD+ comprises an activator of nicotinamide salvage pathways, a precursor of NAD+, an agonist of NAD+, an NAD+ mimetic, an inhibitor of ROS and oxidative stress, an inhibitor of PARP1, an activator of NAD+ dependent SIRT2, an activator of mitochondrial uncoupling, an activator of mitochondrial biogenesis, and/or an activator of transcription and/or activity of genes comprising SIRT2, SIRT1, NAMPT, and/or NMNAT1.

4. The pharmaceutical composition of claim 2, further wherein the two or more active agents of 1(a) also comprise one or more full or partial inhibitors of one or more priming or activation signals of one or more activated Pattern Recognition Receptors (PRRs) and/or a modulator of PRR-activation-induced dysregulation in inflammasome signal transduction pathways, wherein the PRR is an inflammasome.

5. The pharmaceutical composition of claim 4, wherein the one or more full or partial inhibitors of one or more priming or activation signals of one or more activated PRRs comprises a full or partial inhibitor of NLRP1, NLRX1, NLRC5; and/or cGAS-STING.

6. The pharmaceutical composition of claim 4, wherein the one or more full or partial inhibitors of one or more priming or activation signals of one or more activated PRRs comprises a full or partial inhibitor of NLRP3, NLRC4, NLRP1, NLRP6, IFI16, Pyrin, and/or AIM2 and/or their related adaptors or effectors, comprising a phenothiazine dye, 4-phenylbutyrate (4-PBA), 4-phenylbutyric acid, sodium phenyl butyrate, methylene blue, glycine, a xanthine oxidoreductase inhibitor, a bile acid, taurodeoxychcolic acid (TUDCA), naltrexone, a microtubule disruptor/inhibitor, demcolcine, colchicine, an HMG-CoA reductase inhibitors/Statin, simvastatin, an antihistamine, a fibric acid derivative, metformin, propylthiouracil, thiazolidinedione, ketamine, nicotine, ivermectin, albendazole, an H/K inhibitor, amitriptyline, a corticosteroid, niclosamide, riluzole, sunitinib, valproic acid, glibenclamide bortezomib, an antibiotic, a tetracycline, minocycline, gentamycine, levofloxacine, cyclosporin, diminazen or diminazene aceturate, a calcium channel blocker, felodipine, a leukotriene D4 inhibitor, an ACE inhibitor, enalapril, an Angiotensin Receptor 1 inhibitor, resveratrol, anthocyanin, taurine, thioctic acid, rosmarinic acid, sulforaphane, celastrol, berberine, Epigallocatechin gallate, catechin, glucosamine, melatonin, pregnenolone, testosterone, estradiol, estriol, mangiferin, linarin, fisetin, rutin, rutecarpin, selenium, selenomethionin, progesterone, n-acetyl cysteine, acetyl cysteine, Mitotempo, tempol, catalase, SOD, apigenin, quercetin, tempol, silybin/silymarin, apocynin, a zinc compound, silybin, curcumin, alpha lipoic acid, genistein, gedunin, astaxanthin, copper, vitamin C, vitamin E, Vitamin D, calcitriol, niacin, tretinoin, acetaminophen, pregnenolone, a non-steroidal anti-inflammatory drug, β-Ursolic acid, Schinol, glycyrrhizic acid, trichostatinA, docasohexaenoic acid, pathenolide and/or any compound listed in Tables 1, 2, 3, or 4 in FIGS. 1, 2, 3, and 4.

7. The pharmaceutical composition of claim 1, wherein the health condition is SARS-CoV-2 infection and/or COVID-19.

8. The pharmaceutical composition of claim 7, comprising one or more modulators of SARS-CoV-2 associated RAAS dysregulation and one or more modulators of KKS dysregulation, wherein the one or more modulators of SARS-CoV-2 associated RAAS dysregulation and the one or more modulators of KKS dysregulation are each present in therapeutically effective amounts to prevent or treat SARS-CoV-2 infection and/or COVID-19 in a subject in need thereof.

9. The pharmaceutical composition of claim 7, comprising one or more inhibitors of SARS-CoV-2 associated inflammasome activators, wherein the inflammasome is NLRP3, AIM2, and/or NLRP1, and further wherein the one or more inhibitors of SARS-CoV-2 associated inflammasome activators is present in therapeutically effective amounts to prevent or treat SARS-CoV-2 infection and/or COVID-19 in a subject in need thereof.

10. The pharmaceutical composition of claim 7, comprising one or more inhibitors of SARS-CoV-2 associated cytokine dysregulation, comprising a modulator of SARS-CoV-2 associated Interferon I and/or III antiviral response.

11. A method for preventing and/or treating a health condition related to inflammasome NLRP3 dysregulation, RAAS dysregulation, KKS dysregulation, protease dysregulation, and/or cytokine dysregulation in a subject in need thereof, comprising administering a pharmaceutical composition to the subject, wherein the pharmaceutical composition comprises:
a) two or more active agents comprising an inhibitor of inflammasome NLRP3, an inhibitor of one or more priming and/or activation and/or hyperactivation signals of inflammasome NLRP3, and/or an activator of the inhibitor of one or more priming and/or activation and/or hyperactivation signals of inflammasome NLRP3; and/or
b) one or more additional active agents comprising:
i) one or more modulators of Renin Aldosterone Angiotensin System (RAAS) dysregulation, comprising an inhibitor of the ACE/Angiotensin II arm of RAAS and/or an activator of the ACE2/Ang(1-7)/Mas1 arm of RAAS comprising an inhibitor of ACE, an inhibitor of Angiotensin II activation, an activator of ACE2, an activator of Ang(1-7), an inhibitor of AT1R, an inhibitor of ACE gene expression and/or activity, an inhibitor of Renin, an inhibitor Chymase, a mast cell stabilizer, an inhibitor of histamine, an inhibitor of neprolysin, a combined inhibitor of At1R and Neprilysin, an activator of ADAM17 activity and/or gene expression, an activator of MAS1, an activator of MAS1 gene expression and/or activity, an activator of AT2R, and/or an activator of AT2R;
ii) one or more modulators of Kallikrein-Kinin System (KKS) dysregulation;
iii) one or more modulators of protease dysregulation; and/or
iv) one or more modulators of cytokine dysregulation;
wherein the pharmaceutical composition further comprises a pharmaceutically acceptable carrier;
and wherein the active agents are each present in therapeutically effective amounts to prevent and/or treat the health condition related to inflammasome NLRP3 dysregulation, RAAS dysregulation, KKS dysregulation, protease dysregulation, and/or cytokine dysregulation in the subject in need thereof.

12. The method of claim 11, wherein the pharmaceutical composition is further characterized wherein:
a) the two or more active agents of 1(a) comprise a full or partial inhibitor of NLRP3, NLRC4, NLRP1, NLRP6, IFI16, Pyrin, and/or AIM2;
b) the one or more modulators of KKS dysregulation comprise an inhibitor of KKS activation comprising an inhibitor of Neprilysin, a combined inhibitor of At1R and Neprilysin, an inhibitor of ACE and Neprilysin, an inhibitor of Bradykinin B2, an inhibitor of Bradykinin B1, inhibitor of Kallikrein B1(KLKB1), an inhibitor of Kininogen 1 (KNG1), an inhibitor of High-molecular-weight kininogen (HMWK), an inhibitor of Low-molecular-weight kininogen (LMWK), an activator of Kallistatin, an inhibitor of histamine, an activator of SERPINA1, an activator of SERPINA4, and/or an activator of Cystatin A;
c) the one or more modulators of protease dysregulation comprise an inhibitor of a protease comprising Furin, Cathepsin B, Cathepsin C, Cathepsin D, Cathepsin F, Cathepsin G, Cathepsin K, Cathepsin O, Cathepsin S, Cathepsin L, Cathepsin V, Cathepsin X, Cathepsin Z, TMPRSS2, CLEC4M, CAPN2, CAPN1, CD209 and/or TMPRSS4; and
d) the one or more modulator of cytokine dysregulation comprises an activator of IFN-λ, an activator of Type I interferon, an inhibitor of CXCs, an inhibitor of CCL2, and/or an activator of IL-10.

13. The method of claim 12, wherein the activator of IFN-λ and/or activator of Type I interferon are administered to the subject less than 48 hours post-infection.

14. The method of claim 12, further wherein the two or more active agents of 1(a) comprise an activator of cellular autophagy, an activator of mitophagy, an activator of purine salvage pathways, an activator of mitochondrial cellular bioenergetics, an activator of NRF2 gene expression and/or activity, an activator of SIRT1, an activator of ACE2/MAS/ANG(1-7), an ANG(1-7) mimetic, an activator of NLRP3 ubiquitination and degradation, an activator of dopamine receptor-1 (DRD1), and/or an activator of NAD+, further wherein the activator of NAD+ comprises an activator of nicotinamide salvage pathways, a precursor of NAD+, an agonist of NAD+, an NAD+ mimetic, an inhibitor of ROS and oxidative stress, an inhibitor of PARP1, an activator of NAD+ dependent SIRT2, an activator of mitochondrial uncoupling, an activator of mitochondrial biogenesis, and/or an activator of transcription and/or activity of genes comprising SIRT2, SIRT1, NAMPT, and/or NMNAT1.

15. The method of claim 12, further wherein the two or more active agents of 1(a) also comprise one or more full or partial inhibitors of one or more priming or activation signals of one or more activated Pattern Recognition Receptors (PRRs) and/or a modulator of PRR-activation-induced dysregulation in inflammasome signal transduction pathways, wherein the PRR is an inflammasome.

16. The method of claim 15, wherein the one or more full or partial inhibitors of one or more priming or activation signals of one or more activated PRRs comprises a full or partial inhibitor of NLRP1, NLRX1, NLRC5; and/or cGAS-STING.

17. The method of claim 15, wherein the one or more full or partial inhibitors of one or more priming or activation signals of one or more activated PRRs comprises a full or partial inhibitor of NLRP3, NLRC4, NLRP1, NLRP6, IFI16, Pyrin, and/or AIM2 and/or their related adaptors or effectors, comprising a phenothiazine dye, 4-phenylbutyrate (4-PBA), 4-phenylbutyric acid, sodium phenyl butyrate, methylene blue, glycine, a xanthine oxidoreductase inhibitor, a bile acid, taurodeoxychcolic acid (TUDCA), naltrexone, a microtubule disruptor/inhibitor, demcolcine, colchicine, an HMG-CoA reductase inhibitors/Statin, simvastatin, an antihistamine, a fibric acid derivative, metformin, propylthiouracil, thiazolidinedione, ketamine, nicotine, ivermectin, albendazole, an H/K inhibitor, amitriptyline, a corticosteroid, niclosamide, riluzole, sunitinib, valproic acid, glibenclamide bortezomib, an antibiotic, a tetracycline, minocycline, gentamycine, levofloxacine, cyclosporin, diminazen or diminazene aceturate, a calcium channel blocker, felodipine, a leukotriene D4 inhibitor, an ACE inhibitor, enalapril, an Angiotensin Receptor 1 inhibitor, resveratrol, anthocyanin, taurine, thioctic acid, rosmarinic acid, sulforaphane, celastrol, berberine, Epigallocatechin gallate, catechin, glucosamine, melatonin, pregnenolone, testosterone, estradiol, estriol, mangiferin, linarin, fisetin, rutin, rutecarpin, selenium, selenomethionin, progesterone, n-acetyl cysteine, acetyl cysteine, Mitotempo, tempol, catalase, SOD, apigenin, quercetin, tempol, silybin/silymarin, apocynin, a zinc compound, silybin, curcumin, alpha lipoic acid, genistein, gedunin, astaxanthin, copper, vitamin C, vitamin E, Vitamin D, calcitriol, niacin, tretinoin, acetaminophen, pregnenolone, a non-steroidal anti-inflammatory drug, β-Ursolic acid, Schinol, glycyrrhizic acid, trichostatinA, docasohexaenoic acid, pathenolide and/or any compound listed in Tables 1, 2, 3, or 4 in FIGS. 1, 2, 3, and 4.

18. The method of claim 11, wherein the health condition is SARS-CoV-2 infection and/or COVID-19.

19. A method for detecting the presence of at least one SNP in a subject to assess a risk of having or developing a health condition related to inflammasome NLRP3 dysregulation, RAAS dysregulation, KKS dysregulation, protease dysregulation, and/or cytokine dysregulation, comprising the steps of:
i) collecting a sample from the subject;
ii) detecting the presence of at least one SNP within a gene for a modulator of inflammasome NLRP3 dysregulation, RAAS dysregulation, KKS dysregulation, protease dysregulation, and/or cytokine dysregulation, wherein the gene is comprising ACE, ACE2, ADAM17, ADAM9, ADAMTS13, ADORA2A, AGT, AGTR, AGTR1, AGTR2, AIM2, ANAPC1, ANO6, BDKRB1, BDKRB2, BID, BIRC2, BIRC3, BIRC5, BIRC6, BRCC3, BTK, BTK, CAMK2A, CAMP, CAPN1, CAPN2, CASP1, CASP10, CASP11, CASP12, CASP14, CASP2, CASP3, CASP4, CASP5, CASP6, CASP7, CASP8, CASP9, CAST, CAT, CBL, CCL2, CD209, CFL2, CFLAR, CFLAR-AS1, CHMP4A, CHMP4B, CHMP4C, CHUK, CLEC4M, CSTA, CTSB, CTSC, CTSD, CTSF, CTSG, CTSH, CTSK, CTSL, CTSO, CTSS, CTSV, CTSX, CTSZ, CXCL10, CXCL2, CXCL3, CPLD, DDX15, DDX19, DDX5, DNMT1, DPP4, FADD, FURIN, GSDMA, GSDMB, GSDMC, GSDMD, GSDME, GSPT1, GSPT2, H1-1, HIF1A, HIF1AN, HIF3A, HMGB1, HSP90, HSP90AA1, HSP90AB4P, HSP90B1, HSP90B2P, IFIT1, IFNA1, IFNAR1, IFNAR2, IFNLR1, IKBKB, IKBKE, IKBKG, IKK, IL10, IL10RA, IL10RB, IL15, IL17, IL17A, IL18, IL18BP, IL1B, IL1R1, IL1RL1, IL21, IL9, IRF3, IRF7, KEAP, KL, KLKB1, KNG1, LRRK2, LTB4R, MAP3K7, MAPK1, MAPK3, MAPKAPK2, MAS1, MAVS, MIR20A, MIR20B, MIR21, MIR223, MIR30C, MIR30C1, MIR30C5, MIR495, MIR9, MLKL, MME, NAMPT, NEK6, NEK7, NEK9, NFATC3, NFE2L2, NFKB, NLRC3, NLRC4, NLRC5, NLRP1, NLRP10, NLRP11, NLRP12, NLRP13, NLRP14, NLRP2, NLRP3, NLRP4, NLRP5, NLRP6, NLRP7, NLRP8, NLRP9, NLRX1, NMNAT1, NOX, NOX2, NOX4, NR1H4, P2RX1, P2RX2, P2RX2, P2RX3, P2RX3, P2RX4, P2RX5, P2RX5-TAX1BP3, P2RX6, P2RX6P, P2RX7, P2RY1, P2RY10, P2RY11, P2RY12, P2RY13, P2RY14, P2RY2, P2RY4, P2RY6, P2RY8, PADI4, PARP, PARP1, PELI1, PELI2, PELI3, PEP, PRCP, PYCARD, RIPK1, RIPK2, RIPK3, RNF31, SENP6, SENP7, SERPINA1, SERPINA4, SESN2, SIRT1, SIRT2, SLC44A2, SOCS1, SOD, SOD1, SUGT1, TAB1, TAB3, TBK1, TBKBP1, TF, TFP1, TFPI, THOP1, TICAM1, TMPRSS4, TMRPSS2, TNF, TNFAIP1, TNFAIP2, TNFAIP3, TRADD, TRAF2, TRIB3, TRIM31, TRPA4, TRPM2, TRPV1, TRPV4, USP18, VDAC1, VDR, XDH, and/or ZBP1,
and combinations thereof in the sample;
wherein the presence of the SNP indicates a risk of having or developing a health condition related to inflammasome NLRP3 dysregulation, RAAS dysregulation, KKS dysregulation, protease dysregulation, and/or cytokine dysregulation in the subject.

20. The method of claim 19, wherein the health condition is SARS-CoV-2 infection and/or COVID-19.
